SEQUENCE LISTING (20 sequences)

SEQ ID NO: 1 - protein, 485 aa, Artificial Sequence
  synthetic construct
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTEFGNYDYLMYADYDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 2 - protein, 483 aa, artificial sequence
  synthetic construct
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTEFGNYDYLMYADYDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 3 - DNA, 1449 bp, Artificial sequence
  synthetic sequence
catcataacggtacgaacgggacaatgatgcaatactttgaatggtatctacctaatgacggaaatcattggaatcgattaaactctgatgcgagtaaccttaaaagcaaagggattacagcggtgtggattcctccagcatggaagggcgcttctcaaaatgacgtaggatacggagcctatgacctgtatgatctgggagaatttaatcaaaaaggtaccgtccgtacaaaatatggaacacgtagtcagttacaagctgcggtaacctccttaaaaaataatggaattcaagtatatggtgacgttgttatgaatcacaaaggtggcgcagacgctactgaaatggtaagggccgttgaagtgaatcccaataaccgtaaccaagaagtgactggtgaatataccattgaagcttggactagatttgattttccagggcgaggaaatactcattctagctttaaatggagatggtatcattttgatggtgtggattgggatcagtcacgtagactgaacaatcgcatctataaatttagaggcaaagcttgggattgggaagttgatacggaatttggtaattatgattatttaatgtacgctgattatgatatggatcacccagaagtagtaaatgaattaagaaattggggtgtttggtacacaaacacattaggactcgatggatttagaatagatgcggttaaacatataaagtatagctttacgcgcgattggattaatcacgttagaagtgcaacaggtaaaaatatgtttgcggttgctgagttttggaagaatgatttaggtgcaattgaaaactatctgcagaaaacaaactggaaccattcagtctttgatgtgccgttacattataatctttataatgcatcaaaaagcggagggaactatgatatgcgaaacatatttaatggaacggttgttcaacgacatccaagtcatgctgtaacatttgttgataatcatgattcgcagcctgaagaagcattagaatcttttgttgaagaatggtttaaaccattagcgtatgcgcttacattaacgcgtgaacaaggatacccttctgtattttacggagattattatgggattccaacacatggagtgccagcaatgagatcaaaaatcgatccgattttagaagcacgtcaaaagtatgcatacggaccccagcacgattatcttgaccacccggatgtgatcggatggacgagggaaggtgacagctcccatcctaaatcaggtttggccactttaatcacggacggacccggcggatcaaagcggatgtatgccggcctgaaaaatgccggcgagacatggtatgacataacgggcaaccgttcagatactgtaaaaatcggatctgacggctggggagagtttcatgtaaacgatgggtccgtctccatttatgttcagaaa

SEQ ID NO: 4 - protein, 485 aa, Bacillus sp. 707
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 5 - DNA, 1455 bp, artificial sequence
  synthetic construct
catcataacggtacgaacgggacaatgatgcaatactttgaatggtatctacctaatgacggaaatcattggaatcgattaaactctgatgcgagtaaccttaaaagcaaagggattacagcggtgtggattcctccagcatggaagggcgcttctcaaaatgacgtaggatacggagcctatgacctgtatgatctgggagaatttaatcaaaaaggtaccgtccgtacaaaatatggaacacgtagtcagttacaagctgcggtaacctccttaaaaaataatggaattcaagtatatggtgacgttgttatgaatcacaaaggtggcgcagacgctactgaaatggtaagggccgttgaagtgaatcccaataaccgtaaccaagaagtgactggtgaatataccattgaagcttggactagatttgattttccagggcgaggaaatactcattctagctttaaatggagatggtatcattttgatggtgtggattgggatcagtcacgtagactgaacaatcgcatctataaatttagaggccatggcaaagcttgggattgggaagttgatacggaatttggtaattatgattatttaatgtacgctgattatgatatggatcacccagaagtagtaaatgaattaagaaattggggtgtttggtacacaaacacattaggactcgatggatttagaatagatgcggttaaacatataaagtatagctttacgcgcgattggattaatcacgttagaagtgcaacaggtaaaaatatgtttgcggttgctgagttttggaagaatgatttaggtgcaattgaaaactatctgcagaaaacaaactggaaccattcagtctttgatgtgccgttacattataatctttataatgcatcaaaaagcggagggaactatgatatgcgaaacatatttaatggaacggttgttcaacgacatccaagtcatgctgtaacatttgttgataatcatgattcgcagcctgaagaagcattagaatcttttgttgaagaatggtttaaaccattagcgtatgcgcttacattaacgcgtgaacaaggatacccttctgtattttacggagattattatgggattccaacacatggagtgccagcaatgagatcaaaaatcgatccgattttagaagcacgtcaaaagtatgcatacggaccccagcacgattatcttgaccacccggatgtgatcggatggacgagggaaggtgacagctcccatcctaaatcaggtttggccactttaatcacggacggacccggcggatcaaagcggatgtatgccggcctgaaaaatgccggcgagacatggtatgacataacgggcaaccgttcagatactgtaaaaatcggatctgacggctggggagagtttcatgtaaacgatgggtccgtctccatttatgttcagaaa

SEQ ID NO: 6 - DNA, 1449 bp, artificial sequence
  synthetic construct
catcataacggtacgaacgggacaatgatgcaatactttgaatggtatctacctaatgacggaaatcattggaatcgattaaactctgatgcgagtaaccttaaaagcaaagggattacagcggtgtggattcctccagcatggaagggcgcttctcaaaatgacgtaggatacggagcctatgacctgtatgatctgggagaatttaatcaaaaaggtaccgtccgtacaaaatatggaacacgtagtcagttacaagctgcggtaacctccttaaaaaataatggaattcaagtatatggtgacgttgttatgaatcacaaaggtggcgcagacgctactgaaatggtaagggccgttgaagtgaatcccaataaccgtaaccaagaagtgactggtgaatataccattgaagcttggactagatttgattttccagggcgaggaaatactcattctagctttaaatggagatggtatcattttgatggtgtggattgggatcagtcacgtagactgaacaatcgcatctataaatttagaggcaaagcttgggattgggaagttgatacggaaaatggtaattatgattatttaatgtacgctgatattgatatggatcacccagaagtagtaaatgaattaagaaattggggtgtttggtacacaaacacattaggactcgatggatttagaatagatgcggttaaacatataaagtatagctttacgcgcgattggattaatcacgttagaagtgcaacaggtaaaaatatgtttgcggttgctgagttttggaagaatgatttaggtgcaattgaaaactatctgcagaaaacaaactggaaccattcagtctttgatgtgccgttacattataatctttataatgcatcaaaaagcggagggaactatgatatgcgaaacatatttaatggaacggttgttcaacgacatccaagtcatgctgtaacatttgttgataatcatgattcgcagcctgaagaagcattagaatcttttgttgaagaatggtttaaaccattagcgtatgcgcttacattaacgcgtgaacaaggatacccttctgtattttacggagattattatgggattccaacacatggagtgccagcaatgagatcaaaaatcgatccgattttagaagcacgtcaaaagtatgcatacggaccccagcacgattatcttgaccacccggatgtgatcggatggacgagggaaggtgacagctcccatcctaaatcaggtttggccactttaatcacggacggacccggcggatcaaagcggatgtatgccggcctgaaaaatgccggcgagacatggtatgacataacgggcaaccgttcagatactgtaaaaatcggatctgacggctggggagagtttcatgtaaacgatgggtccgtctccatttatgttcagaaa

SEQ ID NO: 7 - protein, 485 aa, Bacillus sp
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 8 - protein, 485 aa, Bacillus sp
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 9 - protein, 485 aa, Bacillus sp
HHNGTNGTMMQYFEWYLPNDGNHWNRLRDDAANLKSKGITAVWIPPAWKGTSQNDVGYGAYDLYDLGEFNQKGTVRTKYGTRNQLQAAVTSLKNNGIQVYGDVVMNHKGGADGTEIVNAVEVNRSNRNQETSGEYAIEAWTKFDFPGRGNNHSSFKWRWYHFDGTDWDQSRQLQNKIYKFRGTGKAWDWEVDTENGNYDYLMYADVDMDHPEVIHELRNWGVWYTNTLNLDGFRIDAVKHIKYSFTRDWLTHVRNTTGKPMFAVAEFWKNDLGAIENYLNKTSWNHSVFDVPLHYNLYNASNSGGYYDMRNILNGSVVQKHPTHAVTFVDNHDSQPGEALESFVQQWFKPLAYALVLTREQGYPSVFYGDYYGIPTHGVPAMKSKIDPLLQARQTFAYGTQHDYFDHHDIIGWTREGNSSHPNSGLATIMSDGPGGNKWMYVGKNKAGQVWRDITGNRTGTVTINADGWGNFSVNGGSVSVWVKQ

SEQ ID NO: 10 - protein, 485 aa, Bacillus sp.
HHNGTNGTMMQYFEWHLPNDGNHWNRLRDDAANLKSKGITAVWIPPAWKGTSQNDVGYGAYDLYDLGEFNQKGTVRTKYGTRSQLQGAVTSLKNNGIQVYGDVVMNHKGGADGTEMVNAVEVNRSNRNQEISGEYTIEAWTKFDFPGRGNTHSNFKWRWYHFDGTDWDQSRQLQNKIYKFRGTGKAWDWEVDIENGNYDYLMYADIDMDHPEVINELRNWGVWYTNTLNLDGFRIDAVKHIKYSYTRDWLTHVRNTTGKPMFAVAEFWKNDLAAIENYLNKTSWNHSVFDVPLHYNLYNASNSGGYFDMRNILNGSVVQKHPIHAVTFVDNHDSQPGEALESFVQSWFKPLAYALILTREQGYPSVFYGDYYGIPTHGVPSMKSKIDPLLQARQTYAYGTQHDYFDHHDIIGWTREGDSSHPNSGLATIMSDGPGGNKWMYVGKHKAGQVWRDITGNRSGTVTINADGWGNFTVNGGAVSVWVKQ

SEQ ID NO: 11 - protein, 485 aa, bacillus sp
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 12 - protein, 483 aa, artificial sequence
  synthetic construct
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 13 - protein, 483 aa, artificial sequence
  synthetic construct
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSEFGNYDYLMYADYDMDHPEVVNELRRWGEWYTNTLNLDGFRIDAVKHIKFSFTRDWLTHVRNATGKGMPAVAEFWKNDLGALENYLNKTNWNHSVFDVPLHYNLYNASNGGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 14 - protein, 483 aa, artificial sequence
  synthetic construct
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSEFGNYDYLMYADYDMDHPEVVNELRRWGEWYTNTLNLDGFRIDAVKHIKFSFTRDWLTHVRNATGKGMPAVAEFWKNDLGALENYLNKTNWNHSVFDVPLHYNLYNASNGGGNYDMAKLLNGTVVQKHPMHAVTFVDNHDSQPNESLESFVQEWFKPLAYALILTREQGYPSVFYGDYYGIPTHSVPAMKAKIDPILEARQNFAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 15 - protein, 483 aa, artificial sequence
  synthetic construct
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 16 - protein, 483 aa, artificial sequenece
  synthetic construct
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 17 - DNA, 27 bp, Artificial sequence
  synthetic construct
caatccaagagaaccctgatacggatg

SEQ ID NO: 18 - DNA, 23 bp, Artificial sequence
  synthetic construct
cggaacgcctggctgacaacacg

SEQ ID NO: 19 - DNA, 23 bp, Artificial sequence
  synthetic construct
tggggtgtttggtacacaaacac

SEQ ID NO: 20 - DNA, 22 bp, Artificial sequence
  synthetic construct
ccaatcgcgcgtaaagctatac

(19) United States
(12) Patent Application Publication
(10) Pub. No.: US 2017/0121695 A1
(43) Pub. Date: May 4, 2017

(54) ALPHA-AMYLASE VARIANTS AND POLYNUCLEOTIDES ENCODING SAME

(71) Applicant: NOVOZYMES A/S, Bagsvaerd (DK)

(72) Inventors: Carsten Andersen, Vaerloese (DK); Chakshusmathi Ghadiyaram, Bangalore (IN); Rajendra Kulothungan Sainathan, Bangalore (IN); Padmavathi Balumuri, Chennai (IN); Padma Venkatachalam Iyer, Mumbai (IN); Iben Damager, Valby (DK); Astrid Munch, Frederiksberg (DK); Subith Krishna, Bangalore (IN)

(73) Assignee: NOVOZYMES A/S, Bagsvaerd (DK)

(21) Appl. No.: 15/317,823

(22) PCT Filed: Jun. 12, 2015
(86) PCT No.: PCT/EP2015/063133

§ 371 Date: Dec. 9, 2016

(30) Foreign Application Priority Data
Jun. 12, 2014 (IN) 2877/CHE/2014
Aug. 14, 2014 (EP) 14180943.4

(51) Int. Cl. C12N 9/28; C11D 11/00; C11D 1/22; C11D 3/386

(52) U.S. Cl. CPC C12N 9/2417; C11D 3/38618; C11D 11/0017; C11D 1/22; C12Y 302/01001

(57) ABSTRACT

The present invention relates to variants having alpha-amylase activity and polynucleotides encoding the variants. The invention also relates to nucleic acid constructs, vectors, and host cells comprising the polynucleotides, compositions comprising the variants, as well as methods of producing and using the variants.

### REFERENCE TO A SEQUENCE LISTING

This application contains a Sequence Listing in computer readable form, which is incorporated herein by reference.

### BACKGROUND OF THE INVENTION

Field of the Invention

The present invention relates to alpha-amylase variants (polypeptides having alpha-amylase activity), nucleic acids encoding the alpha-amylases, methods of producing the alpha-amylases, compositions comprising the alpha-amylases and methods of using the alpha-amylases.

Description of the Related Art

Alpha-amylases (alpha-1,4-glucan-4-glucanohydrolases, E.C. 3.2.1.1) constitute a group of enzymes, which catalyses hydrolysis of starch and other linear and branched 1,4-glucosidic oligo- and polysaccharides.

There is a long history of industrial use of alpha-amylases in several known applications such as detergent, baking, brewing, starch liquefaction and saccharification e.g. in preparation of high fructose syrups or as part of ethanol production from starch. These and other applications of alpha-amylases are known and utilize alpha-amylases derived from microorganisms, in particular bacterial alpha-amylases.

Among the first bacterial alpha-amylases to be used were an alpha-amylase from B. licheniformis, also known as Termamyl which have been extensively characterized and the crystal structure has been determined for this enzyme. Bacillus amylases, such as Termamyl, AA560 (WO 2000/060060) and SP707 (described by Tsukamoto et al., 1988, Biochem. Biophys. Res. Comm. 151: 25-31) form a particular group of alpha-amylases that have found use in detergents. These amylases have been modified to improve the stability in detergents. WO 96/23873 e.g. disclose to delete the amino acids 181+182 or the amino acids 183+184 of SP707 (SEQ ID NO: 7 of WO 96/23873) to improve the stability of this amylase. WO 96/23873 further discloses to modify the SP707 amylase by substituting M202 with e.g. a leucine to stabilize the molecule towards oxidation. Thus, it is known to modify amylases to improve certain properties. Most recently, for environmental reasons, it has become increasingly important to lower the temperature in washing, dishwashing and/or cleaning processes. Despite the efficiency of current detergent enzyme compositions, there are many stains that are difficult to completely remove not least due to the increased use of low (e.g., cold water) wash temperatures and shorter washing cycles. Thus, there is a need for amylolytic enzymes that can function under low temperature and at the same time preserve or increase desirable properties of the alpha-amylase, such as specific activity (amylolytic activity), stability, stain removal effect and/or wash performance. The inventors of the present invention has previously found that the amylase variant of SEQ ID NO: 2 has very good stability in detergents (disclosed in European patent application no. 13194116.3 as SEQ ID NO:1 having a deletion of (H183+G184) and substitutions of N195F+I206Y). It is an object of the present invention to improve the wash performance of this variant.

Thus, it is an object of the present invention to provide polypeptides having alpha-amylase activity (alpha-amylases) which have high performance, in particular high wash performance at low temperatures in laundry washing and/or dishwashing. It is a further object of the present invention to provide alpha-amylases which have high stability in detergent compositions, in particular in liquid laundry and/or dishwash detergent compositions. It is a further object to provide alpha-amylases which have high stability in powder detergent compositions and/or which have high amylase activity after storage in detergents. Particularly, it is an object of the present invention to provide alpha-amylases which both have high stability in detergent compositions and have high wash performance at low temperature such as at 15° C. which improved wash performance is determined according to the section “Wash performance of alpha-amylases using Automatic Mechanical Stress Assay” using either model detergent A or J. In particular, it is an object of the present invention to provide alpha-amylases with improved wash performance at 15° C. compared to the parent alpha-amylase of SEQ ID NO: 2, or alternatively, compared to other closely related alpha-amylases, such as e.g. the SP707 alpha-amylase (SEQ ID NO:4).

### SUMMARY OF THE INVENTION

The present invention relates to an alpha-amylase variant comprising a) a deletion and/or a substitution at two or three or four positions corresponding to positions R181, G182, H183 and G184 of the mature polypeptide of SEQ ID NO: 1, and b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 or 174 of SEQ ID NO: 1 and the alpha-amylase variant has at least 95% sequence identity but less than 100% sequence identity to the polypeptide having the amino acid sequence of SEQ ID NO: 1 and wherein the variant has alpha-amylase activity.

The present invention further relates to isolated polynucleotides encoding the variants; nucleic acid constructs, vectors, and host cells comprising the polynucleotides; compositions comprising the variants; and methods of producing the variants.

The present invention further relates to compositions such as detergent compositions comprising said variants and to uses of the variants.

### DEFINITIONS

A-, B- and C-domains: The structure of alpha-amylases comprises three distinct domains A, B and C, see, e.g., Machius et al., 1995, J. Mol. Biol. 246: 545-559. The term “domain” means a region of a polypeptide that in itself forms a distinct and independent substructure of the whole molecule. Alpha-amylases consist of a beta/alpha-8 barrel harboring the active site residues, which is denoted the A-domain, a rather long loop between the beta-sheet 3 and alpha-helix 3, which is denoted the B-domain (together; “A and B domain”), and a C-domain and in some cases also a carbohydrate binding domain (e.g., WO 2005/001064; Machius et al., supra).

The domains of an alpha-amylase can be determined by structure analysis such as using crystallographically techniques. An alternative method for determining the domains of an alpha-amylase is by sequence alignment of the amino acid sequence of the alpha-amylase with another alpha-amylase for which the domains have been determined. The sequence that aligns with, e.g., the C-domain sequence in the alpha-amylase for which the C-domain has been determined can be considered the C-domain for the given alpha-amylase.

A and B domain: The term “A and B domain” as used herein means these two domains taken as one unit, whereas the C domain is another unit of the alpha-amylases. Thus, the amino acid sequence of the “A and B domain” is understood as one sequence or one part of a sequence of an alpha-amylase comprising an “A and B domain” and other domains (such as the C domain). As used herein, the “A and B domain” of an alpha-amylase corresponds to amino acids 1-399 of SEQ ID NO: 4.

Alpha-amylase: The term “alpha-amylase” is synonymous with the term “polypeptides having alpha-amylase activity”. “Alpha-amylase activity” means the activity of alpha-1,4-glucan-4-glucanohydrolases, E.C. 3.2.1.1, which constitute a group of enzymes, catalyzing hydrolysis of starch and other linear and branched 1,4-glucosidic oligo- and polysaccharides. For purposes of the present invention, alpha-amylase activity is determined according to the procedure described in the Methods. In one aspect, the alpha-amylases of the present invention have at least 20%, e.g., at least 40%, at least 50%, at least 60%, at least 70%, at least 80%, at least 90%, at least 95%, or at least 100% of the alpha-amylase activity of the mature polypeptide of SEQ ID NO: 2.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

C domain: As used herein, the “C domain” of an alpha-amylase corresponds to amino acids 400-485 of SEQ ID NO: 4. Thus, the C domain of an alpha amylase may be found by alignment of said alpha-amylase with the alpha-amylase of SEQ ID NO: 4. The part of said alpha-amylase that aligns with amino acids 400-485 of SEQ ID NO: 4 is according to the present invention “the C domain” of the alpha-amylase.

Corresponding to: The term “corresponding to” as used herein, refers to way of determining the specific amino acid of a sequence wherein reference is made to a specific amino acid sequence. E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NO: 1, or any other sequence listed herein, has been described elsewhere herein. Alternative alignment methods may be used, and are well-known for the skilled person.

Detergent composition: The term “detergent composition” as used herein, refers to a composition suitable for use within the field of detergents, such as for use in laundry and dish wash. A detergent composition may be in the form of a liquid or powder form, and may be suitable for both handwash or automated wash. Thus, the term “detergent composition” includes otherwise indicated by context, granular or powder-form all-purpose or heavy-duty washing agents, especially the so-called heavy-duty liquid (HDL) types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, soap bars, mouthwashes, denture cleaners, car or carpet shampoos, bathroom cleaners; hair shampoos and hair-rinses; shower gels, foam baths; metal cleaners; as well as cleaning auxiliaries such as bleach additives and “stain-stick” or pre-treat types. The terms “detergent composition” and “detergent formulation” are used in reference to mixtures which are intended for use in a wash medium for the cleaning of soiled objects. In some embodiments, the term is used in reference to laundering fabrics and/or garments (e.g., “laundry detergents”). In alternative embodiments, the term refers to other detergents, such as those used to clean dishes, cutlery, etc. (e.g., “dishwashing detergents”). It is not intended that the present invention be limited to any particular detergent formulation or composition. The term “detergent composition” is not intended to be limited to compositions that contain surfactants. It is intended that in addition to the variants herein described, the detergents compositions may comprise, e.g., surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti corrosion agents, enzyme inhibitors or stabilizers, enzyme activators, transferase(s), hydrolytic enzymes, oxido reductases, bluing agents and fluorescent dyes, antioxidants, and/or solubilizers.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of a mature polypeptide; wherein the fragment has alpha-amylase activity.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Immunological cross reactivity: The term “a polypeptide having immunological cross reactivity with an antibody” as used herein, refers to any polypeptide which is bound by an antibody raised against the polypeptide of SEQ ID NO: 1 or 2. When a polypeptide not necessarily having the sequence as set forth in SEQ ID NO: 1 or 2 is bound by an antibody, and thereby provides cross reactivity, it indicates that the polypeptide may have similar characteristics as the polypeptides of SEQ ID NO: 1 or 2. Determination of cross reactivity may be done by ELISA comprising the steps of (i) adhering the polypeptide of interest to the ELISA plate; (ii) adding the antibody raised against the polypeptide of SEQ ID NO: 1 or 2; (iii) adding a secondary labeled antibody binging the antibody raised against the polypeptide of SEQ ID NO: 1 or 2; and (iv) measuring the signal from the bound secondary antibody. Other methods of determining the immunological cross reactivity may be used and is within the knowledge of the skilled person.

Improved property: The term “improved property” means a characteristic associated with a polypeptide of the present invention which is improved compared to the mature polypeptide of SEQ ID NO: 2. Such improved properties include, but are not limited to, catalytic efficiency, catalytic rate, chemical stability, chelator stability, oxidation stability, pH activity, pH stability, specific activity, detergent stability, substrate binding, substrate cleavage, substrate specificity, substrate stability, surface properties, thermal activity, and thermo stability, and improved wash performance, particularly improved wash performance at low temperatures, such as temperatures between 5° C. and 40° C. such as at 15° C. Another property that may be improved is the stability of the molecule during storage in detergent compositions, in particular in liquid detergent compositions. The improvement in stability or wash performance is relative to the stability of the amylase of SEQ ID NO: 2 or relative to the polypeptide of SEQ ID NO: 1 having identical alterations of a) as the variant.

Wash performance: In the present context the term “wash performance” is used as an enzyme's ability to remove starch or starch-containing stains present on the object to be cleaned during e.g. laundry or hard surface cleaning, such as dish wash. The term “wash performance” includes cleaning in general e.g. hard surface cleaning as in dish wash, but also wash performance on textiles such as laundry, and also industrial and institutional cleaning. The wash performance may be quantified by calculating the so-called Intensity value.

Improved wash performance: The term “improved wash performance” is defined herein as displaying an alteration of the wash performance of an amylase of the present invention relative to the wash performance of the amylase of SEQ ID NO: 2, or relative to the polypeptide of SEQ ID NO: 1 having identical alterations of a) as the variant. The alteration may e.g. be seen as increased stain removal. Improved wash performance is determined according to Example 1. The wash performance is improved if the Improvement Factor (IF) is above 1.0, preferably above 1.05 in one or more of the conditions listed in Example 1. I.e. either in model detergent A at 15° C. where the alpha-amylase variant concentration is 0.2 mg/L, or in model detergent A at 40° C. where the alpha-amylase variant concentration is 0.05 mg/L, or in model detergent J at 15° C. where the alpha-amylase variant concentration is 0.2 mg/L, or in model detergent J at 30° C. where the alpha-amylase variant concentration is 0.05 mg/L. The wash conditions are described in Example 1. The term “wash performance” includes cleaning in general e.g. hard surface cleaning as in dish wash, but also wash performance on textiles such as laundry, and also industrial and institutional cleaning. Improved wash performance may be measured by comparing the delta intensities as described in the definition herein.

Delta intensity: The terms “Delta intensity” or “Delta intensity value” are defined herein as the result of a intensity measurement of a test material, e.g. a swatch CS-28 (Center For Testmaterials BV, P.O. Box 120, 3133 KT Vlaardingen, the Netherlands) or a hard surface. The swatch is measured with a portion of the swatch, washed under identical conditions, as background. The delta intensity is the intensity value of the test material washed with amylase subtracting the intensity value of the test material washed without amylase.

Textile: Textile sample CS-28 (rice starch on cotton) is obtained from Center For Testmaterials BV, P.O. Box 120, 3133 KT Vlaardingen, the Netherlands.

Low temperature: “Low temperature” is a temperature of 5-40° C., such as 5-35° C., preferably 5-30° C., more preferably 5-25° C., more preferably 5-20° C., most preferably 5-15° C., and in particular 5-10° C. In a preferred embodiment, “Low temperature” is a temperature of 10-35° C., preferably 10-30° C., more preferably 10-25° C., most preferably 10-20° C., and in particular 10-15° C. Most preferred, low temperature means 15° C.

Intensity value: The wash performance is measured as the brightness expressed as the intensity of the light reflected from the sample when illuminated with white light. When the sample is stained the intensity of the reflected light is lower, than that of a clean sample. Therefore the intensity of the reflected light can be used to measure wash performance, where a higher intensity value correlates with higher wash performance. Color measurements are made with a professional flatbed scanner (EPSON Expression 10000XL, EPSON) used to capture an image of the washed textile.

To extract a value for the light intensity from the scanned images, 48→24 Bit Color pixel values from the image are converted into values for red, green and blue (rgb). The intensity value (Int) is calculated by adding the rgb values together as vectors and then taking the length of the resulting vector:

Int=\sqrt{r²+g²+b²}

Improvement Factor: Improvement Factor (IF) is the ratio of delta intensity of enzyme sample over the delta intensity of backbone or reference, i.e., SEQ ID NO:2.

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 485 of SEQ ID NO: 1. In another aspect, the mature polypeptide is amino acids 1 to 483 of SEQ ID NO: 2. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having alpha-amylase activity.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent alpha-amylase: The term “parent” or “parent alpha-amylase” means an alpha-amylase to which an alteration is made to produce enzyme variants. The amylase having SEQ ID NO: 1 or 2 may e.g. be a parent for the claimed alpha-amylase variants.

Polynucleotide: The term “polynucleotide” or “polynucleotide encoding” as used herein refers to a polynucleotide that encodes a mature polypeptide having alpha-amylase activity.

Resulting variant: The term “resulting variant” as used herein, refers to a variant that has been produced by a method according to the present invention, i.e. the variant comprises any one or more of the alterations, such as substitutions and/or deletions, as described herein.

The resulting variant has the same meaning and purpose as the term “variant”, “variant according to the invention” and other terms used similarly herein, and thus, is the variant that is the result of the production method.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows:

(Identical Residues×100)/(Length of Alignment−Total Number of Gaps in Alignment)

Variant: The term “variant” means a polypeptide having alpha-amylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the alpha-amylase activity of the mature polypeptide of SEQ ID NO: 1. The variants of the present invention are preferably variants of the parent alpha-amylase of SEQ ID NO: 1 or an alpha-amylase having at least 90% identity hereto, such as at least 95% identity hereto. They may also be variants of other parents, such as SEQ ID NO: 2, 4, 7, 8, 9, 10, 11, 12, 13, 14, 15 or 16.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

Wild-type alpha-amylase: The term “wild-type” alpha-amylase means an alpha-amylase expressed by a naturally occurring microorganism, such as a bacterium, archaea, yeast, or filamentous fungus found in nature.

### Conventions for Designation of Variants

For purposes of the present invention, the mature polypeptide disclosed in SEQ ID NO: 1 is used to determine the corresponding amino acid residue in another alpha-amylase. The amino acid sequence of another alpha-amylase is aligned with the mature polypeptide disclosed in SEQ ID NO: 1 and based on the alignment, the amino acid position number corresponding to any amino acid residue in the mature polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another alpha-amylase may be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms may be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity may be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, may be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures may be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms may additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions.

For an amino acid substitution, the following nomenclature is used: Original amino acid, position, substituted amino acid. Accordingly, the substitution of threonine at position 226 with alanine is designated as “Thr226Ala” or “T226A”. In situations where the amino acid at a given position may be substituted for any other amino acid it is designated T226A;C;D;E;F;G;H;I;K;L;M;N;P;Q;R;S;W;V;Y. Accordingly, this means that threonine at position 226 may be substituted with one amino acid selected from the group of A, C, D, E, F, G, H, I, K, L, M, N, P, Q, R, S, W, V or Y. Likewise, in situations where the amino acid at a given position may be substituted for one amino acid selected from a specific group of amino acids, e.g. where the threonine at position 226 may be substituted with any of tyrosine, phenylalanine or histidine it is designated T226Y;F;H. The different alterations at a given position may also be separated by a comma, e.g., “Arg170Tyr,Glu” or “R170Y,E” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions.

For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions.

For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple Alterations.

Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively. The term “multiple modifications” may also be used herein. Such term have the same meaning and purpose as “multiple alterations” and may be used interchangeably.

### DETAILED DESCRIPTION OF THE INVENTION

### Alpha-Amylase Variants

The present invention relates to an alpha-amylase variant comprising a) a deletion and/or a substitution at two or three or four positions corresponding to positions R181, G182, H183 and G184 of the mature polypeptide of SEQ ID NO: 1, and b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In another aspect, the present invention relates to an alpha-amylase variant comprising

a) a pairwise deletion at amino acid positions corresponding to positions R181+G182, R181+G184, G182+H183, or G182+G184 of the mature polypeptide of SEQ ID NO: 1, and
b) a substitution at one or more positions, said positions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 70%, such as at least 75%, such as at least 80%, such as at least 85%, such as at least 90%, such as at least 95% sequence identity but less than 100% sequence identity to the polypeptide having the amino acid sequence of SEQ ID NO: 1 and wherein the variant has alpha-amylase activity.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a preferred embodiment b) comprises one or more of the following substitutions and/or deletions: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, where * denotes a deletion, when using SEQ ID NO: 1 for numbering.

In a preferred embodiment a) is a deletion of amino acids H183+G184 and b) comprises one or more of the following substitutions and/or deletions: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, where * denotes a deletion, when using SEQ ID NO: 1 for numbering.

In another embodiment a) is a deletion of amino acids R181+G182 and b) comprises one or more of the following substitutions and/or deletions: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, where * denotes a deletion, when using SEQ ID NO: 1 for numbering.

In another embodiment a) is a deletion of amino acids R181+H183 and b) comprises one or more of the following substitutions and/or deletions: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, where * denotes a deletion, when using SEQ ID NO: 1 for numbering.

In another embodiment a) is a deletion of amino acids R181+G184 and b) comprises one or more of the following substitutions and/or deletions: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, where * denotes a deletion, when using SEQ ID NO: 1 for numbering.

In another embodiment a) is a deletion of amino acids G182+H183 and b) comprises one or more of the following substitutions and/or deletions: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, where * denotes a deletion, when using SEQ ID NO: 1 for numbering.

In another embodiment a) is a deletion of amino acids G182+G184 and b) comprises one or more of the following substitutions and/or deletions: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, where * denotes a deletion, when using SEQ ID NO: 1 for numbering.

In another preferred embodiment, b) comprises one or more of the following combinations of substitutions and/or deletions: H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51 L+N174S+A447V, G50A+A51T+L173F, G50A+R172Q+L173I, G50A+A51V+L173F+N174D, G50A+R172D+L173T, G50A+A51T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G, G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S, R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L, R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51 L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51 T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51 T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S.

In another preferred embodiment, the alterations of b) consist of one of the following combinations of alterations: H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51 T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51 L+N174S+A447V, G50A+A51T+L173F, G50A+R172Q+L173I, G50A+A51V+L173F+N174D, G50A+R172D+L173T, G50A+A51T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V, Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S, R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L, R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174 D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51 I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51 L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51 T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51 T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S when using SEQ ID NO: 1 for numbering.

In another preferred embodiment a) is a deletion of amino acids H183+G184 and the alterations of b) comprises of one of the following combinations of alterations: H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51 L+N174S+A447V, G50A+A51T+L173F, G50A+R172Q+L173I, G50A+A51V+L173F+N174D, G50A+R172D+L173T, G50A+A51T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V, Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S, R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L, R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51 L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51 T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51 T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S when using SEQ ID NO: 1 for numbering.

In another preferred embodiment a) is a deletion of amino acids R181+G182 and the alterations of b) comprises of one of the following combinations of alterations: H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51 T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51 L+N174S+A447V, G50A+A51T+L173F, G50A+R172Q+L173I, G50A+A51V+L173F+N174D, G50A+R172D+L173T, G50A+A51T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S, R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L, R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51 I+R172D+L173T, G50A+N174Q+A204T, A51 L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51 L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51 T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S when using SEQ ID NO: 1 for numbering. In another preferred embodiment a) is a deletion of amino acids R181+H183 and the alterations of b) comprises of one of the following combinations of alterations: H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51 L+N174S+A447V, G50A+A51T+L173F, G50A+R172Q+L173I, G50A+A51V+L173F+N174D, G50A+R172D+L173T, G50A+A51T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98 L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V, Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S, R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51 L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51 T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51 T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S when using SEQ ID NO: 1 for numbering.

In another preferred embodiment a) is a deletion of amino acids R181+G184 and the alterations of b) comprises of one of the following combinations of alterations: H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51 L+N174S+A447V, G50A+A51T+L173F, G50A+R172Q+L173I, G50A+A51V+L173F+N174D, G50A+R172D+L173T, G50A+A51T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98 L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S, R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51 L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51 T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51 T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S when using SEQ ID NO: 1 for numbering.

In another preferred embodiment a) is a deletion of amino acids G182+G184 and the alterations of b) comprises of one of the following combinations of alterations: H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51 T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51 L+N174S+A447V, G50A+A51T+L173F, G50A+R172Q+L173I, G50A+A51V+L173F+N174D, G50A+R172D+L173T, G50A+A51T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V, Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S, R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51 L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S when using SEQ ID NO: 1 for numbering.

In a particular embodiment, the variant has an Improvement Factor (IF) of at least 1.1 when determined in a Model A detergent at a temperature of 15° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: Y363L, Y160L, W167L, W167F, K72S, K72R, T40K, T40M, H1*+H2*+G7S, Q361S, Q361A, E345S, E276S, W268F, V291A, V56A, V56T, R171E, K302A, L429A, A354V, K347M, K347A, N270Y+Y295N+N299T, Y295N+N299T, A113Y+R171E, T40K+K179S, H1L+R171E+D192E, T40K+R171E, R171E+H324L+Q361A+Y363L, R171E+Q361A+Y363L, A113Y+R171E+E391L, R171E+D192E+Y363L, A113Y+R171E+Y363L+E391L+K423P, 408H, K423N, I430M, W140Y, R320A, W140Y+R320A, R172Y, A113N, R142K, R172L, R172LR172S, A113Y, A113L, R142S, R172P, R172T, R172V, R172A, L173V, N174Y, N174G, Y243E, Y243S, Y243G, Y243D, V165G, Y243T, P211D, K269L, K269N, K269I, K269V, K269M, E391A, E391M, K179S, E391L, E391H, K179A, K179T, H1G, H1S, H1L, K423L, K423H, K423P, K423T, L217F, K302R, N296Q, H1N, N197A, V165M, D192A, D192E, N281Q, N281S, V165A, V165G, M208S, A186M, H321A, H324R, H324W, S323E, N128T, T193G, T193A, T193N, H1*+H2E, H1*+H2V, Q280S, H1*+H2A, G304V, I430E, G149Q, G149A, Q169G, Q169M, N215A, N311L, L351C, N215G, L351H, N311E, N219S, N219E, N219T, H1*+N3C, A274T, L351S, G182A+V213T+V214S, H210S, H210Y, P211T, P211T+V213T, G182N+V213S, H210S+V214T, P211T, V213T+V214S, K269Q+A274W, K269H+A274E, K269Q+A274Y, K269Q+A274L, K269Q+A274M, K269Q+A274S, K269Q+A274G, K269V+A274Y, K269L+A274G, K269L+A274W, K269Q+A274R, K269R+A274G, K269H+A274C, R172Q+N174Q, R172Q+N174S, R172Q+L173F+N174Y, G50A+A51T+N174E, R172N, R172Q+N174T+A204T, N174S, G50A+A51T+R172Q+N174Q+E471K, G50A+R172Q, R172K+L173I+N174S, G50A+A51V+R172N+N174S, R172Q+L173F+A204T, G50A+A51T+R172Q, G50A+R172Q+N174T, R172Q+N174T, G50A+R172Q+N174D, N174D+A204V, G50A+R172N+N174S, G50A+R172N+N174T, G73V+R172Q+N174E, R172N+L173T+N174Q+S303R, G50S+R172K+N174D, G50A+A51T+K72H+R172Q+N174Q+A204V, R172Q, L173T+N174Q+A204V, R172K+L173F, G50A+R172Q+L173T+N174D, G50A+N174E, G50A+A51T+R172Q+L173V, G50A+R172D+N174D, G50A+R172D+N174Q, R172D+N174T, G50A+A51V+R172N+N174T+A204T, R172E, G304Q+E346P+D476K+G477A, E346P+G477Q, E346P+G477T, G304S+E345D+E346P+D476K+G477Q, E346P+D476Q+G477T, E346P+D476K+G477S, E345D+D476K+G477S, E346T+D476K+G477A, E345D+E346P+D476G, K302Q+E346P+D476K+G477S, G304A+E345D+E346P+D476G+G477K, G304Q+E346P+D476G, E346P+D476G+G477K, G304A+E346P+D476K+G477A, K302N+E345D+D476Q+G477Q, G304A+D476K+G477S, E345D+E346P+G477Q, L173F+N174Q, R172Q+L173F, G50A+N174S, G50A+R172Q+L173V+N174S, G50A+A51V+R172D+L173F, G50A+A51I+R172D+N174*, R172D+L173F, R172D, A51V+R172Q+A204V, G50A+A51V+R172D+L173F+N174Q+A204T, G50S+R172N+N174Q, G50A+R172Q+L173I+N174T+A204T, L173F, T136N+R172Q+N174D+A204T, G50A+R172Q+A204V, G50A+R172N+A204T, A51T+L173T+N174S+A204T, L173I+N174D, W48L+A51T+R172Q+N174S+A204T, G50A+A51V+R172E+L173F+N174T+A204V, G50A+R172Q+L444P, R172Q+L173T+N174S, L173F+A204T, G50A+A51T+R172N+L173V, G50A+R172Q+N174S+A204T, R172Q+L173F+N174D, G50A+A51V+R172Q, R172Q+N174*, G50A+A51T+R172D, G50A+A51T+R172N+N174D+G182V, G50A+R172E+L173F, L173V+N174T, N174T, G50A+A51T+R172Q+N174E, G50A+R172Q+L173F+N174Q, R172E+L173F+N174Q, A51I+R172E+N174T, G50A+A51I+A204V, R172Q+N174D, G50A+L173F+N174S, G50A+R172E+L173V+N174T, G50A+A51L+N174S+A447V, G50A+A51T+L173F, A274E+G304Q+G305S+E345D+D476K+G477Q, E346P+D476Q+G477S, E345D+E346P+D476G+G477T, E346T+D476K, E345D+D476Q+G477K, G304Q+E345D+E346T+D476G+G477S, E346T, G305S+E346T+D476Q+G477Q, E345D+E346T+D476Q+G477Q, K302Q+E345D+E346P+D476K+G477Q, E346T+G477K, E346P+G477S, K302Q+E346P+D476K+G477Q, G304A+E346P+D476G+G477A, E345D+D476Q+G477S, G304S+E346P+D476K+G477S, G304A+E345D+E346T+D476Q+G477Q, E346T+D476Q+G477A, R118L, R118S+G182A+T193K, R118P+G182A, R118P+G182S, R118S+E134D, E134D+K179L+G182N, R118S, R118S+G182A, R118S+N128L+G182S, G182S+A186V, R118D+K179L+G182A, R118P+G182N, R118S+E134D+G182S, E134D+A186E, N128F+K179L, N128Q+K179L+A186S, R118D, G304Q+E346P+D476G+G477S, E345D+D476G+G477S, D476K+G477Q, G304A+E345D+D476Q+G477Q, R172E+L173T+N174T, G50A+A51V+L173F+N174D, G50A+R172D+L173T, A51V+R172Q+N174S+A204V, G50A+R172D+L173T+N174S, G50A+N174D+A204T, R172Q+L173V, G50A+A51T+R172N+L173I+N174Q, G50A+L173T+N174S+A204V, R172Q+L173F+N174D+A204T, G50A+A51T+R172Q+A204V, R172E+L173F+N174D, A51V+N174T+A204V, R172K+A204V, A51T+R172Q+L173T, G50A+A51V+R172Q+A204V, G50A+A51Q+R172Q, A51T+R172N+A204V, G50A+A51T+R172E+L173I, N174Q+A204V, G50A+A51V+L173F+N174*, R172E+N174D+A204T, A51T+R172N+L173*, R172N+L173F+N174Q+V489G, R172E+L173F, G50A+A51T+A87S+R172N+L173T+N174D, G50A+R172N+L173T+N174D, A51T+R172Q, G50A+R172E+L173F+N174D, G67E+D112N+R172D+L173F+N174S+S466F, G50A+R172D+N174T, G50A+A51T+R172Q+N174T, G50A+R172D, G50A+N174E+A204T, R172Q+L173V+A204T, A51V+R172Q+L173F+N174S, A51T+R172D+N174Q, G50A+A51T+R172E+N174S, G50A+A51T+R172Q+N174Q, G50A+A51T+R172Q+L173F+N174T, A51V+L173V+A204V, G149Q+R171E, A113Y+G149Q+R171E+D192E+Q280S, H1L+A113Y+R171E+D192E, R171E+D192E+Q280S+Q361A+Y363L+E391L+K423P, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, R172Q+N174D+G304Q+E345D+E346T+D476G+G477S, K302N+E346T+D476Q+G477T, K302Q+D476G+G477A, S255I+E345D+G477Q, E346T+G477A, K302Q+E346P+D476K+G477A, G304A+E345D+E346P+D476Q+G477S, A47G+G67E+A111T+D112H+R245L+E346P+D476G+G477T, G304A+E346T+D476K+G477T, G304S+E345D+D476K+G477Q, K302N+E345D+D476G, A113T+G304S+E345D+E346P+D476G+G477Q, R320S+H324Q, R320S+S323M, G7E+R320M+S323M, G7Q+Q98S+R320S+H324Q, Q98A+R320A+S323N, Q98T+R320A+H324L, G7E+Q98T+R320A, G7L+R320S+S323M+V489G, G7D+Q98A+R320M, G7E+Q98A+R320M, R320A+S323N, G7K+R320A+S323M+H324L, G7L+P211Q+S303G+R320A, G7Q+R320S+S323M, Q98A+A265S+R320A, G7N+R320A, Q98N+R320A+S323N, R320M, G7A+R320M, G7P+R320S+S323K, G7Q+R320A+S323N, G7L+Q98A+R320A+S323M, R320A+H324L, G7K+R320M+H321Q, R320S+S323N, G7N+R320A+S323N, G7Q+R320S+H321N+S323M, R320A+H324Q, G304A+E346P+D476Q+G477T, K302Q+E346P+D476K, K302Q+E346P+G477Q, K302Q+D476G+G477Q, G304A+E345D+D476Q, E345D+E346P+D476Q+G477S, G304Q+E345D+E346T, E345D+E346T+D476G+G477S, V75I+R320A+S323N, G7X+Q98T+R320A, R320T, G7N+Q98S+R320M+S323N, G7Q+R320A+S323R, G7E+Q98A+K302R+R320A+S323N, G7A+R320S+H321W+S323M, A87D+R320S+S323M, R320S+S323M+H324L, Q98K+K269N+R320A+S323M+H324Q, G7K+R320A+P408Q, G7K+R320A+H324L, R320A+H321W+S323M+H324Q, G7L+R320M+S323N, N28S+R320M+S323N, Q98S+R320A, G7E+R320A, T5*+G4*+N3*+R320A, G7E+R142I+R320A+E391K+A392P+R393*+Q394S, G7L+R320A+S323M, G7E+R320A+P320Q+S323M, G7K+R320A+S323N+P364S, G7Q+R320A+H321Q+S323M, R320V+D476Y, G7N+R320M, G7H+Q98S+R320A+S323M+H324L, G7Q+R320S+H321N, R320A+H321N+S323N, G7E+R320M, G7Q+Q98T+R320A+S323M, Q98T+R320S+S323M, R320M+S323M+H324Q, G7K+R320A, Q98A+R320S+S323M, G7K+T246M+R320A+S323M, G7N+M9V+Q98A+R320S+S323M, G7Q+Q98A+R320M+S323M, G7Q+Q86H+R320A+S323M, G50A+A51Q+R172Q+G304Q+E345D+E346T+D476G+G477S, R172E+N174D+A204T+G304Q+E345D+E346T+D476G+G477S, G50A+A51V+R172Q+A204V+G304Q+E345D+E346T+D476G+G477S, A51T+R172N+A204V+G304Q+E345D+E346T+D476G+G477S, G50A+A51T+A87S+R172N+L173T+N174D+G304Q+E345D+E346T+D476G+G477S, A51T+R172D+N174Q+G304Q+E345D+E346T+D476G+G477S, G50A+A51T+R172E+N174S+G304Q+E345D+E346T+D476G+G477S, G50A+A51T+R172Q+N174S+A204V, G50A+R172D+N174S, A51V+R172E+N174*, G50A+R172E+L173F+N174T, G50A+R172N, G50A+R172N+L173V, A51V+R172D, L173T+N174T+A204T, G50A+R172E+L173V+N174S, R172E+N174T, N28D+G109S+A119P+R172D+L173V+N174S, R172E+N174D, A51I+R172E+L173F, R172Q+A204V, G50A+A51T+R172E+N174T, R172Q+L173F+N174T, R172N+N174T+A204V, R172K+N174S, L173T+N174S+A204V, R172D+N174S, R172D+L173V+N174D, G50A+A51T+R172Q+L173I, R172Q+L173T+N174Q, R172Q+N174E, G50A+A51T+R172E+A204V, R172D+L173F+N174D, G50A+R172E+N174T, L173F+N174Q+A204V, G50A+R172N+A204T, R172E+L173I+A204T, G50A+R172Q+L173V, A51T+R172D+N174D, R172K+L173V+N174S+A204T, R172E+L173T+N174S+A204T, G50A+A51T+R172E, G50A+A51V+R172D+N174T, R172D+L173I+N174S, A51T+R172E+N174T, R172D+L173F+A447T+T453N, L173T+N174T+A204V, R172N+N174D, G50A+L173I+L173I+N174T+N174T+N219K+W220F, G50A+R172E+L173F+N174Q, G50A+A51V+R172Q+N174Q, G50A+R172N+N174E, R172N+N174T+H324N, G50A+R172Q+L173T+N174Q, R172K+L173V+N174S+A204T+L429F, G50A+R172E+L173F+N174T, R172D+L173V, R172E+L173T+N174S, R172D+L173I+N174S, A51V+R172Q+L173T, G50A+A51T+R172Q+L173V+A204V, G50A+A51T+R172D+L173F+N174T+K423P, N174E, T40I+A51L+R172D+L173F, G50A+R172E+L173F+N174Q+A204T, R172K+L173F+N174Q+A204V, R158G+N174E, A51T+N174D+A204V, R172N+L173I, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, A113L+R172Q+N174D, R118D+W167F+R172Q+N174D, R118D+R172Q+N174D, W167F+R172Q+N174D, A204T+G304Q+E345D+E346T+D476G+G477S, R172N+L173F+N174Q+G304Q+E345D+E346T+D476G+G477S, A113L+W167F+R172Q+Y363L, T40K+G50A+R172Q+I430E, T40K+E346T, T40K+K72R+W167F, W167F+R172Q+E346T+Y363L, K72R+A113L+W167F+R172Q+N215G+Y363L, W167F+R172Q+E346T, A113L+W167F+R172Q+N215G, S255I+R320A+H321N+S323N+E345D+G477Q, G50A+A113L+W167F+R172Q, G50A+W167F+R172Q, G50A+R118D+R172Q, G50A+A113L+R118D+R172Q, G50A+A113L+W167F+R172Q, G50A+R172D+L173F+E471K, G50A+A51T+R172D+E345D+E346P+D476G, R171E+D192E+E345D+E346P+D476G, G50A+R172D+N174D+E345D+E346P+D476G, G50A+R172D+N174T+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477Q, R171E+D192E+S255I+E345D+G477Q, G50A+R172D+N174D+S255I+E345D+G477Q, G50A+A51V+R172N+N174T+A204T+S255I+E345D, G50A+R172D+N174T+S255I+E345D+G477Q, R172K+A204V+S255I+E345D+G477Q, R172K+A204V+S255I+E345D+K423P+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, W140Y+R172D+N174T, G50A+W140Y+R172N+N174T, G50A+A51V+W140Y+P146S+L173F+N174D, A113L+S255I+E345D+G477Q+V489G, A113L+R118D+W167F+S255I+E345D+G477Q, K72R+A113L+S255I+E345D+G477Q, K72R+A113L+W167F+S255I+E345D+G477Q, W167F+S255I+E345D+G477Q, R118D+W167F+S255I+E345D+G477Q, Q98N+Q169M+R320A+S323M, Q98N+M208Y+V213T+V214T+L217M+R320A+S323M, R172E+N174D+A204T+G304Q+E345D+E346T+E391L+D476G+G477S, R172E+N174D+A204T+G304Q+E345D+E346T+E391L, R172E+N174D+A204T+G304Q+R320A+H321N+S323N+E345D+E346T+D476G+G477S, G50A+A51T+A87S+Q169M+R172N+L173T+N174D+G304Q+E345D+E346T+D476G+G477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+R172N+N174T+R320A+H321N+S323N, G50A+A51V+L173F+N174D+R320A+H321N+S323N, W167F+E345D+D476K+G477S, A113L+W167F+E345D+D476K+G477S, R172E+N174S+G304Q+R320A+H321N+S323N+E345D+E346T+D476G+G477S, G50A+A51T+R172E+N174S+G304Q+R320A+H321N+S323N+E345D+E346T+D476G+G477S, G50A+A51T+R172E+N174S+M208Y+V213T+V214T+L217M+G304Q+E345D+E346T+D476G+G477S, R172E+N174D+A204T+G304Q+R320A+S323N+E345D+E346T+D476G+G477S, G50A+A51V+W167F+R172E+L173F+N174T+A204V+R320A+S323N, G50A+A51V+W167F+R172E+L173F+N174T+M208Y+V213S+V214T+L217M, G50A+A51V+W1 67F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+M208Y+V213S+V214T+L217M, G50A+A51T+W167F+R172N+L173V+M208Y+V213T+V214T+L217M, T40K+K72R+W167F+R320A+S323N, T40K+K72R+W140Y+W167F, V165G, P211T+V213T, P211T, G50A+R172N+A204T, G50A+R172E+L173F+N174T, R172D+L173I+N174S, R172Q+N174T, T40K+K72R+W167F+M208Y+V213T+V214T+L217M+R320A+S323N, G50A+A51I+R172D+L173V, G50A+A51L+R172N+L173V+A204T, R172D+L173T+N174D+A204T, L173T+A204T, A51I+R172D+N174T, A51T+R172K+L173I+N174S, G50A+R172E+N174S, R172N+L173V, G50A+R172K+N174Q, T5K+R172E+L173F, G50A+A51T+R172D+N174S, N25D+R172N+A204T, R172D+L173T+N174D, L173F+N174D+A204S, G50A+L173T+N174S, G50A+A51T+R172N+A204T, G50A+A51S+R172D+N174Q, G50A+D112N, G50A+A51V+A113L+R172E+L173F+N174T+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477SR172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51 T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51 T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, A51T+W167F+R172N+A204V+G304Q+E345D+E346T, 51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, G7K+T40K+W167F+R320A+S323N+P364S, G7K+T40K+K72R+W167F+R320A+S323N+P364S, when using SEQ ID NO:1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.3 when determined in a Model A detergent at a temperature of 15° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: W167F, K72S, K72R, T40K, T40M, H1*+H2*+G7S, E345S, V291A, V56A, V56T, R171E, A354V, A113Y+R171E, H1L+R171E+D192E, T40K+R171E, R171E+H324L+Q361A+Y363L, R171E+Q361A+Y363L, A113Y+R171E+E391L, A113Y+R171E+Y363L+E391L+K423P, W140Y, W140Y+R320A, R172Y, R142K, R172S, A113Y, A113L, R172T, R172V, Y243E, Y243S, Y243G, Y243D, V165G, P211D, K269M, K179S, E391H, K179T, V165M, V165G, T193G, Q169M, N215G, G50A+A51T+N174E, G50A+R172Q, G50A+A51V+R172N+N174S, G50A+R172Q+N174T, R172Q, L173T+N174Q+A204V, E346T+D476K+G477A, E345D+E346P+D476G, K302Q+E346P+D476K+G477S, G304A+E345D+E346P+D476G+G477K, R172Q+L173F, G50A+N174S, G50A+R172Q+L173V+N174S, G50A+A51V+R172D+L173F, G50A+A51V+R172D+L173F+N174Q+A204T, G50A+A51V+R172E+L173F+N174T+A204V, G50A+A51T+R172N+L173V, R172Q+L173F+N174D, G50A+R172E+L173F, L173V+N174T, N174T, G50A+R172Q+L173F+N174Q, R172E+L173F+N174Q, A51I+R172E+N174T, G50A+A51I+A204V, R172Q+N174D, G50A+L173F+N174S, G50A+R172E+L173V+N174T, G50A+A51L+N174S+A447V, G50A+A51T+L173F, A274E+G304Q+G305S+E345D+D476K+G477Q, E346P+D476Q+G477S, G304Q+E345D+E346T+D476G+G477S, E346T, R118S+G182A+T193K, R118P+G182A, R118P+G182S, R118S+E134D, R118D+K179L+G182A, N128Q+K179L+A186S, R118D, G304Q+E346P+D476G+G477S, G50A+R172D+L173T, R172Q+L173V, G50A+A51T+R172N+L173I+N174Q, R172E+L173F+N174D, R172K+A204V, A51T+R172Q+L173T, G50A+A51Q+R172Q, G50A+A51T+R172E+L173I, R172E+N174D+A204T, A51T+R172N+L173*, G50A+A51T+A87S+R172N+L173T+N174D, G67E+D112N+R172D+L173F+N174S+S466F, G50A+R172D+N174T, G50A+A51T+R172Q+N174T, G50A+R172D, G50A+N174E+A204T, R172Q+L173V+A204T, A51T+R172D+N174Q, G50A+A51T+R172E+N174S, G50A+A51T+R172Q+N174Q, R171E+D192E+Q280S+Q361A+Y363L+E391L+K423P, G50A+A51V+R172D+L173F+N174Q+A204T+G304Q+E345D+E346T+D476G+G477S, G50A+R172Q+L173I+N174T+A204T+G304Q+E345D+E346T+D476G+G477S, K302Q+D476G+G477A, S255I+E345D+G477Q, G304S+E345D+D476K+G477Q, A113T+G304S+E345D+E346P+D476G+G477Q, G7Q+Q98S+R320S+H324Q, R320A+S323N, G7L+P211Q+S303G+R320A, G7Q+R320A+S323N, G7K+R320M+H321Q, R320S+S323N, G7X+Q98T+R320A, R320T, Q98K+K269N+R320A+S323M+H324Q, G7K+R320A+P408Q, G7L+R320M+S323N, N28S+R320M+S323N, T5*+G4*+N3*+R320A, G7E+R142I+R320A+E391K+A392P+R393*+Q394S, G7L+R320A+S323M, G7K+R320A+S323N+P364S, G7Q+R320A+H321Q+S323M, G7H+Q98S+R320A+S323M+H324L, G7Q+R320S+H321N, R320A+H321N+S323N, G7Q+Q98T+R320A+S323M, G50A+A51Q+R172Q+G304Q+E345D+E346T+D476G+G477S, R172E+N174D+A204T+G304Q+E345D+E346T+D476G+G477S, G50A+A51V+R172Q+A204V+G304Q+E345D+E346T+D476G+G477S, A51T+R172N+A204V+G304Q+E345D+E346T+D476G+G477S, G50A+A51T+A87S+R172N+L173T+N174D+G304Q+E345D+E346T+D476G+G477S, A51T+R172D+N174Q+G304Q+E345D+E346T+D476G+G477S, G50A+A51T+R172E+N174S+G304Q+E345D+E346T+D476G+G477S, G50A+A51T+R172Q+N174S+A204V, G50A+R172N+L173V, A51V+R172D, R172E+N174T, G50A+A51T+R172E+N174T, R172D+L173V+N174D, G50A+A51T+R172Q+L173I, R172Q+N174E, R172E+L173I+A204T, A51T+R172D+N174D, R172E+L173T+N174S+A204T, L173T+N174T+A204V, R172N+N174D, G50A+R172E+L173F+N174Q, G50A+R172N+N174E, R172K+L173V+N174S+A204T+L429F, R172D+L173V, R172D+L173I+N174S, A51V+R172Q+L173T, G50A+A51T+R172Q+L173V+A204V, R172N+L173I, G50A+A51V+R118D+R172N+N174T+A204T, G50A+A51V+A113L+R118D+R172D+L173F, G50A+A113L+W167F+R172Q+L173I+N174T+A204T, G50A+A113L+R118D+R172Q+L173I+N174T+A204T, G50A+A113L+R172Q+L173I+N174T+A2 04T, G50A+A51V+R118D+W167F+R172E+L173F+N174T+A204V, G50A+A51V+W167F+R172E+L173F+N174T+A204V, G50A+A51V+A113L+R118D+R172E+L173F+N174T+A204V, G50A+A51T+W167F+R172N+L173V, G50A+A51T+A113L+R172N+L173V, G50A+A51T+R118D+R172N+L173V, R118D+W167F+R172Q+N174D, R118D+R172Q+N174D, W167F+R172Q+N174D, A204T+G304Q+E345D+E346T+D476G+G477S, R172N+L173F+N174Q+G304Q+E345D+E346T+D476G+G477S, A113L+W167F+R172Q+Y363L, T40K+G50A+R172Q+I430E, T40K+K72R+W167F, W167F+R172Q+E346T+Y363L, K72R+A113L+W167F+R172Q+N215G+Y363L, S255I+R320A+H321N+S323N+E345D+G477Q, G50A+A113L+W167F+R172Q, G50A+W167F+R172Q, G50A+A113L+R118D+R172Q, G50A+A113L+W167F+R172Q, G50A+R172D+L173F+E471K, G50A+A51T+R172D+S255I+E345D+G477Q, G50A+R172D+N174Q+S255I+E345D+G477Q, R171E+D192E+S255I+E345D+G477Q, G50A+R172D+N174D+S255I+E345D+G477Q, G50A+R172D+N174T+S255I+E345D+G477Q, G50A+A51T+W140Y+R172D, G50A+A51V+W140Y+R172E+L173F+N174T+A204V, G50A+W140Y+R172D+N174Q, G50A+A51T+W140Y+R172N+L173V, W140Y+R172D+N174T, A113L+R118D+W167F+S255I+E345D+G477Q, K72R+A113L+S255I+E345D+G477Q, K72R+A113L+W167F+S255I+E345D+G477Q, R118D+W167F+S255I+E345D+G477Q, Q98N+M208Y+V213T+V214T+L217M+R320A+S323M, R172E+N174D+A204T+G304Q+E345D+E346T+E391L+D476G+G477S, R172E+N174D+A204T+G304Q+E345D+E346T+E391L, R172E+N174D+A204T+G304Q+R320A+H321N+S323N+E345D+E346T+D476G+G477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+L173F+N174D+R320A+H321N+S323N, W167F+E345D+D476K+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+M208Y+V213S+V214T+L217M, V165G, G50A+A51I+R172D+L173V, R172D+L173T+N174D+A204T, G50A+R172E+N174S, R172D+L173T+N174D, G50A+A51T+R172N+A204T, G50A+A51V+A113L+R172E+L173F+N174T+A204V+E346T+D476K+G477A, T40K+K72R+Q98N+R320A+S323M, T40K+K72R+A113L+W167F, T40K+G50A+W167F+R172N+N174S+A204T, K72R+W167F+E346T+D476K+G477A+Y482F, G50A+A51V+K72R+W167F+R172E+L173F+N174T+A204V, T40K+G50A+A51V+K72R+W167F+R172E+L173F+N174T+A204V, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W 167F+R172N+L173, +N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+K72R+W167F+N174D+A204T, W140Y+W167F+N174D, G50A+W140Y+W167F+N174D+A204T, T40K+K72R+W167F+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, G50A+A51V+W140Y+P146S+W167F+R172E+L173F+N174T+A204V+R320A+S323N, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+E345D+L405V+T414K, G50A+A51V+R172N+N174T+A204T+E345D+D476K+G477S, G50A+R172D+N174Q+E345D+D476K+G477S, R172D+N174T+K302Q+E346P+D476K+G477S, R172N+N174Q+E345D+D406A+I411M+N446K, G50S+R172N+N174Q+E345D, G50A+N174D+A204T+S255I+E345D+G477Q, G50A+N174D+A204T+G304S+E345D+D476K+G477Q, N174D+A204T+G304S+E345D+D476K+G477Q, R172K+A204V+E345D+D476K, G50A+R172N+A204T+E346T+D476K+G477A, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51T+R172N+L173V+R320A+S323M+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, G50A+A51T+R172E+A204T+E346T+G477A, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+W167F+S255I+E345D+G477Q, G50A+A51T+R172E+A204T+E346T+D476K+G477A, L173T+A204T+K302Q+E346P+D476K+G477S, R172D+N174Q+E346T+D476K+G477A, G50A+R172D+N174Q+A204V+E345D+D476K+G477S, L173T+A204T+S255I+E345D+G477Q, G50A+R172D+N174Q+A204V+R320A+S323N, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+R172D+N174D+M208Y+V213S+V214T+L217M+E346T+D476K+G477A, G50A+A51V+R172E+N174S+M208Y+V213S+V214T+L217M, G50A+R118P+R172D+N174Q+G184S+E346T+D476K+G477A, R118P+L173T+G184S+A204T, G50A+A51V+R118P+R172D+N174E+G184S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172N+L173V+K302Q+E346P+D476K+G477S, R172N+L173V+E345D+D476K+G477S, R172N+L173V+S255I+E345D+G477Q, R172N+L173V+R320A+S323N, G50A+R118P+R172D+N174Q+E346T+D476K+G477A, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, T5K+R172E+L173F+K302Q+E346P+D476K+G477S, T5K+R172E+L173F+K302Q+E346P, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+R172D+N174D+E346T+D476K, G50A+R172N+N174T+E346T+D476K, R118P+W140Y, G7K+T40K+K72R+W167F+R320A+S323N+P364S, when using SEQ ID NO:1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.5 when determined in a Model A detergent at a temperature of 15° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: V291A, V56T, R171E, R172S, R172T, R172V, G50A+R172E+L173F, L173V+N174T, G50A+R172Q+L173F+N174Q, R172E+L173F+N174Q, G50A+A51I+A204V, R172Q+N174D, G50A+R172E+L173V+N174T, G50A+A51T+L173F, G304Q+E345D+E346T+D476G+G477S, E346T, R118P+G182A, N128Q+K179L+A186S, R118D, G50A+A51Q+R172Q, R172E+N174D+A204T, A51T+R172D+N174Q, G7L+R320A+S323M, G7K+R320A+S323N+P364S, G7H+Q98S+R320A+S323M+H324L, R320A+H321N+S323N, R172E+N174D+A204T+G304Q+E345D+E346T+D476G+G477S, G50A+A51V+R172Q+A204V+G304Q+E345D+E346T+D476G+G477S, A51T+R172N+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, R172D+L173V+N174D, R172Q+N174E, R172E+L173T+N174S+A204T, G50A+R172E+L173F+N174Q, G50A+A51T+R172Q+L173V+A204V, R118D+W167F+R172Q+N174D, R118D+R172Q+N174D, R172N+L173F+N174Q+G304Q+E345D+E346T+D476G+G477S, A113L+W167F+R172Q+Y363L, T40K+G50A+R172Q+I430E, W167F+R172Q+E346T+Y363L, G50A+A113L+W167F+R172Q, G50A+W167F+R172Q, G50A+A51T+W140Y+R172D, R172E+N174D+A204T+G304Q+R320A+H321N+S323N+E345D+E346T+D476G+G477S, G50A+A51V+W167F+R172E+L173F+N174T+A204V+R320A+S323N, T40K+G50A+A51V+K72R+R172N+N174T+A204T, W140Y+R172E+N174D+A204T+G304Q+E345D+E346T+D476G+G477S, T40K+G50A+W167F+R172N+A204T, G50A+W140Y+R172N+A204T, T40K+G50A+K72R+W167F+R172D+N174T+E345D+G477Q, T40K+G50A+A51V+W167F+R172D+N174E, G50A+A51V+R172D+N174E+M208Y+V213S+V214T+L217M, T40K+G50A+R172D+N174D+E346T+D476K+G477A, G50A+R118P+R172D+N174Q+E346T+D476K+G477A, T40K+K72R+R118P+W167F+G184S+M208Y+V213S+V214T+L217M, Y243D, V165G, R118S+E134D, G50A+A51T+W167F+R172N+L173V, A113L+R118D+W167F+S255I+E345D+G477Q, G50A+A51I+R172D+L173V, W140Y+W167F+N174D, T40K+W167F+R172E+N174D+A204T+G304Q+E345D+E346T+D476G+G477S, A51T+W140Y+R172N+A204V+G304Q+E345D+E346T+D476G+G477S, G50A+W140Y+W167F+R172N+A204T, W167F+R172N+L173F+N174Q+G304Q+E345D+E346T+D476G+G477S, T40K+G50A+W167F+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, G50A+A51V+W140Y+W167F+R172E+L173F+N174T+M208Y+V213S+V214T+L217M, R172D+N174T+K302Q+E346P+D476K+G477S, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E346T+D476K+G477A, G50A+A51V+R172N+N174T+A204T+M208Y+V213S+V214T+L217M, A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, L173T+A204T+E346T+G477A, G50A+A51V+R172D+N174E+E346T+G477A, G50A+A51V+R172D+N174E+R320S+S323N+D476Y, G7Q+T40K+K72R+W167F, R172D+N174Q+E346T+D476K+G477A, G50A+R172D+N174Q+A204V+E345D+D476K+G477S, T40K+G50A+A51V+K72R+W167F+R172D+N174E, T40K+G50A+K72R+W167F+L173T+A204V, G50A+R118P+R172D+N174D+G184S+E346T+D476K+G477A, R172E+L173F+N174D+R320A+S323N, G50A+R172D+N174Q+G304S+E345D+I464T+G465E+D476K+G477Q, G50A+A51T+Q169M+R172E+N174S+M208Y+V213S+V214T+L217M+G304Q+E345D+E346T+D476G+G477S, R172E+L173F+N174D+M208Y+V213S+V214T+L217M, when using SEQ ID NO: 1 for numbering.

In a particular embodiment, the variant has an Improvement Factor (IF) of at least 1.1 when determined in a Model A detergent at a temperature of 40° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: L173T+A204T+R320V+D476Y, G50A+A51V+R172D+N174E+E346T+G477A, G50A+A51V+R172D+N174E+R320V+D476Y, G50A+A51V+W167F+R172E+L173F+N174T+A204V+G304Q+E345D+E346T+D476G+G477T, G7K+T40K+K72R+W167F, W140Y+W167F+A204V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+G304S+E345D+D476K+G477Q, G50A+R172D+N174Q+A204V+E345D+E346P+D476G, G50A+A51T+L85F+R172E+A204T+E345D+E346P+D476G, R172D+N174E+K302Q+E346P+D 476K+G477S, G50A+A51V+R172D+N174E+K302Q+E346P+D476K+G477S, G50A+A51V+R172D+N174E+E346T+D476K+G477A, G50A+A51V+R172D+N174E+G304S+E345D+D476K+G477Q, G50A+A51V+R172D+N174E+E345D+E346P+D476G, G50A+R172D+N174D+M208Y+V213S+V214T+L217M+E346T+D476K+G477A, G50A+A51V+N174S+M208Y+V213S+V214T+L217M, G50A+R118P+R172D+N174Q+G184S+E346T+D476K+G477A, G50A+A51V+R118P+N174S+G184S, T40K+G50A+A51V+W167F+N174S, T40K+G50A+A51V+K72R+W167F+R172E+N174S, T40K+G50A+A51V+K72R+W167F+R172D+N174E, G50A+R172D+N174Q+M208Y+V213S+V214T+L217M+E346T+D476K+G477A, G50A+A51V+R172D+N174E+M208Y+V213S+V214T+L217M, R172N+L173V+E346T+D476K+G477A, R172N+L173V+E345D+E346P+D476G, G50A+R172D+N174 D+R320A+S323M+A442V+D476Y, G50A+R172D+N174Q+G304S+E345D+I464T+G465E+D476K+G477Q, G50A+R172D+N174Q+E346T, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+M208Y+V213S+V214T+L217M, G50A+R172N+N174T+E346T+D476K, when using SEQ ID NO: 1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.3 when determined in a Model A detergent at a temperature of 40° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: T40K, V56T, R171E, A113Y, K269L, K269M, E391A, E391L, H1L, H324L, G149A, K269Q+A274S, L173F, T136N+R172Q+N174D+A204T, G50A+R172E, E345D+E346T+D476Q+G477A, G50A+R172D+N174T+E345D+E346P+D476G, G50A+N174T+E345D+E346P+D476G, R172K+A204V+G304S+E345D+D476K+G477Q, G50A+R172N+A204T+E346T+D476K+G477A, G50A+R172N+A204T+E346T+D432A+D476K+G477A, A51T+R172N+A204V+M208Y+V213S+V214T+L217M+G304Q+E345D+E346T+D476G+G477S, G50A+R172N+A204T+M208Y+V213S+V214T+L217M, G50A+A51V+W167F+R172E+L173F+N174T+A204V+M208Y+V213T+V214T+L217M, G50A+A51T+R172E+A204T+E346T+D476K+G477A, R172D+N174Q+E346T+D476K+G477A, G50A+R172D+N174D+R320A+S323M+A442V+D476Y, when using SEQ ID NO: 1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.1 when determined in a Model J detergent at a temperature of 15° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: Y363L, Y160H, Y160L, W167L, W167F, Q169K, K72S, K72R, T40K, T40M, H1*+H2*+G7S, Q361S, Q361A, E345S, W268F, V291A, V56A, V56T, R171E, R171S, K302A, L351A, L429A, A354V, K347M, K347A, K483R, T282G, N270Y+Y295N, A113Y+R171E, T40K+K179S, R171E+E391L, R171E+Y363L, H1L+R171E+D192E, T40K+R171E, R171E+H324L+Q361A+Y363L, R171E+Q361A+Y363L, R171E+D192E+Y363L, A113Y+R171E+Y363L+E391L+K423P, K423N, I430M, R158A, W140Y, R320A, W140Y+R320A, R172Y, R142K, R172L, R172L, R172S, A113Y, A113L, R172T, R172V, R172A, L173V, Y243E, Y243F, Y243S, Y243G, Y243D, V165G, Y243T, N299K, P211D, K269L, K269N, K269I, K269V, K269M, R118G, R118P, K179M, E391A, E391M, K179S, E391L, E391K, E391H, K179T, H1G, H1W, H1R, H1K, H1S, H1L, K423L, K423H, K423P, K423T, K302R, H1N, N197A, V165M, D192A, D192E, V165A, V165G, V165L, M208A, A186M, H321V, Q98V, H324R, H324W, S323K, H324L, H324Q, H1*+H2E, H1*+H2V, Q280S, H1*+H2A, G304V, V410R, G304A, I430E, G149Q, Q169M, R181G, K461R, N311S, N311L, L351C, N215G, N311E, L351Q, N215G, N311D, D416L, A274W, H210S, H210S+P408Q, H210Y, P211T, P211T+V213T, H210S+V214T, P211T, V213T+V214S, K269Q+A274Y, K269Q+A274G, K269N+A274M, R172Q+N174Q, G50A+R172Q+L173F, R172Q+N174S, R172Q+L173F+N174Y, G50A+A51T+N174E, R172Q+N174T+A204T, N174S, R172Q+L173T+N174D, G50A+R172Q, R172K+L173I+N174S, R172Q+L173F+A204T, G50A+R172Q+N174T, R172Q+N174T, G50A+R172Q+N174D, N174D+A204V, G50A+R172N+N174S, G50A+R172N+N174T, G73V+R172Q+N174E, R172N+L173T+N174Q+S303R, G50S+R172K+N174D, N174T, G50A+A51T+K72H+R172Q+N174Q+A204V, A204V, R172Q, L173T+N174Q+A204V, R172K+L173F, G50A+R172Q+L173T+N174D, G50A+N174E, G50A+A51T+R172Q+L173V, R172Q+L173I+N174S, L173V+N174S, G50A+R172D+N174D, G50A+R172D+N174Q, R172D+N174T, G50A+L173V+A204T, G50A+A51V+R172N+N174T+A204T, E345D+E346P+D476K+G477S, G304Q+D476G+G477K, E346P+D476K+G477S, E345D+D476K+G477S, E346T+D476K+G477A, E345D+E346P+D476G, K302Q+E346P+D476K+G477S, E346T+D476G+G477T, K302N+E345D+D476Q+G477Q, E345D+E346P+G477Q, L173F+N174Q, R172Q+L173F, G50A+N174S, G50A+R172Q+L173V+N174S, G50A+A51V+R172D+L173F, G50A+A51I+R172D+N174*, R172D+L173F, R172D, A51V+R172Q+A204V, G50A+R172K, G50A+R172Q+N174S, G50A+A51V+R172D+L173F+N174Q+A204T, G50S+R172N+N174Q, G50A+R172Q+L173I+N174T+A204T, G50A+R172Q+A204V, L173I+N174D, W48L+A51T+R172Q+N174S+A204T, G50A+A51V+R172E+L173F+N174T+A204V, G50A+R172Q+L444P, R172Q+L173T+N174S, L173F+A204T, A51I+R172D, G50A+R172Q+N174S+A204T, R172Q+L173F+N174D, G50A+A51V+R172Q, R172Q+N174*, G50A+A51T+R172D, G50A+A51T+R172N+N174D+G182V, R172N+N174S, G50A+R172Q+L173F+N174Q, R172E+L173F+N174Q, A51I+R172E+N174T, G50A+A51I+A204V, R172Q+N174D, G50A+R172E+L173V+N174T, A274E+G304Q+G305S+E345D+D476K+G477Q, E346P+D476Q+G477S, E345D+E346P+D476G+G477T, G304Q+E345D+E346T+D476G+G477S, E345D+E346T+D476G+G477A, E346P+D476Q, G304Q+E346T+D476K+G477Q, K302N+G304Q+E346P+D476K, G304A+E346K+G477K, G273V+G304S+E346P+D476G+G477T, K302Q+E345D+E346P+D476K+G477Q, E346P+G477S, G305S+E346P+D476Q+G477T, E345D+E346P+D476Q+G477A, E346T+D476K+G477S, E345D+D476Q+G477S, G304S+E346P+D476K+G477S, E346P+D476K+G477T, G304A+E345D+E346T+D476Q+G477Q, E346T+D476Q+G477A, R118L, R118S+G182A+T193K, R118P+G182A, R118P+G182S, R118S+E134D, E134D+K179L+G182N, R118S, R118S+G182A, R118S+N128L+G182S, E134D, G182S+A186V, R118D+K179L+G182A, R118P+G182N, K179L, R118D+G182N, R118S+E134D+G182S, E134D+A186E, R118S+N128Q+K179L, G182A+P432H, N128F+K179L, K179L+G182A, N128Q+A442V, K179L+A186S, N128Q+K179L+A186S, R118D, E346T+D476Q+G477K, E345D+D476G+G477S, K302Q+G304S+E345D+D476G+G477Q, E345D+E346P+G477S, R172E+L173T+N174T, G50A+R172D+L173T+N174S, G50A+N174 D+A204T, R172Q+L173V, G50A+A51T+R172N+L173I+N174Q, G50A+L173T+N174S+A204V, R172Q+L173F+N174D+A204T, G50A+A51T+R172Q+A204V, G50A+L173F+N174Q+A204V, A51T+L173F+N174Q+A204V, G50A+A51V+L173F+N174E+E471K, R172E+L173F+N174D, A51V+N174T+A204V, R172K+A204V, G50A+A51V+R172Q+A204V, G50A+A51Q+R172Q, A51T+R172N+A204V, G50A+A51T+R172E+L173I, N174Q+A204V, G50A+A51V+L173F+N174*, R172E+N174D+A204T, A51T+R172N+L173*, R172N+L173F+N174Q+V489G, R172E+L173F, G50A+A51T+A87S+R172N+L173T+N174D, G50A+R172N+L173T+N174D, A51T+R172Q, G50A+R172E+L173F+N174D, G50A+R172D+N174T, G50A+A51T+R172Q+N174T, G50A+R172D, A51I+N174D, G50A+N174E+A204T, R172Q+L173V+A204T, A51V+R172Q+L173F+N174S, A51T+R172D+N174Q, G50A+A51T+R172E+N174S, R172E+N174S, G50A+A51T+R172Q+N174Q, G50A+A51T+R172Q+L173F+N174T, A51V+L173V+A204V, E391L+K423P, T40K+V56T+R171E, G149Q+R171E, A113Y+G149Q+R171E+D192E+Q280S, H1L+A113Y+R171E+D192E, R171E+D192E+Q280S+Q361A+Y363L+E391L+K423P, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, R172Q+N174D+G304Q+E345D+E346T+D476G+G477S, K302Q+D476G+G477A, G304A+E346P+D476G+G477S, S255I+E345D+G477Q, E346T+G477A, G304A+E345D, K302Q+E346P+D476K+G477A, K302Q+E346P+G477A, G304S+E345D+D476K+G477Q, K302N+E345D+D476G, A113T+G304S+E345D+E346P+D476G+G477Q, R320S+S323M, G7E+R320M+S323M, D112H+R320A+S323N, K108E+G110D+A111T+D112H+R320S+S323M, R320A+S323N, G7Q+Q98S+R320S+H324Q, G7E+R320A+H324Q, Q98A+R320A+S323N, Q98T+R320A+H324L, G7E+Q98T+R320A, G7E+Q98A+R320M, G7N+R320S, R320A+S323N, G7L+P211Q+S303G+R320A, Q98A+A265S+R320A, G7E+R320A+S323M, G7A+Q98L+R320M, Q98N+R320A+S323N, R320M, G7A+R320M, G7P+R320S+S323K, G7Q+R320A+S323N, G7L+Q98A+R320A+S323M, R320A+H324L, G7S+R320A+S323N+H324Q, Q98S+R320A+S323N, G7K+R320M+H321Q, R320S+S323N, G7N+R320A+S323N, R320M+S323N, G7Q+R320S+H321N+S323M, R320A+H324Q, K302Q+E346P+D476K, K302Q+E346P+G477Q, K302Q+D476G+G477Q, E345D+E346P+D476Q+G477S, G304Q+E345D+E346T, E345D+E346T+D476G+G477S, D112N+R320M+S323M+E449G, V75I+R320A+S323N, G7X+Q98T+R320A, R320T, G7N+Q98S+R320M+S323N, G7Q+R320A+S323R, G7E+Q98A+K302R+R320A+S323N, G7A+R320S+H321W+S323M, A87D+R320S+S323M, Q98K+K269N+R320A+S323M+H324Q, G7K+R320A+P408Q, G7K+R320A+H324L, Q98N+R320S+S323N, R320A+H321W+S323M+H324Q, R320S+S323M+H324Q, G7L+R320M+S323N, N28S+R320M+S323N, Q98S+R320A, G7E+R320A, T5*+G4*+N3*+R320A, G7E+R142I+R320A+E391K+A392P+R393*+Q394S, G7L+R320A+S323M, G7E+R320A+P320Q+S323M, G7K+R320A+S323N+P364S, G7Q+R320A+H321Q+S323MR320V+D476Y, G7N+R320M, G7H+Q98S+R320A+S323M+H324L, G7Q+R320S+H321N, R320A+H321N+S323N, G7E+R320M, Q98A+R320A+S323M, G7Q+Q98T+R320A+S323M, R320M+S323M+H324Q, G7K+R320A, G7K+T246M+R320A+S323M, G7Q+Q98A+R320M+S323M, G7Q+Q86H+R320A+S323M, G50A+A51Q+R172Q+G304Q+E345D+E346T+D476G+G477S, R172E+N174D+A204T+G304Q+E345D+E346T+D476G+G477S, G50A+A51V+R172Q+A204V+G304Q+E345D+E346T+D476G+G477S, A51T+R172N+A204V+G304Q+E345D+E346T+D476G+G477S, G50A+A51T+A87S+R172N+L173T+N174D+G304Q+E345D+E346T+D476G+G477S, A51T+R172D+N174Q+G304Q+E345D+E346T+D476G+G477S, G50A+A51T+R172E+N174S+G304Q+E345D+E346T+D476G+G477S, G50A+A51T+R172Q+N174S+A204V, G50A+R172D+N174S, A51V+R172D, L173T+N174T+A204T, G50A+R172E+L173V+N174S, R172E+N174T, N28D+G109S+A119P+R172D+L173V+N174S, R172E+N174D, A51I+R172E+L173F, R172Q+A204V, G50A+A51T+R172E+N174T, R172N+N174T+A204V, R172K+N174S, R172D+N174S, R172D+L173V+N174D, G50A+A51T+R172Q+L173I, R172Q+N174E, G50A+R172E+L173T+N174S, G50A+A51T+R172E+A204V, R172D+N174D, L173V+N174D, R172D+L173F+N174D, G50A+R172E+N174T, L173F+N174Q+A204V, L173T+N174S+A204V+T255K, G50A+R172N+A204T, R172E+L173I+A204T, G50A+R172Q+L173V, A51T+R172D+N174D, R172K+L173V+N174S+A204T, R172E+L173T+N174S+A204T, G50A+A51V+R172D+N174T, R172D+L173I+N174S, L173T+N174T+A204V, R172N+N174D, G50A+L173I+L173I+N174T+N174T+N219K+W220F, G50A+R172E+L173F+N174Q, R172Q+N174T, G50A+R172N+N174E, R172N+N174T+H324N, G50A+R172Q+L173T+N174Q, R172K+L173V+N174S+A204T+L429F, G50A+R172E+L173F+N174T, R172D+L173V, R172E+L173T+N174S, R172D+L173I+N174S, A51V+R172Q+L173T, G50A+A51T+R172Q+L173V+A204V, G50A+A51T+R172D+L173F+N174T+K423P, N174E, T40I+A51L+R172D+L173F, G50A+R172E+L173F+N174Q+A204T, R172K+L173F+N174Q+A204V, R172N+L173I, G50A+R118D+W167F+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51T+A113L+W167F+R172N+L173V, G50A+A51T+W167F+R172N+L173V, G50A+A51T+A113L+R172N+L173V, G50A+A51T+R118D+R172N+L173V, A113L+R172Q+N174D, R118D+W167F+R172Q+N174D, R118D+R172Q+N174D, W167F+R172Q+N174D, A204T+G304Q+E345D+E346T+D476G+G477S, R172N+L173F+N174Q+G304Q+E345D+E346T+D476G+G477S, A113L+W167F+R172Q+Y363L, T40K+G50A+R172Q+I430E, T40K+E346T, T40K+K72R+W167F, W167F+R172Q+E346T+Y363L, K72R+A113L+W167F+R172Q+N215G+Y363L, W167F+R172Q+E346T, A113L+W167F+R172Q+N215G, S255I+R320A+H321N+S323N+E345D+G477Q, G50A+A113L+W167F+R172Q, G50A+W167F+R172Q, G50A+A113L+R118D+R172Q, G50A+A113L+W167F+R172QG50A+R172D+L173F+E471K, G50A+A51T+R172D+E345D+E346P+D476G, G50A+R172D+N174Q+E345D+E346P+D476G, R171E+D192E+E345D+E346P+D476G, G50A+R172D+N174D+E345D+E346P+D476G, G50A+R172D+N174T+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477Q, R171E+D192E+S255I+E345D+G477Q, G50A+R172D+N174D+S255I+E345D+G477Q, G50A+A51V+R172N+N174T+A204T+S255I+E345D, G50A+R172D+N174T+S255I+E345D+G477Q, R172K+A204V+S255I+E345D+G477Q, R172K+A204V+S255I+E345D+K423P+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51T+W140Y+R172N+L173V, W140Y+R172D+N174T, G50A+W140Y+R172N+N174T, G50A+A51V+W140Y+P146S+L173F+N174D, A113L+S255I+E345D+G477Q+V489G, A113L+R118D+W167F+S255I+E345D+G477Q, K72R+A113L+S255I+E345D+G477Q, K72R+A113L+W167F+S255I+E345D+G477Q, Q98N+Q169M+R320A+S323M, Q98N+R320A+H321N+S323N, Q98N+M208Y+V213S+V214T+L217M+R320A+S323M, Q98N+M208Y+V213T+V214T+L217M+R320A+S323M, R172E+N174D+A204T+G304Q+E345D+E346T+E391L+D476G+G477S, R172E+N174D+A204T+G304Q+E345D+E346T+E391L, R172E+N174D+A204T+G304Q+R320A+H321N+S323N+E345D+E346T+D476G+G477S, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+M208Y+V213S+V214T+L217M+G304Q+E345D+E346T+D476G+G477S, R172N+M208Y+V213T+V214T+L217M+G304Q+E345D+E346T+D476G+G477S, G50A+A51T+A87S+Q169M+R172N+L173T+N174D+G304Q+E345D+E346T+D476G+G477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S 323N, G50A+R172N+N174T+R320A+H321N+S323N, G50A+A51V+L173F+N174D+R320A+H321N+S323N, W167F+E345D+D476K+G477S, R172E+N174S+G304Q+R320A+H321N+S323N+E345D+E346T+D476G+G477S, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R 172E+N174S+M208Y+V213T+V214T+L217M+G304Q+E345D+E346T+D476G+G477S, R172E+N174D+A204T+G304Q+R320A+S323N+E345D+E346T+D476G+G477S, G50A+A51V+W167F+R172E+L173F+N174T+A204V+R320A+S323N, G50A+A51V+W167F+R172E+L173F+N174T+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+M208Y+V213S+V214T+L217M, G50A+A51T+W167F+R172N+L173V+M208Y+V213T+V214T+L217M, R172N+L173F+N174Q+G304Q+R320A+S323N+E345D+E346T+D476G+G477S, T40K+K72R+W167F+R320A+S323N, T40K+K72R+W140Y+W167F, V165G, N215G, P211T, N174T, G50A+R172N+A204T, R320A+S323N, G7L+A51S+R320S+S323M, G50A+R172E+L173F+N174T, R172D+L173I+N174SR172Q+N174T, T40K+K72R+W167F+M208Y+V213T+V214T+L217M+R320A+S323N, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+L173T+N174S, G50A+A51T+R172N+A204T, G50A+A51S+R172D+N174Q, G50A+D112N, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51T+R172D+R320A+S323N, G50A+R172Q+L173T+E346T+G477A, R172Q+L173T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V, G50A+A51T+R172E+A204T+E346T+D476K+G477A, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+G304S+E345D+D476K+G477Q, G50A+R172D+N174Q+A204V+E345D+E346P+D476G, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, T40K+G50A+R172D+N174D+E346T+D476K+G477A, G50A+R118P+R172D+N174D+G184S+E346T+D476K+G477AR172E+L173F+N174D+G304S+E345D+D476K+G, 477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V+D476Y, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, G50A+R172D+N174D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, G7K+T40K+K72R+W167F+R320A+S323N+P364S, when using SEQ ID NO: 1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.3 when determined in a Model J detergent at a temperature of 15° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: W167F, K72S, K72R, T40K, T40M, Q361S, V291A, V56T, R171E, R171S, H1L+R171E+D192E, T40K+R171E, R171E+H324L+Q361A+Y363L, A113Y+R171E+Y363L+E391L+K423P, W140Y, R172S, A113Y, A113L, R172T, Y243G, Y243D, K269M, R118P, E391A, E391M, K179S, E391L, E391H, K179T, H1G, K423P, H324R, I430E, N215G, H210Y, P211T, H210S+V214T, G50A+R172Q, G73V+R172Q+N174E, G50S+R172K+N174D, G50A+A51T+K72H+R172Q+N174Q+A204V, R172Q, L173T+N174Q+A204V, G50A+A51T+R172Q+L173V, G50A+R172D+N174D, G50A+R172D+N174Q, R172D+N174T, G50A+A51V+R172N+N174T+A204T, E346P+D476K+G477S, E345D+E346P+G477Q, R172Q+L173F, G50A+N174S, G50A+R172Q+L173V+N174S, G50A+A51V+R172D+L173F, R172D+L173F, R172D, G50A+R172K, G50A+A51V+R172D+L173F+N174Q+A204T, G50A+R172Q+L173I+N174T+A204T, G50A+A51V+R172E+L173F+N174T+A204V, R172Q+L173F+N174D, G50A+A51V+R172Q, R172Q+N174*, G50A+A51T+R172D, G50A+R172E+L173V+N174T, A274E+G304Q+G305S+E345D+D476K+G477Q, K302N+G304Q+E346P+D476K, K302Q+E345D+E346P+D476K+G477Q, E346T+D476Q+G477A, R118L, R118S+G182A+T193K, R118P+G182A, R118P+G182S, R118S+E134D, E134D+K179L+G182N, R118S+G182A, G182S+A186V, R118D+K179L+G182A, R118P+G182N, R118S+E134D+G182S, E134D+A186E, N128Q+K179L+A186S, R118D, R172Q+L173V, R172E+L173F+N174D, G50A+A51T+R172E+L173I, R172E+N174D+A204T, A51T+R172N+L173*, R172E+L173F, G50A+A51T+A87S+R172N+L173T+N174D, G50A+R172D+N174T, G50A+R172D, R172Q+L173V+A204T, A51T+R172D+N174Q, G50A+A51T+R172E+N174S, G149Q+R171E, A113Y+G149Q+R171E+D192E+Q280S, H1L+A113Y+R171E+D192E, R171E+D192E+Q280S+Q361A+Y363L+E391L+K423P, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, R172Q+N174D+G304Q+E345D+E346T+D476G+G477S, S255I+E345D+G477Q, E346T+G477A, G304S+E345D+D476K+G477Q, A113T+G304S+E345D+E346P+D476G+G477Q, G7Q+Q98S+R320S+H324Q, Q98A+R320A+S323N, G7N+R320S, R320A+S323N, G7L+P211Q+S303G+R320A, Q98A+A265S+R320A, R320M, G7P+R320S+S323K, G7Q+R320A+S323N, R320A+H324Q, G7X+Q98T+R320A, G7E+Q98A+K302R+R320A+S323N, G7K+R320A+P408Q, G7L+R320M+S323N, G7L+R320A+S323M, G7K+R320A+S323N+P364S, G7Q+R320A+H321Q+S323M, R320V+D476Y, G7H+Q98S+R320A+S323M+H324L, G7Q+R320S+H321N, R320A+H321N+S323N, Q98A+R320A+S323M, G7Q+Q98T+R320A+S323M, R172E+N174D+A204T+G304Q+E345D+E346T+D476G+G477S, G50A+A51V+R172Q+A204V+G304Q+E345D+E346T+D476G+G477S, A51T+R172N+A204V+G304Q+E345D+E346T+D476G+G477S, G50A+A51T+A87S+R172N+L173T+N174D+G304Q+E345D+E346T+D476G+G477S, A51T+R172D+N174Q+G304Q+E345D+E346T+D476G+G477S, G50A+A51T+R172E+N174S+G304Q+E345D+E346T+D476G+G477S, R172Q+A204V, R172N+N174T+A204V, R172D+N174S, R172D+L173V+N174D, G50A+A51T+R172E+A204V, R172D+L173F+N174D, G50A+R172E+N174T, L173F+N174Q+A204V, G50A+R172N+A204T, R172E+L173I+A204T, A51T+R172D+N174D, R172K+L173V+N174S+A204T, R172E+L173T+N174S+A204T, G50A+A51V+R172D+N174T, R172D+L173I+N174S, L173T+N174T+A204V, R172N+N174D, G50A+A51T+R172Q+L173V+A204V, G50A+A51V+R118D+R172N+N174T+A204T, G50A+A51V+A113L+R118D+R172D+L173F, G50A+A113L+W167F+R172Q+L173I+N174T+A204T, G50A+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51T+A113L+W167F+R172N+L173V, G50A+A51T+W167F+R172N+L173V, G50A+A51T+A113L+R172N+L173V, R118D+W167F+R172Q+N174D, W167F+R172Q+N174D, A204T+G304Q+E345D+E346T+D476G+G477S, R172N+L173F+N174Q+G304Q+E345D+E346T+D476G+G477S, A113L+W167F+R172Q+Y363L, T40K+G50A+R172Q+I430E, T40K+K72R+W167F, W167F+R172Q+E346T+Y363L, K72R+A113L+W167F+R172Q+N215G+Y363L, G50A+R172D+L173F+E471K, G50A+A51T+R172D+S255I+E345D+G477Q, G50A+R172D+N174T+S255I+E345D+G477Q, R172K+A204V+S255I+E345D+G477Q, R172K+A204V+S255I+E345D+K423P+G477Q, G50A+A51T+W140Y+R172D, G50A+A51V+W140Y+R172E+L173F+N174T+A204V, G50A+W140Y+R172D+N174Q, G50A+W140Y+R172N+N174T, G50A+A51V+W140Y+P146S+L173F+N174D, A113L+R118D+W167F+S255I+E345D+G477Q, K72R+A113L+W167F+S255I+E345D+G477Q, Q98N+M208Y+V213T+V214T+L217M+R320A+S323M, R172E+N174D+A204T+G304Q+E345D+E346T+E391L+D476G+G477S, R172E+N174D+A204T+G304Q+R320A+H321N+S323N+E345D+E346T+D476G+G477S, A51T+R172N+A204V+G304Q+R320A+H321N+S323N+E345D+E346T+D476G+G477S, A51T+R172N+M208Y+V213S+V214T+L217M+G304Q+E345D+E346T+D476G+G477S, R172N+M208Y+V213T+V214T+L217M+G304Q+E345D+E346T+D476G+G477S, G50A+A51T+A87S+Q169M+R172N+L173T+N174D+G304Q+E345D+E346T+D476G+G477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+R172N+N174T+R320A+H321N+S323N, G50A+A51T+R172E+N174S+G304Q+R320A+H321N+S323N+E345D+E346T+D476G+G477S, G50A+A51T+A113L+R172E+N174S+G304Q+E345D+E346T+V474T, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+M208Y+V213S+V214T+L217M, G50A+A51T+W167F+R172N+L173V+M208Y+V213T+V214T+L217M, R172N+L173F+N174Q+G304Q+R320A+S323N+E345D+E346T+D476G+G477S, T40K+K72R+W167F+R320A+S323N, R172D+L173I+N174S, G50A+A51I+R172D+L173V, G50A+A51L+R172N+L173V+A204T, R172D+L173T+N174D+A204T, R172N+L173V, G50A+R172K+N174Q, T5K+R172E+L173F, R172D+L173T+N174D, G50A+A51V+A113L+R172E+L173F+N174T+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+R118D+W167F, T40K+A51T+R172N+A204V+G304Q+E345D+E346T+D476G+G477S, T40K+G50A+W167F+R172N+N174S+A204T, K72R+W167F+E346T+D476K+G477A+Y482F, G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+K72R+W167F+N174D+A204T, W140Y+W167F+N174D, G50A+W140Y+W167F+N174D+A204T, T40K+K72R+W167F+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W167F+R172D+N174T+A204V+S255I+E345D+G477Q, W140Y+W167F+R172D+N174T+A204V+S255I+E345D+E346P+G477Q, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+E345D+D476K+G477S, G50A+R172D+N174Q+E345D+D476K+G477S, R172D+N174T+K302Q+E346P+D476K+G477S, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, T40K+K72R+W167F+S255I+E345D+G477Q, T40K+K72R+W167F+G304S+E345D+D476K+G477Q, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213S+V214T+L217M+G304Q+E345D+E346T+D476G+G47 7S, A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, G50A+A51V+W167F+R172E+L173F+N174T+A204V+M208Y+V213T+V214T+L217M+R320A+S323N, G50A+A51V+R172N+N174T+A204T+E346T+G477A, G50A+A51T+R172N+L173V+R320A+S323M+D476Y, G50A+A51T+R172D+R320A+S323N, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, T40K+K72R+W167F+E346T+G477A, T40K+K72R+W167F+R320V+D476Y, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+W167F+A204V, R172K+M208Y+V213T+V214T+L217M, R172K+M208Y+V213S+V214T+L217M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+R172Q+L173T+G304Q+E345D+E346T+D476G+G477T, G50A+A51T+L85F+R172N+L173V+Q319K+R320A+S323N, R172D+N174Q+E346T+D476K+G477A, G50A+R172D+N174Q+A204V+E345D+D476K+G477S, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, G50A+R172D+N174Q+M208Y+V213S+V214T+L217M+E346T+D476K+G477A, T40K+G50A+R172D+N174D+E346T+D476K+G477A, G50A+R118P+R172D+N174D+G184S+E346T+D476K+G477A, R172E+L173F+N174D+E346T+G477A, R172N+L173V+E345D+D476K+G477S, R172N+L173V+S255I+E345D+G477Q, G50A+R118P+R172D+N174Q+E346T+D476K+G477A, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+T246M+S255I+E345D+G477Q, T5K+R172E+L173F+K302Q+E346P+D476K+G477S, T5K+R172E+L173F+K302Q+E346P, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+M208Y+V213S+V214T+L217M, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, G50A+R172N+N174T+E346T+D476K, R118P+W140Y, when using SEQ ID NO: 1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.5 when determined in a Model J detergent at a temperature of 15° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: Y363L, W167F, R171E, W140Y, R118P, E391A, E391L, E391H, K179T, G50A+A51T+K72H+R172Q+N174Q+A204V, G50A+R172D+N174D, G50A+R172D+N174Q, G50A+A51V+R172D+L173F, R118S+G182A+T193K, R118P+G182A, R118P+G182S, N128Q+K179L+A186S, R118D, R171E+D192E+Q280S+Q361A+Y363L+E391L+K423P, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G7L+P211Q+S303G+R320A, G7L+R320A+S323M, G7K+R320A+S323N+P364S, R320A+H321N+S323N, R172E+N174D+A204T+G304Q+E345D+E346T+D476G+G477S, G50A+A51V+R172Q+A204V+G304Q+E345D+E346T+D476G+G477S, A51T+R172N+A204V+G304Q+E345D+E346T+D476G+G477S, G50A+A51T+A87S+R172N+L173T+N174D+G304Q+E345D+E346T+D476G+G477S, A51T+R172D+N174Q+G304Q+E345D+E346T+D476G+G477S, G50A+A51T+R172Q+L173V+A204V, G50A+A51V+A113L+R118D+R172D+L173F, G50A+A51V+W167F+R172E+L173F+N174T+A204V, G50A+A51T+W167F+R172N+L173V, W167F+R172Q+E346T+Y363L, G50A+A51T+W140Y+R172D, G50A+W140Y+R172N+N174T, R172E+N174D+A204T+G304Q+E345D+E346T+E391L+D476G+G477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51T+R172E+N174S+G304Q+R320A+H321N+S323N+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, T40K+K72R+W167F+R320A+S323N, G50A+A51I+R172D+L173V, R172D+L173T+N174D+A204T, R172N+L173V, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+G50A+A51V+K72R+R172N+N174T+A204T, T40K+K72R+R172E+N174D+A204T+G304Q+E345D+E346T+D476G+G477S, T40K+K72R+R172N+L173F+N174Q+G304Q+E345D+E346T+D476G+G477S, G50A+A51V+R172D+L173F+A204V+K302Q+D476G+G477S, T40K+K72R+W167F+R172K+A204V, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+W167F+R172D+N174T+E345D+E346P+D476G, T40K+G50A+K72R+W167F+R172D+N174T+E345D+G477Q, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51V+A113L+R172E+L173F+N174T+A204V+E345D+L405V+T414K, R172D+N174T+K302Q+E346P+D476K+G477S, R172K+A204V+E345D+D476K, R172K+A204V+E345D+D476K+G477S, R172E+N174D+A204T+M208Y+V213S+V214T+L217M+G304Q+E345D+E346T+D476G+G47 7S, G50A+A51V+W167F+R172E+L173F+N174T+A204V+E346T+G477A, G50A+A51V+A113L+R172E+L173F+N174T+A204V+E346T+G477A, T40K+K72R+W167F+E346T+G477A, L173T+A204T+E346T+G477A, G50A+A51V+R172D+N174E+E346T+G477A, G50A+A51V+R172D+N174E+R320V+D476Y, G50A+A51V+R172D+N174E+R320S+S323N+D476YG50A+A51V+W167F+R172E+L173F+N1 74T+A204V+G304, +E345D+E346T+D476G+G477T, G7Q+T40K+K72R+W167F, W167F+Q169M+R172E+N174S, W140Y+W167F+A204V, G7K+W167F+R320M+H321Q, G7K+K72R+W167F+R320M+H321Q, R172D+N174Q+E346T+D476K+G477A, G50A+A51V+R172D+N174E+E346T+D476K+G477A, R172E+A204T+G304S+E345D+D476K+G477Q, G50A+R172D+N174D+M208Y+V213S+V214T+L217M+E346T+D476K+G477A, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, G50A+R118P+R172D+N174D+G184S+E346T+D476K+G477A, R172N+L173V+E345D+D476K+G477S, R172N+L173V+S255I+E345D+G477Q, G50A+R118P+R172D+N174Q+E346T+D476K+G477A, G50A+R172D+N174 D+R320A+S323M+A442V+D476Y, G50A+R172D+N174Q+G304S+E345D+I464T+G465E+D476K+G477Q, G50A+R172D+N174Q+R320A+S323N+Y482F, G50A+R172D+N174Q+E346T, G50A+R172D+N174Q+R320V, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, T40K+K72R+R118P+W167F+G184S+M208Y+V213S+V214T+L217M, G50A+R172D+N174D+E346T+D476K, when using SEQ ID NO: 1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.1 when determined in a Model J detergent at a temperature of 30° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: T40K, Q361S, Q361A, E345S, E276S, W268F, V291A, V56A, V56T, R171E, K302A, L429A, A354V, K347M, A113Y+R171E, T40K+K179S, R171E+E391L, H1L+R171E+D192E, T40K+R171E, E471N, W140Y, R320A, A113Q, R172Y, A113N, R142K, R172L, R172L, R172S, A113Y, A113L, R142T, R172T, R172V, R172A, N174L, I241F, N174Y, N174G, Y243E, Y243I, Y243S, Y243G, Y243D, V165G, Y243T, N299K, Y160Q, P211D, K269L, K269N, K269R, F155Y, K269I, K269V, K269M, R118G, R118P, K179P, K179R, E391A, E391M, E391L, E391H, K179T, H1G, H1R, H1K, H1S, H1L, L217M, K423L, K423H, K423P, K423T, L217F, K302R, N296Q, H1N, D192E, N281Q, V165A, V165G, A186V, N281L, N281V, A186T, H321L, H324R, H324L, G7L, N128H, G182N, T193G, H1*+H2E, H1*+H2V, Q280S, G304R, V410R, G304A, G305S, I430E, G149Q, G149A, D476G, Q169M, R171H, N311S, N215G, N311E, G182A+V213T+V214S, H210S, H210Y, P211T+V213T, G182N+V213S, H210S+V214T, K269Q+A274W, K269H+A274C, K269N+A274M, G50A+R172Q+L173F, R172Q+L173F+N174Y, N174S, G50A+R172Q, R172K+L173I+N174S, G50A+A51T+R172Q, G50A+R172Q+L173T, G50A+R172Q+L173V, G50A+R172Q+N174D, G50A+R172N+N174S, G50A+R172N+N174T, R172Q, L173T+N174Q+A204V, G50A+R172Q+L173T+N174D, G50A+A51V+R172N+N174T+A204T, K302Q+E346P+D476K+G477S, G304Q+E346P+D476G, G304A+E346P+D476K+G477A, E346T+D476G+G477T, K302N+E345D+D476Q+G477Q, E345D+E346P+G477Q, R172Q+L173F, G50A+N174S, N174D, G50A+R172Q+A204T, G50A+R172Q+N174Q, G50A+A51V+R172D+L173F, G50A+A51I+R172D+N174*, R172D+L173F, A51V+R172Q+A204V, G50A+R172K, G50A+R172Q+N174S, G50S+R172N+N174Q, G50A+R172Q+L173I+N174T+A204T, G50A+R172N+A204T, L173I+N174D, W48L+A51T+R172Q+N174S+A204T, G50A+A51V+R172E+L173F+N174T+A204V, G50A+R172Q+L444P, L173F+A204T, R172Q+L173F+N174D, G50A+A51V+R172Q, R172Q+N174*, G50A+A51T+R172D, G50A+R172E+L173F, L173V+N174T, G50A+A51T+R172Q+N174E, G50A+R172Q+L173F+N174Q, R172E+L173F+N174Q, A51I+R172E+N174T, G50A+A51I+A204V, R172Q+N174D, G50A+L173F+N174S, G50A+R172E+L173V+N174T, G50A+A51T+L173F, A274E+G304Q+G305S+E345D+D476K+G477Q, E345D+D476Q+G477K, G304A+E345D+D476Q+G477A, G304Q+E345D+E346T+D476G+G477S, E346T, K302N+E345D+D476G+G477K, K302N+G304Q+E346P+D476K, G304Q+E345D+E346T+D476G+G477T, K302N+E346P+D476Q+G477K, E345D+D476Q, G304S+E345D+E346T+D476Q+G477A, E346P+D476K+G477T, G304A+E345D+E346T+D476Q+G477Q, G304S+E346P+D476K+G477K, K302Q+E346P+D476K+G477T, E346T+D476Q+G477A, G304A+E345D+E346P+D476K+G477Q, R118S+G182A+T193K, R118P+G182A, E134D+K179L+G182N, G4V+E134D+K179L, E134D+A186E, N128F+K179L, N128Q+A442V, N128Q+K179L+A186S, R118D, E345D+D476Q+G477A, G304Q+E346P+D476G+G477S, E346T+D476K+G477Q+V489G, E346T+D476Q+G477K, E345D+D476G+G477S, D476K+G477Q, K302Q+G304S+E345D+D476G+G477Q, E345D+D476Q+G477Q, G50A+R172Q+L173I, G50A+A51V+L173F+N174D, A51V+R172Q+N174S+A204V, G50A+N174D+A204T, G50A+A51T+R172N+L173I+N174Q, G50A+L173T+N174S+A204V, G50A+A51T+R172Q+A204V, G50A+L173F+N174Q+A204V, R172K+A204V, G50A+A51V+R172Q+A204V, G50A+A51Q+R172Q, A51T+R172N+A204V, G50A+A51T+R172E+L173I, N174Q+A204V, G50A+A51V+L173F+N174*, R172E+N174D+A204T, A51T+R172N+L173*, R172N+L173F+N174Q+V489G, G50A+R172S+N174H, G50A+A51T+A87S+R172N+L173T+N174D, G50A+R172E+L173F+N174D, G50A+R172D+N174T, G50A+A51T+R172Q+N174T, G50A+R172D, G50A+N174E+A204T, R172Q+L173V+A204T, G50A+A51T+R172E+N174S, G50A+A51T+R172Q+N174Q, A51V+L173V+A204V, A113Y+G149Q+R171E+D192E+Q280S, G50A+R172Q+N174T+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, R172Q+N174D+G304Q+E345D+E346T+D476G+G477S, K302Q+G304A+E345D+E346T+D476Q+G477Q, G305A+E345D, K302Q+E345D+D476K+G477Q, S255I+E345D+G477Q, E346T+G477A, G304A+E345D, K302Q+E346P+D476K+G477A, G305S+E345D+D476K+G477Q, K302N+E345D+E346P+D476Q+G477S, G304A+E346T+D476K+G477T, G304S+E345D+D476K+G477Q, R320S+S323M, K108E+G110D+A111T+D112H+R320S+S323M, Q98S+R320S, R320A+S323N, G7K+R320M+H321Q, R320S+S323N, R320A+H324Q, G304A+E346P+D476Q+G477T, K302Q+G304Q+D476K+G477K, G305S+E346P+D476K+G477Q, G304Q+E345D+E346T, R320A+H321W+S323M+H324Q, R320S+S323M+H324Q, Q98S+R320A, T5*+G4*+N3*+R320A, G7L+R320A+S323M, G7K+R320A+S323N+P364S, R320V+D476Y, G7H+Q98S+R320A+S323M+H324L, R320A+H321N+S323N, G7Q+Q98T+R320A+S323M, R172E+N174D+A204T+G304Q+E345D+E346T+D476G+G477S, G50A+A51V+R172Q+A204V+G304Q+E345D+E346T+D476G+G477S, A51T+R172N+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172N+L173V, R172E+N174T, R172Q+A204V, G50A+A51T+R172E+N174T, R172Q+L173F+N174T, R172K+N174S, R172Q+N174E, G50A+A51T+R172E+A204V, L173F+N174Q+A204V, L173T+N174S+A204V+T255K, G50A+R172N+A204T, R172E+L173I+A204T, G50A+R172Q+L173V, A51T+R172D+N174D, R172N+N174D, G50A+A51V+R172Q+N174Q, G50A+R172Q+L173T+N174Q, R172K+L173V+N174S+A204T+L429F, R172K+L173T, G50A+A51T+R172Q+L173V+A204V, R172Q+L173T, R172K+L173F+N174Q+A204V, R158G+N174E, A51T+N174D+A204V, G50A+R118D+W167F+R172D+N174Q, G50A+A51V+W167F+R172N+N174T+A204T, G50A+A51V+A113L+R172N+N174T+A204T, G50A+A51V+A113L+R118D+R172D+L173F, G50A+W167F+R172Q+L173I+N174T+A204T, G50A+A113L+R172Q+L173I+N174T+A204T, G50A+A51V+W167F+R172E+L173F+N174T+A204V, G50A+A51V+A113L+R172E+L173F+N174T+A204V, G50A+A51T+A113L+W167F+R172N+L173V, G50A+A51T+W167F+R172N+L173V, G50A+A51T+A113L+R172N+L173V, W167F+R172Q+N174D, A204T+G304Q+E345D+E346T+D476G+G477S, R172N+L173F+N174Q+G304Q+E345D+E346T+D476G+G477S, T40K+G50A+R172Q+I430E, T40K+E346T, T40K+K72R+W167F, A113L+W167F+R172Q+N215G, G50A+A113L+W167F+R172Q, G50A+W167F+R172Q, G50A+A113L+W167F+R172Q, G50A+A51T+R172D+E345D+E346P+D476G, G50A+R172D+N174Q+E345D+E346P+D476G, R171E+D192E+E345D+E346P+D476G, G50A+R172D+N174D+E345D+E346P+D476G, G50A+R172D+N174T+E345D+E346P+D476G, G50A+R172N+N174T+E345D+E346P+D476G, G50A+A51V+R172N+N174T+A204T+S255I+E345D, R172K+A204V+S255I+E345D+G477Q, R172K+A204V+S255I+E345D+K423P+G477Q, G50A+A51T+W140Y+R172D, G50A+W140Y+R172D+N174Q, G50A+A51V+W140Y+R172N+N174T+A204T, G50A+W140Y+R172N+N174T, G50A+A51V+W140Y+P146S+L173F+N174D, A113L+S255I+E345D+G477Q+V489G, K72R+A113L+W167F+S255I+E345D+G477Q, W167F+S255I+E345D+G477Q, Q98N+M208Y+V213S+V214T+L217M+R320A+S323M, Q98N+M208Y+V213T+V214T+L217M+R320A+S323M, R172E+N174D+A204T+G304Q+E345D+E346T+E391L+D476G+G477S, G50A+A51T+A87S+Q169M+R172N+L173T+N174D+G304Q+E345D+E346T+A427V+D476G+G477S, G50A+R172D+N174Q+R320A+H321N+S323N, G50A+A51T+R172E+N174S+G304Q+E345D+E346T+E391L+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+M208Y+V213S+V214T+L217M, G50A+A51V+W167F+R172E+L173F+N174T+M208Y+V213T+V214T+L217M, G50A+A51T+W167F+R172N+L173V+M208Y+V213T+V214T+L217M, T40K+K72R+W167F+R320A+S323N, T40K+K72R+W140Y+W167F, L173F, Y160L, V165G, G50A+R172Q+L173V, G50A+R172N+A204T, G50A+A51I+R172D+L173V, R172D+L173T+N174D+A204T, R172N+L173V, T5K+R172E+L173F, A47S+G50A+R172K+L173F+N174D, R172D+L173T+N174D, G50A+A51T+R172N+L173V+E345D+E346P+D476G, N174D+A204T+E346T+D476K+G477A, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+G50A+W167F+R172N+N174S+A204T, T40K+K72R+E345D+E346P+D476G, K72R+W167F+E346T+D476K+G477A+Y482F, T40K+K72R+Q98N+W167F+R320A+S323M, T40K+G50A+A51V+R172N+N174T+A204T, T40K+G50A+A51V+K72R+R172N+N174T+A204T, T40K+K72R+R172N+L173F+N174Q+G304Q+E345D+E346T+D476G+G477S, G50A+R172N+N174T+E346T+D476K+G477A, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, W140Y+R172E+N174D+A204T+G304Q+E345D+E346T+D476G+G477S, T40K+G50A+W167F+R172N+A204T, G50A+W140Y+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, T40K+K72R+W167F+S255I+E345D+G477Q, T40K+K72R+W167F+G304S+E345D+D476K+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N1 74T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+R172N+N174T+A204T+E346T+G477A, R172N+L173V+E346T+G477A, G50A+A51T+R172N+L173V+R320A+S323M+D476Y, G50A+R172Q+L173T+E346T+G477A, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, T40K+K72R+W167F+E346T+G477A, T40K+K72R+W167F+R320V+D476Y, G50A+A51V+R172E+N174S+E346T+G477A, G50A+A51T+R172E+A204T+R320V+N457T+D476Y, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+E346T+G477A, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, T40K+K72R+W167Y, A51T+K72R+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+R172D+N174D+R320V, R172D+N174Q+E346T+D476K+G477A, G50A+R172D+N174Q+A204V+E345D+D476K+G477S, L173T+A204T+G304S+E345D+D476K+G477Q, G50A+R172D+N174Q+A204V+E345D+E346P+D476G, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+G304S+E345D+D476K+G477Q, G50A+A51V+R172D+N174E+E345D+E346P+D476G, G50A+A51T+R172E+A204T+E345D+D476K+G477S, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+N174S+G184S, G50A+R118P+R172E+G184S+A204T, T40K+G50A+A51V+W167F+N174S, T40K+G50A+A51V+K72R+W167F+R172E+N174S, T40K+G50A+A51V+K72R+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+R172T+N174S+E346T, G50A+R118P+R172D+N174Q+E346T+D476K+G477A, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+E346T, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+R172N+L173F+N174Q+G304Q+E345D+E346T+D476G, G50A+R172N+N174T+E346T+D476K, K72R+W167F+E346T+D476K, R118P+W140Y, G7K+T40K+W167F+R320A+S323N+P364S, when using SEQ ID NO: 1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.3 when determined in a Model J detergent at a temperature of 30° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: Y363L, Q361S, Q361A, E345S, E276S, V291A, V56A, V56T, R171E, T40K+R171E, G149Q+R171E+Q361A+Y363L+K423P, W140Y, R172Y, A113N, R142K, R172L, R172L, R172S, A113Y, A113L, R172T, R172V, Y243S, Y243G, Y243D, V165G, Y243T, K269V, K269M, E391M, E391L, H1K, H1S, H1L, L217M, K423T, L217F, N296Q, H1N, V165A, G304R, V410R, G305S, I430E, G149Q, N215G, N174S, G50A+R172Q, G50A+R172Q+L173T, G50A+A51V+R172D+L173F, G50A+A51I+R172D+N174*, R172D+L173F, G50A+R172K, G50A+R172Q+N174S, G50S+R172N+N174Q, G50A+R172Q+L173I+N174T+A204T, G50A+R172E+L173F, L173V+N174T, G50A+A51T+R172Q+N174E, G50A+R172Q+L173F+N174Q, R172E+L173F+N174Q, A51I+R172E+N174T, G50A+A51I+A204V, R172Q+N174D, G50A+L173F+N174S, G50A+R172E+L173V+N174T, G50A+A51T+L173F, E345D+D476Q+G477K, G304Q+E345D+E346T+D476G+G477S, E346T, E345D+D476Q, G304A+E345D+E346T+D476Q+G477Q, R118S+G182A+T193K, E345D+D476G+G477S, G50A+N174E+A204T, G50A+A51T+R172E+N174S, K302Q+G304A+E345D+E346T+D476Q+G477Q, S255I+E345D+G477Q, G304S+E345D+D476K+G477Q, R320V+D476Y, G50A+A51V+R172Q+A204V+G304Q+E345D+E346T+D476G+G477S, A51T+R172N+A204V+G304Q+E345D+E346T+D476G+G477S, G50A+A51T+A87S+R172N+L173T+N174D+G304Q+E345D+E346T+D476G+G477S, R172K+L173F+N174Q+A204V, G50A+A51V+W167F+R172N+N174T+A204T, G50A+A51V+A113L+R172N+N174T+A204T, G50A+A113L+R172Q+L173I+N174T+A204T, G50A+A51V+W167F+R172E+L173F+N174T+A204V, G50A+A51V+A113L+R172E+L173F+N174T+A204V, G50A+A51T+A113L+W167F+R172N+L173V, G50A+A51T+W167F+R172N+L173V, R172N+L173F+N174Q+G304Q+E345D+E346T+D476G+G477S, T40K+K72R+W167F, K72R+A113L+W167F+S255I+E345D+G477Q, Q98N+M208Y+V213T+V214T+L217M+R320A+S323M, G50A+A51T+R172E+N174S+G304Q+E345D+E346T+E391L+D476G+G477S, G50A+A51T+Q169M+R172E+N174S+G304Q+E345D+E346T+D476G+G477S, T40K+G50A+A51V+K72R+R172N+N174T+A204T, G50A+A51V+L173Q+R172*+N174T+G305S+E346P+G477T, W167F+R172N+L173F+N174Q+G304Q+E345D+E346T+D476G+G477S, T40K+G50A+W167F+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, W167F+R172D+N174T+A204V+S255I+E345D+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, G50S+R172N+N174Q+E345D, R172K+A204V+K302Q+E346P+D476K+G477S, R172K+A204V+E345D+D476K, G50A+R172N+A204T+E345D+D476K+G477S, T40K+K72R+W167F+S255I+E345D+G477Q, T40K+K72R+W167F+G304S+E345D+D476K+G477Q, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, L173T+A204T+R320V+D476Y, G50A+A51V+R172D+N174E+E346T+G477A, G50A+A51V+W167F+R172E+L173F+N174T+A204V+G304Q+E345D+E346T+D476G+G477T, G50A+A51T+R172N+L173V+G304Q+E345D+E346T+D476G+G477T, G50A+R172Q+L173T+G304Q+E345D+E346T+D476G+G477T, R172D+N174E+K302Q+E346P+D476K+G477S, G50A+A51V+R172D+N174E+K302Q+E346P+D476K+G477S, G50A+A51V+R172D+N174E+G304S+E345D+D476K+G477Q, G50A+A51V+R172D+N174E+E345D+E346P+D476G, G50A+A51T+R172E+A204T+E345D+D476K+G477S, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, G50A+R118P+R172D+N174Q+G184S+E346T+D476K+G477A, T40K+G50A+A51V+K72R+W167F+R172D+N174E, T40K+G50A+K72R+W167F+L173T+A204V, G50A+R172S+N174S+E346T+D476K+G477A, R172E+L173F+N174D+K302Q+E346P+D476K+G477S, R172E+L173F+N174D+E346T+D476K+G477A, R172E+L173F+N174D+E345D+D476K+G477S, R172E+L173F+N174D+S255I+E345D+G477Q, T5K+R172E+L173F+K302Q+E346P+D476K+G477S, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, T40K+K72R+W167F+R172N+L173F+N174Q+G304Q+E345D+E346T+G465K+D476G, K72R+W167F+E346T+D476K, when using SEQ ID NO: 1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In a particular embodiment, the variant has an Improvement Factor (IF) of at least 1.5 when determined in a Model J detergent at a temperature of 30° C.

In a preferred embodiment, the variant comprises a) a pairwise deletion of the amino acids corresponding to R181+G182, R181+H183, R181+G184, G182+H183, G182+G184, H183+G184, preferably G182+H183, and b) one of the following combinations of alterations: E345S, V56T, R171E, R172Y, A113N, R142K, R172L, R172L, A113Y, A113L, Y243G, E391M, H1K, H1L, I430E, G50A+R172E+L173F, L173V+N174T, G50A+R172Q+L173F+N174Q, R172E+L173F+N174Q, G50A+A51I+A204V, R172Q+N174D, G50A+R172E+L173V+N174T, G50A+A51T+L173F, E345D+D476Q+G477K, G304Q+E345D+E346T+D476G+G477S, E346T, R172N+L173F+N174Q+G304Q+E345D+E346T+D476G+G477S, L173F, Y160L, V165G, G50A+A51I+R172D+L173V, R172D+L173T+N174D+A204T, R172N+L173V, T5K+R172E+L173F, R172D+L173T+N174D, G50A+A51T+R172N+L173V+E345D+E346P+D476G, N174D+A204T+E346T+D476K+G477A, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+G50A+W167F+R172N+N174S+A204T, T40K+K72R+E345D+E346P+D476G, T40K+K72R+R172N+L173F+N174Q+G304Q+E345D+E346T+D476G+G477S, G50A+L173T+N174E+A204T+E345D+S424Q, L173F+N174D+A354V, G50A+A51V+R172D+L173F+A204V+K302Q+D476G+G477S, W140Y+R172E+N174D+A204T+G304Q+E345D+E346T+D476G+G477S, G50A+W140Y+R172N+A204T, T40K+G50A+W167F+R172D+N174T+E345D+E346P+D476G, W167F+R172D+N174T+A204V+S255I+E345D+G477Q, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+E345D+L405V+T414K, N174D+A204T+K302Q+E346P+D476K+G477S, R172D+N174T+K302Q+E346P+D476K+G477S, R172N+N174Q+E345D+D406A+I411M+N446K, G50A+N174D+A204T+E345D+D476K+G477S, N174D+A204T+G304S+E345D+D476K+G477Q, R172K+A204V+G304S+E345D+D476K+G477Q, G50A+R172N+A204T+E346T+D432A+D476K+G477A, G50A+R172N+A204T+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, R172E+N174D+A204T+M208Y+V213T+V214T+L217M+G304Q+E345D+E346T+D476G+G477S, A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, R172N+L173V+E346T+G477A, G50A+A51T+R172N+L173V+R320A+S323M+D476Y, G50A+R172Q+L173T+E346T+G477A, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, G50A+A51V+R172E+N174S+E346T+G477A, G50A+A51T+R172E+A204T+R320V+N457T+D476Y, G50A+A51V+R172D+N174E+R320V+D476Y, G50A+R172D+N174Q+E346T+G477A, G50A+R172D+N174Q+R320V+D476Y, G50A+R172N+A204T+K302Q+E346P+D476K+G477S, G7K+T40K+K72R+W167F, G50A+A51V+R172N+N174T+A204T+G304Q+E345D+E346T+D476G+G477T, G50A+R172Q+L173T+G304Q+E345D+E346T+D476G+G477T, G50A+R172D+N174D+R320V, L173T+A204T+G304S+E345D+D476K+G477Q, G50A+R118P+R172D+N174Q+G184S+E346T+D476K+G477A, G50A+A51V+R118P+N174S+G184S, G50A+R118P+R172E+G184S+A204T, T40K+G50A+A51V+W167F+N174S, T40K+W167F+L173T+A204T, G50A+R172S+N174S+E346T+D476K+G477A, G50A+R118P+R172D+N174Q+E346T+D476K+G477A, G50A+R172D+N174Q+G304S+E345D+I464T+G465E+D476K+G477Q, G50A+R172D+N174Q+E346T, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+T246M+S255I+E345D+G477Q, G50A+A51T+K72R+W167F+Q169M+R172E+N174S+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, N174D+E345D+E346P+D476K, T40K+K72R+R172N+L173F+N174Q+G304Q+E345D+E346T+D476G, G50A+R172N+N174T+E346T+D476K, R118P+W140Y, G7K+T40K+W167F+R320A+S323N+P364S, when using SEQ ID NO: 1 for numbering.

In one embodiment, the parent alpha-amylase has the amino acid sequence set forth in SEQ ID NO: 1, SEQ ID NO: 2, SEQ ID NO: 4, SEQ ID NO: 7, SEQ ID NO: 8, SEQ ID NO: 9, SEQ ID NO: 10, SEQ ID NO:11, SEQ ID NO: 12, SEQ ID NO:13, SEQ ID NO: 14, SEQ ID NO: 15, or SEQ ID NO: 16.

In one embodiment the alpha-amylase variants of the invention are isolated variants.

In a preferred embodiment a) comprises a pairwise deletion of the amino acids corresponding to H183+G184. In another embodiment a) comprises a pairwise deletion of the amino acids corresponding to R181+G182.

In another embodiment a) comprises a pairwise deletion of the amino acids corresponding to R181+H183.

In another embodiment a) comprises a pairwise deletion of the amino acids corresponding to R181+G184.

In another embodiment a) comprises a pairwise deletion of the amino acids corresponding to G182+H183.

In another embodiment a) comprises a pairwise deletion of the amino acids corresponding to G182+G184.

In another embodiment, a) further comprises a substitution at one or both of the non deleted positions of R181, G182, H183 and G184.

In an embodiment, the variant of the invention has at least 90% sequence identity but less than 100% sequence identity to the polypeptide having the amino acid sequence of SEQ ID NO: 1. In another embodiment, the variant has at least 91% sequence identity but less than 100% sequence identity to the polypeptide having the amino acid sequence of SEQ ID NO: 1. In another embodiment, the variant has at least 92% sequence identity but less than 100% sequence identity to the polypeptide having the amino acid sequence of SEQ ID NO: 1. In another embodiment, the variant has at least 93% sequence identity but less than 100% sequence identity to the polypeptide having the amino acid sequence of SEQ ID NO: 1. In another embodiment, the variant has at least 94% sequence identity but less than 100% sequence identity to the polypeptide having the amino acid sequence of SEQ ID NO: 1. In another embodiment, the variant has at least 95% sequence identity but less than 100% sequence identity to the polypeptide having the amino acid sequence of SEQ ID NO: 1. In one embodiment, the variant has at least 96% sequence identity but less than 100% sequence identity to the polypeptide having the amino acid sequence of SEQ ID NO: 1. In another embodiment, the variant has at least 97% sequence identity but less than 100% sequence identity to the polypeptide having the amino acid sequence of SEQ ID NO: 1. In yet another embodiment, the variant has at least 98% sequence identity but less than 100% sequence identity to the polypeptide having the amino acid sequence of SEQ ID NO: 1. In another embodiment, the variant has at least 99% sequence identity but less than 100% sequence identity to the polypeptide having the amino acid sequence of SEQ ID NO: 1.

In an embodiment, the variant of the invention has at least 90% sequence identity but less than 100% sequence identity to the polypeptide having the amino acid sequence of SEQ ID NO: 4. In another embodiment, the variant has at least 91% sequence identity but less than 100% sequence identity to the polypeptide having the amino acid sequence of SEQ ID NO: 4. In another embodiment, the variant has at least 92% sequence identity but less than 100% sequence identity to the polypeptide having the amino acid sequence of SEQ ID NO: 4. In another embodiment, the variant has at least 93% sequence identity but less than 100% sequence identity to the polypeptide having the amino acid sequence of SEQ ID NO: 4. In another embodiment, the variant has at least 94% sequence identity but less than 100% sequence identity to the polypeptide having the amino acid sequence of SEQ ID NO: 4. In another embodiment, the variant has at least 95% sequence identity but less than 100% sequence identity to the polypeptide having the amino acid sequence of SEQ ID NO: 4. In one embodiment, the variant has at least 96% sequence identity but less than 100% sequence identity to the polypeptide having the amino acid sequence of SEQ ID NO: 4. In another embodiment, the variant has at least 97% sequence identity but less than 100% sequence identity to the polypeptide having the amino acid sequence of SEQ ID NO: 4. In yet another embodiment, the variant has at least 98% sequence identity but less than 100% sequence identity to the polypeptide having the amino acid sequence of SEQ ID NO: 4. In another embodiment, the variant has at least 99% sequence identity but less than 100% sequence identity to the polypeptide having the amino acid sequence of SEQ ID NO: 4.

In another embodiment, the variant has at least 90% sequence identity but less than 100% sequence identity to the polypeptide having the amino acid sequence of SEQ ID NO: 7. In another embodiment, the variant has at least 91% sequence identity but less than 100% sequence identity to the polypeptide having the amino acid sequence of SEQ ID NO: 7. In another embodiment, the variant has at least 92% sequence identity but less than 100% sequence identity to the polypeptide having the amino acid sequence of SEQ ID NO: 7. In another embodiment, the variant has at least 93% sequence identity but less than 100% sequence identity to the polypeptide having the amino acid sequence of SEQ ID NO: 7. In another embodiment, the variant has at least 94% sequence identity but less than 100% sequence identity to the polypeptide having the amino acid sequence of SEQ ID NO: 7. In another embodiment, the variant has at least 95% sequence identity but less than 100% sequence identity to the polypeptide having the amino acid sequence of SEQ ID NO: 7. In another embodiment, the variant has at least 96% sequence identity but less than 100% sequence identity to the polypeptide having the amino acid sequence of SEQ ID NO: 7. In another embodiment, the variant has at least 97% sequence identity but less than 100% sequence identity to the polypeptide having the amino acid sequence of SEQ ID NO: 7. In yet another embodiment, the variant has at least 98% sequence identity but less than 100% sequence identity to the polypeptide having the amino acid sequence of SEQ ID NO: 7. In another embodiment, the variant has at least 99% sequence identity but less than 100% sequence identity to the polypeptide having the amino acid sequence of SEQ ID NO: 7.

In another embodiment, the variant has at least 90% sequence identity but less than 100% sequence identity to the polypeptide having the amino acid sequence of SEQ ID NO: 8. In another embodiment, the variant has at least 91% sequence identity but less than 100% sequence identity to the polypeptide having the amino acid sequence of SEQ ID NO: 8. In another embodiment, the variant has at least 92% sequence identity but less than 100% sequence identity to the polypeptide having the amino acid sequence of SEQ ID NO: 8. In another embodiment, the variant has at least 93% sequence identity but less than 100% sequence identity to the polypeptide having the amino acid sequence of SEQ ID NO: 8. In another embodiment, the variant has at least 94% sequence identity but less than 100% sequence identity to the polypeptide having the amino acid sequence of SEQ ID NO: 8. In another embodiment, the variant has at least 95% sequence identity but less than 100% sequence identity to the polypeptide having the amino acid sequence of SEQ ID NO: 8. In another embodiment, the variant has at least 96% sequence identity but less than 100% sequence identity to the polypeptide having the amino acid sequence of SEQ ID NO: 8. In another embodiment, the variant has at least 97% sequence identity but less than 100% sequence identity to the polypeptide having the amino acid sequence of SEQ ID NO: 8. In yet another embodiment, the variant has at least 98% sequence identity but less than 100% sequence identity to the polypeptide having the amino acid sequence of SEQ ID NO: 8. In another embodiment, the variant has at least 99% sequence identity but less than 100% sequence identity to the polypeptide having the amino acid sequence of SEQ ID NO: 8.

In another embodiment, the variant has at least 90% sequence identity but less than 100% sequence identity to the polypeptide having the amino acid sequence of SEQ ID NO:9. In another embodiment, the variant has at least 91% sequence identity but less than 100% sequence identity to the polypeptide having the amino acid sequence of SEQ ID NO:9. In another embodiment, the variant has at least 92% sequence identity but less than 100% sequence identity to the polypeptide having the amino acid sequence of SEQ ID NO:9. In another embodiment, the variant has at least 93% sequence identity but less than 100% sequence identity to the polypeptide having the amino acid sequence of SEQ ID NO:9. In another embodiment, the variant has at least 94% sequence identity but less than 100% sequence identity to the polypeptide having the amino acid sequence of SEQ ID NO:9. In another embodiment, the variant has at least 95% sequence identity but less than 100% sequence identity to the polypeptide having the amino acid sequence of SEQ ID NO:9. In another embodiment, the variant has at least 96% sequence identity but less than 100% sequence identity to the polypeptide having the amino acid sequence of SEQ ID NO:9. In another embodiment, the variant has at least 97% sequence identity but less than 100% sequence identity to the polypeptide having the amino acid sequence of SEQ ID NO:9. In yet another embodiment, the variant has at least 98% sequence identity but less than 100% sequence identity to the polypeptide having the amino acid sequence of SEQ ID NO:9. In another embodiment, the variant has at least 99% sequence identity but less than 100% sequence identity to the polypeptide having the amino acid sequence of SEQ ID NO:9.

In another embodiment, the variant has at least 90% sequence identity but less than 100% sequence identity to the polypeptide having the amino acid sequence of SEQ ID NO:10. In another embodiment, the variant has at least 91% sequence identity but less than 100% sequence identity to the polypeptide having the amino acid sequence of SEQ ID NO:10. In another embodiment, the variant has at least 92% sequence identity but less than 100% sequence identity to the polypeptide having the amino acid sequence of SEQ ID NO:10. In another embodiment, the variant has at least 93% sequence identity but less than 100% sequence identity to the polypeptide having the amino acid sequence of SEQ ID NO:10. In another embodiment, the variant has at least 94% sequence identity but less than 100% sequence identity to the polypeptide having the amino acid sequence of SEQ ID NO:10. In another embodiment, the variant has at least 95% sequence identity but less than 100% sequence identity to the polypeptide having the amino acid sequence of SEQ ID NO:10. In another embodiment, the variant has at least 96% sequence identity but less than 100% sequence identity to the polypeptide having the amino acid sequence of SEQ ID NO:10. In another embodiment, the variant has at least 97% sequence identity but less than 100% sequence identity to the polypeptide having the amino acid sequence of SEQ ID NO:10. In yet another embodiment, the variant has at least 98% sequence identity but less than 100% sequence identity to the polypeptide having the amino acid sequence of SEQ ID NO:10. In another embodiment, the variant has at least 99% sequence identity but less than 100% sequence identity to the polypeptide having the amino acid sequence of SEQ ID NO:10.

In another embodiment, the variant has at least 90% sequence identity but less than 100% sequence identity to the polypeptide having the amino acid sequence of SEQ ID NO:11. In another embodiment, the variant has at least 91% sequence identity but less than 100% sequence identity to the polypeptide having the amino acid sequence of SEQ ID NO:11. In another embodiment, the variant has at least 92% sequence identity but less than 100% sequence identity to the polypeptide having the amino acid sequence of SEQ ID NO:11. In another embodiment, the variant has at least 93% sequence identity but less than 100% sequence identity to the polypeptide having the amino acid sequence of SEQ ID NO:11. In another embodiment, the variant has at least 94% sequence identity but less than 100% sequence identity to the polypeptide having the amino acid sequence of SEQ ID NO:11. In another embodiment, the variant has at least 95% sequence identity but less than 100% sequence identity to the polypeptide having the amino acid sequence of SEQ ID NO:11. In another embodiment, the variant has at least 96% sequence identity but less than 100% sequence identity to the polypeptide having the amino acid sequence of SEQ ID NO:11. In another embodiment, the variant has at least 97% sequence identity but less than 100% sequence identity to the polypeptide having the amino acid sequence of SEQ ID NO:11. In yet another embodiment, the variant has at least 98% sequence identity but less than 100% sequence identity to the polypeptide having the amino acid sequence of SEQ ID NO:11. In another embodiment, the variant has at least 99% sequence identity but less than 100% sequence identity to the polypeptide having the amino acid sequence of SEQ ID NO:11.

In another embodiment, the variant has at least 90% sequence identity but less than 100% sequence identity to the polypeptide having the amino acid sequence of SEQ ID NO:12. In another embodiment, the variant has at least 91% sequence identity but less than 100% sequence identity to the polypeptide having the amino acid sequence of SEQ ID NO:12. In another embodiment, the variant has at least 92% sequence identity but less than 100% sequence identity to the polypeptide having the amino acid sequence of SEQ ID NO:12. In another embodiment, the variant has at least 93% sequence identity but less than 100% sequence identity to the polypeptide having the amino acid sequence of SEQ ID NO:12. In another embodiment, the variant has at least 94% sequence identity but less than 100% sequence identity to the polypeptide having the amino acid sequence of SEQ ID NO:12. In another embodiment, the variant has at least 95% sequence identity but less than 100% sequence identity to the polypeptide having the amino acid sequence of SEQ ID NO:12. In another embodiment, the variant has at least 96% sequence identity but less than 100% sequence identity to the polypeptide having the amino acid sequence of SEQ ID NO:12. In another embodiment, the variant has at least 97% sequence identity but less than 100% sequence identity to the polypeptide having the amino acid sequence of SEQ ID NO:12. In yet another embodiment, the variant has at least 98% sequence identity but less than 100% sequence identity to the polypeptide having the amino acid sequence of SEQ ID NO:12. In another embodiment, the variant has at least 99% sequence identity but less than 100% sequence identity to the polypeptide having the amino acid sequence of SEQ ID NO:12.

In another embodiment, the variant has at least 90% sequence identity but less than 100% sequence identity to the polypeptide having the amino acid sequence of SEQ ID NO:13. In another embodiment, the variant has at least 91% sequence identity but less than 100% sequence identity to the polypeptide having the amino acid sequence of SEQ ID NO:13. In another embodiment, the variant has at least 92% sequence identity but less than 100% sequence identity to the polypeptide having the amino acid sequence of SEQ ID NO:13. In another embodiment, the variant has at least 93% sequence identity but less than 100% sequence identity to the polypeptide having the amino acid sequence of SEQ ID NO:13. In another embodiment, the variant has at least 94% sequence identity but less than 100% sequence identity to the polypeptide having the amino acid sequence of SEQ ID NO:13. In another embodiment, the variant has at least 95% sequence identity but less than 100% sequence identity to the polypeptide having the amino acid sequence of SEQ ID NO:13. In another embodiment, the variant has at least 96% sequence identity but less than 100% sequence identity to the polypeptide having the amino acid sequence of SEQ ID NO:13. In another embodiment, the variant has at least 97% sequence identity but less than 100% sequence identity to the polypeptide having the amino acid sequence of SEQ ID NO:13. In yet another embodiment, the variant has at least 98% sequence identity but less than 100% sequence identity to the polypeptide having the amino acid sequence of SEQ ID NO:13. In another embodiment, the variant has at least 99% sequence identity but less than 100% sequence identity to the polypeptide having the amino acid sequence of SEQ ID NO:13.

In another embodiment, the variant has at least 90% sequence identity but less than 100% sequence identity to the polypeptide having the amino acid sequence of SEQ ID NO:14. In another embodiment, the variant has at least 91% sequence identity but less than 100% sequence identity to the polypeptide having the amino acid sequence of SEQ ID NO:14. In another embodiment, the variant has at least 92% sequence identity but less than 100% sequence identity to the polypeptide having the amino acid sequence of SEQ ID NO:14. In another embodiment, the variant has at least 93% sequence identity but less than 100% sequence identity to the polypeptide having the amino acid sequence of SEQ ID NO:14. In another embodiment, the variant has at least 94% sequence identity but less than 100% sequence identity to the polypeptide having the amino acid sequence of SEQ ID NO:14. In another embodiment, the variant has at least 95% sequence identity but less than 100% sequence identity to the polypeptide having the amino acid sequence of SEQ ID NO:14. In another embodiment, the variant has at least 96% sequence identity but less than 100% sequence identity to the polypeptide having the amino acid sequence of SEQ ID NO:14. In another embodiment, the variant has at least 97% sequence identity but less than 100% sequence identity to the polypeptide having the amino acid sequence of SEQ ID NO:14. In yet another embodiment, the variant has at least 98% sequence identity but less than 100% sequence identity to the polypeptide having the amino acid sequence of SEQ ID NO:14. In another embodiment, the variant has at least 99% sequence identity but less than 100% sequence identity to the polypeptide having the amino acid sequence of SEQ ID NO:14.

In another embodiment, the variant has at least 90% sequence identity but less than 100% sequence identity to the polypeptide having the amino acid sequence of SEQ ID NO:15. In another embodiment, the variant has at least 91% sequence identity but less than 100% sequence identity to the polypeptide having the amino acid sequence of SEQ ID NO:15. In another embodiment, the variant has at least 92% sequence identity but less than 100% sequence identity to the polypeptide having the amino acid sequence of SEQ ID NO:15. In another embodiment, the variant has at least 93% sequence identity but less than 100% sequence identity to the polypeptide having the amino acid sequence of SEQ ID NO:15. In another embodiment, the variant has at least 94% sequence identity but less than 100% sequence identity to the polypeptide having the amino acid sequence of SEQ ID NO:15. In another embodiment, the variant has at least 95% sequence identity but less than 100% sequence identity to the polypeptide having the amino acid sequence of SEQ ID NO:15. In another embodiment, the variant has at least 96% sequence identity but less than 100% sequence identity to the polypeptide having the amino acid sequence of SEQ ID NO:15. In another embodiment, the variant has at least 97% sequence identity but less than 100% sequence identity to the polypeptide having the amino acid sequence of SEQ ID NO:15. In yet another embodiment, the variant has at least 98% sequence identity but less than 100% sequence identity to the polypeptide having the amino acid sequence of SEQ ID NO:15. In another embodiment, the variant has at least 99% sequence identity but less than 100% sequence identity to the polypeptide having the amino acid sequence of SEQ ID NO:15.

In another embodiment, the variant has at least 90% sequence identity but less than 100% sequence identity to the polypeptide having the amino acid sequence of SEQ ID NO:16. In another embodiment, the variant has at least 91% sequence identity but less than 100% sequence identity to the polypeptide having the amino acid sequence of SEQ ID NO:16. In another embodiment, the variant has at least 92% sequence identity but less than 100% sequence identity to the polypeptide having the amino acid sequence of SEQ ID NO:16. In another embodiment, the variant has at least 93% sequence identity but less than 100% sequence identity to the polypeptide having the amino acid sequence of SEQ ID NO:16. In another embodiment, the variant has at least 94% sequence identity but less than 100% sequence identity to the polypeptide having the amino acid sequence of SEQ ID NO:16. In another embodiment, the variant has at least 95% sequence identity but less than 100% sequence identity to the polypeptide having the amino acid sequence of SEQ ID NO:16. In another embodiment, the variant has at least 96% sequence identity but less than 100% sequence identity to the polypeptide having the amino acid sequence of SEQ ID NO:16. In another embodiment, the variant has at least 97% sequence identity but less than 100% sequence identity to the polypeptide having the amino acid sequence of SEQ ID NO:16. In yet another embodiment, the variant has at least 98% sequence identity but less than 100% sequence identity to the polypeptide having the amino acid sequence of SEQ ID NO:16. In another embodiment, the variant has at least 99% sequence identity but less than 100% sequence identity to the polypeptide having the amino acid sequence of SEQ ID NO:16.

Hereby variants are obtained which have improved wash performance compared to the parent alpha-amylase, e.g. the alpha-amylase of SEQ ID NO: 2 or compared to the alpha-amylase of SEQ ID NO: 1 having corresponding alterations with respect to a) as the variant of the invention. I.e., the wash performance of the variant of the invention may preferably be compared to the parent alpha-amylase having the same alterations as the variant with respect to the alterations of a). So, if the variant of the invention is a variant of the alpha-amylase of SEQ ID NO: 1, the variant comprising a) a deletion of 181*+182* and b) one or more of the substitutions of the invention, then the wash performance preferably may be compared to and improved over SEQ ID NO: 1 having a deletion of amino acids 181*+182*. These variants also have the advantage that they have improved wash performance at low temperatures over alpha-amylases known in the art, such as the SP707 alpha-amylase (SEQ ID NO: 4 herein) having corresponding alterations with respect to a) as the variant of the invention. The wash performance is considered improved if it is improved in one or more of the conditions of Example 1, i.e. if the Improvement Factor (IF) is above 1.0 in either of:
a) model detergent A at 15° C. where the alpha-amylase variant concentration is 0.2 mg/L wash solution,
b) model detergent A at 40° C. where the alpha-amylase variant concentration is 0.05 mg/L wash solution,
c) model detergent J at 15° C. where the alpha-amylase variant concentration is 0.2 mg/L wash solution, or in
d) model detergent J at 30° C. where the alpha-amylase variant concentration is 0.05 mg/L wash solution. The wash conditions are described in Example 1.
   Preferably, the Improvement Factor (IF) is above 1.05.

In another embodiment, the variant is a variant of a parent alpha-amylase which is encoded by a polynucleotide that hybridizes under medium stringency conditions, medium-high stringency conditions, high stringency conditions, or very high stringency conditions with (i) the mature polypeptide coding sequence of SEQ ID NO: 3 or (ii) the full-length complement of (i).

In a preferred embodiment, the invention relates to a polynucleotide that hybridizes under high stringency conditions with (i) the mature polypeptide coding sequence of SEQ ID NO: 3 or (ii) the full-length complement of (i). In another preferred embodiment, the invention relates to a polynucleotide that hybridizes under very high stringency conditions with (i) the mature polypeptide coding sequence of SEQ ID NO: 3 or (ii) the full-length complement of (i).

The polynucleotide of SEQ ID NO: 3 or a subsequence thereof, as well as the polypeptides of SEQ ID NO: 1 and 2 or a fragment thereof, may be used to design nucleic acid probes to identify and clone DNA encoding polypeptides having alpha-amylase activity from strains of different genera or species according to methods well-known in the art. In particular, such probes may be used for hybridization with the genomic DNA or cDNA of a cell of interest, following standard Southern blotting procedures, in order to identify and isolate the corresponding gene therein. Such probes may be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes may be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having alpha-amylase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 3 or a subsequence thereof, the carrier material is used in a Southern blot.

For purposes of the present invention, hybridization indicates that the polynucleotide hybridizes to a labeled nucleic acid probe corresponding to (i) SEQ ID NO: 3; (ii) the mature polypeptide coding sequence of SEQ ID NO: 3; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In another embodiment, the present invention relates to an alpha-amylase variant having alpha-amylase activity encoded by a polynucleotide having a sequence identity to the mature polypeptide coding sequence of SEQ ID NO: 3 of at least 70%, such as at least 80%, or at least 90%, such as at least 95%, at least 96%, at least 97%, at least 98%, at least 99%, or 100%.

In other embodiments the present invention relates to an alpha-amylase variant having alpha-amylase activity and having at least 95% sequence identity to SEQ ID NO: 1 which polypeptide is encoded by a polynucleotide having a sequence identity to the mature polypeptide coding sequence of SEQ ID NO: 3 of at least 70%, such as at least 80% or at least 85% or at least 90%.

The present invention also provides a method of improving the wash performance of a parent alpha-amylase having the amino acid sequence of SEQ ID NO: 1 or having at least 90% sequence identity thereto, said method comprising the steps of:

a) substituting and/or deleting two or more amino acids at positions in the parent alpha-amylase corresponding to positions R181, G182, H183 and G184 of the mature polypeptide of SEQ ID NO: 1, and

b) introducing into the parent alpha-amylase a substitution at one or more of the following positions: 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173, or 174 of the mature polypeptide of SEQ ID NO: 1, wherein the resulting variant has at least 95%, such as at least 97%, but less than 100% sequence identity with the mature polypeptide of SEQ ID NO: 1, and wherein the resulting variant has alpha-amylase activity and an improved wash performance compared to the parent alpha-amylase.

In one embodiment the step a) comprises deleting two of said amino acids at positions R181, G182, H183 and G184, such as the amino acids corresponding to R181+G182, R181+H183, R181+G184, G182+H183, G182+G184 or H183+G184 of SEQ ID NO: 1. In a preferred embodiment the amino acids at positions 183+184 are deleted.

In one embodiment b) comprises or consists of the substitutions or deletions selected from the group consisting of: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51 L+N174S+A447V, G50A+A51T+L173F, G50A+R172Q+L173I, G50A+A51V+L173F+N174D, G50A+R172D+L173T, G50A+A51T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V, Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476 G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51 L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174 D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S, when SEQ ID NO: 1 is used for numbering.

In a preferred embodiment, the variant has an improved property relative to the parent alpha-amylase, wherein the improved property is selected from the group consisting of catalytic efficiency, catalytic rate, chemical stability, oxidation stability, pH activity, pH stability, specific activity, stability under storage conditions, substrate binding, substrate cleavage, substrate specificity, substrate stability, surface properties, thermal activity, thermo stability, preferably improved washing performance at low temperature, detergent stability and chelator stability.

In another embodiment, the variant has equal or improved stability in liquid detergents relative to the alpha-amylase of SEQ ID NO: 2. In another embodiment, the variant has equal or improved stability in liquid detergents relative to the alpha-amylase of SEQ ID NO: 1 having corresponding alterations with respect to a) as the variant of the invention. I.e., the detergent stability of the variant of the invention may preferably be compared to the alpha-amylase of SEQ ID NO: 1 having the same alterations as the variant with respect to the alterations at positions corresponding to 181, 182, 183 and 184 of the alpha-amylase of SEQ ID NO: 1.

In one embodiment, the number of amino acid substitutions and/or deletions introduced into the polypeptide of SEQ ID NO: 1 is up to 30, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In an embodiment, the number of amino acid substitutions and/or deletions introduced into the polypeptide of SEQ ID NO: 1 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide may be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. Essential amino acids in the sequence of amino acids of SEQ ID NO: 2 are located at positions D236, E266 and D333, which are the catalytic residues. In addition, the Y58, H107, R234, H240, H332 are critical for forming the active site. Thus, amino acids D236, E266, D333, Y58, H107, R234, H240, H332 should preferable not be mutated.

Single or multiple amino acid substitutions, deletions, and/or insertions may be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods may be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides may be recovered from the host cells and rapidly sequenced using standard methods in the art. These methods allow the rapid determination of the importance of individual amino acid residues in a polypeptide.

### Parent Alpha-Amylases

In a preferred embodiment, the variant is a variant of a parent alpha-amylase selected from the group consisting of:
a. a polypeptide having at least 90% sequence identity to the mature polypeptide of SEQ ID NO: 1;
b. a polypeptide having at least 90% sequence identity to the mature polypeptide of SEQ ID NO: 4;
c. a fragment of the mature polypeptide of SEQ ID NO: 1, which has alpha-amylase activity;
d. a fragment of the mature polypeptide of SEQ ID NO: 4, which has alpha-amylase activity;
e. a polypeptide having immunological cross reactivity with an antibody raised against the mature polypeptide of SEQ ID NO: 1 or 4.

In other embodiments, the parent alpha-amylase has at least 91%, or at least 92%, or at least 93%, or at least 94%, or at least 95%, or at least 96%, such as at least 97%, at least 98%, or at least 99% sequence identity to SEQ ID NO: 1.

In other embodiments, the parent alpha-amylase has at least 91%, or at least 92%, or at least 93%, or at least 94%, or at least 95%, or at least 96%, such as at least 97%, at least 98%, or at least 99% sequence identity to SEQ ID NO: 4.

In another aspect, the parent alpha-amylase may be (a) a polypeptide having at least 90% sequence identity to the mature polypeptide of SEQ ID NO: 2; (b) a fragment of the mature polypeptide of SEQ ID NO: 2, which has alpha-amylase activity or (c) a polypeptide having immunological cross reactivity with an antibody raised against the mature polypeptide of SEQ ID NO: 2.

In another aspect, the parent alpha-amylase may be (a) a polypeptide having at least 90% sequence identity to the mature polypeptide of SEQ ID NO: 7; (b) a fragment of the mature polypeptide of SEQ ID NO: 7, which has alpha-amylase activity or (c) a polypeptide having immunological cross reactivity with an antibody raised against the mature polypeptide of SEQ ID NO 7.

In another aspect, the parent alpha-amylase may be (a) a polypeptide having at least 90% sequence identity to the mature polypeptide of SEQ ID NO: 8; (b) a fragment of the mature polypeptide of SEQ ID NO: 8, which has alpha-amylase activity or (c) a polypeptide having immunological cross reactivity with an antibody raised against the mature polypeptide of SEQ ID NO: 8.

In another aspect, the parent alpha-amylase may be (a) a polypeptide having at least 90% sequence identity to the mature polypeptide of SEQ ID NO: 9; (b) a fragment of the mature polypeptide of SEQ ID NO: 9, which has alpha-amylase activity or (c) a polypeptide having immunological cross reactivity with an antibody raised against the mature polypeptide of SEQ ID NO: 9.

In another aspect, the parent alpha-amylase may be (a) a polypeptide having at least 90% sequence identity to the mature polypeptide of SEQ ID NO: 10; (b) a fragment of the mature polypeptide of SEQ ID NO: 10, which has alpha-amylase activity or (c) a polypeptide having immunological cross reactivity with an antibody raised against the mature polypeptide of SEQ ID NO: 10.

In another aspect, the parent alpha-amylase may be (a) a polypeptide having at least 90% sequence identity to the mature polypeptide of SEQ ID NO: 11; (b) a fragment of the mature polypeptide of SEQ ID NO: 11, which has alpha-amylase activity or (c) a polypeptide having immunological cross reactivity with an antibody raised against the mature polypeptide of SEQ ID NO: 11.

In another aspect, the parent alpha-amylase may be (a) a polypeptide having at least 90% sequence identity to the mature polypeptide of SEQ ID NO: 12; (b) a fragment of the mature polypeptide of SEQ ID NO: 12, which has alpha-amylase activity or (c) a polypeptide having immunological cross reactivity with an antibody raised against the mature polypeptide of SEQ ID NO: 12.

In another aspect, the parent alpha-amylase may be (a) a polypeptide having at least 90% sequence identity to the mature polypeptide of SEQ ID NO: 13; (b) a fragment of the mature polypeptide of SEQ ID NO: 13, which has alpha-amylase activity or (c) a polypeptide having immunological cross reactivity with an antibody raised against the mature polypeptide of SEQ ID NO: 13.

In another aspect, the parent alpha-amylase may be (a) a polypeptide having at least 90% sequence identity to the mature polypeptide of SEQ ID NO: 14; (b) a fragment of the mature polypeptide of SEQ ID NO: 14, which has alpha-amylase activity or (c) a polypeptide having immunological cross reactivity with an antibody raised against the mature polypeptide of SEQ ID NO: 14.

In another aspect, the parent alpha-amylase may be (a) a polypeptide having at least 90% sequence identity to the mature polypeptide of SEQ ID NO: 15; (b) a fragment of the mature polypeptide of SEQ ID NO: 15, which has alpha-amylase activity or (c) a polypeptide having immunological cross reactivity with an antibody raised against the mature polypeptide of SEQ ID NO: 15.

In another aspect, the parent alpha-amylase may be (a) a polypeptide having at least 90% sequence identity to the mature polypeptide of SEQ ID NO: 16; (b) a fragment of the mature polypeptide of SEQ ID NO: 16, which has alpha-amylase activity or (c) a polypeptide having immunological cross reactivity with an antibody raised against the mature polypeptide of SEQ ID NO: 16.

In one embodiment, the parent alpha-amylase comprises the substitutions N195F+I206Y using SEQ ID NO: 1 for numbering. I.e. the parent alpha-amylase may be any of SEQ ID NOs: 4, 7, 8, 9, 10, 11, 12, 13, 14, 15 or 16 having the substitutions of N195F+I206Y using SEQ ID NO: 1 for numbering.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 1.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 2.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 4.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 7.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 8.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 9.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 10.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 11.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 12.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 13.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 14.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 15.

In one aspect, the amino acid sequence of the parent alpha-amylase differs by no more than 10 amino acids, e.g., 1, 2, 3, 4, 5, 6, 7, 8, or 9, from the mature polypeptide of SEQ ID NO: 16.

In another aspect, the parent alpha-amylase comprises or consists of the amino acid sequence of SEQ ID NO: 1. In another aspect, the parent alpha-amylase comprises or consists of the amino acid sequence of SEQ ID NO: 2.

In yet another embodiment, the parent alpha-amylase is an allelic variant of the mature polypeptide of SEQ ID NO: 1.

The parent alpha-amylase may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide may further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent alpha-amylase may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

The parent alpha-amylase may be a bacterial alpha-amylase. For example, the parent alpha-amylase may be a Gram-positive bacterial polypeptide such as a Bacillus alpha-amylase. The alpha-amylases of SEQ ID NOs 1 and 2 as well as the variants hereof may be artificially manufactured by methods known in the art.

### Preparation of Variants

The present invention also relates to methods for obtaining a variant having alpha-amylase activity, comprising introducing into a parent alpha-amylase having at least 95% sequence identity to SEQ ID NO: 1 a) substitution and/or deletion of two or more positions in the parent alpha-amylase said positions corresponding to positions R181, G182, H183 and G184 of the mature polypeptide of SEQ ID NO: 1, and b) a substitution at one or more positions said substitutions corresponding to positions a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173, or 174 of the mature polypeptide of SEQ ID NO: 1, wherein the resulting variant has at least 95%, such as at least 97%, but less than 100% sequence identity with the mature polypeptide of SEQ ID NO: 1, wherein the variant has alpha-amylase activity; and recovering the variant. The deletions and/or substitutions of a) and b) may be as described above.

The variants may be prepared using any mutagenesis procedure known in the art, such as site-directed mutagenesis, synthetic gene construction, semi-synthetic gene construction, random mutagenesis, shuffling, etc. Likewise, the polypeptides of SEQ ID NO:1, 2, 4, 5, 6, 7, 8, 9, 10, 11, 12, 13, 14, 15 and 16 may be produced by synthetic gene construction by means known to the skilled person.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure may be used in the present invention. There are many commercial kits available that can be used to prepare variants.

Synthetic gene construction entails in vitro synthesis of a designed polynucleotide molecule to encode a polypeptide of interest. Gene synthesis may be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions may be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that may be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods may be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a variant of the invention. Thus, in one aspect, the present invention relates to polynucleotides encoding a variant comprising (a) a deletion and/or a substitution at two or more positions corresponding to positions R181, G182, H183 and G184 of the mature polypeptide of SEQ ID NO: 1, and (b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity. In one embodiment the polynucleotides are isolated polynucleotides.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

Thus, in one aspect, the present invention relates to a nucleic acid construct comprising a polynucleotide encoding a variant comprising (a) a deletion and/or a substitution at two or more positions corresponding to positions R181, G182, H183 and G184 of the mature polypeptide of SEQ ID NO: 1, and (b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter comprises transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently comprise a signal peptide coding sequence naturally linked in translation reading frame with the segment of the coding sequence that encodes the polypeptide. Alternatively, the 5′-end of the coding sequence may comprise a signal peptide coding sequence that is foreign to the coding sequence. A foreign signal peptide coding sequence may be required where the coding sequence does not naturally comprise a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. Thus, the present invention relates to recombinant expression vectors comprising a polynucleotide encoding a variant comprising (a) a deletion and/or a substitution at two or more positions corresponding to positions R181, G182, H183 and G184 of the mature polypeptide of SEQ ID NO: 1, and (b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity, the expression vector further comprising a promoter, and transcriptional and translational stop signals.

The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may comprise any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably comprises one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably comprises an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may comprise additional polynucleotides for directing integration by homologous recombination into the genome of the host cell at a precise location(s) in the chromosome(s). To increase the likelihood of integration at a precise location, the integrational elements should comprise a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

More than one copy of a polynucleotide of the present invention may be inserted into a host cell to increase production of a polypeptide. An increase in the copy number of the polynucleotide may be obtained by integrating at least one additional copy of the sequence into the host cell genome or by including an amplifiable selectable marker gene with the polynucleotide where cells comprising amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a variant of the present invention. Thus, the present invention relates to recombinant host cells, comprising a polynucleotide encoding a variant comprising (a) a deletion and/or a substitution at two or more positions corresponding to positions R181, G182, H183 and G184 of the mature polypeptide of SEQ ID NO: 1, and (b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity, wherein the polynucleotide is operably linked to one or more control sequences that direct the production of a variant on the present invention.

A construct or vector comprising a polynucleotide is introduced into a host cell so that the construct or vector is maintained as a chromosomal integrant or as a self-replicating extrachromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

### Methods of Production

The present invention also relates to methods of producing a polypeptide of the present invention comprising (a) cultivating a recombinant host cell of the present invention under conditions conducive for production of the polypeptide; and optionally, (b) recovering the polypeptide. Thus, the present invention relates to methods of producing a variant comprising (a) a deletion and/or a substitution at two or more positions corresponding to positions R181, G182, H183 and G184 of the mature polypeptide of SEQ ID NO: 1, and (b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity, wherein the method comprises the steps of (i) cultivating a recombinant host cell comprising an expression vector, polynucleotide, or nucleic acid construct encoding the variant, under conditions conducive for production of the variant; and optionally, (b) recovering the variant.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides having alpha amylase activity. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. Thus, the present invention relates to a fermentation broth formulation or a cell composition comprising a variant comprising (a) a deletion and/or a substitution at two or more positions corresponding to positions R181, G182, H183 and G184 of the mature polypeptide of SEQ ID NO: 1, and (b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity.

The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth may comprise unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth comprises spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition comprises an organic acid(s), and optionally further comprises killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition comprises the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may comprise insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising an alpha-amylase variant of the present invention. Thus, the present invention relates to compositions comprising a variant comprising (a) a deletion and/or a substitution at two or more positions corresponding to positions R181, G182, H183 and G184 of the mature polypeptide of SEQ ID NO: 1, and (b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity.

Preferably, the compositions are enriched in such a polypeptide. The term “enriched” indicates that the alpha-amylase activity of the composition has been increased, e.g., with an enrichment factor of at least 1.1.

The compositions may comprise a polypeptide of the present invention as the major enzymatic component, e.g., a mono-component composition. Alternatively, the compositions may comprise multiple enzymatic activities, such as one or more (e.g., several) enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

### Detergent Compositions

In one embodiment, the invention is directed to detergent compositions comprising an alpha-amylase variant of the present invention in combination with one or more additional cleaning composition components. Thus, the present invention relates to detergent compositions comprising a variant comprising (a) a deletion and/or a substitution at two or more positions corresponding to positions R181, G182, H183 and G184 of the mature polypeptide of SEQ ID NO: 1, and (b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity in combination with one or more additional cleaning composition components.

In an embodiment, the detergent is a liquid detergent composition. In another embodiment the detergent composition is a powder detergent composition.

The detergent composition may be a laundry detergent composition or a dishwash detergent composition.

The choice of additional components is within the skill of the artisan and includes conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment of the present invention, the variant of the present invention may be added to a detergent composition in an amount corresponding to 0.001-100 mg of protein, such as 0.01-100 mg of protein, preferably 0.005-50 mg of protein, more preferably 0.01-25 mg of protein, even more preferably 0.05-10 mg of protein, most preferably 0.05-5 mg of protein, and even most preferably 0.01-1 mg of protein per liter of wash liquor.

A composition for use in automatic dishwash (ADW), for example, may include 0.0001%-50%, such as 0.001%-20%, such as 0.01%-10%, such as 0.05-5% of enzyme protein by weight of the composition.

A composition for use in laundry granulation, for example, may include 0.0001%-50%, such as 0.001%-20%, such as 0.01%-10%, such as 0.05%-5% of enzyme protein by weight of the composition.

A composition for use in laundry liquid, for example, may include 0.0001%-10%, such as 0.001-7%, such as 0.1%-5% of enzyme protein by weight of the composition.

The enzyme(s) of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in, for example, WO92/19709 and WO92/19708.

In certain markets different wash conditions and, as such, different types of detergents are used. This is disclosed in e.g. EP 1 025 240. For example, In Asia (Japan) a low detergent concentration system is used, while the United States uses a medium detergent concentration system, and Europe uses a high detergent concentration system.

A low detergent concentration system includes detergents where less than about 800 ppm of detergent components are present in the wash water. Japanese detergents are typically considered low detergent concentration system as they have approximately 667 ppm of detergent components present in the wash water.

A medium detergent concentration includes detergents where between about 800 ppm and about 2000 ppm of detergent components are present in the wash water. North American detergents are generally considered to be medium detergent concentration systems as they have approximately 975 ppm of detergent components present in the wash water.

A high detergent concentration system includes detergents where greater than about 2000 ppm of detergent components are present in the wash water. European detergents are generally considered to be high detergent concentration systems as they have approximately 4500-5000 ppm of detergent components in the wash water.

Latin American detergents are generally high suds phosphate builder detergents and the range of detergents used in Latin America can fall in both the medium and high detergent concentrations as they range from 1500 ppm to 6000 ppm of detergent components in the wash water. Such detergent compositions are all embodiments of the invention.

A polypeptide of the present invention may also be incorporated in the detergent formulations disclosed in WO97/07202, which is hereby incorporated by reference.

Examples are given below of preferred uses of the compositions of the present invention. The dosage of the composition and other conditions under which the composition is used may be determined on the basis of methods known in the art.

### Surfactants

The detergent composition may comprise one or more surfactants, which my be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually comprise from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually comprise from about 0% to about 40% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually comprise from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually comprise from about 0% to about 40% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually comprise from about 0% to about 40% by weight of a zwitterionic surfactant. Non-limiting examples of zwitterionic surfactants include betaine, alkyldimethylbetaine, sulfobetaine, and combinations thereof.

The detergent composition may also comprise one or more isoprenoid surfactants as disclosed in US 20130072416 or US 20130072415.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems comprising substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may comprise 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may comprise about 0-65% by weight, such as about 10% to about 40% of a detergent builder or co-builder, or a mixture thereof. In a dish wash detergent, the level of builder is typically 40-65%, particularly 50-65%. The builder and/or co-builder may particularly be a chelating agent (ie. a chelator) that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also comprise 0-50% by weight, such as about 10% to about 40%, of a detergent co-builder, or a mixture thereof. The detergent composition may include include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

Chelating agents or chelators are chemicals which form molecules with certain metal ions, inactivating the ions so that they cannot react with other elements thus a binding agent that suppresses chemical activity by forming chelates. Chelation is the formation or presence of two or more separate bindings between a ligand and a single central atom. The ligand may be any organic compound, a silicate or a phosphate. In the present context the term “chelating agents” comprises chelants, chelating agent, chelating agents, complexing agents, or sequestering agents that forms water-soluble complexes with metal ions such as calcium and magnesium. The chelate effect describes the enhanced affinity of chelating ligands for a metal ion compared to the affinity of a collection of similar nonchelating ligands for the same metal. Chelating agents having binding capacity with metal ions, in particular calcium (Ca2+) ions, and has been used widely in detergents and compositions in general for wash, such as laundry or dish wash. Chelating agents have however shown themselves to inhibit enzymatic activity. The term chelating agent is used in the present application interchangeably with “complexing agent” or “chelating agent” or “chelant”.

Since most alpha-amylases are calcium sensitive the presence of chelating agents these may impair the enzyme activity. The calcium sensitivity of alpha-amylases can be determined by incubating a given alpha-amylase in the presence of a strong chelating agent and analyze the impact of this incubation on the activity of the alpha-amylase in question. A calcium sensitive alpha-amylase will lose a major part or all of its activity during the incubation. Chelating agent may be present in the composition in an amount from 0.0001 wt % to 20 wt %, preferably from 0.01 to 10 wt %, more preferably from 0.1 to 5 wt %.

Strong chelating agents may be but are not limited to the following: ethylene-diamine-tetraacetic acid (EDTA), diethylene triamine penta methylene phosphonic acid (DTMPA, DTPMP), hydroxy-ethane diphosphonic acid (HEDP), ethylenediamine N,N′-disuccinic acid (EDDS), methyl glycine di-acetic acid (MGDA), diethylene triamine penta acetic acid (DTPA), propylene diamine tetraacetic acid (PDTA), 2-hydroxypyridine-N-oxide (HPNO), methyl glycine diacetic acid (MGDA), glutamic acid N,N-diacetic acid (N,N-dicarboxymethyl glutamic acid tetrasodium salt (GLDA) and nitrilotriacetic acid (NTA) or mixtures thereof. The chelating agents may be present in their acid form or a salt, preferably the chelating agents may be present as a sodium, ammonium or potassium salt.

Characterizing chelating agents: As mentioned the chelate effect or the chelating effect describes the enhanced affinity of chelating ligands for a metal ion compared to the affinity of a collection of similar nonchelating ligands for the same metal. However, the strength of this chelate effect can be determined by various types of assays or measure methods thereby differentiating or ranking the chelating agents according to their chelating effect (or strength).

In an assay the chelating agents may be characterized by their ability to reduce the concentration of free calcium ions (Ca2+) from 2.0 mM to 0.10 mM or less at pH 8.0, e.g. by using a test based on the method described by M. K. Nagarajan et al., JAOCS, Vol. 61, no. 9 (September 1984), pp. 1475-1478.

For reference, a chelator having the same ability to reduce the concentration of free calcium ions (Ca2+) from 2.0 mM to 0.10 mM at pH as EDTA at equal concentrations of the chelator are said to be strong chelators.

Bleaching Systems: The detergent may contain 0-20% by weight, such as about 0% to about 10%, of a bleaching system. Any bleaching system known in the art for use in laundry+dish wash+I&I detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

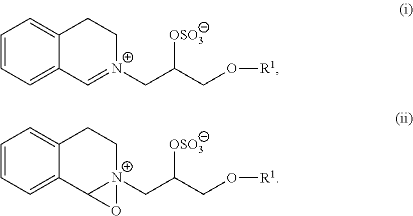

(iii) and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g. in WO2007/087258, WO2007/087244, WO2007/087259 and WO2007/087242. Suitable photobleaches may for example be sulfonated zinc phthalocyanine.

Polymers: The detergent may comprise 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

Fabric hueing agents: The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Additional Enzymes

The detergent additive as well as the detergent composition may comprise one or more additional enzymes such as a protease, lipase, cutinase, an amylase, carbohydrase, cellulase, pectinase, mannanase, arabinase, galactanase, xylanase, oxidase, e.g., a laccase, and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes N5), Celluclean™ (Novozymes N5), Celluclean Classic™ (Novozymes N5), Cellusoft™ (Novozymes N5), Whitezyme™ (Novozymes N5), Clazinase™, and Puradax HA™ (Genencor International Inc.), and KAC-500(B)™ (Kao Corporation).

Mannanases:

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes N5).

Peroxidases/Oxidases:

Suitable peroxidases/oxidases include those of plant, bacterial or fungal origin.

Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

Proteases:

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Preferenz™, Purafect MAO, Purafect Ox®, Purafect OxPO, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases:

Suitable amylases which can be used together with the enzymes of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q2645.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I

wherein the variants optionally further comprises a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants:

The detergent compositions of the present invention may also comprise dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents:

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent:

The detergent compositions of the present invention may preferably also comprise additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

Soil Release Polymers:

The detergent compositions of the present invention may also comprise one or more soil release polymers which aid the removal of soils from fabrics such as cotton and polyester based fabrics, in particular the removal of hydrophobic soils from polyester based fabrics. The soil release polymers may for example be nonionic or anionic terephthalate based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents:

The detergent compositions of the present invention may also comprise one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid. There are a number of detergent formulation forms such as layers (same or different phases), pouches, as well as forms for machine dosing unit.

Pouches can be configured as single or multicompartments. It can be of any form, shape and material which is suitable for hold the composition, e.g. without allowing the release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blend compositions comprising hydrolytically degradable and water soluble polymer blends such as polyactide and polyvinyl alcohol (known under the Trade reference M8630 as sold by Chris Craft In. Prod. Of Gary, Ind., US) plus plasticisers like glycerol, ethylene glycerol, Propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids (US2009/0011970 A1).

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Laundry Soap Bars

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they comprise, and the term laundry soap bar includes those comprising soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may comprise one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na+, K+ or NH4+ and the organic anion may be for example formate, acetate, citrate or lactate such that the salt of a monovalent cation and an organic anion may be, for example, sodium formate.

The laundry soap bar may also comprise complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g. a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, an enzyme, optionally one or more additional enzymes, a protease inhibitor, and a salt of a monovalent cation and an organic anion may be prepared and the mixture is then plodded. The enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

### Granular Detergent Formulations

A granular detergent may be formulated as described in WO09/092699, EP1705241, EP1382668, WO07/001262, U.S. Pat. No. 6,472,364, WO04/074419 or WO09/102854. Other useful detergent formulations are described in WO09/124162, WO09/124163, WO09/117340, WO09/117341, WO09/117342, WO09/072069, WO09/063355, WO09/132870, WO09/121757, WO09/112296, WO09/112298, WO09/103822, WO09/087033, WO09/050026, WO09/047125, WO09/047126, WO09/047127, WO09/047128, WO09/021784, WO09/010375, WO09/000605, WO09/122125, WO09/095645, WO09/040544, WO09/040545, WO09/024780, WO09/004295, WO09/004294, WO09/121725, WO09/115391, WO09/115392, WO09/074398, WO09/074403, WO09/068501, WO09/065770, WO09/021813, WO09/030632, and WO09/015951, WO2011025615, WO2011016958, WO2011005803, WO2011005623, WO2011005730, WO2011005844, WO2011005904, WO2011005630, WO2011005830, WO2011005912, WO2011005905, WO2011005910, WO2011005813, WO2010135238, WO2010120863, WO2010108002, WO2010111365, WO2010108000, WO2010107635, WO2010090915, WO2010033976, WO2010033746, WO2010033747, WO2010033897, WO2010033979, WO2010030540, WO2010030541, WO2010030539, WO2010024467, WO2010024469, WO2010024470, WO2010025161, WO2010014395, WO2010044905, WO2010145887, WO2010142503, WO2010122051, WO2010102861, WO2010099997, WO2010084039, WO2010076292, WO2010069742, WO2010069718, WO2010069957, WO2010057784, WO2010054986, WO2010018043, WO2010003783, WO2010003792, WO2011023716, WO2010142539, WO2010118959, WO2010115813, WO2010105942, WO2010105961, WO2010105962, WO2010094356, WO2010084203, WO2010078979, WO2010072456, WO2010069905, WO2010076165, WO2010072603, WO2010066486, WO2010066631, WO2010066632, WO2010063689, WO2010060821, WO2010049187, WO2010031607, WO2010000636.

### Method of Producing the Composition

The present invention also relates to methods of producing the composition. The method may be relevant for the (storage) stability of the detergent composition: e.g. Soap bar premix method WO2009155557.

### Uses

The present invention is directed to methods for using the alpha-amylase variants, or compositions thereof, in a cleaning process such as laundry or hard surface cleaning including automated dish wash.

The soils and stains that are important for cleaning are composed of many different substances, and a range of different enzymes, all with different substrate specificities, have been developed for use in detergents both in relation to laundry and hard surface cleaning, such as dishwashing. These enzymes are considered to provide an enzyme detergency benefit, since they specifically improve stain removal in the cleaning process that they are used in, compared to the same process without enzymes. Stain removing enzymes that are known in the art include enzymes such as proteases, amylases, lipases, cutinases, cellulases, endoglucanases, xyloglucanases, pectinases, pectin lyases, xanthanases, peroxidaes, haloperoxygenases, catalases and mannanases.

In one aspect, the invention concerns the use of alpha-amylases variants of the present invention in detergent compositions, for use in cleaning hard-surfaces, such as dish wash, or in laundering or for stain removal.

Another aspect of the invention is the use of the detergent composition comprising an alpha-amylase variant of the present invention together with one or more surfactants and optionally one or more detergent components, selected from the list comprising of hydrotropes, builders and co-builders, bleaching systems, polymers, fabric hueing agents and adjunct materials, or any mixture thereof in detergent compositions and in detergent applications.

A further aspect is the use of the detergent composition comprising an alpha-amylase of the present invention together with one or more surfactants, and one or more additional enzymes selected from the group comprising of proteases, lipases, cutinases, cellulases, endoglucanases, xyloglucanases, pectinases, pectin lyases, xanthanases, peroxidaes, haloperoxygenases, catalases and mannanases, or any mixture thereof in detergent compositions and in detergent applications.

In another aspect, the invention relates to a laundering process which may be for household laundering as well as industrial laundering. Furthermore, the invention relates to a process for the laundering of textiles (e.g. fabrics, garments, cloths etc.) where the process comprises treating the textile with a washing solution containing a detergent composition and an alpha-amylase of the present invention. The laundering may for example be carried out using a household or an industrial washing machine or be carried out by hand using a detergent composition comprising an alpha-amylase variant of the invention.

In another aspect, the invention relates to a dish wash process which may be for household dish wash as well as industrial dish wash. Furthermore, the invention relates to a process for the washing of hard surfaces (e.g. cutlery such as knives, forks, spoons; crockery such as plates, glasses, bowls; and pans) where the process comprises treating the hard surface with a washing solution comprising a detergent composition and a variant of the present invention. The hard surface washing may for example be carried out using a household or an industrial dishwasher or be carried out by hand using a detergent composition containing an alpha-amylase of the invention, optionally together with one or more further enzymes selected from the group comprising of proteases, amylases, lipases, cutinases, cellulases, endoglucanases, xyloglucanases, pectinases, pectin lyases, xanthanases, peroxidaes, haloperoxygenases, catalases, mannanases, or any mixture thereof.

In a further aspect, the invention relates to a method for removing a stain from a surface comprising contacting the surface with a composition comprising an alpha-amylase of the present invention together with one or more surfactants and optionally one or more detergent components, selected from the list comprising of hydrotropes, builders and co-builders, bleaching systems, polymers, fabric hueing agents and adjunct materials, or any mixture thereof in detergent compositions and in detergent applications. A further aspect is a method for removing a stain from a surface comprising contacting the surface with a composition comprising an alpha-amylase variant of the present invention together with one or more surfactants, one or more additional enzymes selected from the group comprising of proteases, lipases, cutinases, cellulases, endoglucanases, xyloglucanases, pectinases, pectin lyases, xanthanases, peroxidaes, haloperoxygenases, catalases and mannanases, or any mixture thereof in detergent compositions and in detergent applications.

### EXAMPLES

Amylase Expression:

the alpha-amylase variants of the present invention may be expressed by the methods well known in literature, typically involving transforming the gene coding for the amylase into the Bacillus strains such as B. subtilis and growing the organism in shake flask. Details are described below.

Strain:

E.g. B. subtilis or B. licheniformis, carrying the amylase in an expression cassette either on a plasmid or integrated on the bacillus chromosome, e.g. in the Pel or Amy locus.

Amylase Expression:

Bacillus organisms comprising the gene of alpha-amylase variant of the present invention was inoculated in LB broth containing chloramphenicol (8 μg/ml) and grown overnight at 37° C. For expression of alpha-amylase, 1-2% of overnight culture was added to 300 ml of GBT-16 medium in 1000 ml baffled flask and grown at 37° C. for 72 h.

Media:

GBT-16

| | |
|---|---|
| Glycerol | 5% w/v |
| Tryptone | 0.5% w/v |
| Beef Extract | 0.5% w/v |
| Sodium Nitrate | 1% w/v |
| Na₂HPO₄ | 1.7% w/v |
| KH₂PO₄ | 0.4% w/v |
| NH₄Cl | 0.1% w/v |
| NaCl | 0.05% w/v |

pH was adjusted to pH 7 and the media was autoclaved.
Autoclaved separately and added just before inoculation
1.47% CaCl₂—0.4 ml for 100 ml media
2.465% MgSO₄.7H₂O—0.4 ml for 100 ml media
1.39% FeSO₄—0.04 ml for 100 ml media
0.2% Na₂MoO₄.2H₂O—0.04 ml for 100 ml media
Vitamin Mix (containing 0.25% Thiamine and 0.25% Ascorbic Acid)—0.4 ml of Vitamin Mix for 100 ml media
Trace Elements (containing 0.5% MnCl₂.4H₂O, 0.2% ZnCl₂ and 0.1% CuSO₄.5H₂O)—0.04 ml of Trace solution for 100 ml media

### Example 1—Construction of Variants

Genomic DNA prepared from the strain containing LASB0000 (SEQ ID NO: 6) gene or the gene of SEQ ID NO: 3 at the Pel locus was used as template for generating the site-directed mutants.

Mutagenic forward primer and PnMi4490 (CAATCCAAGAGAACCCTGATACGGATG—SEQ ID NO: 17) reverse primer was used to generate a 3.8 kb fragment. This fragment was used as a megaprimer along with PnMi4491 (CGGAACGCCTGGCTGACAACACG—SEQ ID NO: 18) forward primer to get 6 kb insertion cassette. To enable integration in the Pel locus by double cross-over upon transformation, along with the LASB0000 (SEQ ID NO: 6) and cat genes the cassette contained upstream and downstream Pel sequences at the ends. For combining mutations that are well separated in the linear amino acid sequence (e.g. Variant1: G50A+A51T+R172D+N195F+I206Y and Variant2: N195F+I206Y+E345D+E346P+D476G), Splicing by overlap extension (SOE) PCR was carried out. Two overlapping DNA fragments for SOE PCR were generated using internal primers (Assemb19: TGGGGTGTTTGGTACACAAACAC forward (SEQ ID NO: 19) and Assemb20: CCAATCGCGCGTAAAGCTATAC reverse (SEQ ID NO: 20)) along with PnMi4490 or PnMi4491 respectively. For example, PnMi4491 and Assemb20 were used for Variant1 and Assemb19 and PnMi4490 were used for Variant2. Selection was done on LB Agar containing chloramphenicol and the mutation was confirmed by DNA sequencing of amylase gene.

Expression of amylase gene was under a triple promoter system (as described in WO 99/43835) consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence controlling the amylase expression, and the signal sequence of the B. licheniformis amylase signal to direct export out of the cells.

### Purification of Amylases:

Fermentation broths were centrifuged at 13131 g for 25 minutes to remove the cell mass, and then filtered using a 0.7 micro meter Glass filter GF-F, Whatman using Tarsons filtration assembly. Purification of amylase was done by a two-step protocol i.e. hydrophobic interaction chromatography (HIC) followed by gel filtration. Broth containing 1 M ammonium sulphate was loaded onto 20 ml column containing Methyl HIC Support from Biorad, pre-equilibrated with equilibration buffer (50 mM HEPES pH 8 containing 1M ammonium sulfate, 1 mM calcium chloride). After loading the broth, the column was washed with wash buffer (50 mM HEPES pH 8 containing 0.85M ammonium sulfate, 1 mM calcium chloride) and the elution was done with elution buffer (50 mM HEPES pH 8, 1 mM calcium chloride). Fractions containing amylase protein were pooled and passed onto Sephadex G25 (Sigma) column (200 ml column volume), pre-equilibrated with buffer containing 50 mM HEPES pH 8 and 1 mM calcium chloride.

### Example 2—Assays for Alpha-Amylase Activity

pNP-G7 Assay

The alpha-amylase activity may be determined by a method employing the G7-pNP substrate. G7-pNP which is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at λ=405 nm (400-420 nm.). Kits containing G7-pNP substrate and alpha-Glucosidase is manufactured by Roche/Hitachi (cat. No. 11876473).

### Reagents:

The G7-pNP substrate from this kit contains 22 mM 4,6-ethylidene-G7-pNP and 52.4 mM HEPES (2-[4-(2-hydroxyethyl)-1-piperazinyl]-ethanesulfonic acid), pH 7.0).

The alpha-Glucosidase reagent contains 52.4 mM HEPES, 87 mM NaCl, 12.6 mM MgCl₂, 0.075 mM CaCl₂, ≧4 kU/L alpha-glucosidase).

The substrate working solution is made by mixing 1 mL of the alpha-Glucosidase reagent with 0.2 mL of the G7-pNP substrate. This substrate working solution is made immediately before use.

Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O), (n=9-10))), 1 mM CaCl2, pH8.0.

### Procedure:

The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Phadebas Activity Assay:

The alpha-amylase activity may also be determined by a method using the Phadebas substrate (from for example Magle Life Sciences, Lund, Sweden). A Phadebas tablet includes interlinked starch polymers that are in the form of globular microspheres that are insoluble in water. A blue dye is covalently bound to these microspheres. The interlinked starch polymers in the microsphere are degraded at a speed that is proportional to the alpha-amylase activity. When the alpha-amylase degrades the starch polymers, the released blue dye is water soluble and concentration of dye can be determined by measuring absorbance at 620 nm. The concentration of blue is proportional to the alpha-amylase activity in the sample.

The amylase sample to be analysed is diluted in activity buffer with the desired pH. One substrate tablet is suspended in 5 mL activity buffer and mixed on magnetic stirrer. During mixing of substrate transfer 150 μl to microtiter plate (MTP) or PCR-MTP. Add 30 μl diluted amylase sample to 150 μl substrate and mix. Incubate for 15 minutes at 37° C. The reaction is stopped by adding 30 μl 1M NaOH and mix. Centrifuge MTP for 5 minutes at 4000×g. Transfer 100 μl to new MTP and measure absorbance at 620 nm.

The amylase sample should be diluted so that the absorbance at 620 nm is between 0 and 2.2, and is within the linear range of the activity assay.

### Reducing Sugar Activity Assay:

The alpha-amylase activity may also be determined by reducing sugar assay with for example corn starch substrate. The number of reducing ends formed by the alpha-amylase hydrolysing the alpha-1,4-glycosidic linkages in starch is determined by reaction with p-Hydroxybenzoic acid hydrazide (PHBAH). After reaction with PHBAH the number of reducing ends can be measured by absorbance at 405 nm and the concentration of reducing ends is proportional to the alpha-amylase activity in the sample.

The corns starch substrate (3 mg/ml) is solubilised by cooking for 5 minutes in milliQ water and cooled down before assay. For the stop solution prepare a Ka-Na-tartrate/NaOH solution (K—Na-tartrate (Merck 8087) 50 g/l, NaOH 20 g/l) and prepare freshly the stop solution by adding p-Hydroxybenzoic acid hydrazide (PHBAH, Sigma H9882) to Ka-Na-tartrate/NaOH solution to 15 mg/ml.

In PCR-MTP 50 μl activity buffer is mixed with 50 μl substrate. Add 50 μl diluted enzyme and mix. Incubate at the desired temperature in PCR machine for 5 minutes. Reaction is stopped by adding 75 μl stop solution (Ka-Na-tartrate/NaOH/PHBAH). Incubate in PCR machine for 10 minutes at 95° C. Transfer 150 μl to new MTP and measure absorbance at 405 nm.

The amylase sample should be diluted so that the absorbance at 405 nm is between 0 and 2.2, and is within the linear range of the activity assay.

### EnzChek® Assay:

For the determination of residual amylase activity an EnzChek® Ultra Amylase Assay Kit (E33651, Invitrogen, La Jolla, Calif., USA) may be used.

The substrate is a corn starch derivative, DQ™ starch, which is corn starch labeled with BODIPY® FL dye to such a degree that fluorescence is quenched. One vial containing approx. 1 mg lyophilized substrate is dissolved in 100 microliters of 50 mM sodium acetate (pH 4.0). The vial is vortexed for 20 seconds and left at room temperature, in the dark, with occasional mixing until dissolved. Then 900 microliters of 100 mM acetate, 0.01% (w/v) TRITON® X100, 0.125 mM CaCl₂, pH 5.5 is added, vortexed thoroughly and stored at room temperature, in the dark until ready to use. The stock substrate working solution is prepared by diluting 10-fold in residual activity buffer (100 mM acetate, 0.01% (w/v) TRITON® X100, 0.125 mM CaCl₂, pH 5.5). Immediately after incubation the enzyme is diluted to a concentration of 10-20 ng enzyme protein/ml in 100 mM acetate, 0.01% (W/v) TRITON® X100, 0.125 mM CaCl₂, pH 5.5.

For the assay, 25 microliters of the substrate working solution is mixed for 10 second with 25 microliters of the diluted enzyme in a black 384 well microtiter plate. The fluorescence intensity is measured (excitation: 485 nm, emission: 555 nm) once every minute for 15 minutes in each well at 25° C. and the Vₘₐₓ is calculated as the slope of the plot of fluorescence intensity against time. The plot should be linear and the residual activity assay has been adjusted so that the diluted reference enzyme solution is within the linear range of the activity assay.

### Reference Alpha-Amylase

The reference alpha-amylase may be the amylase of SEQ ID NO: 2. If not stated otherwise it is the pNP-G7 assay that is used for determining alpha-amylase activity of the variants.

### Example 3—Wash Performance of Alpha-Amylases Using Automatic Mechanical Stress Assay (AMSA)

In order to assess the wash performance of the alpha-amylases in a detergent base composition, washing experiments may be performed using Automatic Mechanical Stress Assay (AMSA). With the AMSA test the wash performance of a large quantity of small volume enzyme-detergent solutions can be examined. The AMSA plate has a number of slots for test solutions and a lid firmly squeezing the textile swatch to be washed against all the slot openings. During the washing time, the plate, test solutions, textile and lid are vigorously shaken to bring the test solution in contact with the textile and apply mechanical stress in a regular, periodic oscillating manner. For further description see WO 02/42740, especially the paragraph “Special method embodiments” at page 23-24.

### General Wash Performance Description

A test solution comprising water (6° dH), 0.79 g/L detergent, e.g. model detergent J as described below, and the enzyme of the invention at concentration of 0 or 0.05 or 0.2 mg enzyme protein/L, is prepared. Fabrics stained with starch (CS-28 from Center For Test materials BV, P.O. Box 120, 3133 KT, Vlaardingen, The Netherlands) is added and washed for 20 minutes at 15° C. and 30° C., or alternatively 20 minutes at 15° C. and 40° C. as specified in the examples. After thorough rinse under running tap water and drying in the dark, the light intensity values of the stained fabrics are subsequently measured as a measure for wash performance. The test with 0 mg enzyme protein/L is used as a blank and corresponds to the contribution from the detergent. Preferably mechanical action is applied during the wash step, e.g. in the form of shaking, rotating or stirring the wash solution with the fabrics. The AMSA wash performance experiments may be conducted under the experimental conditions specified below:

TABLE A
Experimental condition

| | |
|---|---|
| Detergent | Liquid Model detergent J (see Table B) |
| Detergent dosage | 0.79 g/L |
| Test solution volume | 160 micro L |
| pH | As is |
| Wash time | 20 minutes |
| Temperature | 15° C. or 30° C. |
| Water hardness | 6° dH |
| Enzyme concentration | 0.2 mg (15° C. wash) or 0.05 (30° C. wash) mg |
| in test | enzyme protein/L |
| Test material | CS-28 (Rice starch cotton) |

TABLE B
Model detergent J

| | Content of compound | % active component |
|---|---|---|
| Compound | (% w/w) | (% w/w) |
| LAS | 5.15 | 5.00 |
| AS | 5.00 | 4.50 |
| AEOS | 14.18 | 10.00 |
| Coco fatty acid | 1.00 | 1.00 |
| AEO | 5.00 | 5.00 |
| MEA | 0.30 | 0.30 |
| MPG | 3.00 | 3.00 |
| Ethanol | 1.50 | 1.35 |
| DTPA (as Na5 salt) | 0.25 | 0.10 |
| Sodium citrate | 4.00 | 4.00 |
| Sodium formate | 1.00 | 1.00 |
| Sodium hydroxide | 0.66 | 0.66 |
| H₂O, ion exchanged | 58.95 | 58.95 |

Water hardness was adjusted to 6° dH by addition of CaCl2, MgCl2, and NaHCO3 (Ca²⁺:Mg²⁺:HCO3⁻=2:1:4.5) to the test system. After washing the textiles were flushed in tap water and dried.

TABLE C
Experimental condition

| | |
|---|---|
| Detergent | Liquid Model detergent A (see Table D) |
| Detergent dosage | 3.33 g/L |
| Test solution volume | 160 micro L |
| pH | As is |
| Wash time | 20 minutes |
| Temperature | 15° C. or 40° C. |
| Water hardness | 15° dH |
| Enzyme concentration | 0.2 mg (15° C. wash) or 0.05 (40° C. wash) mg |
| in test | enzyme protein/L |
| Test material | CS-28 (Rice starch cotton) |

TABLE D
Model detergent A

| | Content of compound | % active component |
|---|---|---|
| Compound | (% w/w) | (% w/w) |
| LAS | 12.00 | 11.60 |
| AEOS, SLES | 17.63 | 4.90 |
| Soy fatty acid | 2.75 | 2.48 |
| Coco fatty acid | 2.75 | 2.80 |
| AEO | 11.00 | 11.00 |
| Sodium hydroxide | 1.75 | 1.80 |
| Ethanol/Propan-2-ol | 3.00 | 2.70/0.30 |
| MPG | 6.00 | 6.00 |
| Glycerol | 1.71 | 1.70 |
| TEA | 3.33 | 3.30 |
| Sodium formate | 1.00 | 1.00 |
| Sodium citrate | 2.00 | 2.00 |
| DTMPA | 0.48 | 0.20 |
| PCA | 0.46 | 0.18 |
| Phenoxy ethanol | 0.50 | 0.50 |
| H₂O, ion exchanged | 33.64 | 33.64 |

Water hardness was adjusted to 15° dH by addition of CaCl2, MgCl2, and NaHCO3 (Ca²⁺:Mg²⁺:HCO3⁻=4:1:7.5) to the test system. After washing the textiles were flushed in tap water and dried.

The wash performance is measured as the brightness expressed as the intensity of the light reflected from the sample when illuminated with white light. When the sample is stained the intensity of the reflected light is lower, than that of a clean sample. Therefore the intensity of the reflected light can be used to measure wash performance.

Color measurements are made with a professional flatbed scanner (EPSON Expression 10000XL, EPSON) used to capture an image of the washed textile.

To extract a value for the light intensity from the scanned images, 48→24 Bit Color pixel values from the image are converted into values for red, green and blue (RGB). The intensity value (Int) is calculated by adding the RGB values together as vectors and then taking the length of the resulting vector:

Int=\sqrt{r²+g²+b²}

### Textile:

Textile sample CS-28 (rice starch on cotton) is obtained from Center For Testmaterials BV, P.O. Box 120, 3133 KT Vlaardingen, the Netherlands.

TABLE E
Wash performance of variants

| | Improvement | Improvement | Improvement | Improvement |
|---|---|---|---|---|
| | factor (IF) | factor (IF) | factor (IF) | factor (IF) |
| | in Model | in Model | in Model | in Model |
| VARIANTS - | detergent A | detergent A | detergent J | detergent J |
| of SEQ ID NO: 2 using SEQ | 15° C., 0.2 mg | 40° C. 0.05 mg | 15° C., 0.2 mg | 30° C. 0.05 mg |
| ID NO: 1 for numbering | EP | EP | EP | EP |
| Reference: | 1 | 1 | 1 | 1 |
| SEQ ID NO: 2 | | | | |
| SEQ ID NO: 2 + Y363L | 1.15 | 1.2 | 1.55 | 1.32 |
| SEQ ID NO: 2 + Y160H | 0.96 | 1.12 | 1.19 | 1.08 |
| SEQ ID NO: 2 + Y160L | 1.1 | 1.06 | 1.27 | 1.01 |
| SEQ ID NO: 2 + W167L | 1.29 | 1.1 | 1.16 | 1.07 |
| SEQ ID NO: 2 + W167F | 1.38 | 1.25 | 1.86 | 1.29 |
| SEQ ID NO: 2 + Q169K | 1.08 | 1.03 | 1.26 | 0.94 |
| SEQ ID NO: 2 + Q169R | 1.08 | 0.99 | 1.03 | 1.09 |
| SEQ ID NO: 2 + K72S | 1.49 | 1.11 | 1.33 | 1.02 |
| SEQ ID NO: 2 + K72R | 1.4 | 1.23 | 1.33 | 0.98 |
| SEQ ID NO: 2 + M200L | 1.03 | 0.89 | 1.09 | 0.51 |
| SEQ ID NO: 2 + T40K | 1.39 | 1.31 | 1.45 | 1.24 |
| SEQ ID NO: 2 + T40M | 1.3 | 1.18 | 1.31 | 1.04 |
| SEQ ID NO: 2 + H1* + H2* + G7S | 1.4 | 1.1 | 1.22 | 0.65 |
| SEQ ID NO: 2 + Q361S | 1.23 | 1.18 | 1.3 | 1.32 |
| SEQ ID NO: 2 + Q361A | 1.24 | 1.2 | 1.24 | 1.38 |
| SEQ ID NO: 2 + E345S | 1.31 | 1.23 | 1.23 | 1.55 |
| SEQ ID NO: 2 + E276S | 1.15 | 1.16 | 0.95 | 1.36 |
| SEQ ID NO: 2 + W268F | 1.22 | 1.06 | 1.15 | 1.26 |
| SEQ ID NO: 2 + W268L | 1.06 | 0.72 | 0.93 | 0.8 |
| SEQ ID NO: 2 + V291A | 1.54 | 1.27 | 1.39 | 1.33 |
| SEQ ID NO: 2 + V56A | 1.34 | 1.16 | 1.21 | 1.34 |
| SEQ ID NO: 2 + V56T | 1.68 | 1.49 | 1.44 | 1.66 |
| SEQ ID NO: 2 + R171E | 2.11 | 1.51 | 1.95 | 1.76 |
| SEQ ID NO: 2 + R171S | 0.96 | 0.99 | 1.31 | 1.02 |
| SEQ ID NO: 2 + K302A | 1.18 | 1.15 | 1.23 | 1.13 |
| SEQ ID NO: 2 + L351A | 1.06 | 0.96 | 1.12 | 1.02 |
| SEQ ID NO: 2 + L353A | 0.99 | 1.03 | 0.99 | 0.89 |
| SEQ ID NO: 2 + M307L | 0.98 | 1.04 | 0.9 | 0.91 |
| SEQ ID NO: 2 + L429A | 1.29 | 1.12 | 1.15 | 1.2 |
| SEQ ID NO: 2 + S478A | 1.07 | 1.04 | 1.07 | 1.06 |
| SEQ ID NO: 2 + A354V | 1.3 | 1.08 | 1.23 | 1.26 |
| SEQ ID NO: 2 + K349M | 1.12 | 0.71 | 1.19 | 1.12 |
| SEQ ID NO: 2 + K349A | 1.14 | 0.97 | 1.13 | 1.06 |
| SEQ ID NO: 2 + Y351W | 0.98 | 0.99 | 0.84 | 1.01 |
| SEQ ID NO: 2 + K483R | 1.07 | 0.97 | 1.2 | 0.93 |
| SEQ ID NO: 2 + E346G | 0.99 | 1.07 | 0.94 | 1.01 |
| SEQ ID NO: 2 + N270G | 0.95 | 1.29 | 1 | 1.01 |
| SEQ ID NO: 2 + T282G | 0.98 | 1.24 | 1.17 | 0.94 |
| SEQ ID NO: 2 + | 0.86 | 1.11 | 0.79 | 1.05 |
| K269G + N270G | | | | |
| SEQ ID NO: 2 + H421R | 0.83 | 0.86 | 0.74 | 1.04 |
| SEQ ID NO: 2 + E276N | 0.74 | 1.18 | 0.76 | 0.89 |
| SEQ ID NO: 2 + T40K + K72R | 1.08 | 0.99 | 1.05 | 1.04 |
| SEQ ID NO: 2 + | 0.93 | 1.09 | 1.22 | 0.93 |
| N270Y + Y295N | | | | |
| SEQ ID NO: 2 + | 1.18 | 1.07 | 1.05 | 1.04 |
| N270Y + Y295N + N299T | | | | |
| SEQ ID NO: 2 + | 1.07 | 1.01 | 0.97 | 0.98 |
| K269S + N270Y + A274K + Y295N + | | | | |
| N299T | | | | |
| SEQ ID NO: 2 + Y295N | 1.04 | 1.12 | 0.73 | 1.05 |
| SEQ ID NO: 2 + | 1.1 | 0.95 | 0.6 | 0.81 |
| Y295N + N299T | | | | |
| SEQ ID NO: 2 + | 1.32 | 1.02 | 1.12 | 1.15 |
| A113Y + R171E | | | | |
| SEQ ID NO: 2 + | 1.08 | 0.82 | 1.06 | 0.85 |
| V56T + A113Y + R171E | | | | |
| SEQ ID NO: 2 + T40K + K179S | 1.23 | 1.05 | 1.1 | 1.1 |
| SEQ ID NO: 2 + R171E + E391L | 1.02 | 0.92 | 1.14 | 1.14 |
| SEQ ID NO: 2 + R171E + Y363L | 1.06 | 0.6 | 1.2 | 0.48 |
| SEQ ID NO: 2 + | 1.35 | 1.01 | 1.37 | 1.25 |
| H1L + R171E + D192E | | | | |
| SEQ ID NO: 2 + T40K + R171E | 1.35 | 0.97 | 1.38 | 1.38 |
| SEQ ID NO: 2 + | 1.32 | 0.87 | 1.34 | 0.63 |
| R171E + H324L + Q361A + Y363L | | | | |
| SEQ ID NO: 2 + | 1.41 | 0.74 | 1.2 | 0.45 |
| R171E + Q361A + Y363L | | | | |
| SEQ ID NO: 2 + | 1.39 | 0.95 | 1.07 | 0.97 |
| A113Y + R171E + E391L | | | | |
| SEQ ID NO: 2 + | 1.24 | 0.66 | 1.18 | 0.87 |
| R171E + D192E + Y363L | | | | |
| SEQ ID NO: 2 + | 1.05 | 0.46 | 0.84 | 0.4 |
| R171E + K423P | | | | |
| SEQ ID NO: 2 + | 1.49 | 0.76 | 1.32 | 0.68 |
| A113Y + R171E + Y363L + E391L + | | | | |
| K423P | | | | |
| SEQ ID NO: 2 + S36D | 0.93 | 1.07 | 0.94 | 0.74 |
| SEQ ID NO: 2 + R118K | 0.93 | 1.06 | 0.92 | 0.88 |
| SEQ ID NO: 2 + R158K | 1.06 | 0.77 | 0.96 | 0.91 |
| SEQ ID NO: 2 + R320K | 1.09 | 0.83 | 0.92 | 0.9 |
| SEQ ID NO: 2 + R383K | 1.01 | 1.15 | 0.93 | 0.89 |
| SEQ ID NO: 2 + P398R | 1.04 | 0.96 | 0.9 | 0.83 |
| SEQ ID NO: 2 + P408H | 1.25 | 1 | 1.09 | 0.98 |
| SEQ ID NO: 2 + K423N | 1.26 | 0.74 | 1.1 | 0.99 |
| SEQ ID NO: 2 + I430M | 1.26 | 0.87 | 1.11 | 1.04 |
| SEQ ID NO: 2 + L444R | 0.93 | 1.04 | 0.83 | 0.93 |
| SEQ ID NO: 2 + K461T | 0.92 | 0.98 | 1.04 | 0.94 |
| SEQ ID NO: 2 + E471N | 1.03 | 1.01 | 0.89 | 1.1 |
| SEQ ID NO: 2 + R142Q | 1 | 0.95 | 1.06 | 0.85 |
| SEQ ID NO: 2 + R158A | 1.04 | 0.88 | 1.11 | 0.98 |
| SEQ ID NO: 2 + W140Y | 1.3 | 1.08 | 1.58 | 1.32 |
| SEQ ID NO: 2 + R320A | 1.2 | 0.97 | 1.28 | 1.1 |
| SEQ ID NO: 2 + | 1.4 | 1.08 | 1.22 | 0.94 |
| W140Y + R320A | | | | |
| SEQ ID NO: 2 + A113H | 1.06 | 1.05 | 1.04 | 1.02 |
| SEQ ID NO: 2 + A113Q | 0.99 | 0.91 | 0.84 | 1.18 |
| SEQ ID NO: 2 + R172Y | 1.34 | 1.09 | 1.15 | 1.57 |
| SEQ ID NO: 2 + A113S | 1.01 | 1.1 | 0.83 | 1.08 |
| SEQ ID NO: 2 + A113N | 1.28 | 1.22 | 1.05 | 1.63 |
| SEQ ID NO: 2 + R142K | 1.41 | 1.17 | 1.28 | 1.9 |
| SEQ ID NO: 2 + R172L | 1.26 | 1.14 | 1.28 | 1.52 |
| SEQ ID NO: 2 + R172S | 1.53 | 1.19 | 1.43 | 1.41 |
| SEQ ID NO: 2 + A113Y | 1.48 | 1.35 | 1.38 | 2.01 |
| SEQ ID NO: 2 + A113L | 1.38 | 1.21 | 1.42 | 1.87 |
| SEQ ID NO: 2 + R142T | 1.02 | 0.9 | 0.89 | 1.12 |
| SEQ ID NO: 2 + R142S | 1.13 | 0.99 | 0.94 | 1.06 |
| SEQ ID NO: 2 + R172P | 1.21 | 1.15 | 1.04 | 1.07 |
| SEQ ID NO: 2 + R172T | 1.51 | 1.15 | 1.39 | 1.36 |
| SEQ ID NO: 2 + R172V | 1.53 | 1.08 | 1.19 | 1.31 |
| SEQ ID NO: 2 + R172A | 1.13 | 1.02 | 1.16 | 1.13 |
| SEQ ID NO: 2 + R172M | 0.79 | 1.02 | 0.88 | 1.09 |
| SEQ ID NO: 2 + L173V | 1.18 | 1.11 | 1.1 | 0.96 |
| SEQ ID NO: 2 + L173S | 1.02 | 0.83 | 0.78 | 1.01 |
| SEQ ID NO: 2 + N174L | 1.08 | 0.84 | 0.98 | 1.22 |
| SEQ ID NO: 2 + I241F | 0.97 | 1.07 | 1.07 | 1.23 |
| SEQ ID NO: 2 + N174Y | 1.2 | 1.16 | 1.03 | 1.2 |
| SEQ ID NO: 2 + L173T | 1.05 | 0.98 | 0.81 | 0.96 |
| SEQ ID NO: 2 + N174G | 1.14 | 1.11 | 1.06 | 1.27 |
| SEQ ID NO: 2 + I241Q | 1 | 1 | 1.02 | 0.85 |
| SEQ ID NO: 2 + Y243E | 1.33 | 1.19 | 1.28 | 1.27 |
| SEQ ID NO: 2 + Y243L | 1.05 | 0.99 | 1.01 | 1.07 |
| SEQ ID NO: 2 + Y243I | 1 | 1.08 | 0.83 | 1.14 |
| SEQ ID NO: 2 + Y243F | 0.98 | 1.03 | 1.1 | 0.92 |
| SEQ ID NO: 2 + Y243S | 1.32 | 1.2 | 1.14 | 1.31 |
| SEQ ID NO: 2 + Y243G | 1.44 | 1.21 | 1.34 | 1.53 |
| SEQ ID NO: 2 + Y243D | 1.5 | 1.22 | 1.32 | 1.43 |
| SEQ ID NO: 2 + V165G | 1.5 | 1.18 | 1.28 | 1.36 |
| SEQ ID NO: 2 + Y243T | 1.24 | 1.16 | 1.21 | 1.35 |
| SEQ ID NO: 2 + N299G | 0.81 | 1.18 | 0.95 | 1.04 |
| SEQ ID NO: 2 + N299K | 1.03 | 1.15 | 1.17 | 1.12 |
| SEQ ID NO: 2 + N299R | 0.64 | 1.02 | 0.72 | 0.76 |
| SEQ ID NO: 2 + Y160R | 0.94 | 1.18 | 0.92 | 0.92 |
| SEQ ID NO: 2 + Y160Q | 1.02 | 1.16 | 1.07 | 1.12 |
| SEQ ID NO: 2 + Y160G | 0.93 | 1.11 | 0.93 | 0.93 |
| SEQ ID NO: 2 + P211E | 1.01 | 1.11 | 1.03 | 0.91 |
| SEQ ID NO: 2 + P211G | 0.88 | 1.16 | 0.86 | 0.9 |
| SEQ ID NO: 2 + P211V | 1.05 | 1.29 | 1.01 | 1.02 |
| SEQ ID NO: 2 + H210M | 0.96 | 1.23 | 0.95 | 0.94 |
| SEQ ID NO: 2 + P211D | 1.3 | 1 | 1.25 | 1.11 |
| SEQ ID NO: 2 + P211A | 0.86 | 1.13 | 0.81 | 0.9 |
| SEQ ID NO: 2 + K269A | 0.88 | 1.02 | 0.87 | 0.75 |
| SEQ ID NO: 2 + P211W | 0.83 | 1.09 | 0.74 | 1.01 |
| SEQ ID NO: 2 + K269L | 1.17 | 1.31 | 1.15 | 1.13 |
| SEQ ID NO: 2 + K269N | 1.1 | 1.09 | 1.12 | 1.29 |
| SEQ ID NO: 2 + K269R | 0.97 | 1.18 | 0.95 | 1.19 |
| SEQ ID NO: 2 + F155Y | 0.97 | 0.96 | 0.94 | 1.26 |
| SEQ ID NO: 2 + K269I | 1.14 | 0.95 | 1.22 | 1.25 |
| SEQ ID NO: 2 + K269V | 1.2 | 1.23 | 1.14 | 1.31 |
| SEQ ID NO: 2 + K269M | 1.32 | 1.34 | 1.42 | 1.31 |
| SEQ ID NO: 2 + N270A | 0.86 | 1.11 | 1.04 | 1.09 |
| SEQ ID NO: 2 + N270P | 0.73 | 1.07 | 0.84 | 0.9 |
| SEQ ID NO: 2 + R118G | 1.07 | 1.1 | 1.28 | 1.1 |
| SEQ ID NO: 2 + V213T | 0.79 | 1.06 | 0.96 | 0.98 |
| SEQ ID NO: 2 + R118P | 1.09 | 0.97 | 1.5 | 1.16 |
| SEQ ID NO: 2 + K179M | 1 | 1.04 | 1.29 | 0.99 |
| SEQ ID NO: 2 + K179P | 0.98 | 0.98 | 1.09 | 1.11 |
| SEQ ID NO: 2 + K179R | 0.99 | 1.09 | 1.04 | 1.12 |
| SEQ ID NO: 2 + E391S | 0.93 | 1 | 0.68 | 0.81 |
| SEQ ID NO: 2 + E391A | 1.2 | 1.31 | 1.52 | 1.23 |
| SEQ ID NO: 2 + E391T | 1.06 | 1.08 | 0.99 | 1 |
| SEQ ID NO: 2 + E391M | 1.28 | 1.2 | 1.34 | 2.27 |
| SEQ ID NO: 2 + K179S | 1.43 | 1.17 | 1.34 | 1.09 |
| SEQ ID NO: 2 + E391R | 0.81 | 0.99 | 1.02 | 1 |
| SEQ ID NO: 2 + E391L | 1.19 | 1.35 | 1.58 | 1.42 |
| SEQ ID NO: 2 + E391K | 0.96 | 1.09 | 1.14 | 0.9 |
| SEQ ID NO: 2 + E391H | 1.31 | 1.29 | 1.5 | 1.25 |
| SEQ ID NO: 2 + E391I | 0.92 | 1 | 0.97 | 0.8 |
| SEQ ID NO: 2 + K179A | 1.1 | 1.07 | 1.04 | 0.95 |
| SEQ ID NO: 2 + K179T | 1.43 | 1.21 | 1.85 | 1.19 |
| SEQ ID NO: 2 + H1G | 1.28 | 1.19 | 1.45 | 1.13 |
| SEQ ID NO: 2 + H1W | 1.06 | 1.03 | 1.19 | 0.97 |
| SEQ ID NO: 2 + H1R | 0.97 | 1.05 | 1.12 | 1.18 |
| SEQ ID NO: 2 + H1K | 1.04 | 1.11 | 1.22 | 1.51 |
| SEQ ID NO: 2 + H1S | 1.18 | 1.09 | 1.25 | 1.43 |
| SEQ ID NO: 2 + H1L | 1.22 | 1.37 | 1.28 | 1.6 |
| SEQ ID NO: 2 + L217M | 0.99 | 1.22 | 1.08 | 1.39 |
| SEQ ID NO: 2 + K423W | 1.04 | 0.91 | 1.06 | 1 |
| SEQ ID NO: 2 + K423R | 1 | 0.98 | 0.9 | 1 |
| SEQ ID NO: 2 + K423L | 1.15 | 1.04 | 1.1 | 1.12 |
| SEQ ID NO: 2 + K423H | 1.29 | 1.15 | 1.27 | 1.18 |
| SEQ ID NO: 2 + K423P | 1.24 | 1.13 | 1.34 | 1.2 |
| SEQ ID NO: 2 + K423T | 1.16 | 1.12 | 1.15 | 1.38 |
| SEQ ID NO: 2 + L217F | 1.17 | 1.19 | 1.07 | 1.32 |
| SEQ ID NO: 2 + K302R | 1.25 | 1.22 | 1.22 | 1.29 |
| SEQ ID NO: 2 + N296Q | 1.11 | 1.08 | 1.03 | 1.33 |
| SEQ ID NO: 2 + H1N | 1.22 | 1.19 | 1.18 | 1.41 |
| SEQ ID NO: 2 + N197A | 1.17 | 1.07 | 1.17 | 1.08 |
| SEQ ID NO: 2 + V165M | 1.31 | 0.96 | 1.29 | 1.06 |
| SEQ ID NO: 2 + A186L | 1.01 | 1 | 1.04 | 0.93 |
| SEQ ID NO: 2 + D192A | 1.1 | 1.08 | 1.24 | 1.01 |
| SEQ ID NO: 2 + D192E | 1.26 | 1.1 | 1.27 | 1.19 |
| SEQ ID NO: 2 + N283Q | 1.11 | 1.08 | 1.05 | 1.14 |
| SEQ ID NO: 2 + N283S | 1.1 | 0.95 | 1.05 | 1.06 |
| SEQ ID NO: 2 + V165A | 1.23 | 1.1 | 1.25 | 1.48 |
| SEQ ID NO: 2 + A186F | 0.89 | 1.03 | 0.95 | 0.95 |
| SEQ ID NO: 2 + N281H | 0.99 | 1 | 1.05 | 1.07 |
| SEQ ID NO: 2 + V165L | 0.97 | 1.06 | 1.15 | 1.07 |
| SEQ ID NO: 2 + A186V | 1.05 | 1.02 | 1 | 1.2 |
| SEQ ID NO: 2 + N283L | 1.03 | 0.86 | 0.84 | 1.13 |
| SEQ ID NO: 2 + M208A | 1.07 | 1.19 | 1.14 | 0.97 |
| SEQ ID NO: 2 + M208S | 1.13 | 1.07 | 1.05 | 1.09 |
| SEQ ID NO: 2 + N283D | 1.06 | 0.96 | 0.99 | 0.86 |
| SEQ ID NO: 2 + N283V | 0.97 | 0.95 | 0.91 | 1.14 |
| SEQ ID NO: 2 + A186T | 0.99 | 0.96 | 0.97 | 1.12 |
| SEQ ID NO: 2 + M208G | 0.85 | 1.07 | 1.03 | 1.02 |
| SEQ ID NO: 2 + V165Q | 1.05 | 1.09 | 0.96 | 1.04 |
| SEQ ID NO: 2 + A186M | 1.28 | 1.03 | 1.17 | 0.98 |
| SEQ ID NO: 2 + M208V | 0.99 | 1.04 | 0.88 | 1.04 |
| SEQ ID NO: 2 + V165S | 0.86 | 1.05 | 0.97 | 1.05 |
| SEQ ID NO: 2 + M208W | 0.99 | 1.09 | 0.86 | 0.92 |
| SEQ ID NO: 2 + N283A | 1.05 | 1.05 | 0.93 | 0.93 |
| SEQ ID NO: 2 + H321V | 0.99 | 0.79 | 1.26 | 0.96 |
| SEQ ID NO: 2 + H2C | 0.97 | 0.98 | 1.06 | 0.87 |
| SEQ ID NO: 2 + Q98V | 0.91 | 1.08 | 1.13 | 1.07 |
| SEQ ID NO: 2 + H321A | 1.12 | 1.09 | 1.03 | 0.99 |
| SEQ ID NO: 2 + H321L | 0.97 | 1.06 | 0.86 | 1.1 |
| SEQ ID NO: 2 + H324R | 1.12 | 0.95 | 1.33 | 1.12 |
| SEQ ID NO: 2 + H324W | 1.12 | 0.88 | 1.26 | 1 |
| SEQ ID NO: 2 + S323K | 1.03 | 1.04 | 1.12 | 1.02 |
| SEQ ID NO: 2 + S323E | 1.12 | 0.99 | 0.93 | 0.94 |
| SEQ ID NO: 2 + H324L | 1.01 | 1.43 | 1.16 | 1.21 |
| SEQ ID NO: 2 + H324Q | 1 | 1.04 | 1.11 | 0.89 |
| SEQ ID NO: 2 + S323W | 0.94 | 1.03 | 0.93 | 1.07 |
| SEQ ID NO: 2 + H324A | 0.96 | 1.02 | 1.06 | 1 |
| SEQ ID NO: 2 + N128G | 0.79 | 0.97 | 0.87 | 1.07 |
| SEQ ID NO: 2 + N128V | 0.71 | 0.98 | 0.83 | 1.05 |
| SEQ ID NO: 2 + N128L | 0.96 | 1.07 | 0.97 | 0.88 |
| SEQ ID NO: 2 + G7L | 0.96 | 0.77 | 0.92 | 1.12 |
| SEQ ID NO: 2 + N128T | 1.11 | 0.88 | 1.03 | 1.03 |
| SEQ ID NO: 2 + N128H | 0.95 | 0.97 | 0.73 | 1.23 |
| SEQ ID NO: 2 + G182N | 1.08 | 0.98 | 0.86 | 1.14 |
| SEQ ID NO: 2 + G182Q | 1 | 1.01 | 0.94 | 0.97 |
| SEQ ID NO: 2 + T193V | 1.09 | 0.94 | 0.85 | 0.9 |
| SEQ ID NO: 2 + N128M | 1.07 | 1.13 | 1.06 | 1.08 |
| SEQ ID NO: 2 + G182A | 0.98 | 0.94 | 0.87 | 1.05 |
| SEQ ID NO: 2 + T193G | 1.33 | 0.98 | 1.06 | 1.18 |
| SEQ ID NO: 2 + T193A | 1.12 | 0.95 | 0.92 | 0.99 |
| SEQ ID NO: 2 + T193N | 1.12 | 0.99 | 1.03 | 0.84 |
| SEQ ID NO: 2 + T193M | 1.06 | 1 | 0.92 | 0.96 |
| SEQ ID NO: 2 + T193S | 1.08 | 0.89 | 0.97 | 0.95 |
| SEQ ID NO: 2 + H1* + H2E | 1.16 | 1.25 | 1.12 | 1.14 |
| SEQ ID NO: 2 + Q280R | 0.93 | 1.06 | 0.78 | 1.01 |
| SEQ ID NO: 2 + H1* + H2V | 1.14 | 1.26 | 1.15 | 1.11 |
| SEQ ID NO: 2 + Q280S | 1.2 | 1.22 | 1.21 | 1.12 |
| SEQ ID NO: 2 + H1* + H2A | 1.25 | 1.24 | 1.15 | 1.07 |
| SEQ ID NO: 2 + Q280K | 0.97 | 1.05 | 1.02 | 0.93 |
| SEQ ID NO: 2 + G304R | 1.04 | 1.11 | 1.07 | 1.36 |
| SEQ ID NO: 2 + G304V | 1.12 | 1.03 | 1.15 | 1.04 |
| SEQ ID NO: 2 + V410R | 0.94 | 1.11 | 1.1 | 1.34 |
| SEQ ID NO: 2 + G304A | 1.06 | 1.09 | 1.12 | 1.27 |
| SEQ ID NO: 2 + I430A | 1.02 | 0.95 | 0.95 | 1.02 |
| SEQ ID NO: 2 + Y203M | 0.94 | 0.85 | 1.02 | 0.84 |
| SEQ ID NO: 2 + G305S | 1.09 | 1.08 | 1.08 | 1.41 |
| SEQ ID NO: 2 + I430L | 1.08 | 1.1 | 0.96 | 1 |
| SEQ ID NO: 2 + I430E | 1.29 | 1.26 | 1.44 | 1.57 |
| SEQ ID NO: 2 + G149Q | 1.19 | 1.18 | 1.13 | 1.48 |
| SEQ ID NO: 2 + G149R | 1.07 | 1.13 | 1 | 0.94 |
| SEQ ID NO: 2 + G149K | 0.97 | 1.13 | 0.92 | 0.92 |
| SEQ ID NO: 2 + G149A | 1.1 | 1.33 | 0.96 | 1.27 |
| SEQ ID NO: 2 + G149L | 0.77 | 1.15 | 0.81 | 1.01 |
| SEQ ID NO: 2 + D476G | 0.77 | 1.14 | 0.7 | 1.19 |
| SEQ ID NO: 2 + E346A | 1.09 | 1.06 | 1.03 | 0.95 |
| SEQ ID NO: 2 + Q169G | 1.14 | 0.9 | 1.01 | 0.85 |
| SEQ ID NO: 2 + Q169M | 1.42 | 1.12 | 1.25 | 1.2 |
| SEQ ID NO: 2 + R181G | 1.04 | 0.95 | 1.18 | 1.03 |
| SEQ ID NO: 2 + R171G | 1 | 0.97 | 0.99 | 1 |
| SEQ ID NO: 2 + K463R | 0.86 | 0.98 | 1.13 | 0.77 |
| SEQ ID NO: 2 + R171H | 1.01 | 1.04 | 0.99 | 1.21 |
| SEQ ID NO: 2 + N311H | 0.87 | 0.91 | 1.01 | 0.82 |
| SEQ ID NO: 2 + N311S | 1.09 | 0.98 | 1.26 | 1.13 |
| SEQ ID NO: 2 + N215S | 1.04 | 0.95 | 1.04 | 0.92 |
| SEQ ID NO: 2 + N215A | 1.16 | 1.03 | 1.02 | 1.05 |
| SEQ ID NO: 2 + N311L | 1.15 | 0.95 | 1.11 | 0.9 |
| SEQ ID NO: 2 + L351C | 1.25 | 1 | 1.22 | 0.94 |
| SEQ ID NO: 2 + N215G | 1.37 | 1.03 | 1.35 | 1.37 |
| SEQ ID NO: 2 + L351H | 1.12 | 1.01 | 0.96 | 0.75 |
| SEQ ID NO: 2 + N215D | 1.03 | 0.95 | 1.05 | 0.87 |
| SEQ ID NO: 2 + N311E | 1.28 | 0.9 | 1.16 | 1.11 |
| SEQ ID NO: 2 + N311F | 0.96 | 1.04 | 0.89 | 0.99 |
| SEQ ID NO: 2 + L351Q | 0.87 | 1.03 | 1.18 | 1.06 |
| SEQ ID NO: 2 + N311W | 0.77 | 0.95 | 0.93 | 1.01 |
| SEQ ID NO: 2 + N311D | 1.08 | 1 | 1.16 | 0.97 |
| SEQ ID NO: 2 + N219S | 1.23 | 1.14 | 0.99 | 1.04 |
| SEQ ID NO: 2 + N219E | 1.25 | 1.04 | 1 | 1.07 |
| SEQ ID NO: 2 + N219T | 1.18 | 1.09 | 0.94 | 0.99 |
| SEQ ID NO: 2 + H1* + N3C | 1.16 | 0.95 | 0.83 | 0.83 |
| SEQ ID NO: 2 + L355V | 1.04 | 1.09 | 0.94 | 0.93 |
| SEQ ID NO: 2 + D418L | 1.05 | 1 | 1.15 | 0.94 |
| SEQ ID NO: 2 + H1* + N3G | 0.85 | 1 | 0.78 | 0.78 |
| SEQ ID NO: 2 + H1* + N3A | 1.03 | 0.93 | 0.97 | 0.76 |
| SEQ ID NO: 2 + A274W | 1.07 | 1.03 | 1.11 | 1.02 |
| SEQ ID NO: 2 + G133Q | 0.87 | 1.01 | 1.05 | 0.82 |
| SEQ ID NO: 2 + A274T | 1.15 | 0.93 | 1.06 | 0.97 |
| SEQ ID NO: 2 + L351S | 1.17 | 0.95 | 1.03 | 0.97 |
| SEQ ID NO: 2 + A274G | 1.01 | 0.98 | 0.92 | 0.92 |
| SEQ ID NO: 2 + H1* + N3P | 0.87 | 1.01 | 0.56 | 1.01 |
| SEQ ID NO: 2 + H2R + N3* | 0.84 | 1.03 | 0.77 | 0.97 |
| SEQ ID NO: 2 + A274S | 1.09 | 0.98 | 1.04 | 1.09 |
| SEQ ID NO: 2 + | 1.1 | 1.02 | 1.08 | 1.21 |
| G182A + V213T + V214S | | | | |
| SEQ ID NO: 2 + H210S | 1.24 | 1.07 | 1.28 | 1.22 |
| SEQ ID NO: 2 + V213S | 0.89 | 0.98 | 1.06 | 0.93 |
| SEQ ID NO: 2 + G182S | 0.91 | 1 | 0.89 | 0.76 |
| SEQ ID NO: 2 + | 1.02 | 0.91 | 1.1 | 1.05 |
| H210S + P408Q | | | | |
| SEQ ID NO: 2 + V214I | 1.02 | 1.06 | 1.03 | 1.05 |
| SEQ ID NO: 2 + H210Y | 1.24 | 1.09 | 1.41 | 1.11 |
| SEQ ID NO: 2 + | 1.08 | 0.97 | 1.07 | 0.86 |
| G182A + H210Y + V214S | | | | |
| SEQ ID NO: 2 + P211T | 1.19 | 1.01 | 1.33 | 1.05 |
| SEQ ID NO: 2 + | 0.76 | 1.03 | 1.03 | 0.89 |
| G182S + H210S + V214S | | | | |
| SEQ ID NO: 2 + P211T + V213T | 1.18 | 1.12 | 1.14 | 1.25 |
| SEQ ID NO: 2 + | 0.97 | 1.04 | 0.72 | 0.86 |
| G182S + H210Y + E210D | | | | |
| SEQ ID NO: 2 + | 1.02 | 0.94 | 1.06 | 0.92 |
| G182N + A186E + V214S | | | | |
| SEQ ID NO: 2 + V213T + V214T | 1.01 | 1.18 | 1.09 | 1.03 |
| SEQ ID NO: 2 + P211H + V214I | 0.95 | 1.06 | 1.05 | 0.93 |
| SEQ ID NO: 2 + | 1.11 | 1.08 | 0.9 | 1.25 |
| G182N + V213S | | | | |
| SEQ ID NO: 2 + | 0.91 | 1.04 | 0.95 | 1.04 |
| G182S + V213T + V214S | | | | |
| SEQ ID NO: 2 + | 0.81 | 1.07 | 0.77 | 0.85 |
| G182S + E210D + V214I | | | | |
| SEQ ID NO: 2 + | 1.26 | 1.11 | 1.35 | 1.13 |
| H210S + V214T | | | | |
| SEQ ID NO: 2 + V213T + V214S | 1.24 | 1.02 | 1.17 | 0.97 |
| SEQ ID NO: 2 + G182S | 0.99 | 1.08 | 0.91 | 0.99 |
| SEQ ID NO: 2 + V214S | 0.85 | 1.05 | 0.88 | 0.82 |
| SEQ ID NO: 2 + | 1.03 | 1.05 | 0.95 | 0.8 |
| G182A + V213T + V214I | | | | |
| SEQ ID NO: 2 + | 1.28 | 1.11 | 1.09 | 1.12 |
| K269Q + A274W | | | | |
| SEQ ID NO: 2 + | 1.02 | 1.04 | 0.94 | 0.94 |
| K269H + A274W | | | | |
| SEQ ID NO: 2 + | 1.1 | 0.98 | 0.95 | 1.04 |
| K269H + A274E | | | | |
| SEQ ID NO: 2 + | 1.02 | 0.86 | 1.06 | 0.91 |
| K269Q + A274C | | | | |
| SEQ ID NO: 2 + | 1.19 | 1.05 | 1.26 | 1.06 |
| K269Q + A274Y | | | | |
| SEQ ID NO: 2 + | 1.12 | 0.99 | 0.95 | 0.94 |
| K269Q + A274L | | | | |
| SEQ ID NO: 2 + | 1.11 | 0.96 | 1.02 | 0.97 |
| K269Q + A274M | | | | |
| SEQ ID NO: 2 + | 1.06 | 0.96 | 1.03 | 1.02 |
| K269H + A274S | | | | |
| SEQ ID NO: 2 + | 1.1 | 1.33 | 0.98 | 1.06 |
| K269Q + A274S | | | | |
| SEQ ID NO: 2 + K269L + A274R | 0.91 | 1.04 | 0.85 | 0.63 |
| SEQ ID NO: 2 + K269L + A274S | 0.97 | 1.04 | 1.07 | 0.85 |
| SEQ ID NO: 2 + K269L + A274L | 1.06 | 1.09 | 1.09 | 1.01 |
| SEQ ID NO: 2 + | 1.22 | 1.08 | 1.1 | 1 |
| K269Q + A274G | | | | |
| SEQ ID NO: 2 + | 1.24 | 1.07 | 1.05 | 0.87 |
| K269V + A274Y | | | | |
| SEQ ID NO: 2 + | 1.21 | 0.88 | 1 | 0.99 |
| K269L + A274G | | | | |
| SEQ ID NO: 2 + | 1.16 | 1.07 | 0.93 | 0.88 |
| K269L + A274W | | | | |
| SEQ ID NO: 2 + | 1.26 | 1.02 | 0.95 | 0.85 |
| K269Q + A274R | | | | |
| SEQ ID NO: 2 + | 1.25 | 1.05 | 0.98 | 0.86 |
| K269R + A274G | | | | |
| SEQ ID NO: 2 + | 1.08 | 0.98 | 1.02 | 1.04 |
| K269Q + A274H | | | | |
| SEQ ID NO: 2 + A274L | 1.07 | 1 | 0.95 | 0.93 |
| SEQ ID NO: 2 + | 0.92 | 1.05 | 0.75 | 0.84 |
| K269H + A274R | | | | |
| SEQ ID NO: 2 + | 1.07 | 0.91 | 0.88 | 0.98 |
| K269Q + A274V | | | | |
| SEQ ID NO: 2 + | 1.13 | 1.06 | 1.07 | 1.12 |
| K269H + A274C | | | | |
| SEQ ID NO: 2 + K269Q | 1.02 | 0.74 | 0.85 | 0.63 |
| SEQ ID NO: 2 + | 0.93 | 0.97 | 0.89 | 1.07 |
| K269R + A274W | | | | |
| SEQ ID NO: 2 + | 1.06 | 1.02 | 1.12 | 1.14 |
| K269N + A274M | | | | |
| SEQ ID NO: 2 + | 1.01 | 1.05 | 1.02 | 1.09 |
| K269N + A274C | | | | |
| SEQ ID NO: 2 + | 1.26 | 1 | 1.25 | 1.02 |
| R172Q + N174Q | | | | |
| SEQ ID NO: 2 + R172K | 0.98 | 0.91 | 0.88 | 1.07 |
| SEQ ID NO: 2 + | 0.93 | 0.98 | 1.21 | 1.29 |
| G50A + R172Q + L173F | | | | |
| SEQ ID NO: 2 + | 1.25 | 1.09 | 1.14 | 0.94 |
| R172Q + N174S | | | | |
| SEQ ID NO: 2 + | 1.29 | 1.04 | 1.11 | 1.12 |
| R172Q + L173F + N174Y | | | | |
| SEQ ID NO: 2 + | 1.04 | 0.99 | 0.95 | 0.94 |
| A51V + R172Q + N174S | | | | |
| SEQ ID NO: 2 + | 1.34 | 1.1 | 1.2 | 0.48 |
| G50A + A51T + N174E | | | | |
| SEQ ID NO: 2 + R172N | 1.26 | 1.07 | 1.07 | 0.96 |
| SEQ ID NO: 2 + | 1.18 | 0.98 | 1.1 | 1.01 |
| R172Q + N174T + A204T | | | | |
| SEQ ID NO: 2 + G50A + L173F | 0.83 | 1.07 | 0.75 | 0.77 |
| SEQ ID NO: 2 + N174S | 1.23 | 1.16 | 1.1 | 1.35 |
| SEQ ID NO: 2 + | 0.97 | 0.98 | 1.03 | 0.75 |
| G50A + A51T + L173F + N174T | | | | |
| SEQ ID NO: 2 + | 1.02 | 1 | 1.08 | 0.91 |
| R172Q + L173I + N174T | | | | |
| SEQ ID NO: 2 + | 1.13 | 1.06 | 1.08 | 1.05 |
| G50A + A51T + R172Q + N174Q + | | | | |
| E471K | | | | |
| SEQ ID NO: 2 + R172Q + L173I | 1.03 | 0.98 | 1.04 | 1.06 |
| SEQ ID NO: 2 + | 1 | 0.97 | 1.13 | 0.8 |
| R172Q + L173T + N174D | | | | |
| SEQ ID NO: 2 + G50A + R172Q | 1.42 | 1.13 | 1.41 | 1.36 |
| SEQ ID NO: 2 + G50A | 0.9 | 1.01 | 0.61 | 0.87 |
| SEQ ID NO: 2 + | 1.11 | 1.09 | 1.18 | 1.19 |
| R172K + L173I + N174S | | | | |
| SEQ ID NO: 2 + | 1.05 | 1.03 | 0.99 | 0.82 |
| N174S + A204T | | | | |
| SEQ ID NO: 2 + | 1 | 1.04 | 0.88 | 1.03 |
| G50A + L173T + N174Q | | | | |
| SEQ ID NO: 2 + | 1.38 | 1.09 | 1 | 1.02 |
| G50A + A51V + R172N + N174S | | | | |
| SEQ ID NO: 2 + | 1.13 | 0.99 | 1.11 | 0.92 |
| R172Q + L173F + A204T | | | | |
| SEQ ID NO: 2 + | 1.23 | 1.14 | 1.05 | 1.24 |
| G50A + A51T + R172Q | | | | |
| SEQ ID NO: 2 + | 1.07 | 0.93 | 1 | 0.81 |
| G50A + L173F + N174Q | | | | |
| SEQ ID NO: 2 + | 0.83 | 1.03 | 0.66 | 0.63 |
| Q169H + L173F | | | | |
| SEQ ID NO: 2 + | 1.38 | 1.13 | 1.21 | 1.07 |
| G50A + R172Q + N174T | | | | |
| SEQ ID NO: 2 + | 1.23 | 1.06 | 1.17 | 0.98 |
| R172Q + N174T | | | | |
| SEQ ID NO: 2 + | 1 | 1.12 | 0.97 | 1.3 |
| G50A + R172Q + L173T | | | | |
| SEQ ID NO: 2 + | 1.28 | 1.05 | 1.21 | 1.18 |
| G50A + R172Q + N174D | | | | |
| SEQ ID NO: 2 + | 1.12 | 1.14 | 1.22 | 0.92 |
| N174D + A204V | | | | |
| SEQ ID NO: 2 + | 1.05 | 1.08 | 1.06 | 0.78 |
| G50A + L173F + N174T | | | | |
| SEQ ID NO: 2 + | 1.15 | 0.93 | 1.12 | 1.27 |
| G50A + R172N + N174S | | | | |
| SEQ ID NO: 2 + | 1.21 | 1.16 | 1.19 | 1.1 |
| G50A + R172N + N174T | | | | |
| SEQ ID NO: 2 + | 1.22 | 0.97 | 1.42 | 0.99 |
| G73V + R172Q + N174E + A521 | | | | |
| SEQ ID NO: 2 + | 1.1 | 0.98 | 1.2 | 1.09 |
| R172N + L173T + N174Q + S303R | | | | |
| SEQ ID NO: 2 + | 1.11 | 1.08 | 1.32 | 0.97 |
| G50S + R172K + N174D | | | | |
| SEQ ID NO: 2 + | 1.12 | 1.19 | 1.5 | 0.97 |
| G50A + A51T + K72H + R172Q + | | | | |
| N174Q + A204V | | | | |
| SEQ ID NO: 2 + A204V | 1.07 | 1.06 | 1.25 | 0.95 |
| SEQ ID NO: 2 + R172Q | 1.37 | 1.14 | 1.41 | 1.13 |
| SEQ ID NO: 2 + | 1.3 | 1.12 | 1.4 | 1.14 |
| L173T + N174Q + A204V | | | | |
| SEQ ID NO: 2 + R172K + L173F | 1.2 | 0.94 | 1.14 | 1.05 |
| SEQ ID NO: 2 + | 1.09 | 1.07 | 1.04 | 1.03 |
| G50A + L173V + N174S | | | | |
| SEQ ID NO: 2 + | 1.2 | 1.02 | 1.27 | 1.15 |
| G50A + R172Q + L173T + N174D | | | | |
| SEQ ID NO: 2 + G50A + N174E | 1.12 | 1.06 | 1.18 | 1.05 |
| SEQ ID NO: 2 + G50A + L173T | 0.85 | 1.09 | 0.93 | 0.71 |
| SEQ ID NO: 2 + | 1.15 | 1.13 | 1.3 | 1.05 |
| G50A + A51T + R172Q + L173V | | | | |
| SEQ ID NO: 2 + | 1.05 | 0.94 | 1.27 | 0.86 |
| R172Q + L173I + N174S | | | | |
| SEQ ID NO: 2 + L173V + N174S | 0.9 | 0.94 | 1.27 | 0.88 |
| SEQ ID NO: 2 + | 0.96 | 1.05 | 1.06 | 0.99 |
| G50A + N174T + A204V | | | | |
| SEQ ID NO: 2 + | 1.26 | 1.23 | 1.62 | 1.01 |
| G50A + R172D + N174D | | | | |
| SEQ ID NO: 2 + | 1.27 | 1.1 | 1.67 | 0.95 |
| G50A + R172D + N174Q | | | | |
| SEQ ID NO: 2 + | 1.22 | 1.06 | 1.42 | 0.8 |
| R172D + N174T | | | | |
| SEQ ID NO: 2 + | 1.02 | 1.16 | 1.13 | 0.67 |
| G50A + L173V + A204T | | | | |
| SEQ ID NO: 2 + | 1.23 | 1.24 | 1.41 | 1.13 |
| G50A + A51V + R172N + N174T + | | | | |
| A204T | | | | |
| SEQ ID NO: 2 + N174Q | 0.9 | 1 | 0.98 | 0.79 |
| SEQ ID NO: 2 + G50A + A204T | 0.97 | 0.88 | 0.91 | 1 |
| SEQ ID NO: 2 + R172E | 1.19 | 0.83 | 1.09 | 0.95 |
| SEQ ID NO: 2 + | 1.11 | 1.02 | 0.97 | 0.91 |
| G304Q + E346P + D476K + G477A | | | | |
| SEQ ID NO: 2 + | 1.19 | 0.98 | 1.06 | 0.98 |
| E346P + G477Q | | | | |
| SEQ ID NO: 2 + | 1.12 | 0.9 | 1.01 | 0.91 |
| E346P + G477T | | | | |
| SEQ ID NO: 2 + | 1.11 | 0.97 | 0.97 | 0.79 |
| G304S + E345D + E346P + D476K + | | | | |
| G477Q | | | | |
| SEQ ID NO: 2 + | 1.11 | 1.12 | 1.07 | 1.01 |
| E346P + D476Q + G477T | | | | |
| SEQ ID NO: 2 + | 0.91 | 1.12 | 0.96 | 0.96 |
| G304Q + E345D + E346P + D476G + | | | | |
| G477Q | | | | |
| SEQ ID NO: 2 + | 1.08 | 1.26 | 1.2 | 1.08 |
| E345D + E346P + D476K + G477S | | | | |
| SEQ ID NO: 2 + | 1 | 1.21 | 1.1 | 1.05 |
| G304Q + D476G + G477K | | | | |
| SEQ ID NO: 2 + | 1.27 | 1.22 | 1.3 | 0.96 |
| E346P + D476K + G477S | | | | |
| SEQ ID NO: 2 + | 1.22 | 1.09 | 1.24 | 1.04 |
| E345D + D476K + G477S | | | | |
| SEQ ID NO: 2 + | 1.03 | 0.97 | 1.08 | 1.01 |
| G304A + E345D + E346T + D476K + | | | | |
| G477S | | | | |
| SEQ ID NO: 2 + | 1 | 0.99 | 1 | 1 |
| E346T + D476G + G477S | | | | |
| SEQ ID NO: 2 + | 0.97 | 0.94 | 0.98 | 1.04 |
| E345D + E346T + L444P + G477K | | | | |
| SEQ ID NO: 2 + | 1.49 | 1.18 | 1.28 | 1.09 |
| E346T + D476K + G477A | | | | |
| SEQ ID NO: 2 + | 1.36 | 1.02 | 1.23 | 1.02 |
| E345D + E346P + D476G | | | | |
| SEQ ID NO: 2 + | 1.46 | 1.22 | 1.25 | 1.13 |
| K302Q + E346P + D476K + G477S | | | | |
| SEQ ID NO: 2 + | 1.33 | 1.23 | 1.02 | 1.05 |
| G304A + E345D + E346P + D476G + | | | | |
| G477K | | | | |
| SEQ ID NO: 2 + | 1.16 | 1.27 | 1 | 1.13 |
| G304Q + E346P + D476G | | | | |
| SEQ ID NO: 2 + | 1.22 | 1.23 | 0.94 | 1.02 |
| E346P + D476G + G477K | | | | |
| SEQ ID NO: 2 + | 1.12 | 1.09 | 1.01 | 1.16 |
| G304A + E346P + D476K + G477A | | | | |
| SEQ ID NO: 2 + | 1.02 | 1.11 | 0.97 | 0.91 |
| G304A + E345D + E346T + D476G + | | | | |
| G477K | | | | |
| SEQ ID NO: 2 + | 0.99 | 1 | 1.06 | 0.82 |
| K302Q + E345D + D476K + G477K | | | | |
| SEQ ID NO: 2 + | 1.04 | 1.03 | 0.95 | 1.02 |
| D476Q + G477K | | | | |
| SEQ ID NO: 2 + | 1.05 | 1.1 | 1.23 | 1.26 |
| E346T + D476G + G477T | | | | |
| SEQ ID NO: 2 + | 1.03 | 0.94 | 1.06 | 1.09 |
| E345D + E346T + D476G + G477K | | | | |
| SEQ ID NO: 2 + | 0.94 | 1 | 1.05 | 0.84 |
| G304Q + E346P + D476G + G477K | | | | |
| SEQ ID NO: 2 + | 0.85 | 1.06 | 1.04 | 0.87 |
| K302N + E345D + D476K + G477S | | | | |
| SEQ ID NO: 2 + | 0.95 | 1.16 | 1 | 0.82 |
| K302Q + G304A + E346T + G477K | | | | |
| SEQ ID NO: 2 + | 1.03 | 0.78 | 1.08 | 0.91 |
| E345D + E346P + D476K + G477Q | | | | |
| SEQ ID NO: 2 + | 1.11 | 0.9 | 1.12 | 1.12 |
| K302N + E345D + D476Q + G477Q | | | | |
| SEQ ID NO: 2 + | 0.91 | 1.11 | 0.94 | 0.74 |
| E345D + E346P + D476K + G477T | | | | |
| SEQ ID NO: 2 + | 0.89 | 1.02 | 0.83 | 1 |
| E345D + D476G | | | | |
| SEQ ID NO: 2 + | 1.14 | 0.99 | 1.07 | 0.99 |
| G304A + D476K + G477S | | | | |
| SEQ ID NO: 2 + | 1.28 | 1.04 | 1.38 | 1.11 |
| E345D + E346P + G477Q | | | | |
| SEQ ID NO: 2 + | 1.16 | 0.87 | 1.14 | 0.93 |
| L173F + N174Q | | | | |
| SEQ ID NO: 2 + | 1.3 | 0.83 | 1.4 | 1.17 |
| R172Q + L173F | | | | |
| SEQ ID NO: 2 + G50A + N174S | 1.3 | 0.8 | 1.35 | 1.14 |
| SEQ ID NO: 2 + | 1.32 | 0.17 | 1.3 | 1.01 |
| G50A + R172Q + L173V + N174S | | | | |
| SEQ ID NO: 2 + | 1.09 | 0.54 | 1.04 | 0.99 |
| A51V + R172D + L173F | | | | |
| SEQ ID NO: 2 + N174D | 1.03 | 0.95 | 1.07 | 1.27 |
| SEQ ID NO: 2 + | 1 | 0.85 | 0.93 | 1.14 |
| G50A + R172Q + A204T | | | | |
| SEQ ID NO: 2 + | 1 | 0.87 | 0.98 | 1.13 |
| G50A + R172Q + N174Q | | | | |
| SEQ ID NO: 2 + | 1.31 | 1.06 | 1.56 | 1.43 |
| G50A + A51V + R172D + L173F | | | | |
| SEQ ID NO: 2 + | 1.14 | 1.06 | 1.23 | 1.32 |
| G50A + A51I + R172D + N174* | | | | |
| SEQ ID NO: 2 + R172D + L173F | 1.26 | 0.89 | 1.34 | 1.3 |
| SEQ ID NO: 2 + | 1 | 1.01 | 1.05 | 0.97 |
| R172K + L173F + A204T | | | | |
| SEQ ID NO: 2 + R172D | 1.21 | 0.87 | 1.42 | 1.06 |
| SEQ ID NO: 2 + | 1.16 | 0.8 | 1.11 | 1.11 |
| A51V + R172Q + A204V | | | | |
| SEQ ID NO: 2 + G50A + R172K | 1.01 | 1.08 | 1.4 | 1.31 |
| SEQ ID NO: 2 + | 1.09 | 1.1 | 1.26 | 1.36 |
| G50A + R172Q + N174S | | | | |
| SEQ ID NO: 2 + | 1.3 | 1.05 | 1.36 | 1.05 |
| G50A + A51V + R172D + L173F | | | | |
| N174Q + A204T | | | | |
| SEQ ID NO: 2 + | 1.19 | 1.16 | 1.27 | 1.31 |
| G50S + R172N + N174Q | | | | |
| SEQ ID NO: 2 + | 1.26 | 1.19 | 1.35 | 1.35 |
| G50A + R172Q + L173I + N174T + | | | | |
| A204T | | | | |
| SEQ ID NO: 2 + | 1.02 | 0.87 | 0.86 | 1.08 |
| A51I + R172Q + L173F + A204T | | | | |
| SEQ ID NO: 2 + | 0.98 | 1.01 | 0.86 | 0.99 |
| A51I + R172N + N174Q | | | | |
| SEQ ID NO: 2 + L173F | 1.13 | 1.44 | 0.86 | 0.98 |
| SEQ ID NO: 2 + | 1.13 | 1.31 | 1.04 | 0.93 |
| T136N + R172Q + N174D + A204T | | | | |
| SEQ ID NO: 2 + G50A + R172E | 1.03 | 1.38 | 0.99 | 0.91 |
| SEQ ID NO: 2 + | 1.17 | 1.19 | 1.14 | 1.04 |
| G50A + R172Q + A204V | | | | |
| SEQ ID NO: 2 + L173V + A204T | 1.03 | 0.48 | 0.92 | 0.92 |
| SEQ ID NO: 2 + | 1.08 | 0.8 | 0.97 | 0.93 |
| A51V + L173I + N174S | | | | |
| SEQ ID NO: 2 + | 1.19 | 0.9 | 1.01 | 1.07 |
| A51T + L173T + N174S + A204T + | | | | |
| A583 | | | | |
| SEQ ID NO: 2 + L173I + N174D | 1.28 | 0.87 | 1.25 | 1.16 |
| SEQ ID NO: 2 + | 1.1 | 0.98 | 1.15 | 1.17 |
| W48L + A51T + R172Q + N174S + | | | | |
| A204T | | | | |
| SEQ ID NO: 2 + | 1.37 | 0.87 | 1.42 | 1.26 |
| G50A + A51V + R172E + L173F + | | | | |
| N174T + A204V | | | | |
| SEQ ID NO: 2 + G50A + L173V | 1.09 | 0.7 | 0.99 | 0.97 |
| SEQ ID NO: 2 + | 1.22 | 1.23 | 1.1 | 1.1 |
| G50A + R172Q + L444P | | | | |
| SEQ ID NO: 2 + | 1.27 | 1.04 | 1.13 | 0.83 |
| R172Q + L173T + N174S | | | | |
| SEQ ID NO: 2 + L173I + N174S | 1.03 | 1.05 | 0.88 | 0.98 |
| SEQ ID NO: 2 + L173F + A204T | 1.23 | 1.1 | 1.24 | 1.12 |
| SEQ ID NO: 2 + A51I + R172D | 1.03 | 1.13 | 1.16 | 0.68 |
| SEQ ID NO: 2 + | 0.86 | 0.67 | 1.05 | 1.05 |
| A51I + R172Q + N174E | | | | |
| SEQ ID NO: 2 + | 1.37 | 0.98 | 0.97 | 1.02 |
| G50A + A51T + R172N + L173V | | | | |
| SEQ ID NO: 2 + | 1.15 | 1.15 | 1.24 | 0.96 |
| G50A + R172Q + N174S + A204T | | | | |
| SEQ ID NO: 2 + | 1.41 | 0.99 | 1.32 | 1.16 |
| R172Q + L173F + N174D | | | | |
| SEQ ID NO: 2 + | 1.27 | 1.01 | 1.38 | 1.16 |
| G50A + A51V + R172Q | | | | |
| SEQ ID NO: 2 + R172Q + N174* | 1.14 | 0.97 | 1.31 | 1.15 |
| SEQ ID NO: 2 + | 1.22 | 1.09 | 1.47 | 1.19 |
| G50A + A51T + R172D | | | | |
| SEQ ID NO: 2 + | 1.23 | 1.2 | 1.23 | 1.03 |
| G50A + A51T + R172N + N174D + | | | | |
| G182V | | | | |
| SEQ ID NO: 2 + | 1.01 | 0.93 | 1.19 | 0.93 |
| R172N + N174S | | | | |
| SEQ ID NO: 2 + | 1.66 | 1.02 | 1.08 | 1.54 |
| G50A + R172E + L173F | | | | |
| SEQ ID NO: 2 + L173V + N174T | 1.7 | 1.02 | 1.01 | 1.98 |
| SEQ ID NO: 2 + N174T | 1.4 | 0.95 | 0.61 | 1.02 |
| SEQ ID NO: 2 + | 1.12 | 0.98 | 1.09 | 1.33 |
| G50A + A51T + R172Q + N174E | | | | |
| SEQ ID NO: 2 + | 1.9 | 1.16 | 1.17 | 1.88 |
| G50A + R172Q + L173F + N174Q | | | | |
| SEQ ID NO: 2 + | 1.72 | 0.97 | 1.29 | 1.55 |
| R172E + L173F + N174Q | | | | |
| SEQ ID NO: 2 + | 1.46 | 1.05 | 1.22 | 1.39 |
| A51I + R172E + N174T | | | | |
| SEQ ID NO: 2 + | 1.67 | 1.12 | 1.14 | 1.55 |
| G50A + A51I + A204V | | | | |
| SEQ ID NO: 2 + | 1.98 | 0.94 | 1.28 | 1.91 |
| R172Q + N174D | | | | |
| SEQ ID NO: 2 + | 1.4 | 0.95 | 0.94 | 1.44 |
| G50A + L173F + N174S | | | | |
| SEQ ID NO: 2 + | 1.96 | 1.04 | 1.34 | 1.59 |
| G50A + R172E + L173V + N174T | | | | |
| SEQ ID NO: 2 + | 1.38 | 1.11 | 0.71 | 1.06 |
| G50A + A51L + N174S + A447V | | | | |
| SEQ ID NO: 2 + | 1.54 | 1.21 | 0.96 | 1.62 |
| G50A + A51T + L173F | | | | |
| SEQ ID NO: 2 + | 0.95 | 1.06 | 0.86 | 0.89 |
| K302Q + E345D + D476G | | | | |
| SEQ ID NO: 2 + | 0.82 | 1.02 | 0.59 | 0.69 |
| E345D + E346T | | | | |
| SEQ ID NO: 2 + | 1.41 | 1.16 | 1.31 | 1.14 |
| A274E + G304Q + G305S + E345D + | | | | |
| D476K + G477Q | | | | |
| SEQ ID NO: 2 + | 1.34 | 1.22 | 1.15 | 1.04 |
| E346P + D476Q + G477S | | | | |
| SEQ ID NO: 2 + | 1.08 | 1.11 | 0.88 | 0.93 |
| K302N + E346T + D476K + G477A | | | | |
| SEQ ID NO: 2 + | 1.18 | 1.14 | 1.24 | 1.05 |
| E345D + E346P + D476G + G477T | | | | |
| SEQ ID NO: 2 + | 1.09 | 1.05 | 0.96 | 0.99 |
| G304A + E345D + E346T + D476G + | | | | |
| G477T | | | | |
| SEQ ID NO: 2 + | 1.16 | 1.12 | 1.02 | 0.91 |
| E346T + D476K | | | | |
| SEQ ID NO: 2 + | 1.2 | 1.09 | 0.96 | 1.58 |
| E345D + D476Q + G477K | | | | |
| SEQ ID NO: 2 + | 0.96 | 1.1 | 0.95 | 1.13 |
| G304A + E345D + D476Q + G477A | | | | |
| SEQ ID NO: 2 + | 1.62 | 0.91 | 1.18 | 1.66 |
| G304Q + E345D + E346T + D476G + | | | | |
| G477S | | | | |
| SEQ ID NO: 2 + E346T | 1.83 | 0.94 | 0.88 | 1.58 |
| SEQ ID NO: 2 + | 1.04 | 1.13 | 0.99 | 1.02 |
| K302N + E345D + E346T + D476Q + | | | | |
| G477Q | | | | |
| SEQ ID NO: 2 + | 1.1 | 0.98 | 0.76 | 0.87 |
| G305S + E346T + D476Q + G477Q | | | | |
| SEQ ID NO: 2 + | 0.96 | 1.11 | 0.87 | 1.02 |
| G304Q + E345D + D476G + G477Q | | | | |
| SEQ ID NO: 2 + | 1.11 | 0.98 | 0.86 | 0.83 |
| E345D + E346T + D476Q + G477Q | | | | |
| SEQ ID NO: 2 + | 0.96 | 1.04 | 0.72 | 0.89 |
| G304Q + D476K + G477K | | | | |
| SEQ ID NO: 2 + | 0.97 | 1.09 | 0.83 | 0.92 |
| D112H + G304A + E345D + E346T + | | | | |
| D476Q + G477S | | | | |
| SEQ ID NO: 2 + | 1.06 | 1.08 | 0.87 | 0.98 |
| A113T + K302Q + E346T + D476G + | | | | |
| G477K | | | | |
| SEQ ID NO: 2 + | 1.01 | 1.06 | 0.9 | 1.01 |
| G304Q + E345D + E346P + D476Q + | | | | |
| G477Q | | | | |
| SEQ ID NO: 2 + | 0.56 | 1.05 | 0.42 | 0.81 |
| E345D + E346T + D476G | | | | |
| SEQ ID NO: 2 + | 1.05 | 1.15 | 0.96 | 0.99 |
| G304Q + E346P + D476K + G477S | | | | |
| SEQ ID NO: 2 + | 1.02 | 1.02 | 1.08 | 1.15 |
| K302N + E345D + D476G + G477K | | | | |
| SEQ ID NO: 2 + | 0.89 | 1.08 | 1.03 | 1.04 |
| E346T + D476Q + G477S | | | | |
| SEQ ID NO: 2 + | 0.85 | 1.14 | 0.98 | 0.8 |
| E345D + E346T + D476K + G477K | | | | |
| SEQ ID NO: 2 + | 0.91 | 1.09 | 1.13 | 0.96 |
| E345D + E346T + D476G + G477A | | | | |
| SEQ ID NO: 2 + | 0.99 | 1.12 | 1.17 | 1.07 |
| E346P + D476Q | | | | |
| SEQ ID NO: 2 + | 0.97 | 1.03 | 1.04 | 0.81 |
| E345D + E346P + D476Q + G477K | | | | |
| SEQ ID NO: 2 + | 1.02 | 1.18 | 1.28 | 1.06 |
| G304Q + E346T + D476K + G477Q | | | | |
| SEQ ID NO: 2 + | 1.07 | 1.14 | 1.34 | 1.16 |
| K302N + G304Q + E346P + D476K | | | | |
| SEQ ID NO: 2 + | 0.93 | 1.12 | 1.15 | 1 |
| G304A + E346K + G477K | | | | |
| SEQ ID NO: 2 + | 0.94 | 1.13 | 1.23 | 1.09 |
| G273V + G304S + E346P + D476G + | | | | |
| G477T | | | | |
| SEQ ID NO: 2 + | 1.18 | 1.13 | 1.33 | 1.08 |
| K302Q + E345D + E346P + D476K + | | | | |
| G477Q | | | | |
| SEQ ID NO: 2 + | 1.1 | 0.86 | 0.94 | 0.85 |
| E346T + G477K | | | | |
| SEQ ID NO: 2 + | 1.01 | 1.07 | 1.04 | 0.97 |
| K302Q + E345D + E346T + D476K + | | | | |
| G477K | | | | |
| SEQ ID NO: 2 + | 1.15 | 0.89 | 1.13 | 1.01 |
| E346P + G477S | | | | |
| SEQ ID NO: 2 + | 1.13 | 0.8 | 1.08 | 1.04 |
| K302Q + E346P + D476K + G477Q | | | | |
| SEQ ID NO: 2 + | 0.99 | 0.94 | 1.18 | 0.99 |
| G305S + E346P + D476Q + G477T | | | | |
| SEQ ID NO: 2 + | 1.11 | 0.88 | 1.06 | 1.01 |
| G304A + E346P + D476G + G477A | | | | |
| SEQ ID NO: 2 + | 1.04 | 0.96 | 1.17 | 1.01 |
| E345D + E346P + D476Q + G477A | | | | |
| SEQ ID NO: 2 + | 1.02 | 1.01 | 1.04 | 0.98 |
| G304A + E345D + E346P + D476K | | | | |
| SEQ ID NO: 2 + | 0.7 | 1.04 | 0.66 | 0.91 |
| K302Q + G305A + E345D + D476G + | | | | |
| G477K | | | | |
| SEQ ID NO: 2 + | 0.9 | 0.98 | 1.12 | 0.9 |
| E346T + D476K + G477S | | | | |
| SEQ ID NO: 2 + | 1.12 | 0.99 | 1.16 | 0.99 |
| E345D + D476Q + G477S | | | | |
| SEQ ID NO: 2 + | 1.15 | 0.97 | 1.1 | 1.06 |
| G304S + E346P + D476K + G477S | | | | |
| SEQ ID NO: 2 + | 1.01 | 0.94 | 1.03 | 0.96 |
| E345D + E346T + D476K + G477Q | | | | |
| SEQ ID NO: 2 + | 1.06 | 1.03 | 0.99 | 0.99 |
| E345D + E346P + D476Q | | | | |
| SEQ ID NO: 2 + | 1.02 | 1.1 | 0.93 | 0.93 |
| G304A + E346T | | | | |
| SEQ ID NO: 2 + | 1 | 1.22 | 1.07 | 1.14 |
| G304Q + E345D + E346T + D476G + | | | | |
| G477T | | | | |
| SEQ ID NO: 2 + | 0.93 | 0.87 | 0.89 | 1.26 |
| K302N + E346P + D476Q + G477K | | | | |
| SEQ ID NO: 2 + | 1.01 | 0.92 | 0.82 | 1.35 |
| E345D + D476Q | | | | |
| SEQ ID NO: 2 + | 0.85 | 0.92 | 0.76 | 1.06 |
| G304A + E346T + D476G | | | | |
| SEQ ID NO: 2 + | 0.9 | 0.86 | 1.01 | 1.24 |
| G304S + E345D + E346T + D476Q + | | | | |
| G477A | | | | |
| SEQ ID NO: 2 + | 0.99 | 0.88 | 1.13 | 1.13 |
| E346P + D476K + G477T | | | | |
| SEQ ID NO: 2 + | 1.15 | 0.96 | 1.22 | 1.31 |
| G304A + E345D + E346T + D476Q + | | | | |
| G477Q | | | | |
| SEQ ID NO: 2 + | 1.09 | 0.95 | 0.87 | 1.13 |
| G304S + E346P + D476K + G477K | | | | |
| SEQ ID NO: 2 + | 1.03 | 0.95 | 0.86 | 1.12 |
| K302Q + E346P + D476K + G477T | | | | |
| SEQ ID NO: 2 + | 1.1 | 1.04 | 1.3 | 1.13 |
| E346T + D476Q + G477A | | | | |
| SEQ ID NO: 2 + | 0.88 | 0.94 | 1.09 | 1.11 |
| G304A + E345D + E346P + D476K + | | | | |
| G477Q | | | | |
| SEQ ID NO: 2 + R118L | 1.13 | 0.91 | 1.38 | 1.09 |
| SEQ ID NO: 2 + | 1.34 | 0.98 | 1.54 | 1.49 |
| R118S + G182A + T193K | | | | |
| SEQ ID NO: 2 + | 1.57 | 1.02 | 1.59 | 1.25 |
| R118P + G182A | | | | |
| SEQ ID NO: 2 + | 1.45 | 1.04 | 1.5 | 0.77 |
| R118P + G182S | | | | |
| SEQ ID NO: 2 + | 1.5 | 1.02 | 1.31 | 1 |
| R118S + E134D | | | | |
| SEQ ID NO: 2 + | 1.27 | 1.03 | 1.34 | 1.11 |
| E134D + K179L + G182N | | | | |
| SEQ ID NO: 2 + R118S | 1.27 | 0.98 | 1.21 | 1.07 |
| SEQ ID NO: 2 + | 1.08 | 1.02 | 1.07 | 0.95 |
| R118S + E134D + G182A + A727 | | | | |
| SEQ ID NO: 2 + | 1.29 | 1.11 | 1.33 | 1.09 |
| R118S + G182A | | | | |
| SEQ ID NO: 2 + | 1.28 | 0.94 | 1.29 | 1.08 |
| R118S + N128L + G182S | | | | |
| SEQ ID NO: 2 + N128Y | 0.99 | 0.93 | 1.01 | 1.01 |
| SEQ ID NO: 2 + E134D | 1 | 0.92 | 1.16 | 1.05 |
| SEQ ID NO: 2 + N128Q | 0.99 | 0.97 | 1.01 | 0.91 |
| SEQ ID NO: 2 + | 1.12 | 0.99 | 1.33 | 1.04 |
| G182S + A186V | | | | |
| SEQ ID NO: 2 + | 1.34 | 0.75 | 1.47 | 0.77 |
| R118D + K179L + G182A | | | | |
| SEQ ID NO: 2 + | 1.11 | 0.81 | 1.44 | 0.93 |
| R118P + G182N | | | | |
| SEQ ID NO: 2 + K179L | 0.95 | 1.01 | 1.28 | 1.01 |
| SEQ ID NO: 2 + | 0.98 | 0.93 | 1.01 | 1.15 |
| G4V + E134D + K179L | | | | |
| SEQ ID NO: 2 + | 0.97 | 0.75 | 1.16 | 0.6 |
| R118D + G182N | | | | |
| SEQ ID NO: 2 + | 1.23 | 1 | 1.31 | 1.08 |
| R118S + E134D + G182S | | | | |
| SEQ ID NO: 2 + | 1.23 | 0.75 | 1.32 | 1.25 |
| E134D + A186E | | | | |
| SEQ ID NO: 2 + | 1.03 | 0.36 | 1.18 | 0.76 |
| R118S + N128Q + K179L | | | | |
| SEQ ID NO: 2 + | 1.01 | 0.81 | 0.97 | 0.78 |
| N128Y + G182A + A186E | | | | |
| SEQ ID NO: 2 + | 0.9 | 0.73 | 1.01 | 0.69 |
| R118P + E134D + G182A | | | | |
| SEQ ID NO: 2 + E134D + K179L | 0.96 | 0.6 | 1.01 | 0.63 |
| SEQ ID NO: 2 + | 1.01 | 0.95 | 1.13 | 0.94 |
| G182A + P432H | | | | |
| SEQ ID NO: 2 + N128F + K179L | 1.28 | 0.7 | 1.28 | 1.12 |
| SEQ ID NO: 2 + | 1.01 | 0.41 | 1.19 | 0.96 |
| K179L + G182A | | | | |
| SEQ ID NO: 2 + | 1.06 | 1.06 | 1.25 | 1.1 |
| N128Q + A442V | | | | |
| SEQ ID NO: 2 + K179L + A186S | 0.96 | 0.67 | 1.12 | 0.92 |
| SEQ ID NO: 2 + | 1.52 | 0.89 | 1.57 | 1.19 |
| N128Q + K179L + A186S | | | | |
| SEQ ID NO: 2 + R118D | 1.58 | 0.79 | 1.74 | 1.23 |
| SEQ ID NO: 2 + | 0.83 | 0.9 | 1.02 | 1.22 |
| E345D + D476Q + G477A | | | | |
| SEQ ID NO: 2 + | 1.3 | 0.99 | 0.9 | 1.18 |
| G304Q + E346P + D476G + G477S | | | | |
| SEQ ID NO: 2 + | 0.94 | 0.9 | 1.06 | 1.09 |
| E346T + D476Q | | | | |
| SEQ ID NO: 2 + | 0.89 | 0.92 | 0.92 | 1.03 |
| E346P + D476K + G477A | | | | |
| SEQ ID NO: 2 + | 0.9 | 0.95 | 0.89 | 1.13 |
| E346T + D476K + G477Q | | | | |
| SEQ ID NO: 2 + | 1.08 | 0.95 | 1.2 | 1.21 |
| E346T + D476Q + G477K | | | | |
| SEQ ID NO: 2 + | 1.24 | 0.97 | 1.24 | 1.31 |
| E345D + D476G + G477S | | | | |
| SEQ ID NO: 2 + | 0.75 | 1.01 | 0.88 | 0.86 |
| E345D + E346P + D476G + G477K | | | | |
| SEQ ID NO: 2 + | 1.19 | 1.05 | 0.89 | 1.23 |
| D476K + G477Q | | | | |
| SEQ ID NO: 2 + | 1 | 0.85 | 0.96 | 0.93 |
| E345D + E346P + D476Q + G477Q | | | | |
| SEQ ID NO: 2 + | 0.89 | 1.02 | 1.02 | 1.05 |
| G304A + E345D + D476K + G477S | | | | |
| SEQ ID NO: 2 + | 0.87 | 1.12 | 0.84 | 0.98 |
| G304Q + E345D + E346P + D476G | | | | |
| SEQ ID NO: 2 + | 1.01 | 1.04 | 0.98 | 1.07 |
| G304Q + E345D + E346T + G477Q | | | | |
| SEQ ID NO: 2 + | 0.87 | 1.01 | 0.89 | 0.92 |
| A47P + K108T + A113P + E346P + | | | | |
| D476G + G477S | | | | |
| SEQ ID NO: 2 + | 0.94 | 1.05 | 0.91 | 1 |
| E345D + D476K + G477Q | | | | |
| SEQ ID NO: 2 + | 1.03 | 1.03 | 0.92 | 0.97 |
| K302Q + E345D + E346T + D476K + | | | | |
| G477Q | | | | |
| SEQ ID NO: 2 + | 1.08 | 1.03 | 1.13 | 1.2 |
| K302Q + G304S + E345D + D476G + | | | | |
| G477Q | | | | |
| SEQ ID NO: 2 + | 0.89 | 0.89 | 0.96 | 1.07 |
| E346T + D476G + G477Q | | | | |
| SEQ ID NO: 2 + | 1 | 0.72 | 1.08 | 1.15 |
| E345D + D476Q + G477Q | | | | |
| SEQ ID NO: 2 + | 1.04 | 0.96 | 1.12 | 0.96 |
| E345D + E346P + G477S | | | | |
| SEQ ID NO: 2 + | 1.05 | 1 | 1.07 | 0.93 |
| E345D + D476G + G477Q | | | | |
| SEQ ID NO: 2 + | 0.89 | 1.05 | 0.96 | 1 |
| E345D + E346P + D476G + G477S | | | | |
| SEQ ID NO: 2 + | 1.04 | 0.99 | 0.95 | 0.89 |
| G304A + E345D + E346P + D476K + | | | | |
| G477S + Y480F | | | | |
| SEQ ID NO: 2 + | 1.07 | 0.94 | 1.08 | 0.91 |
| G304A + E345D + E346P + G477T + | | | | |
| Y480F | | | | |
| SEQ ID NO: 2 + | 1.03 | 1.04 | 1.08 | 1.01 |
| E345D + E346P | | | | |
| SEQ ID NO: 2 + | 1.15 | 0.98 | 1.06 | 0.96 |
| G304A + E345D + D476Q + G477Q | | | | |
| SEQ ID NO: 2 + | 1.06 | 0.95 | 0.93 | 0.87 |
| E346P + D476G + G477Q + Y480F | | | | |
| SEQ ID NO: 2 + | 1.29 | 1.03 | 1.18 | 1.04 |
| R172E + L173T + N174T | | | | |
| SEQ ID NO: 2 + | 1.03 | 0.97 | 1.08 | 1.1 |
| G50A + R172Q + L173I | | | | |
| SEQ ID NO: 2 + | 1.19 | 1.04 | 1.07 | 1.19 |
| G50A + A51V + L173F + N174D | | | | |
| SEQ ID NO: 2 + | 1.31 | 1.04 | 1.01 | 1.04 |
| G50A + R172D + L173T | | | | |
| SEQ ID NO: 2 + | 1.08 | 0.95 | 1.06 | 1.01 |
| G50A + A51T + R172Q + L173F + | | | | |
| N174Q | | | | |
| SEQ ID NO: 2 + | 1.01 | 0.96 | 1.08 | 1.03 |
| R172N + A204V | | | | |
| SEQ ID NO: 2 + L173V + A204V | 1.07 | 0.99 | 0.95 | 1.02 |
| SEQ ID NO: 2 + N174T + A204T | 1.08 | 1.05 | 0.97 | 0.95 |
| SEQ ID NO: 2 + | 1.17 | 0.96 | 0.96 | 1.18 |
| A51V + R172Q + N174S + A204V | | | | |
| SEQ ID NO: 2 + | 1.09 | 1.04 | 1.07 | 0.94 |
| G50A + R172Q + N174Q + A204T | | | | |
| SEQ ID NO: 2 + | 1.22 | 1.02 | 1.23 | 1 |
| G50A + R172D + L173T + N174S | | | | |
| SEQ ID NO: 2 + | 0.99 | 1.03 | 0.96 | 1 |
| G50A + L173F + N174E | | | | |
| SEQ ID NO: 2 + | 1.22 | 1.17 | 1.21 | 1.1 |
| G50A + N174D + A204T | | | | |
| SEQ ID NO: 2 + | 0.93 | 0.97 | 1.03 | 1 |
| G50A + R172D + L173T + N174D | | | | |
| SEQ ID NO: 2 + | 1.42 | 0.81 | 1.32 | 1.03 |
| R172Q + L173V | | | | |
| SEQ ID NO: 2 + | 1.48 | 0.89 | 1.28 | 1.15 |
| G50A + A51T + R172N + L173I + | | | | |
| N174Q | | | | |
| SEQ ID NO: 2 + | 1.2 | 1.23 | 1.2 | 1.19 |
| G50A + L173T + N174S + A204V | | | | |
| SEQ ID NO: 2 + | 1.1 | 1 | 1.21 | 0.97 |
| R172Q + L173F + N174D + A204T | | | | |
| SEQ ID NO: 2 + | 1.15 | 1.03 | 1.29 | 1.12 |
| G50A + A51T + R172Q + A204V | | | | |
| SEQ ID NO: 2 + | 1.09 | 0.8 | 1.16 | 1.12 |
| G50A + L173F + N174Q + A204V | | | | |
| SEQ ID NO: 2 + | 1.06 | 0.97 | 1.18 | 1.05 |
| A51T + L173F + N174Q + A204V + | | | | |
| A521 | | | | |
| SEQ ID NO: 2 + | 1.08 | 0.84 | 1.21 | 1.03 |
| G50A + A51V + L173F + N174E + | | | | |
| E471K | | | | |
| SEQ ID NO: 2 + | 1.36 | 0.87 | 1.37 | 1.04 |
| R172E + L173F + N174D | | | | |
| SEQ ID NO: 2 + | 1.13 | 1.07 | 1.24 | 1.08 |
| A51V + N174T + A204V | | | | |
| SEQ ID NO: 2 + | 1.34 | 1.19 | 1.25 | 1.15 |
| R172K + A204V | | | | |
| SEQ ID NO: 2 + | 1.06 | 0.99 | 1.06 | 0.98 |
| R172E + L173F + A204V | | | | |
| SEQ ID NO: 2 + | 1.3 | 0.64 | 0.92 | 0.9 |
| A51T + R172Q + L173T | | | | |
| SEQ ID NO: 2 + | 1.23 | 1.11 | 1.15 | 1.18 |
| G50A + A51V + R172Q + A204V | | | | |
| SEQ ID NO: 2 + | 1.53 | 0.88 | 1.21 | 1.11 |
| G50A + A51Q + R172Q | | | | |
| SEQ ID NO: 2 + | 1.25 | 0.99 | 1.22 | 1.13 |
| A51T + R172N + A204V | | | | |
| SEQ ID NO: 2 + | 1.43 | 1.08 | 1.42 | 1.18 |
| G50A + A51T + R172E + L173I | | | | |
| SEQ ID NO: 2 + | 1.05 | 0.77 | 1.07 | 0.91 |
| A51L + R172N + N174S + A204T | | | | |
| SEQ ID NO: 2 + | 1.13 | 1.03 | 1.18 | 1.14 |
| N174Q + A204V | | | | |
| SEQ ID NO: 2 + | 1.2 | 1.16 | 1.15 | 1.12 |
| G50A + A51V + L173F + N174* | | | | |
| SEQ ID NO: 2 + | 1.6 | 1 | 1.34 | 1.11 |
| R172E + N174D + A204T | | | | |
| SEQ ID NO: 2 + | 1.41 | 0.79 | 1.32 | 1.18 |
| A51T + R172N + L173* | | | | |
| SEQ ID NO: 2 + | 1.2 | 1.01 | 1.12 | 1.25 |
| R172N + L173F + N174Q | | | | |
| SEQ ID NO: 2 + R172E + L173F | 1.16 | 0.95 | 1.3 | 1.08 |
| SEQ ID NO: 2 + | 1.04 | 1.02 | 0.94 | 1.17 |
| G50A + R172S + N174H | | | | |
| SEQ ID NO: 2 + | 1.34 | 0.97 | 1.31 | 1.27 |
| G50A + A51T + A87S + R172N + | | | | |
| L173T + N174D + A730 | | | | |
| SEQ ID NO: 2 + | 1.16 | 0.97 | 1.28 | 1.03 |
| G50A + R172N + L173T + N174D | | | | |
| SEQ ID NO: 2 + A51T + R172Q | 1.29 | 0.61 | 1.21 | 1.02 |
| SEQ ID NO: 2 + | 1.2 | 1.04 | 1.11 | 1.11 |
| G50A + R172E + L173F + N174D | | | | |
| SEQ ID NO: 2 + | 1.38 | 0.89 | 1.03 | 1.04 |
| G67E + D112N + R172D + L173F + | | | | |
| N174S + S466F | | | | |
| SEQ ID NO: 2 + | 1.48 | 1.09 | 1.38 | 1.18 |
| G50A + R172D + N174T | | | | |
| SEQ ID NO: 2 + | 1.37 | 1.02 | 1.23 | 1.21 |
| G50A + A51T + R172Q + N174T | | | | |
| SEQ ID NO: 2 + G50A + R172D | 1.49 | 0.94 | 1.38 | 1.28 |
| SEQ ID NO: 2 + A51I + N174D | 0.84 | 1.01 | 1.11 | 0.94 |
| SEQ ID NO: 2 + | 1.39 | 1.09 | 1.26 | 1.36 |
| G50A + N174E + A204T | | | | |
| SEQ ID NO: 2 + | 1.43 | 1.12 | 1.44 | 1.23 |
| R172Q + L173V + A204T | | | | |
| SEQ ID NO: 2 + | 1.07 | 1.05 | 0.96 | 1 |
| R172Q + L173V + A204V | | | | |
| SEQ ID NO: 2 + | 1.05 | 0.96 | 0.98 | 0.89 |
| G50A + A51T + L173F + N174S | | | | |
| SEQ ID NO: 2 + | 1.18 | 0.97 | 1.11 | 0.97 |
| A51V + R172Q + L173F + N174S | | | | |
| SEQ ID NO: 2 + | 1.56 | 1.03 | 1.49 | 1 |
| A51T + R172D + N174Q | | | | |
| SEQ ID NO: 2 + | 1.49 | 1.07 | 1.43 | 1.31 |
| G50A + A51T + R172E + N174S | | | | |
| SEQ ID NO: 2 + | 0.97 | 0.9 | 1.12 | 0.93 |
| R172E + N174S | | | | |
| SEQ ID NO: 2 + | 1.35 | 1.07 | 1.28 | 1.23 |
| G50A + A51T + R172Q + N174Q | | | | |
| SEQ ID NO: 2 + | 1.14 | 0.98 | 1.13 | 0.99 |
| G50A + A51T + R172Q + L173F + | | | | |
| N174T | | | | |
| SEQ ID NO: 2 + | 1.23 | 0.9 | 1.28 | 1.13 |
| A51V + L173V + A204V | | | | |
| SEQ ID NO: 2 + A204T | 1.05 | 0.9 | 0.97 | 1.05 |
| SEQ ID NO: 2 + | 0.95 | 1.03 | 0.89 | 0.95 |
| H1L + A113Y + G149Q + K302R + | | | | |
| H324L + E345S + E391L | | | | |
| SEQ ID NO: 2 + E391L + K423P | 1.06 | 1.01 | 1.14 | 1.03 |
| SEQ ID NO: 2 + | 1.02 | 0.92 | 1.25 | 1.01 |
| T40K + V56T + R171E | | | | |
| SEQ ID NO: 2 + | 0.94 | 0.89 | 0.96 | 1.01 |
| H1L + T40K + V56T + E391L + K423P | | | | |
| SEQ ID NO: 2 + | 1.13 | 0.87 | 1.37 | 0.91 |
| G149Q + R171E | | | | |
| SEQ ID NO: 2 + | 1.1 | 1.02 | 1.34 | 1.14 |
| A113Y + G149Q + R171E + D192E + | | | | |
| Q280S | | | | |
| SEQ ID NO: 2 + | 1.21 | 0.98 | 1.36 | 1.03 |
| H1L + A113Y + R171E + D192E | | | | |
| SEQ ID NO: 2 + | 1.38 | 0.8 | 1.56 | 1.01 |
| R171E + D192E + Q280S + Q361A + | | | | |
| Y363L + E391L + K423P | | | | |
| SEQ ID NO: 2 + | 0.9 | 1.09 | 0.92 | 1.04 |
| D192E + Q280S | | | | |
| SEQ ID NO: 2 + | 0.97 | 0.91 | 1.14 | 1.13 |
| G50A + R172Q + N174T + G304Q + | | | | |
| E345D + E346T + D476G + G477S | | | | |
| SEQ ID NO: 2 + | 1.15 | 0.95 | 1.41 | 1.08 |
| G50A + R172D + N174D + G304Q + | | | | |
| E345D + E346T + D476G + G477S | | | | |
| SEQ ID NO: 2 + | 1.16 | 0.97 | 1.47 | 1.05 |
| G50A + A51V + R172D + L173F + | | | | |
| G304Q + E345D + E346T + D476G + | | | | |
| G477S | | | | |
| SEQ ID NO: 2 + | 1.33 | 0.99 | 1.6 | 1.12 |
| G50A + A51V + R172D + L173F + | | | | |
| N174Q + A204T + G304Q + E345D + | | | | |
| E346T + D476G + G477S | | | | |
| SEQ ID NO: 2 + | 1.4 | 1.12 | 1.6 | 1.25 |
| G50A + R172Q + L173I + N174T + | | | | |
| A204T + G304Q + E345D + E346T + | | | | |
| D476G + G477S | | | | |
| SEQ ID NO: 2 + | 1.15 | 0.95 | 1.32 | 1.1 |
| R172Q + N174D + G304Q + E345D + | | | | |
| E346T + D476G + G477S | | | | |
| SEQ ID NO: 2 + | 0.81 | 0.84 | 0.8 | 1.34 |
| K302Q + G304A + E345D + E346T + | | | | |
| D476Q + G477Q | | | | |
| SEQ ID NO: 2 + | 0.76 | 1.09 | 0.68 | 0.87 |
| G304Q + E346T | | | | |
| SEQ ID NO: 2 + | 0.71 | 1.21 | 0.65 | 0.75 |
| K302N + D476G | | | | |
| SEQ ID NO: 2 + | 0.82 | 1.05 | 0.79 | 0.76 |
| K302Q + D476Q + G477Q | | | | |
| SEQ ID NO: 2 + | 0.88 | 1.11 | 0.87 | 0.79 |
| G304Q + E345D + E346P + D476G + | | | | |
| G477T | | | | |
| SEQ ID NO: 2 + | 0.93 | 1.27 | 0.74 | 0.82 |
| G305R + E346P + E449K | | | | |
| SEQ ID NO: 2 + | 1.06 | 1.3 | 1.02 | 0.99 |
| E345D + E346T + D476Q + G477A | | | | |
| SEQ ID NO: 2 + | 1.11 | 0.93 | 0.95 | 1.08 |
| K302N + E346T + D476Q + G477T | | | | |
| SEQ ID NO: 2 + | 0.89 | 0.95 | 0.89 | 1.26 |
| G305A + E345D | | | | |
| SEQ ID NO: 2 + | 0.91 | 1 | 0.79 | 0.9 |
| K302Q + G304A + G305A + E346P | | | | |
| SEQ ID NO: 2 + | 0.74 | 1.11 | 0.78 | 0.95 |
| G304Q + E345D + E346T + D476G | | | | |
| SEQ ID NO: 2 + | 1.3 | 1.07 | 1.15 | 1.01 |
| K302Q + D476G + G477A | | | | |
| SEQ ID NO: 2 + | 1.07 | 0.94 | 1.02 | 1.22 |
| K302Q + E345D + D476K + G477Q | | | | |
| SEQ ID NO: 2 + | 0.65 | 1.19 | 0.98 | 1.06 |
| G304Q + E345D + E346T + D476Q + | | | | |
| G477T | | | | |
| SEQ ID NO: 2 + | 0.93 | 0.96 | 1.14 | 1.03 |
| G304A + E346P + D476G + G477S | | | | |
| SEQ ID NO: 2 + | 1.36 | 1.15 | 1.39 | 1.46 |
| S255I + E345D + G477Q | | | | |
| SEQ ID NO: 2 + | 0.93 | 1.25 | 0.81 | 0.86 |
| G305N + E346P + D476K + G477T | | | | |
| SEQ ID NO: 2 + | 0.93 | 1.23 | 0.89 | 0.92 |
| K302Q + D476Q + G477S | | | | |
| SEQ ID NO: 2 + | 1.19 | 1.13 | 1.3 | 1.22 |
| E346T + G477A | | | | |
| SEQ ID NO: 2 + | 1.08 | 1.03 | 1.12 | 1.14 |
| G304A + E345D | | | | |
| SEQ ID NO: 2 + | 1.21 | 1.03 | 1.15 | 1.15 |
| K302Q + E346P + D476K + G477A | | | | |
| SEQ ID NO: 2 + | 0.81 | 1.09 | 0.81 | 0.9 |
| G304A + E346P + D476K + G477Q | | | | |
| SEQ ID NO: 2 + | 1 | 0.89 | 0.95 | 0.94 |
| G304A + E345D + E346T + D476Q + | | | | |
| G477K | | | | |
| SEQ ID NO: 2 + | 1.13 | 0.86 | 1.03 | 1.09 |
| G304A + E345D + E346P + D476Q + | | | | |
| G477S | | | | |
| SEQ ID NO: 2 + | 1 | 1.18 | 0.98 | 0.86 |
| G304A + E345D + E346T + D476K + | | | | |
| G477A | | | | |
| SEQ ID NO: 2 + | 1.06 | 1.11 | 1.22 | 1.08 |
| K302Q + E346P + G477A | | | | |
| SEQ ID NO: 2 + | 0.94 | 1.14 | 0.87 | 1.1 |
| G305S + E345D + D476K + G477Q | | | | |
| SEQ ID NO: 2 + | 1.16 | 0.84 | 0.92 | 1.04 |
| A47G + G67E + A111T + D112H + | | | | |
| R245L + E346P + D476G + G477T | | | | |
| SEQ ID NO: 2 + | 1.02 | 0.94 | 0.85 | 0.86 |
| A113V + E346P + D476G + G477K | | | | |
| SEQ ID NO: 2 + | 1.02 | 0.85 | 1.04 | 1.12 |
| K302N + E345D + E346P + D476Q + | | | | |
| G477S | | | | |
| SEQ ID NO: 2 + | 1.2 | 0.93 | 0.95 | 1.1 |
| G304A + E346T + D476K + G477T | | | | |
| SEQ ID NO: 2 + | 1.06 | 0.99 | 1 | 0.86 |
| E346P + G477K | | | | |
| SEQ ID NO: 2 + | 1.38 | 1.23 | 1.37 | 1.31 |
| G304S + E345D + D476K + G477Q | | | | |
| SEQ ID NO: 2 + | 1.15 | 1.08 | 1.16 | 1.01 |
| K302N + E345D + D476G | | | | |
| SEQ ID NO: 2 + | 0.95 | 0.95 | 1.09 | 0.93 |
| D476G + G477S | | | | |
| SEQ ID NO: 2 + | 1.45 | 1.03 | 1.39 | 0.85 |
| A113T + G304S + E345D + E346P + | | | | |
| D476G + G477Q | | | | |
| SEQ ID NO: 2 + | 0.99 | 1.18 | 1.02 | 0.85 |
| E345D + E346P + D476G + G477A | | | | |
| SEQ ID NO: 2 + | 0.97 | 1.01 | 1.03 | 1 |
| E345D + E346P + D476Q + G477T | | | | |
| SEQ ID NO: 2 + | 1.25 | 1.11 | 1.05 | 1.05 |
| R320S + H324Q | | | | |
| SEQ ID NO: 2 + | 1.1 | 1.1 | 1.26 | 1.18 |
| R320S + S323M | | | | |
| SEQ ID NO: 2 + | 1.2 | 1.01 | 1.14 | 0.95 |
| G7E + R320M + S323M | | | | |
| SEQ ID NO: 2 + | 1 | 1.03 | 1.21 | 0.91 |
| D112H + R320A + S323N | | | | |
| SEQ ID NO: 2 + | 1.01 | 0.99 | 1.19 | 1.12 |
| K108E + G110D + A111T + D112H + | | | | |
| R320S + S323M | | | | |
| SEQ ID NO: 2 + | 1.32 | 1.07 | 1.46 | 0.86 |
| G7Q + Q98S + R320S + H324Q | | | | |
| SEQ ID NO: 2 + | 1.01 | 0.86 | 1.22 | 0.87 |
| G7E + R320A + H324Q | | | | |
| SEQ ID NO: 2 + Q98S + R320S | 1.01 | 0.88 | 1.03 | 1.11 |
| SEQ ID NO: 2 + | 1.26 | 1 | 1.33 | 0.89 |
| Q98A + R320A + S323N | | | | |
| SEQ ID NO: 2 + | 1.06 | 0.79 | 1.09 | 1.01 |
| G7L + Q98N + R320A + S323N | | | | |
| SEQ ID NO: 2 + | 1 | 0.98 | 0.83 | 0.82 |
| G7A + R320S + S323M | | | | |
| SEQ ID NO: 2 + | 1.02 | 0.95 | 0.95 | 0.81 |
| G7K + R320A + S323N | | | | |
| SEQ ID NO: 2 + | 1.26 | 1.01 | 1.21 | 1.01 |
| Q98T + R320A + H324L | | | | |
| SEQ ID NO: 2 + | 1.23 | 0.84 | 1.19 | 0.91 |
| G7E + Q98T + R320A | | | | |
| SEQ ID NO: 2 + | 1.22 | 0.86 | 1 | 0.89 |
| G7L + R320S + S323M | | | | |
| SEQ ID NO: 2 + | 1.26 | 0.63 | 1.08 | 0.89 |
| G7D + Q98A + R320M | | | | |
| SEQ ID NO: 2 + | 1.05 | 0.68 | 0.95 | 0.84 |
| G7N + Q98L + R320A + S323M | | | | |
| SEQ ID NO: 2 + | 1.25 | 0.88 | 1.11 | 0.83 |
| G7E + Q98A + R320M | | | | |
| SEQ ID NO: 2 + G7N + R320S | 1.08 | 1.06 | 1.3 | 1 |
| SEQ ID NO: 2 + | 1.4 | 1.29 | 1.46 | 1.13 |
| R320A + S323N | | | | |
| SEQ ID NO: 2 + | 1.12 | 1 | 1.06 | 1.09 |
| G7K + R320A + S323M + H324L | | | | |
| SEQ ID NO: 2 + | 1.45 | 0.76 | 1.53 | 1.02 |
| G7L + P211Q + S303G + R320A | | | | |
| SEQ ID NO: 2 + | 1.06 | 0.8 | 0.84 | 0.76 |
| G7N + R320A + S323M | | | | |
| SEQ ID NO: 2 + | 1.14 | 0.87 | 0.93 | 0.86 |
| G7Q + R320S + S323M | | | | |
| SEQ ID NO: 2 + | 1.19 | 0.83 | 1.35 | 0.98 |
| Q98A + A265S + R320A | | | | |
| SEQ ID NO: 2 + G7N + R320A | 1.11 | 0.83 | 0.89 | 0.77 |
| SEQ ID NO: 2 + | 0.83 | 0.87 | 1.17 | 0.87 |
| G7E + R320A + S323M | | | | |
| SEQ ID NO: 2 + | 1.06 | 0.96 | 1.17 | 1.09 |
| G7A + Q98L + R320M | | | | |
| SEQ ID NO: 2 + | 1.24 | 1.03 | 1.29 | 1.06 |
| Q98N + R320A + S323N | | | | |
| SEQ ID NO: 2 + R320M | 1.17 | 0.89 | 1.44 | 0.95 |
| SEQ ID NO: 2 + G7A + R320M | 1.18 | 0.91 | 1.2 | 0.76 |
| SEQ ID NO: 2 + | 1.26 | 0.94 | 1.43 | 1.03 |
| G7P + R320S + S323K | | | | |
| SEQ ID NO: 2 + | 1.31 | 0.85 | 1.37 | 0.94 |
| G7Q + R320A + S323N | | | | |
| SEQ ID NO: 2 + | 0.86 | 1.03 | 1.07 | 1.06 |
| R320A + S323M | | | | |
| SEQ ID NO: 2 + | 1.12 | 0.85 | 1.15 | 0.77 |
| G7L + Q98A + R320A + S323M | | | | |
| SEQ ID NO: 2 + | 1.05 | 0.49 | 1.03 | 0.76 |
| G7Q + R320M + S323N + H324W | | | | |
| SEQ ID NO: 2 + | 1.17 | 1 | 1.28 | 1.06 |
| R320A + H324L | | | | |
| SEQ ID NO: 2 + | 1.06 | 0.82 | 1.17 | 0.95 |
| G7S + R320A + S323N + H324Q | | | | |
| SEQ ID NO: 2 + R320S | 0.88 | 0.96 | 1.08 | 1.08 |
| SEQ ID NO: 2 + | 0.94 | 0.96 | 1.12 | 0.97 |
| Q98S + R320A + S323N | | | | |
| SEQ ID NO: 2 + G7L + R320A | 1.08 | 0.94 | 1.09 | 0.96 |
| SEQ ID NO: 2 + | 0.97 | 0.99 | 1.09 | 1.04 |
| Q98L + R320M + S323M | | | | |
| SEQ ID NO: 2 + | 1.36 | 0.99 | 1.21 | 1.13 |
| G7K + R320M + H321Q | | | | |
| SEQ ID NO: 2 + | 1.33 | 1.16 | 1.29 | 1.22 |
| R320S + S323N | | | | |
| SEQ ID NO: 2 + | 0.97 | 0.95 | 1.02 | 0.87 |
| R320A + S323M + H324Q | | | | |
| SEQ ID NO: 2 + | 1.15 | 0.94 | 1.25 | 1.08 |
| G7N + R320A + S323N | | | | |
| SEQ ID NO: 2 + | 1.06 | 0.96 | 1.14 | 1.09 |
| R320M + S323N | | | | |
| SEQ ID NO: 2 + | 1.27 | 1 | 1.21 | 1.04 |
| G7Q + R320S + H321N + S323M | | | | |
| SEQ ID NO: 2 + | 1.25 | 1.11 | 1.31 | 1.12 |
| R320A + H324Q | | | | |
| SEQ ID NO: 2 + | 1.14 | 1.09 | 0.96 | 1.14 |
| G304A + E346P + D476Q + G477T | | | | |
| SEQ ID NO: 2 + | 1.13 | 1.09 | 1.1 | 0.93 |
| K302Q + E346P + D476K | | | | |
| SEQ ID NO: 2 + | 1.09 | 1.11 | 1.05 | 0.9 |
| G304S + E346P + D476Q + G477S | | | | |
| SEQ ID NO: 2 + | 1.12 | 1.1 | 1.1 | 1.07 |
| K302Q + E346P + G477Q | | | | |
| SEQ ID NO: 2 + | 1.22 | 1.12 | 1.15 | 1.05 |
| K302Q + D476G + G477Q | | | | |
| SEQ ID NO: 2 + | 1.09 | 1.04 | 0.9 | 1.07 |
| G305S + E345D + E346P + G477Q | | | | |
| SEQ ID NO: 2 + | 1.16 | 1.05 | 1.06 | 1.03 |
| G304A + E345D + D476Q | | | | |
| SEQ ID NO: 2 + | 0.58 | 1.08 | 0.87 | 0.93 |
| G304Q + E345D + E346P + D476K + | | | | |
| G477Q | | | | |
| SEQ ID NO: 2 + | 1.05 | 1.01 | 1 | 1.19 |
| K302Q + G304Q + D476K + G477K | | | | |
| SEQ ID NO: 2 + | 1 | 1 | 0.93 | 1.09 |
| G304S + E345D + E346T + D476K | | | | |
| SEQ ID NO: 2 + | 0.86 | 1.02 | 0.82 | 1.04 |
| N125H + E345D + E346P + D476K + | | | | |
| G477A | | | | |
| SEQ ID NO: 2 + | 0.98 | 1.09 | 1 | 1.1 |
| G305S + E346P + D476K + G477Q | | | | |
| SEQ ID NO: 2 + | 0.9 | 1.04 | 0.82 | 1.02 |
| K302Q + E346T + D476K + G477S | | | | |
| SEQ ID NO: 2 + | 1.1 | 1.1 | 1.11 | 1.06 |
| E345D + E346P + D476Q + G477S | | | | |
| SEQ ID NO: 2 + | 1.1 | 1.08 | 1.12 | 1.1 |
| G304Q + E345D + E346T | | | | |
| SEQ ID NO: 2 + | 1.02 | 1.06 | 1.02 | 0.87 |
| E345D + E346P + G477K | | | | |
| SEQ ID NO: 2 + | 1.04 | 1.13 | 1.08 | 0.95 |
| S466F + D476K | | | | |
| SEQ ID NO: 2 + | 1.03 | 1.01 | 1 | 0.95 |
| G305S + E346P | | | | |
| SEQ ID NO: 2 + | 0.98 | 1.03 | 1 | 1.03 |
| K302Q + E346T + D476K + G477Q | | | | |
| SEQ ID NO: 2 + | 0.79 | 0.96 | 1 | 0.9 |
| K302Q + G304A + E346P | | | | |
| SEQ ID NO: 2 + | 1.17 | 1.1 | 1.19 | 1.08 |
| E345D + E346T + D476G + G477S | | | | |
| SEQ ID NO: 2 + | 0.95 | 0.95 | 1.19 | 0.7 |
| D112N + R320M + S323M + E449G | | | | |
| SEQ ID NO: 2 + | 0.93 | 0.74 | 1.02 | 0.6 |
| G7E + R320A + S323N | | | | |
| SEQ ID NO: 2 + | 1.16 | 0.78 | 1.11 | 0.73 |
| V75I + R320A + S323N | | | | |
| SEQ ID NO: 2 + | 0.92 | 1.02 | 0.89 | 0.81 |
| H2Y + R320A + S323M | | | | |
| SEQ ID NO: 2 + | 1.05 | 0.81 | 0.99 | 0.77 |
| G7Q + R320A + S323M | | | | |
| SEQ ID NO: 2 + | 1.07 | 0.79 | 1.02 | 0.87 |
| G7L + R320S + S323M | | | | |
| SEQ ID NO: 2 + | 1.4 | 1.05 | 1.34 | 0.99 |
| G7Q + Q98T + R320A | | | | |
| SEQ ID NO: 2 + | 1.01 | 0.96 | 0.78 | 0.62 |
| Q86L + R320A + S323M | | | | |
| SEQ ID NO: 2 + R320T | 1.4 | 1.13 | 1.15 | 0.92 |
| SEQ ID NO: 2 + | 1.28 | 0.97 | 1.25 | 0.83 |
| G7N + Q98S + R320M + S323N | | | | |
| SEQ ID NO: 2 + | 1.16 | 1.09 | 1.17 | 0.94 |
| G7Q + R320A + S323R | | | | |
| SEQ ID NO: 2 + | 1.29 | 0.95 | 1.3 | 1.01 |
| G7E + Q98A + K302R + R320A + | | | | |
| S323N | | | | |
| SEQ ID NO: 2 + | 1.19 | 0.88 | 1.23 | 0.9 |
| G7A + R320S + H321W + S323M | | | | |
| SEQ ID NO: 2 + | 1.2 | 0.87 | 1.11 | 0.76 |
| A87D + R320S + S323M | | | | |
| SEQ ID NO: 2 + | 1.22 | 0.89 | 0.94 | 1.01 |
| R320S + S323M + H324L | | | | |
| SEQ ID NO: 2 + | 1.3 | 1 | 1.17 | 0.88 |
| Q98K + K269N + R320A + S323M + | | | | |
| H324Q | | | | |
| SEQ ID NO: 2 + | 1.47 | 0.95 | 1.42 | 0.98 |
| G7K + R320A + P408Q | | | | |
| SEQ ID NO: 2 + | 1.29 | 0.97 | 1.14 | 0.93 |
| G7K + R320A + H324L | | | | |
| SEQ ID NO: 2 + | 0.89 | 0.77 | 1.1 | 0.94 |
| Q98N + R320S + S323N | | | | |
| SEQ ID NO: 2 + | 1.19 | 0.97 | 1.1 | 1.28 |
| R320A + H321W + S323M + H324Q | | | | |
| SEQ ID NO: 2 + | 1.07 | 1.04 | 1.12 | 1.1 |
| R320S + S323M + H324Q | | | | |
| SEQ ID NO: 2 + | 1.43 | 0.96 | 1.38 | 1 |
| G7L + R320M + S323N | | | | |
| SEQ ID NO: 2 + | 1.37 | 1.12 | 1.27 | 1.05 |
| N28S + R320M + S323N | | | | |
| SEQ ID NO: 2 + Q98S + R320A | 1.22 | 1.1 | 1.29 | 1.18 |
| SEQ ID NO: 2 + G7E + R320A | 1.1 | 0.87 | 1.15 | 0.96 |
| SEQ ID NO: 2 + | 1.35 | 1.03 | 1.29 | 1.15 |
| T5* + G4* + N3* + R320A | | | | |
| SEQ ID NO: 2 + | 1.51 | 1.04 | 1.51 | 1.14 |
| G7L + R320A + S323M | | | | |
| SEQ ID NO: 2 + | 1.19 | 0.73 | 1.18 | 0.87 |
| G7E + R320A + P320Q + S323M | | | | |
| SEQ ID NO: 2 + | 1.64 | 1.11 | 1.58 | 1.17 |
| G7K + R320A + S323N + P364S | | | | |
| SEQ ID NO: 2 + | 1.37 | 1.07 | 1.41 | 1.01 |
| G7Q + R320A + H321Q + S323M | | | | |
| SEQ ID NO: 2 + | 1.28 | 1.2 | 1.44 | 1.39 |
| R320V + D476Y | | | | |
| SEQ ID NO: 2 + G7N + R320M | 1.18 | 0.78 | 1.23 | 0.91 |
| SEQ ID NO: 2 + | 1.52 | 0.91 | 1.45 | 1.15 |
| G7H + Q98S + R320A + S323M + | | | | |
| H324L | | | | |
| SEQ ID NO: 2 + | 1.43 | 0.95 | 1.45 | 0.88 |
| G7Q + R320S + H321N | | | | |
| SEQ ID NO: 2 + | 1.56 | 1.05 | 1.57 | 1.27 |
| R320A + H321N + S323N | | | | |
| SEQ ID NO: 2 + G7E + R320M | 1.12 | 1.01 | 1.16 | 0.85 |
| SEQ ID NO: 2 + | 1.08 | 1.18 | 1.31 | 0.92 |
| Q98A + R320A + S323M | | | | |
| SEQ ID NO: 2 + G7K + R320M | 1.01 | 0.88 | 1.04 | 0.88 |
| SEQ ID NO: 2 + | 1.39 | 0.86 | 1.45 | 1.23 |
| G7Q + Q98T + R320A + S323M | | | | |
| SEQ ID NO: 2 + | 1.2 | 1.05 | 1.03 | 0.95 |
| Q98T + R320S + S323M | | | | |
| SEQ ID NO: 2 + | 1.2 | 0.99 | 1.18 | 1.09 |
| R320M + S323M + H324Q | | | | |
| SEQ ID NO: 2 + G7K + R320A | 1.2 | 1.04 | 1.14 | 1 |
| SEQ ID NO: 2 + | 1.16 | 0.98 | 1.01 | 0.81 |
| Q98A + R320S + S323M | | | | |
| SEQ ID NO: 2 + | 1.21 | 1.03 | 1.13 | 0.68 |
| G7K + T246M + R320A + S323M | | | | |
| SEQ ID NO: 2 + | 1.28 | 0.96 | 1.04 | 0.77 |
| G7N + M9V + Q98A + R320S + S323M | | | | |
| SEQ ID NO: 2 + | 1.24 | 1.03 | 1.22 | 0.93 |
| G7Q + Q98A + R320M + S323M | | | | |
| SEQ ID NO: 2 + | 1.22 | 1.02 | 1.12 | 0.91 |
| G7Q + Q86H + R320A + S323M | | | | |
| SEQ ID NO: 2 + | 1.35 | 0.84 | 1.24 | 0.98 |
| G50A + A51Q + R172Q + G304Q + | | | | |
| E345D + E346T + D476G + G477S | | | | |
| SEQ ID NO: 2 + | 1.8 | 1.18 | 1.67 | 1.27 |
| R172E + N174D + A204T + G304Q + | | | | |
| E345D + E346T + D476G + G477S | | | | |
| SEQ ID NO: 2 + | 1.8 | 1.25 | 1.71 | 1.39 |
| G50A + A51V + R172Q + A204V + | | | | |
| G304Q + E345D + E346T + D476G + | | | | |
| G477S | | | | |
| SEQ ID NO: 2 + | 1.58 | 1.09 | 1.63 | 1.34 |
| A51T + R172N + A204V + G304Q + | | | | |
| E345D + E346T + D476G + G477S | | | | |
| SEQ ID NO: 2 + | 1.63 | 1.11 | 1.68 | 1.3 |
| G50A + A51T + A87S + R172N + | | | | |
| L173T + N174D + G304Q + E345D + | | | | |
| E346T + D476G + G477S | | | | |
| SEQ ID NO: 2 + | 1.49 | 0.84 | 1.59 | 1.03 |
| A51T + R172D + N174Q + G304Q + | | | | |
| E345D + E346T + D476G + G477S | | | | |
| SEQ ID NO: 2 + | 1.56 | 1 | 1.37 | 1.1 |
| G50A + A51T + R172E + N174S + | | | | |
| G304Q + E345D + E346T + D476G + | | | | |
| G477S | | | | |
| SEQ ID NO: 2 + | 1.34 | 1.04 | 1.18 | 1.24 |
| G50A + A51T + R172Q + N174S + | | | | |
| A204V | | | | |
| SEQ ID NO: 2 + | 1.08 | 0.9 | 1.08 | 1.28 |
| G50A + A51T + L173V + A204V | | | | |
| SEQ ID NO: 2 + | 1.26 | 1.1 | 1.28 | 1.03 |
| G50A + R172D + N174S | | | | |
| SEQ ID NO: 2 + | 1.25 | 0.87 | 1.07 | 0.93 |
| A51V + R172E + N174* | | | | |
| SEQ ID NO: 2 + | 0.93 | 1.01 | 0.94 | 0.92 |
| G50A + A51T + L173T + N174Q + | | | | |
| P211T | | | | |
| SEQ ID NO: 2 + G50A + R172N | 1.22 | 0.96 | 0.99 | 1.04 |
| SEQ ID NO: 2 + | 1.3 | 0.96 | 1.07 | 1.14 |
| G50A + R172N + L173V | | | | |
| SEQ ID NO: 2 + A51V + R172D | 1.49 | 0.82 | 1.18 | 0.95 |
| SEQ ID NO: 2 + | 1.2 | 1.17 | 1.26 | 1.04 |
| L173T + N174T + A204T | | | | |
| SEQ ID NO: 2 + | 1.16 | 0.85 | 1.26 | 1 |
| G50A + R172E + L173V + N174S | | | | |
| SEQ ID NO: 2 + | 1.32 | 1 | 1.18 | 1.13 |
| R172E + N174T | | | | |
| SEQ ID NO: 2 + | 1.13 | 0.64 | 1.22 | 1.02 |
| N28D + G109S + A119P + R172D + | | | | |
| L173V + N174S | | | | |
| SEQ ID NO: 2 + | 1.22 | 0.94 | 1.1 | 0.95 |
| R172E + N174D | | | | |
| SEQ ID NO: 2 + | 1.16 | 0.74 | 1.1 | 0.78 |
| A51I + R172E + L173F | | | | |
| SEQ ID NO: 2 + | 1.12 | 0.91 | 1.3 | 1.25 |
| R172Q + A204V | | | | |
| SEQ ID NO: 2 + | 1.38 | 0.87 | 1.13 | 1.14 |
| G50A + A51T + R172E + N174T | | | | |
| SEQ ID NO: 2 + | 1.25 | 1.03 | 1.02 | 1.13 |
| R172Q + L173F + N174T | | | | |
| SEQ ID NO: 2 + | 1.17 | 1.16 | 1.31 | 0.96 |
| R172N + N174T + A204V | | | | |
| SEQ ID NO: 2 + | 1.22 | 1.07 | 1.14 | 1.12 |
| R172K + N174S | | | | |
| SEQ ID NO: 2 + | 1.19 | 1.04 | 1.08 | 1.09 |
| L173T + N174S + A204V | | | | |
| SEQ ID NO: 2 + | 1.29 | 1.06 | 1.35 | 0.76 |
| R172D + N174S | | | | |
| SEQ ID NO: 2 + | 1.58 | 1.07 | 1.41 | 0.88 |
| R172D + L173V + N174D | | | | |
| SEQ ID NO: 2 + | 1.39 | 1.07 | 1.28 | 1.02 |
| G50A + A51T + R172Q + L173I | | | | |
| SEQ ID NO: 2 + | 1.17 | 0.85 | 1.04 | 0.92 |
| R172Q + L173T + N174Q | | | | |
| SEQ ID NO: 2 + | 1.54 | 0.97 | 1.24 | 1.19 |
| R172Q + N174E | | | | |
| SEQ ID NO: 2 + | 1.05 | 0.88 | 1.19 | 0.93 |
| G50A + R172E + L173T + N174S | | | | |
| SEQ ID NO: 2 + | 1.24 | 1.02 | 1.42 | 1.15 |
| G50A + A51T + R172E + A204V | | | | |
| SEQ ID NO: 2 + | 1.09 | 1.01 | 1.28 | 1.03 |
| R172D + N174D | | | | |
| SEQ ID NO: 2 + | 1.05 | 1.06 | 1.11 | 1.04 |
| L173V + N174D | | | | |
| SEQ ID NO: 2 + | 1.25 | 0.88 | 1.47 | 0.86 |
| R172D + L173F + N174D | | | | |
| SEQ ID NO: 2 + | 1.16 | 1.01 | 1.41 | 1.02 |
| G50A + R172E + N174T | | | | |
| SEQ ID NO: 2 + | 1.17 | 1.11 | 1.38 | 1.22 |
| L173F + N174Q + A204V | | | | |
| SEQ ID NO: 2 + | 0.97 | 0.91 | 1.15 | 1.1 |
| L173T + N174S + A204V + T255K | | | | |
| SEQ ID NO: 2 + | 1.25 | 1.23 | 1.47 | 1.23 |
| G50A + R172N + A204T | | | | |
| SEQ ID NO: 2 + | 1.31 | 0.93 | 1.3 | 1.1 |
| R172E + L173I + A204T | | | | |
| SEQ ID NO: 2 + | 1.26 | 0.94 | 1.17 | 1.19 |
| G50A + R172Q + L173V | | | | |
| SEQ ID NO: 2 + | 0.89 | 0.9 | 0.94 | 1.04 |
| G50A + A51T + L173T | | | | |
| SEQ ID NO: 2 + | 1.32 | 0.65 | 1.38 | 1.22 |
| A51T + R172D + N174D | | | | |
| SEQ ID NO: 2 + L173F + N174D | 1.02 | 1.11 | 1.01 | 0.98 |
| SEQ ID NO: 2 + | 1.27 | 0.98 | 1.36 | 1.05 |
| R172K + L173V + N174S + A204T | | | | |
| SEQ ID NO: 2 + | 1.6 | 1.03 | 1.43 | 0.93 |
| R172E + L173T + N174S + A204T | | | | |
| SEQ ID NO: 2 + | 1.13 | 0.91 | 1.07 | 0.95 |
| G50A + A51T + R172E | | | | |
| SEQ ID NO: 2 + | 1.14 | 1.04 | 1.39 | 0.75 |
| G50A + A51V + R172D + N174T | | | | |
| SEQ ID NO: 2 + L173T + N174D | 1.03 | 0.97 | 1.03 | 0.97 |
| SEQ ID NO: 2 + | 1.13 | 1.03 | 1.06 | 0.98 |
| A51T + R172E + N174T | | | | |
| SEQ ID NO: 2 + | 1.13 | 0.63 | 1.07 | 0.82 |
| R172D + L173F + A447T + T453N | | | | |
| SEQ ID NO: 2 + | 1.48 | 0.95 | 1.31 | 0.89 |
| L173T + N174T + A204V | | | | |
| SEQ ID NO: 2 + | 1 | 0.82 | 0.74 | 0.81 |
| G50A + L173F + A204V | | | | |
| SEQ ID NO: 2 + | 1.46 | 1 | 1.3 | 1.11 |
| R172N + N174D | | | | |
| SEQ ID NO: 2 + | 1.16 | 1.25 | 1.21 | 0.77 |
| G50A + L173I + L173I + N174T + | | | | |
| N174T + N219K + W220F | | | | |
| SEQ ID NO: 2 + | 1.51 | 1.19 | 1.23 | 0.98 |
| G50A + R172E + L173F + N174Q + | | | | |
| A762 | | | | |
| SEQ ID NO: 2 + | 1.21 | 1.21 | 1.07 | 1.1 |
| G50A + A51V + R172Q + N174Q | | | | |
| SEQ ID NO: 2 + | 1.48 | 1.01 | 1.15 | 0.99 |
| G50A + R172N + N174E | | | | |
| SEQ ID NO: 2 + | 1.17 | 1.02 | 1.24 | 0.9 |
| R172N + N174T + H324N | | | | |
| SEQ ID NO: 2 + | 1.19 | 1.14 | 1.25 | 1.15 |
| G50A + R172Q + L173T + N174Q | | | | |
| SEQ ID NO: 2 + | 1.39 | 1.12 | 1.25 | 1.13 |
| R172K + L173V + N174S + A204T + | | | | |
| L429F | | | | |
| SEQ ID NO: 2 + | 1.22 | 1 | 1.25 | 0.79 |
| G50A + R172E + L173F + N174T | | | | |
| SEQ ID NO: 2 + | 1.31 | 0.7 | 1.29 | 1.01 |
| R172D + L173V | | | | |
| SEQ ID NO: 2 + R172K + L173T | 0.88 | 0.93 | 0.96 | 1.26 |
| SEQ ID NO: 2 + | 1.29 | 1.08 | 1.22 | 0.75 |
| R172E + L173T + N174S | | | | |
| SEQ ID NO: 2 + | 1.33 | 0.62 | 1.25 | 1.03 |
| R172D + L173I + N174S | | | | |
| SEQ ID NO: 2 + | 1.38 | 0.98 | 1.2 | 0.61 |
| A51V + R172Q + L173T | | | | |
| SEQ ID NO: 2 + | 1.68 | 1.2 | 1.53 | 1.19 |
| G50A + A51T + R172Q + L173V + | | | | |
| A204V | | | | |
| SEQ ID NO: 2 + | 1.26 | 0.83 | 1.23 | 0.85 |
| G50A + A51T + R172D + L173F + | | | | |
| N174T + K423P | | | | |
| SEQ ID NO: 2 + N174E | 1.22 | 1.13 | 1.2 | 0.98 |
| SEQ ID NO: 2 + | 1.04 | 0.91 | 1.02 | 1.18 |
| R172Q + L173T | | | | |
| SEQ ID NO: 2 + | 1.02 | 0.85 | 0.96 | 0.89 |
| G50A + A51N + R172K + N174K | | | | |
| SEQ ID NO: 2 + | 0.83 | 0.96 | 0.77 | 1.01 |
| G50A + N174T + T453M | | | | |
| SEQ ID NO: 2 + | 1.29 | 0.24 | 1.19 | 0.74 |
| T40I + A51L + R172D + L173F | | | | |
| SEQ ID NO: 2 + | 1.13 | 0.83 | 1.1 | 0.83 |
| G50A + R172E + L173F + N174Q + | | | | |
| A204T | | | | |
| SEQ ID NO: 2 + | 1.08 | 0.95 | 1.02 | 0.92 |
| A51V + N174Q + A204V | | | | |
| SEQ ID NO: 2 + | 1.18 | 1.01 | 1.1 | 1.36 |
| R172K + L173F + N174Q + A204V | | | | |
| SEQ ID NO: 2 + | 1.17 | 0.93 | 1.05 | 1.16 |
| R158G + N174E | | | | |
| SEQ ID NO: 2 + | 1.15 | 0.96 | 1.07 | 1.14 |
| A51T + N174D + A204V | | | | |
| SEQ ID NO: 2 + R172N + L173I | 1.3 | 1.02 | 1.25 | 0.87 |
| SEQ ID NO: 2 + | 1.05 | 0.91 | 1.08 | 0.96 |
| A51P + R172Q + N174D | | | | |
| SEQ ID NO: 2 + | 1.17 | 0.75 | 1.04 | 0.53 |
| G50A + A113L + R172D + N174Q | | | | |
| SEQ ID NO: 2 + | 1.26 | 1.04 | 1.19 | 1.13 |
| G50A + R118D + W167F + R172D + | | | | |
| N174Q | | | | |
| SEQ ID NO: 2 + | 1.24 | 0.69 | 0.96 | 0.66 |
| G50A + A51S + A113L + R118D + | | | | |
| R172D + N174Q | | | | |
| SEQ ID NO: 2 + | 1.28 | 0.72 | 1.01 | 0.88 |
| G50A + R118D + R172D + N174Q | | | | |
| SEQ ID NO: 2 + | 1.26 | 0.96 | 1.24 | 1 |
| G50A + A51V + A113L + W167F + | | | | |
| R172N + N174T + A204T | | | | |
| SEQ ID NO: 2 + | 1.24 | 0.95 | 1.23 | 0.52 |
| G50A + A51V + R118D + W167F + | | | | |
| R172N + N174T + A204T | | | | |
| SEQ ID NO: 2 + | 1.07 | 0.96 | 1.2 | 1.32 |
| G50A + A51V + W167F + R172N + | | | | |
| N174T + A204T | | | | |
| SEQ ID NO: 2 + | 0.93 | 0.77 | 1.14 | 0.68 |
| G50A + A51V + A113L + R118D + | | | | |
| R172N + N174T + A204T | | | | |
| SEQ ID NO: 2 + | 1.05 | 0.99 | 1.19 | 1.36 |
| G50A + A51V + A113L + R172N + | | | | |
| N174T + A204T | | | | |
| SEQ ID NO: 2 + | 1.35 | 0.85 | 1.47 | 0.98 |
| G50A + A51V + R118D + R172N + | | | | |
| N174T + A204T | | | | |
| SEQ ID NO: 2 + | 1.35 | 1.11 | 1.62 | 1.28 |
| G50A + A51V + A113L + R118D + | | | | |
| R172D + L173F | | | | |
| SEQ ID NO: 2 + | 1.28 | 0.82 | 1.19 | 0.39 |
| G50A + A51V + R118D + R172D + | | | | |
| L173F | | | | |
| SEQ ID NO: 2 + | 1.19 | 0.74 | 1.25 | 0.4 |
| G50A + A51V + A113L + R118D + | | | | |
| W167F + R172D + L173F | | | | |
| SEQ ID NO: 2 + | 1.42 | 0.99 | 1.32 | 0.97 |
| G50A + A113L + W167F + R172Q + | | | | |
| L173I + N174T + A204T | | | | |
| SEQ ID NO: 2 + | 1.24 | 0.98 | 1.34 | 1.29 |
| G50A + W167F + R172Q + L173I + | | | | |
| N174T + A204T | | | | |
| SEQ ID NO: 2 + | 1.25 | 0.69 | 1.14 | 0.44 |
| G50A + R118D + W167F + R172Q + | | | | |
| L173I + N174T + A204T | | | | |
| SEQ ID NO: 2 + | 1.36 | 0.91 | 1.46 | 0.68 |
| G50A + A113L + R118D + R172Q + | | | | |
| L173I + N174T + A204T | | | | |
| SEQ ID NO: 2 + | 1.3 | 1.13 | 1.45 | 1.32 |
| G50A + A113L + R172Q + L173I + | | | | |
| N174T + A204T | | | | |
| SEQ ID NO: 2 + | 1.25 | 0.94 | 1.33 | 0.3 |
| G50A + R118D + R172Q + L173I + | | | | |
| N174T + A204T | | | | |
| SEQ ID NO: 2 + | 1.39 | 0.86 | 1.16 | 0.2 |
| G50A + A51V + R118D + W167F + | | | | |
| R172E + L173F + N174T + A204V | | | | |
| SEQ ID NO: 2 + | 1.41 | 1.12 | 1.63 | 1.48 |
| G50A + A51V + W167F + R172E + | | | | |
| L173F + N174T + A204V | | | | |
| SEQ ID NO: 2 + | 1.22 | 1.03 | 1.32 | 1.32 |
| G50A + A51V + A113L + R172E + | | | | |
| L173F + N174T + A204V | | | | |
| SEQ ID NO: 2 + | 1.35 | 0.85 | 1.3 | 0.95 |
| G50A + A51V + A113L + R118D + | | | | |
| R172E + L173F + N174T + A204V | | | | |
| SEQ ID NO: 2 + | 1.2 | 0.83 | 0.9 | 0.24 |
| G50A + A51V + R118D + R172E + | | | | |
| L173F + N174T + A204V | | | | |
| SEQ ID NO: 2 + | 1.22 | 1.03 | 1.32 | 1.42 |
| G50A + A51T + A113L + W167F + | | | | |
| R172N + L173V | | | | |
| SEQ ID NO: 2 + | 1.5 | 1.17 | 1.53 | 1.38 |
| G50A + A51T + W167F + R172N + | | | | |
| L173V | | | | |
| SEQ ID NO: 2 + | 1.26 | 0.87 | 0.99 | 0.68 |
| G50A + A51T + R118D + W167F + | | | | |
| R172N + L173V | | | | |
| SEQ ID NO: 2 + | 1.15 | 0.73 | 1.02 | 0.7 |
| G50A + A51T + A113L + R118D + | | | | |
| R172N + L173V | | | | |
| SEQ ID NO: 2 + | 1.38 | 1.1 | 1.44 | 1.16 |
| G50A + A51T + A113L + R172N + | | | | |
| L173V | | | | |
| SEQ ID NO: 2 + | 1.44 | 0.83 | 1.29 | 0.71 |
| G50A + A51T + R118D + R172N + | | | | |
| L173V | | | | |
| SEQ ID NO: 2 + | 1.25 | 1.05 | 1.22 | 1.04 |
| A113L + R172Q + N174D | | | | |
| SEQ ID NO: 2 + | 1.59 | 0.79 | 1.33 | 0.41 |
| R118D + W167F + R172Q + N174D | | | | |
| SEQ ID NO: 2 + | 1.55 | 0.86 | 1.23 | 0.57 |
| R118D + R172Q + N174D | | | | |
| SEQ ID NO: 2 + | 1.31 | 0.97 | 1.31 | 1.14 |
| W167F + R172Q + N174D | | | | |
| SEQ ID NO: 2 + | 1.4 | 1.07 | 1.33 | 1.17 |
| A204T + G304Q + E345D + E346T + | | | | |
| D476G + G477S | | | | |
| SEQ ID NO: 2 + | 1.6 | 1.2 | 1.49 | 1.55 |
| R172N + L173F + N174Q + G304Q + | | | | |
| E345D + E346T + D476G + G477S | | | | |
| SEQ ID NO: 2 + | 1.56 | 1.08 | 1.46 | 0.67 |
| A113L + W167F + R172Q + Y363L | | | | |
| SEQ ID NO: 2 + | 1.51 | 1.2 | 1.43 | 1.26 |
| T40K + G50A + R172Q + I430E | | | | |
| SEQ ID NO: 2 + T40K + E346T | 1.21 | 1.14 | 1.17 | 1.15 |
| SEQ ID NO: 2 + | 1.43 | 1.19 | 1.44 | 1.43 |
| T40K + K72R + W167F | | | | |
| SEQ ID NO: 2 + | 1.54 | 0.74 | 1.57 | 1.02 |
| W167F + R172Q + E346T + Y363L | | | | |
| SEQ ID NO: 2 + | 1.36 | 0.65 | 1.4 | 0.84 |
| K72R + A113L + W167F + R172Q + | | | | |
| N215G + Y363L | | | | |
| SEQ ID NO: 2 + | 1.21 | 0.85 | 1.12 | 0.96 |
| W167F + R172Q + E346T | | | | |
| SEQ ID NO: 2 + | 1.29 | 1.03 | 1.16 | 1.11 |
| A113L + W167F + R172Q + N215G | | | | |
| SEQ ID NO: 2 + | 1.3 | 0.91 | 1.27 | 1 |
| S255I + R320A + H321N + S323N + | | | | |
| E345D + G477Q | | | | |
| SEQ ID NO: 2 + | 1.52 | 1.1 | 1.29 | 1.28 |
| G50A + A113L + W167F + R172Q + | | | | |
| A1066 | | | | |
| SEQ ID NO: 2 + | 1.52 | 1.09 | 1.23 | 1.24 |
| G50A + W167F + R172Q + A1105 | | | | |
| SEQ ID NO: 2 + | 1.21 | 0.71 | 1.05 | 0.63 |
| G50A + R118D + R172Q | | | | |
| SEQ ID NO: 2 + | 1.49 | 0.94 | 1.11 | 0.89 |
| G50A + A113L + R118D + R172Q | | | | |
| SEQ ID NO: 2 + | 1.38 | 0.99 | 1.18 | 1.11 |
| G50A + A113L + W167F + R172Q | | | | |
| SEQ ID NO: 2 + | 1.4 | 1.22 | 1.41 | 1.07 |
| G50A + R172D + L173F + E471K | | | | |
| SEQ ID NO: 2 + | 1.15 | 1.08 | 1.17 | 1.19 |
| G50A + A51T + R172D + E345D + | | | | |
| E346P + D476G | | | | |
| SEQ ID NO: 2 + | 1.05 | 1.21 | 1.2 | 1.19 |
| G50A + R172D + N174Q + E345D + | | | | |
| E346P + D476G | | | | |
| SEQ ID NO: 2 + | 1.11 | 1.18 | 1.23 | 1.15 |
| R171E + D192E + E345D + E346P + | | | | |
| D476G | | | | |
| SEQ ID NO: 2 + | 1.22 | 1.17 | 1.28 | 1.17 |
| G50A + R172D + N174D + E345D + | | | | |
| E346P + D476G | | | | |
| SEQ ID NO: 2 + | 1.01 | 1.06 | 1.03 | 1.01 |
| G50A + A51V + R172N + N174T + | | | | |
| A204T + E345D + E346P + D476G | | | | |
| SEQ ID NO: 2 + | 1.28 | 1.38 | 1.25 | 1.15 |
| G50A + R172D + N174T + E345D + | | | | |
| E346P + D476G | | | | |
| SEQ ID NO: 2 + | 0.82 | 1.35 | 0.9 | 0.88 |
| G50A + N174T + E345D + E346P + | | | | |
| D476G | | | | |
| SEQ ID NO: 2 + | 0.98 | 1.27 | 1 | 1.03 |
| R172K + A204V + E345D + E346P + | | | | |
| D476G | | | | |
| SEQ ID NO: 2 + | 0.93 | 1.26 | 1.08 | 1.14 |
| G50A + R172N + N174T + E345D + | | | | |
| E346P + D476G | | | | |
| SEQ ID NO: 2 + | 0.89 | 1.22 | 1.02 | 0.99 |
| G50A + A51V + L173F + N174D + | | | | |
| E345D + E346P + D476G | | | | |
| SEQ ID NO: 2 + | 1.31 | 1.06 | 1.41 | 0.99 |
| G50A + A51T + R172D + S255I + | | | | |
| E345D + G477Q | | | | |
| SEQ ID NO: 2 + | 1.26 | 0.92 | 1.15 | 1.04 |
| G50A + A51V + R172E + L173F + | | | | |
| N174T + A204V + S255I + E345D + | | | | |
| G477Q | | | | |
| SEQ ID NO: 2 + | 1.32 | 0.94 | 1.25 | 1 |
| G50A + R172D + N174Q + S255I + | | | | |
| E345D + G477Q | | | | |
| SEQ ID NO: 2 + | 1.45 | 0.86 | 1.27 | 0.97 |
| R171E + D192E + S255I + E345D + | | | | |
| G477Q | | | | |
| SEQ ID NO: 2 + | 1.36 | 0.98 | 1.19 | 1 |
| G50A + R172D + N174D + S255I + | | | | |
| E345D + G477Q | | | | |
| SEQ ID NO: 2 + | 1.29 | 1.13 | 1.22 | 1.11 |
| G50A + A51V + R172N + N174T + | | | | |
| A204T + S255I + E345D | | | | |
| SEQ ID NO: 2 + | 1.33 | 0.99 | 1.43 | 1.09 |
| G50A + R172D + N174T + S255I + | | | | |
| E345D + G477Q | | | | |
| SEQ ID NO: 2 + | 1.25 | 1.05 | 1.44 | 1.19 |
| R172K + A204V + S255I + E345D + | | | | |
| G477Q | | | | |
| SEQ ID NO: 2 + | 1.25 | 0.87 | 1.35 | 1.12 |
| R172K + A204V + S255I + E345D + | | | | |
| K423P + G477Q | | | | |
| SEQ ID NO: 2 + | 1.15 | 1.1 | 1.21 | 1.09 |
| G50A + R172N + N174T + S255I + | | | | |
| E345D + G477Q | | | | |
| SEQ ID NO: 2 + | 1.11 | 0.98 | 1.21 | 1.04 |
| G50A + A51V + L173F + N174D + | | | | |
| S255I + E345D + G477Q | | | | |
| SEQ ID NO: 2 + | 1.18 | 0.92 | 1.25 | 1.06 |
| G50A + A51V + L173F + N174D + | | | | |
| S255I + E345D | | | | |
| SEQ ID NO: 2 + | 1.23 | 0.36 | 1.22 | 0.89 |
| G50A + A51T + W140Y + P146S + | | | | |
| R172D | | | | |
| SEQ ID NO: 2 + | 1.55 | 0.83 | 1.59 | 1.16 |
| G50A + A51T + W140Y + R172D | | | | |
| SEQ ID NO: 2 + | 1.42 | 0.5 | 1.36 | 1.06 |
| G50A + A51V + W140Y + R172E + | | | | |
| L173F + N174T + A204V | | | | |
| SEQ ID NO: 2 + | 1.39 | 0.95 | 1.39 | 1.11 |
| G50A + W140Y + R172D + N174Q | | | | |
| SEQ ID NO: 2 + | 1.14 | 0.62 | 1.29 | 0.94 |
| G50A + W140Y + R172D + N174D | | | | |
| SEQ ID NO: 2 + | 1.19 | 0.87 | 1.24 | 0.65 |
| G50A + W140Y + P146S + R172D + | | | | |
| N174D + E471K | | | | |
| SEQ ID NO: 2 + | 1.27 | 0.99 | 1.04 | 1.19 |
| G50A + A51V + W140Y + R172N + | | | | |
| N174T + A204T | | | | |
| SEQ ID NO: 2 + | 1.34 | 0.83 | 1.29 | 1.01 |
| G50A + A51T + W140Y + R172N + | | | | |
| L173V | | | | |
| SEQ ID NO: 2 + | 1.32 | 0.96 | 1.28 | 0.81 |
| W140Y + R172D + N174T | | | | |
| SEQ ID NO: 2 + | 1.17 | 1.11 | 1.51 | 1.28 |
| G50A + W140Y + R172N + N174T | | | | |
| SEQ ID NO: 2 + | 1.25 | 1.07 | 1.49 | 1.28 |
| G50A + A51V + W140Y + P146S + | | | | |
| L173F + N174D | | | | |
| SEQ ID NO: 2 + | 1.29 | 0.96 | 1.24 | 1.28 |
| A113L + S255I + E345D + G477Q | | | | |
| SEQ ID NO: 2 + | 1.5 | 0.75 | 1.39 | 0.38 |
| A113L + R118D + W167F + S255I + | | | | |
| E345D + G477Q | | | | |
| SEQ ID NO: 2 + | 1.31 | 1.01 | 1.13 | 1 |
| K72R + A113L + S255I + E345D + | | | | |
| G477Q | | | | |
| SEQ ID NO: 2 + | 1.41 | 1 | 1.3 | 1.3 |
| K72R + A113L + W167F + S255I + | | | | |
| E345D + G477Q | | | | |
| SEQ ID NO: 2 + | 1.29 | 0.91 | 1.07 | 1.17 |
| W167F + S255I + E345D + G477Q | | | | |
| SEQ ID NO: 2 + | 1.39 | 0.45 | 1.06 | 0.23 |
| R118D + W167F + S255I + E345D + | | | | |
| G477Q | | | | |
| SEQ ID NO: 2 + | 1.06 | 0.78 | 0.84 | 0.74 |
| Y206I + M208Y + V213S + V214T + | | | | |
| L217M | | | | |
| SEQ ID NO: 2 + | 0.95 | 0.93 | 0.98 | 1.06 |
| Q98N + R320A + S323M + A354V | | | | |
| SEQ ID NO: 2 + | 0.95 | 1.15 | 0.87 | 0.95 |
| Q98N + R320A + S323M + E391L | | | | |
| SEQ ID NO: 2 + | 0.82 | 1.15 | 1.02 | 0.98 |
| Q98N + A113L + R320A + S323M | | | | |
| SEQ ID NO: 2 + | 1.14 | 1.29 | 1.26 | 1.04 |
| Q98N + Q169M + R320A + S323M | | | | |
| SEQ ID NO: 2 + | 1.08 | 0.69 | 1.24 | 1.02 |
| Q98N + R320A + H321N + S323N | | | | |
| SEQ ID NO: 2 + Q98N + | 0.97 | 1.04 | 1.12 | 1.15 |
| Y206I + M208Y + V213S + V214T + | | | | |
| L217M + R320A + S323M | | | | |
| SEQ ID NO: 2 + Q98N + | 1.33 | 1.23 | 1.4 | 1.43 |
| Y206I + M208Y + V213T + V214T + | | | | |
| L217M + R320A + S323M | | | | |
| SEQ ID NO: 2 + | 1.31 | 1.17 | 1.57 | 1.22 |
| R172E + N174D + A204T + G304Q + | | | | |
| E345D + E346T + E391L + D476G + | | | | |
| G477S | | | | |
| SEQ ID NO: 2 + | 1.39 | 0.74 | 1.1 | 0.21 |
| R172E + N174D + A204T + G304Q + | | | | |
| E345D + E346T + E391L | | | | |
| SEQ ID NO: 2 + | 1.53 | 1.09 | 1.39 | 1.08 |
| R172E + N174D + A204T + G304Q + | | | | |
| R320A + H321N + S323N + E345D + | | | | |
| E346T + D476G + G477S | | | | |
| SEQ ID NO: 2 + | 0.95 | 0.9 | 1.15 | 0.95 |
| A51T + R172N + A204V + G304Q + | | | | |
| E345D + E346T + E391L + D476G + | | | | |
| G477S | | | | |
| SEQ ID NO: 2 + | 1.01 | 0.81 | 1.3 | 0.88 |
| A51T + R172N + A204V + G304Q + | | | | |
| R320A + H321N + S323N + E345D + | | | | |
| E346T + D476G + G477S | | | | |
| SEQ ID NO: 2 + | 1.02 | 0.99 | 1.37 | 1.08 |
| A51T + R172N + Y206I + M208Y + | | | | |
| V213S + V214T + L217M + G304Q + | | | | |
| E345D + E346T + D476G + G477S | | | | |
| SEQ ID NO: 2 + R172N + Y206I + | 1.07 | 0.93 | 1.39 | 1 |
| M208Y + V213T + V214T + L217M + | | | | |
| G304Q + E345D + E346T + D476G + | | | | |
| G477S | | | | |
| SEQ ID NO: 2 + | 1.13 | 0.98 | 1.46 | 1.02 |
| G50A + A51T + A87S + Q169M + | | | | |
| R172N + L173T + N174D + Y206I + | | | | |
| G304Q + E345D + E346T + D476G + | | | | |
| G477S | | | | |
| SEQ ID NO: 2 + | 1.41 | 1.03 | 1.71 | 1.2 |
| G50A + A51T + A87S + Q169M + | | | | |
| R172N + L173T + N174D + G304Q + | | | | |
| E345D + E346T + A206427V + | | | | |
| D476G + G477S | | | | |
| SEQ ID NO: 2 + | 1.4 | 1.13 | 1.76 | 1.17 |
| G50A + R172D + N174Q + R320A + | | | | |
| H321N + S323N | | | | |
| SEQ ID NO: 2 + | 1.32 | 0.94 | 1.57 | 1.07 |
| G50A + R172D + N174D + R320A + | | | | |
| H321N + S323N | | | | |
| SEQ ID NO: 2 + | 1.28 | 1.05 | 1.45 | 1.02 |
| G50A + A51V + R172N + N174T + | | | | |
| A204T + R320A + H321N + S323N | | | | |
| SEQ ID NO: 2 + | 1.09 | 1.06 | 1.1 | 1.02 |
| G50A + N174T + R320A + H321N + | | | | |
| S323N | | | | |
| SEQ ID NO: 2 + | 1.23 | 0.94 | 1.33 | 0.89 |
| G50A + R172N + N174T + R320A + | | | | |
| H321N + S323N | | | | |
| SEQ ID NO: 2 + | 1.33 | 0.97 | 1.12 | 1.04 |
| G50A + A51V + L173F + N174D + | | | | |
| R320A + H321N + S323N | | | | |
| SEQ ID NO: 2 + | 1.31 | 0.82 | 1.21 | 0.61 |
| W167F + E345D + D476K + G477S | | | | |
| SEQ ID NO: 2 + | 1.1 | 1.13 | 1.02 | 1 |
| A113L + W167F + E345D + D476K + | | | | |
| G477S | | | | |
| SEQ ID NO: 2 + | 1.27 | 0.89 | 1.22 | 0.82 |
| R172E + N174S + G304Q + R320A + | | | | |
| H321N + S323N + E345D + E346T + | | | | |
| D476G + G477S | | | | |
| SEQ ID NO: 2 + | 1.29 | 1.13 | 1.52 | 0.88 |
| G50A + A51T + R172E + N174S + | | | | |
| G304Q + R320A + H321N + S323N + | | | | |
| E345D + E346T + D476G + G477S | | | | |
| SEQ ID NO: 2 + | 1.09 | 1.17 | 1.23 | 1.32 |
| G50A + A51T + R172E + N174S + | | | | |
| G304Q + E345D + E346T + E391L + | | | | |
| D476G + G477S | | | | |
| SEQ ID NO: 2 + | 1.04 | 0.99 | 1.34 | 1.06 |
| G50A + A51T + A113L + R172E + | | | | |
| N174S + G304Q + E345D + E346T + | | | | |
| V474T | | | | |
| SEQ ID NO: 2 + | 0.85 | 0.92 | 1.29 | 1.01 |
| G50A + A51T + A113L + R172E + | | | | |
| N174S + G304V + E345D + E346T + | | | | |
| D476G + G477S | | | | |
| SEQ ID NO: 2 + | 1.06 | 1.07 | 1.39 | 1.3 |
| G50A + A51T + Q169M + R172E + | | | | |
| N174S + G304Q + E345D + E346T + | | | | |
| D476G + G477S | | | | |
| SEQ ID NO: 2 + | 1.34 | 1.16 | 1.64 | 1.26 |
| G50A + A51T + R172E + N174S + | | | | |
| Y206I + M208Y + V213T + V214T + | | | | |
| L217M + G304Q + E345D + E346T + | | | | |
| D476G + G477S | | | | |
| SEQ ID NO: 2 + | 1.22 | 0.44 | 1.24 | 0.61 |
| R172E + N174D + A204T + G304Q + | | | | |
| R320A + S323N + E345D + E346T + | | | | |
| D476G + G477S | | | | |
| SEQ ID NO: 2 + | 1.52 | 1.01 | 1.9 | 0.96 |
| G50A + A51V + W167F + R172E + | | | | |
| L173F + N174T + A204V + R320A + | | | | |
| S323N | | | | |
| SEQ ID NO: 2 + | 1.45 | 1.25 | 1.92 | 1.16 |
| G50A + A51V + W167F + R172E + | | | | |
| L173F + N174T + Y206I + | | | | |
| M208Y + V213S + V214T + L217M | | | | |
| SEQ ID NO: 2 + | 1.46 | 0.9 | 1.68 | 1.11 |
| G50A + A51V + W167F + R172E + | | | | |
| L173F + N174T + Y206I + | | | | |
| M208Y + V213T + V214T + L217M | | | | |
| SEQ ID NO: 2 + | 1.41 | 0.92 | 1.6 | 1.06 |
| G50A + A51T + G149A + W167F + | | | | |
| R172N + L173V | | | | |
| SEQ ID NO: 2 + | 1.33 | 0.68 | 1.5 | 0.82 |
| G50A + A51T + W167F + R172N + | | | | |
| L173V + R320A + S323N | | | | |
| SEQ ID NO: 2 + | 1.32 | 1 | 1.49 | 0.98 |
| G50A + A51T + W167F + R172N + | | | | |
| L173V + Y206I + | | | | |
| M208Y + V213S + V214T + L217M | | | | |
| SEQ ID NO: 2 + | 1.28 | 0.99 | 1.36 | 1.17 |
| G50A + A51T + W167F + R172N + | | | | |
| L173V + Y206I + | | | | |
| M208Y + V213T + V214T + L217M | | | | |
| SEQ ID NO: 2 + | 0.98 | 0.82 | 1.4 | 1 |
| R172N + L173F + N174Q + G304Q + | | | | |
| R320A + S323N + E345D + E346T + | | | | |
| D476G + G477S | | | | |
| SEQ ID NO: 2 + | 1.24 | 1.04 | 1.61 | 1.24 |
| T40K + K72R + W167F + R320A + | | | | |
| S323N | | | | |
| SEQ ID NO: 2 + | 1.25 | 0.93 | 1.29 | 1.14 |
| T40K + K72R + W140Y + W167F | | | | |
| SEQ ID NO: 2 + | 1.21 | 1.14 | 1.26 | 1.01 |
| R118P + E134D | | | | |
| SEQ ID NO: 2 + | 1.11 | 0.78 | 1.00 | 1.09 |
| N128Q + E134D + K179L | | | | |
| SEQ ID NO: 2 + | 0.96 | 1.05 | 0.91 | 0.92 |
| G50A + L173F + N174D + A204T | | | | |
| SEQ ID NO: 2 + | 1.15 | 1.27 | 1.23 | 1.02 |
| G50A + A51V + R172E + N174S | | | | |
| SEQ ID NO: 2 + | 1.24 | 1.10 | 1.27 | 1.14 |
| G50A + A51T + R172E + A204T | | | | |
| SEQ ID NO: 2 + G50A + N174Q | 0.94 | 1.19 | 0.96 | 0.94 |
| SEQ ID NO: 2 + | 1.13 | 1.46 | 1.26 | 0.94 |
| G50A + R172D + N174Q + A204V | | | | |
| SEQ ID NO: 2 + | 0.49 | 1.22 | 0.66 | 0.72 |
| G50A + A51I + L173F | | | | |
| SEQ ID NO: 2 + | 1.11 | 1.19 | 1.27 | 1.02 |
| R172K + N174S + A204G | | | | |
| SEQ ID NO: 2 + A51I + A204T | 0.97 | 1.01 | 1.09 | 1.15 |
| SEQ ID NO: 2 + | 1.34 | 0.71 | 1.54 | 1.25 |
| G50A + R172D + L173V | | | | |
| SEQ ID NO: 2 + | 1.26 | 1.29 | 1.50 | 0.96 |
| G50A + A51V + R172D + N174E | | | | |
| SEQ ID NO: 2 + | 1.34 | 0.93 | 1.56 | 0.90 |
| T40K + K72R + W167F + M208Y + | | | | |
| V213T + V214T + L217M | | | | |
| SEQ ID NO: 2 + | 1.39 | 1.07 | 1.78 | 1.33 |
| T40K + K72R + W167F + M208Y + | | | | |
| V213T + V214T + L217M + R320A + | | | | |
| H324Q | | | | |
| SEQ ID NO: 2 + | 1.13 | 0.98 | 1.39 | 1.00 |
| G50A + R172D + L173F + N174S | | | | |
| SEQ ID NO: 2 + | 1.05 | 1.01 | 1.16 | 1.00 |
| R172E + A204V | | | | |
| SEQ ID NO: 2 + | 1.09 | 0.84 | 1.49 | 1.05 |
| G50A + A51T + R172E + L173V + | | | | |
| N174T | | | | |
| SEQ ID NO: 2 + | 1.08 | 1.16 | 1.06 | 1.07 |
| G50A + A51V + N174Q | | | | |
| SEQ ID NO: 2 + A51T + L173F | 1.14 | 0.96 | 1.27 | 1.18 |
| SEQ ID NO: 2 + | 0.94 | 1.04 | 1.10 | 1.15 |
| G50A + R172K + L173T + N174T | | | | |
| SEQ ID NO: 2 + | 1.12 | 1.15 | 1.03 | 1.11 |
| G50A + L173V + N174H | | | | |
| SEQ ID NO: 2 + | 1.11 | 0.99 | 0.99 | 1.11 |
| G50A + L173T + A204T | | | | |
| SEQ ID NO: 2 + | 1.24 | 1.10 | 1.23 | 1.30 |
| R172N + L173F + N174Q + | | | | |
| Y206I + M208Y + V213S + V214T + | | | | |
| L217M + G304Q + E345D + E346T + | | | | |
| D476G + G477S | | | | |
| SEQ ID NO: 2 + | 1.10 | 1.08 | 1.29 | 1.12 |
| R172N + L173F + N174Q + | | | | |
| Y206I + M208Y + V213T + V214T + | | | | |
| L217M + G304Q + E345D + E346T + | | | | |
| D476G + G477S | | | | |
| SEQ ID NO: 2 + | 1.19 | 0.98 | 1.19 | 1.16 |
| T40K + K72R + W167F + R320A + | | | | |
| H324Q | | | | |
| SEQ ID NO: 2 + | 1.33 | 0.95 | 1.26 | 1.00 |
| T40K + K72R + W167F + R320A + | | | | |
| H321N + S321N | | | | |
| SEQ ID NO: 2 + | 1.37 | 1.03 | 1.30 | 1.17 |
| T40K + K72R + W167F + | | | | |
| Y206I + M208Y + V213S + V214T + | | | | |
| L217M | | | | |
| SEQ ID NO: 2 + A51V + L173F | 0.98 | 1.03 | 1.10 | 0.97 |
| SEQ ID NO: 2 + | 1.58 | 1.14 | 1.41 | 1.41 |
| G50A + R172D + N174D + E346T + | | | | |
| D476K + G477A | | | | |
| SEQ ID NO: 2 + | 1.63 | 1.20 | 1.32 | 1.46 |
| G50A + R172D + N174Q + E346T + | | | | |
| D476K + G477A | | | | |
| SEQ ID NO: 2 + | 1.18 | 1.16 | 0.97 | 1.34 |
| R172D + N174T + E346T + D476K + | | | | |
| G477A | | | | |
| SEQ ID NO: 2 + | 1.13 | 1.13 | 0.75 | 0.89 |
| L173F + N174S + A204T + P408Q | | | | |
| SEQ ID NO: 2 + | 1.14 | 1.05 | 0.81 | 1.19 |
| G50A + A51I + V117I + R172E | | | | |
| SEQ ID NO: 2 + | 1.29 | 1.11 | 1.05 | 1.29 |
| G50A + A51T + L173F + N174D | | | | |
| SEQ ID NO: 2 + | 0.95 | 0.96 | 0.86 | 1.01 |
| A51V + L173F + N174Q | | | | |
| SEQ ID NO: 2 + A51V + L173V | 1.23 | 0.87 | 0.98 | 0.98 |
| SEQ ID NO: 2 + | 1.40 | 0.53 | 1.13 | 0.40 |
| A41E + A51I + R172D + L173T | | | | |
| SEQ ID NO: 2 + | 1.13 | 1.20 | 0.94 | 1.25 |
| G50A + N174Q + A204T | | | | |
| SEQ ID NO: 2 + A51L + R172D | 1.50 | 0.98 | 1.31 | 1.22 |
| SEQ ID NO: 2 + | 1.11 | 1.21 | 1.03 | 1.12 |
| G50A + A111S + L173F + N174T | | | | |
| SEQ ID NO: 2 + | 1.22 | 0.87 | 1.01 | 0.97 |
| A51I + L173T + A204T + L297I | | | | |

TABLE F
Wash performance of variants

| | IF Model | IF Model | IF Model | IF Model |
|---|---|---|---|---|
| VARIANTS of SEQ ID NO: 2 | detergent A | detergent A | detergent J | detergent J |
| using SEQ ID NO: 1 for | 15° C.+, 0.2 mg | 40° C., 0.05 mg | 15° C., 0.2 mg | 30° C., 0.05 mg |
| numbering | EP | EP | EP | EP |
| Reference: | 1 | 1 | 1 | 1 |
| SEQ ID NO: 2 + L173F | 1.0 | 1.0 | 0.9 | 1.1 |
| SEQ ID NO: 2 + K72R | 1.0 | 1.2 | 1.0 | 1.0 |
| SEQ ID NO: 2 + Y160L | 1.0 | 1.1 | 1.0 | 1.1 |
| SEQ ID NO: 2 + N270G | 0.9 | 1.1 | 0.6 | 0.9 |
| SEQ ID NO: 2 + V165G | 1.3 | 1.1 | 1.2 | 1.1 |
| SEQ ID NO: 2 + N215G | 0.8 | 1.0 | 1.1 | 1.0 |
| SEQ ID NO: 2 + P211T + V213T | 1.1 | 1.0 | 0.9 | 0.9 |
| SEQ ID NO: 2 + P211T | 1.1 | 1.1 | 1.2 | 0.9 |
| SEQ ID NO: 2 + | 1.0 | 1.0 | 0.8 | 1.2 |
| G50A + R172Q + L173V | | | | |
| SEQ ID NO: 2 + N174T | 1.0 | 0.9 | 1.1 | 1.0 |
| SEQ ID NO: 2 + | 1.1 | 1.0 | 1.1 | 1.2 |
| G50A + R172N + A204T | | | | |
| SEQ ID NO: 2 + R320A | 1.0 | 1.1 | 1.0 | 1.0 |
| SEQ ID NO: 2 + | 0.7 | 0.9 | 0.8 | 0.7 |
| R320S + H321W + S323M | | | | |
| SEQ ID NO: 2 + | 0.9 | 0.7 | 0.9 | 0.8 |
| G7A + R320A + S323M | | | | |
| SEQ ID NO: 2 + G304A + E346P | 0.8 | 0.9 | 0.9 | 1.0 |
| SEQ ID NO: 2 + | 0.8 | 1.0 | 1.0 | 0.9 |
| G304S + E345D + E346T + D476Q + G477Q | | | | |
| SEQ ID NO: 2 + | 0.6 | 0.9 | 0.4 | 0.7 |
| G305S + E346T + D476K + G477K | | | | |
| SEQ ID NO: 2 + | 0.8 | 0.3 | 1.1 | 0.8 |
| G7L + A51S + R320S + S323M | | | | |
| SEQ ID NO: 2 + | 0.9 | 1.0 | 0.9 | 0.9 |
| R320A + S323M + H324L | | | | |
| SEQ ID NO: 2 + Q98N + R320S | 0.8 | 0.8 | 0.5 | 0.5 |
| SEQ ID NO: 2 + | 0.8 | 0.8 | 0.9 | 0.7 |
| Q98N + R320S + S323M + H324L | | | | |
| SEQ ID NO: 2 + | 0.8 | 0.9 | 0.8 | 0.7 |
| Q98N + R320A + S323M | | | | |
| SEQ ID NO: 2 + | 1.0 | 0.9 | 1.0 | 0.6 |
| G7N + R320M + S323M | | | | |
| SEQ ID NO: 2 + | 0.7 | 0.6 | 0.9 | 0.3 |
| G7L + R320S + S323N + H324W | | | | |
| SEQ ID NO: 2 + | 1.2 | 0.9 | 1.1 | 1.0 |
| G50A + R172E + L173F + N174T | | | | |
| SEQ ID NO: 2 + R172E + N174Q | 0.6 | 1.0 | 0.6 | 0.7 |
| SEQ ID NO: 2 + | 1.2 | 0.9 | 1.4 | 0.7 |
| R172D + L173I + N174S | | | | |
| SEQ ID NO: 2 + R172Q + N174T | 1.1 | 1.0 | 1.2 | 0.8 |
| SEQ ID NO: 2 + R26Q + L173T | 0.7 | 0.9 | 0.7 | 0.8 |
| SEQ ID NO: 2 + | 0.9 | 0.9 | 0.8 | 1.0 |
| R320A + H321N + S323N + E345D + E346P + | | | | |
| D476G | | | | |
| SEQ ID NO: 2 + | 0.8 | 0.9 | 0.9 | 0.8 |
| Q98N + G304V + R320A + S323M | | | | |
| SEQ ID NO: 2 + | 0.9 | 0.9 | 0.9 | 0.8 |
| Q98N + A113L + G304V + R320A + S323M | | | | |
| SEQ ID NO: 2 + | 0.9 | 1.0 | 1.0 | 0.9 |
| G50A + A51V + R172E + L173F + N174T + | | | | |
| A204V + E345D + E346P + D476G | | | | |
| SEQ ID NO: 2 + | 1.2 | 0.6 | 1.1 | 0.8 |
| T40K + K72R + W167F + M208Y + V213T + | | | | |
| V214T + L217M + R320A + S323N | | | | |
| SEQ ID NO: 2 + | 0.6 | 0.8 | 0.7 | 0.8 |
| A51T + L173T + N174Q + A204T | | | | |
| SEQ ID NO: 2 + A51L + N174Q | 0.5 | 0.3 | 0.4 | 0.7 |
| SEQ ID NO: 2 + | 1.5 | 1.1 | 1.6 | 1.1 |
| G50A + A51I + R172D + L173V | | | | |
| SEQ ID NO: 2 + | 1.2 | 0.7 | 1.3 | 1.0 |
| G50A + A51L + R172N + L173V + A204T | | | | |
| SEQ ID NO: 2 + | 1.4 | 0.9 | 1.6 | 1.1 |
| R172D + L173T + N174D + A204T | | | | |
| SEQ ID NO: 2 + A51I + L173F | 1.0 | 0.8 | 1.1 | 1.0 |
| SEQ ID NO: 2 + L173T + A204T | 1.1 | 0.9 | 1.2 | 1.0 |
| SEQ ID NO: 2 + | 1.1 | 0.7 | 1.2 | 1.0 |
| A51I + R172D + N174T | | | | |
| SEQ ID NO: 2 + | 1.1 | 0.9 | 1.1 | 0.9 |
| A51T + R172K + L173I + N174S | | | | |
| SEQ ID NO: 2 + | 1.3 | 1.0 | 1.2 | 1.0 |
| G50A + R172E + N174S | | | | |
| SEQ ID NO: 2 + R172N + L173V | 1.2 | 1.1 | 1.5 | 1.1 |
| SEQ ID NO: 2 + | 1.1 | 1.1 | 1.3 | 1.0 |
| G50A + R172K + N174Q | | | | |
| SEQ ID NO: 2 + | 1.2 | 1.2 | 1.4 | 1.1 |
| T5K + R172E + L173F | | | | |
| SEQ ID NO: 2 + | 1.0 | 1.2 | 1.1 | 1.2 |
| A47S + G50A + R172K + L173F + N174D | | | | |
| SEQ ID NO: 2 + | 1.2 | 0.8 | 1.2 | 1.0 |
| G50A + A51T + R172D + N174S | | | | |
| SEQ ID NO: 2 + | 1.1 | 0.9 | 1.2 | 0.9 |
| N25D + R172N + A204T | | | | |
| SEQ ID NO: 2 + | 1.4 | 1.1 | 1.4 | 1.1 |
| R172D + L173T + N174D | | | | |
| SEQ ID NO: 2 + | 0.9 | 1.1 | 0.9 | 0.9 |
| G50A + A51L + L173T + N174T + A204T | | | | |
| SEQ ID NO: 2 + | 1.2 | 1.1 | 1.0 | 1.0 |
| L173F + N174D + A204S | | | | |
| SEQ ID NO: 2 + | 1.0 | 0.9 | 0.8 | 0.9 |
| A51I + R172K + L173V + N174H + E449K | | | | |
| SEQ ID NO: 2 + | 1.2 | 1.2 | 1.1 | 0.9 |
| G50A + L173T + N174S | | | | |
| SEQ ID NO: 2 + | 1.3 | 0.9 | 1.1 | 1.0 |
| G50A + A51T + R172N + A204T | | | | |
| SEQ ID NO: 2 + | 1.0 | 1.1 | 0.9 | 0.9 |
| G50A + A51F + R172D | | | | |
| SEQ ID NO: 2 + | 1.2 | 0.9 | 1.2 | 0.9 |
| G50A + A51S + R172D + N174Q | | | | |
| SEQ ID NO: 2 + G50A + D112N | 1.1 | 1.1 | 1.1 | 1.0 |
| SEQ ID NO: 2 + | 1.0 | 1.1 | 0.9 | 1.1 |
| G50A + A51T + R172N + L173V + E345D + | | | | |
| E346P + D476G | | | | |
| SEQ ID NO: 2 + | 1.0 | 1.1 | 1.1 | 1.0 |
| G50A + A51T + R172N + L173V + E346T + | | | | |
| D476K + G477A | | | | |
| SEQ ID NO: 2 + | 1.0 | 1.1 | 1.1 | 1.0 |
| G50A + A51V + A113L + R172E + L173F + | | | | |
| N174T + A204V + E345D + E346P + | | | | |
| D476G | | | | |
| SEQ ID NO: 2 + | 1.3 | 1.2 | 1.4 | 1.0 |
| G50A + A51V + A113L + R172E + L173F + | | | | |
| N174T + A204V + E346T + D476K + | | | | |
| G477A | | | | |
| SEQ ID NO: 2 + | 0.9 | 1.1 | 0.7 | 0.9 |
| N174D + A204T + E345D + E346P + E416K | | | | |
| SEQ ID NO: 2 + | 1.0 | 0.9 | 1.0 | 1.1 |
| N174D + A204T + E346T + D476K + G477A | | | | |
| SEQ ID NO: 2 + | 1.0 | 1.0 | 1.2 | 1.0 |
| R172K + A204V + E346T + D476K + G477A | | | | |
| SEQ ID NO: 2 + | 1.1 | 0.9 | 1.3 | 0.9 |
| G4S + Y16D + T40K + G50A + R172N + | | | | |
| N174S + A204T | | | | |
| SEQ ID NO: 2 + | 1.2 | 1.0 | 1.5 | 1.1 |
| T40K + G50A + K72R + W167F + R172N + | | | | |
| N174S + A204T | | | | |
| SEQ ID NO: 2 + | 1.2 | 1.2 | 1.5 | 1.1 |
| G4S + T40K + K72R + Q98N + W167F + | | | | |
| R218K + N226T + R320A + S323M | | | | |
| SEQ ID NO: 2 + | 1.3 | 1.1 | 1.5 | 1.1 |
| T40K + K72R + Q98N + R320A + S323M | | | | |
| SEQ ID NO: 2 + | 1.3 | 1.1 | 1.2 | 1.1 |
| T40K + K72R + A113L + W167F | | | | |
| SEQ ID NO: 2 + | 1.2 | 0.9 | 1.3 | 0.7 |
| T40K + K72R + R118D + W167F | | | | |
| SEQ ID NO: 2 + | 1.2 | 1.0 | 1.3 | 1.0 |
| T40K + A51T + R172N + A204V + G304Q + | | | | |
| E345D + E346T + D476G + G477S | | | | |
| SEQ ID NO: 2 + | 1.3 | 1.1 | 1.4 | 1.1 |
| T40K + G50A + W167F + R172N + N174S + | | | | |
| A204T | | | | |
| SEQ ID NO: 2 + | 0.9 | 0.9 | 1.1 | 1.1 |
| T40K + K72R + E345D + E346P + D476G | | | | |
| SEQ ID NO: 2 + | 1.3 | 1.0 | 1.4 | 1.2 |
| K72R + W167F + E346T + D476K + G477A + | | | | |
| Y482F | | | | |
| SEQ ID NO: 2 + | 1.2 | 1.0 | 1.1 | 1.0 |
| W167F + E346T + D476K + G477A | | | | |
| SEQ ID NO: 2 + | 1.4 | 0.9 | 1.3 | 0.8 |
| G50A + A51V + K72R + W167F + R172E + | | | | |
| L173F + N174T + A204V | | | | |
| SEQ ID NO: 2 + | 1.4 | 0.9 | 1.2 | 0.6 |
| T40K + G50A + A51V + K72R + W167F + | | | | |
| R172E + L173F + N174T + A204V | | | | |
| SEQ ID NO: 2 + | 1.2 | 1.0 | 1.3 | 0.9 |
| K72R + Q98N + W167F + R320A + S323M | | | | |
| SEQ ID NO: 2 + | 1.2 | 1.0 | 1.3 | 1.2 |
| T40K + K72R + Q98N + W167F + R320A + | | | | |
| S323M | | | | |
| SEQ ID NO: 2 + | 1.3 | 0.9 | 1.4 | 1.2 |
| T40K + G50A + A51V + R172N + N174T + | | | | |
| A204T | | | | |
| SEQ ID NO: 2 + | 1.3 | 0.9 | 1.3 | 1.0 |
| T40K + G50A + A51V + W167F + R172N + | | | | |
| N174T + A204T | | | | |
| SEQ ID NO: 2 + | 1.6 | 1.0 | 1.6 | 1.3 |
| T40K + G50A + A51V + K72R + R172N + | | | | |
| N174T + A204T | | | | |
| SEQ ID NO: 2 + | 1.4 | 1.0 | 1.5 | 0.9 |
| T40K + K72R + R172E + N174D + A204T + | | | | |
| G304Q + E345D + E346T + D476G + | | | | |
| G477S | | | | |
| SEQ ID NO: 2 + | 1.3 | 1.0 | 1.4 | 1.0 |
| T40K + K72R + W167F + R172N + L173F + | | | | |
| N174Q + G304Q + E345D + E346T + | | | | |
| D476G + G477S | | | | |
| SEQ ID NO: 2 + | 1.4 | 1.0 | 1.5 | 1.1 |
| T40K + K72R + R172N + L173F + N174Q + | | | | |
| G304Q + E345D + E346T + D476G + | | | | |
| G477S | | | | |
| SEQ ID NO: 2 + | 1.3 | 1.0 | 1.2 | 1.2 |
| G50A + R172N + N174T + E346T + D476K + | | | | |
| G477A | | | | |
| SEQ ID NO: 2 + | 1.3 | 0.7 | 1.2 | 0.8 |
| A51V + R172D + N174S + G305N + G465L + | | | | |
| D467G | | | | |
| SEQ ID NO: 2 + | 1.1 | 1.1 | 1.1 | 1.1 |
| G50A + L173T + N174E + A204T + E345D + | | | | |
| S424Q | | | | |
| SEQ ID NO: 2 + | 1.2 | 1.1 | 1.3 | 1.1 |
| L173F + N174D + A354V | | | | |
| SEQ ID NO: 2 + | 1.1 | 1.0 | 1.3 | 1.2 |
| L173F + N174E + G477S | | | | |
| SEQ ID NO: 2 + | 1.4 | 0.9 | 1.5 | 1.1 |
| G50A + A51V + R172D + L173F + A204V + | | | | |
| K302Q + D476G + G477S | | | | |
| SEQ ID NO: 2 + | 1.4 | 1.0 | 1.4 | 1.3 |
| G50A + A51V + L173Q + R172* + N174T + | | | | |
| G305S + E346P + G477T | | | | |
| SEQ ID NO: 2 + | 1.4 | 0.9 | 1.3 | 1.0 |
| R172D + L173F + N174D + E345D + E346P + | | | | |
| W469L | | | | |
| SEQ ID NO: 2 + | 1.2 | 1.0 | 1.0 | 0.8 |
| T40K + N174D + A204T | | | | |
| SEQ ID NO: 2 + | 1.4 | 1.2 | 1.4 | 1.0 |
| T40K + K72R + W167F + N174D + A204T | | | | |
| SEQ ID NO: 2 + | 1.5 | 1.1 | 1.4 | 0.9 |
| W140Y + W167F + N174D | | | | |
| SEQ ID NO: 2 + | 1.3 | 0.9 | 1.3 | 0.6 |
| G50A + W140Y + W167F + N174D + A204T | | | | |
| SEQ ID NO: 2 + | 1.3 | 1.1 | 1.6 | 0.9 |
| T40K + K72R + W167F + R172K + A204V | | | | |
| SEQ ID NO: 2 + | 1.2 | 0.5 | 1.1 | 0.6 |
| W140Y + P146S + R172K + A204V | | | | |
| SEQ ID NO: 2 + | 1.5 | 0.9 | 1.4 | 0.9 |
| T40K + W167F + R172E + N174D + A204T + | | | | |
| G304Q + E345D + E346T + D476G + | | | | |
| G477S | | | | |
| SEQ ID NO: 2 + | 1.3 | 1.0 | 1.4 | 0.9 |
| T40K + R172E + N174D + A204T + G304Q + | | | | |
| E345D + E346T + D476G + G477S | | | | |
| SEQ ID NO: 2 + | 1.6 | 1.0 | 1.5 | 1.1 |
| W140Y + R172E + N174D + A204T + | | | | |
| G304Q + E345D + E346T + D476G + | | | | |
| G477S | | | | |
| SEQ ID NO: 2 + | 1.5 | 0.9 | 1.5 | 1.0 |
| A51T + W140Y + R172N + A204V + G304Q + | | | | |
| E345D + E346T + D476G + G477S | | | | |
| SEQ ID NO: 2 + | 1.6 | 1.0 | 1.6 | 1.2 |
| T40K + G50A + W167F + R172N + A204T | | | | |
| SEQ ID NO: 2 + | 1.4 | 1.0 | 1.3 | 0.9 |
| T40K + G50A + R172N + A204T | | | | |
| SEQ ID NO: 2 + | 1.6 | 1.2 | 1.4 | 1.1 |
| G50A + W140Y + R172N + A204T | | | | |
| SEQ ID NO: 2 + | 1.5 | 0.9 | 1.3 | 0.8 |
| G50A + W140Y + W167F + R172N + A204T | | | | |
| SEQ ID NO: 2 + | 1.5 | 1.1 | 1.4 | 1.3 |
| W167F + R172N + L173F + N174Q + G304Q + | | | | |
| E345D + E346T + D476G + G477S | | | | |
| SEQ ID NO: 2 + | 1.4 | 1.0 | 1.4 | 1.2 |
| W140Y + R172N + L173F + N174Q + | | | | |
| G304Q + E345D + E346T + D476G + | | | | |
| G477S | | | | |
| SEQ ID NO: 2 + | 1.5 | 1.1 | 1.5 | 1.5 |
| T40K + G50A + W167F + R172D + N174T + | | | | |
| E345D + E346P + D476G | | | | |
| SEQ ID NO: 2 + | 1.7 | 1.0 | 1.7 | 1.2 |
| T40K + G50A + K72R + W167F + R172D + | | | | |
| N174T + E345D + G477Q | | | | |
| SEQ ID NO: 2 + | 1.4 | 1.1 | 1.3 | 1.2 |
| G50A + W140Y + R172D + N174T + E345D + | | | | |
| E346P + D476G | | | | |
| SEQ ID NO: 2 + | 1.5 | 1.1 | 1.4 | 1.4 |
| G50A + W140Y + W167F + R172D + L173F + | | | | |
| N174T + G304Q + E345D + E346P + | | | | |
| D476G | | | | |
| SEQ ID NO: 2 + | 1.5 | 1.0 | 1.4 | 1.3 |
| T40K + K72R + W167F + R172K + A204V + | | | | |
| M208Y + V213S + V214T + L217M + | | | | |
| S255I + R320A + H324Q + E345D + | | | | |
| G477Q | | | | |
| SEQ ID NO: 2 + | 1.3 | 1.0 | 1.3 | 1.2 |
| T40K + W167F + R172K + A204V + M208Y + | | | | |
| V213S + V214T + L217M + S255I + | | | | |
| R320A + H324Q + E345D + G477Q | | | | |
| SEQ ID NO: 2 + | 1.2 | 1.1 | 1.3 | 1.0 |
| W140Y + W167F + R172D + N174T + | | | | |
| A204V + S255I + E345D + E346P + G477Q | | | | |
| SEQ ID NO: 2 + | 1.1 | 0.9 | 1.1 | 1.2 |
| W140Y + W167F + R172D + N174T + | | | | |
| A204V + S255N + E345D + G477Q | | | | |
| SEQ ID NO: 2 + | 1.1 | 1.0 | 1.3 | 1.5 |
| W167F + R172D + N174T + A204V + S255I + | | | | |
| E345D + G477Q | | | | |
| SEQ ID NO: 2 + | 1.2 | 0.9 | 1.3 | 1.2 |
| W140Y + W167F + R172D + N174T + | | | | |
| A204V + S255I + E345D + E346P + G477Q | | | | |
| SEQ ID NO: 2 + | 1.2 | 0.9 | 1.2 | 1.4 |
| T40K + Q98N + W167F + R172K + A204V + | | | | |
| M208Y + V213T + V214T + L217M + | | | | |
| S255I + R320A + S323M + E345D + | | | | |
| T431P | | | | |
| SEQ ID NO: 2 + | 1.3 | 1.0 | 1.3 | 1.5 |
| T40K + K72R + Q98N + W167F + A204V + | | | | |
| M208Y + V213T + V214T + L217M + | | | | |
| S255I + R320A + S323M | | | | |
| SEQ ID NO: 2 + | 1.3 | 1.1 | 1.4 | 1.6 |
| Q98N + W140Y + R172K + A204V + M208Y + | | | | |
| V213T + V214T + L217M + S255I + | | | | |
| R320A + S323M + E345D + V474I | | | | |
| SEQ ID NO: 2 + | 1.3 | 1.0 | 1.4 | 1.3 |
| Q98N + W140Y + R172K + A204V + M208Y + | | | | |
| V213T + V214T + L217M + S255I + | | | | |
| R320A + S323M + E345D + R415K | | | | |
| SEQ ID NO: 2 + | 1.3 | 1.1 | 1.4 | 1.3 |
| Q98N + W140Y + W167F + R172K + A204V + | | | | |
| M208Y + V213T + V214T + L217M + | | | | |
| S255I + R320A + S323M + E345D | | | | |
| SEQ ID NO: 2 + | 1.2 | 0.8 | 1.0 | 0.3 |
| T40K + G50A + A51V + K72R + W167F + | | | | |
| R172E + L173F + N174T + A204V + | | | | |
| R320A + S323N | | | | |
| SEQ ID NO: 2 + | 1.4 | 0.9 | 1.4 | 1.0 |
| G50A + A51V + W140Y + P146S + W167F + | | | | |
| R172E + L173F + N174T + A204V + | | | | |
| R320A + S323N | | | | |
| SEQ ID NO: 2 + | 1.1 | 0.5 | 1.3 | 0.6 |
| T40K + G50A + A51V + K72R + W167F + | | | | |
| R172E + L173F + N174T + M208Y + | | | | |
| V213S + V214T + L217M | | | | |
| SEQ ID NO: 2 + | 1.5 | 1.0 | 1.7 | 1.2 |
| G50A + A51V + W140Y + W167F + R172E + | | | | |
| L173F + N174T + M208Y + V213S + | | | | |
| V214T + L217M | | | | |
| SEQ ID NO: 2 + | 1.4 | 1.1 | 1.6 | 1.6 |
| G50A + A51T + R172D + K302Q + E346P + | | | | |
| D476K + G477S | | | | |
| SEQ ID NO: 2 + | 1.4 | 1.2 | 1.6 | 1.5 |
| G50A + A51T + R172D + E345D + D476K + | | | | |
| G477S | | | | |
| SEQ ID NO: 2 + | 1.3 | 1.2 | 1.4 | 1.6 |
| G50A + A51T + R172N + L173V + E345D + | | | | |
| D476K + G477S | | | | |
| SEQ ID NO: 2 + | 1.2 | 1.1 | 1.4 | 1.3 |
| G50A + A51V + A113L + R172E + L173F + | | | | |
| N174T + A204V + K302Q + E346P + | | | | |
| D476K + G477S | | | | |
| SEQ ID NO: 2 + | 1.4 | 1.1 | 1.7 | 1.6 |
| G50A + A51V + A113L + R172E + L173F + | | | | |
| N174T + A204V + E345D + L405V + | | | | |
| T414K | | | | |
| SEQ ID NO: 2 + | 1.1 | 1.0 | 1.2 | 1.4 |
| G50A + A51V + R172N + N174T + A204T + | | | | |
| K302Q + E346P + D476K + G477S | | | | |
| SEQ ID NO: 2 + | 1.4 | 1.1 | 1.4 | 1.3 |
| G50A + A51V + R172N + N174T + A204T + | | | | |
| E345D + D476K + G477S | | | | |
| SEQ ID NO: 2 + | 1.1 | 1.0 | 1.1 | 1.1 |
| N174D + A204T + K302Q + E346P + D476K + | | | | |
| G477S | | | | |
| SEQ ID NO: 2 + | 1.2 | 1.0 | 1.2 | 1.2 |
| G50A + R172D + N174Q + K302Q + E346P + | | | | |
| D476K + G477S | | | | |
| SEQ ID NO: 2 + | 1.3 | 1.0 | 1.3 | 1.2 |
| G50A + R172D + N174Q + E345D + D476K + | | | | |
| G477S | | | | |
| SEQ ID NO: 2 + | 1.5 | 1.0 | 1.5 | 1.1 |
| R172D + N174T + K302Q + E346P + D476K + | | | | |
| G477S | | | | |
| SEQ ID NO: 2 + | 1.0 | 1.0 | 1.0 | 1.2 |
| G50S + R172N + N174Q + E345D + E346P + | | | | |
| D476G | | | | |
| SEQ ID NO: 2 + | 1.3 | 1.1 | 1.4 | 1.1 |
| R172N + N174Q + E345D + D406A + I411M + | | | | |
| N446K | | | | |
| SEQ ID NO: 2 + | 1.3 | 1.0 | 1.3 | 1.3 |
| G50S + R172N + N174Q + E345D | | | | |
| SEQ ID NO: 2 + | 1.1 | 1.0 | 1.3 | 1.1 |
| G50A + N174D + A204T + E345D + D476K + | | | | |
| G477S | | | | |
| SEQ ID NO: 2 + | 1.3 | 1.0 | 1.4 | 1.2 |
| G50A + N174D + A204T + S255I + E345D + | | | | |
| G477Q | | | | |
| SEQ ID NO: 2 + | 1.3 | 1.1 | 1.4 | 1.2 |
| G50A + N174D + A204T + G304S + E345D + | | | | |
| D476K + G477Q | | | | |
| SEQ ID NO: 2 + | 1.3 | 1.0 | 1.4 | 1.1 |
| N174D + A204T + G304S + E345D + D476K + | | | | |
| G477Q | | | | |
| SEQ ID NO: 2 + | 1.2 | 1.1 | 1.4 | 1.3 |
| R172K + A204V + K302Q + E346P + D476K + | | | | |
| G477S | | | | |
| SEQ ID NO: 2 + | 1.4 | 1.1 | 1.5 | 1.3 |
| R172K + A204V + E345D + D476K | | | | |
| SEQ ID NO: 2 + | 1.2 | 1.0 | 1.5 | 1.2 |
| R172K + A204V + E345D + D476K + G477S | | | | |
| SEQ ID NO: 2 + | 1.2 | 1.4 | 1.3 | 1.1 |
| R172K + A204V + G304S + E345D + D476K + | | | | |
| G477Q | | | | |
| SEQ ID NO: 2 + | 1.3 | 1.3 | 1.2 | 1.2 |
| G50A + R172N + A204T + E346T + D476K + | | | | |
| G477A | | | | |
| SEQ ID NO: 2 + | 1.1 | 1.4 | 1.1 | 1.1 |
| G50A + R172N + A204T + E346T + D432A + | | | | |
| D476K + G477A | | | | |
| SEQ ID NO: 2 + | 1.2 | 1.1 | 1.1 | 1.3 |
| G50A + R172N + A204T + E345D + D476K + | | | | |
| G477S | | | | |
| SEQ ID NO: 2 + | 1.1 | 1.0 | 1.2 | 1.2 |
| G50A + R172N + A204T + G304S + E345D + | | | | |
| D476K + G477Q | | | | |
| SEQ ID NO: 2 + | 1.1 | 1.0 | 1.0 | 1.1 |
| G50A + R172N + A204T + E345D + E346P + | | | | |
| D476G | | | | |
| SEQ ID NO: 2 + | 1.3 | 0.8 | 1.2 | 1.0 |
| G50A + A51V + W167F + R172E + L173F + | | | | |
| N174T + A204V + S255I + E345D + | | | | |
| G477Q | | | | |
| SEQ ID NO: 2 + | 1.4 | 1.0 | 1.3 | 1.3 |
| T40K + K72R + W167F + S255I + E345D + | | | | |
| G477Q | | | | |
| SEQ ID NO: 2 + | 1.4 | 1.1 | 1.3 | 1.3 |
| T40K + K72R + W167F + G304S + E345D + | | | | |
| D476K + G477Q | | | | |
| SEQ ID NO: 2 + | 1.3 | 1.0 | 1.2 | 1.0 |
| T40K + K72R + W167F + K302Q + E346P + | | | | |
| D476K | | | | |
| SEQ ID NO: 2 + | 1.5 | 1.1 | 1.4 | 1.0 |
| G50A + A51V + W167F + R172E + L173F + | | | | |
| N174T + M208Y + V213S + V214T + | | | | |
| L217M + S255I + E345D + G477Q | | | | |
| SEQ ID NO: 2 + | 1.4 | 1.0 | 1.4 | 1.2 |
| G50A + A51V + W167F + R172E + L173F + | | | | |
| N174T + M208Y + V213S + V214T + | | | | |
| L217M + E345D + D476K + G477S | | | | |
| SEQ ID NO: 2 + | 1.4 | 1.2 | 1.3 | 1.4 |
| G50A + A51V + W167F + R172E + L173F + | | | | |
| N174T + M208Y + V213S + V214T + | | | | |
| L217M + E345D + E346P + D476G | | | | |
| SEQ ID NO: 2 + | 1.5 | 1.2 | 1.3 | 1.5 |
| G50A + A51V + W167F + R172E + L173F + | | | | |
| N174T + M208Y + V213S + V214T + | | | | |
| L217M + E346T + D476K + G477A | | | | |
| SEQ ID NO: 2 + | 1.4 | 1.1 | 1.3 | 1.1 |
| G50A + A51V + R172N + N174T + A204T + | | | | |
| M208Y + V213T + V214T + L217M | | | | |
| SEQ ID NO: 2 + | 1.5 | 1.1 | 1.4 | 1.1 |
| G50A + A51V + R172N + N174T + A204T + | | | | |
| M208Y + V213S + V214T + L217M | | | | |
| SEQ ID NO: 2 + | 1.4 | 1.1 | 1.3 | 1.2 |
| N174D + A204V + M208Y + V213T + V214T + | | | | |
| L217M | | | | |
| SEQ ID NO: 2 + | 1.3 | 0.9 | 1.2 | 1.1 |
| R172E + N174D + A204T + M208Y + V213T + | | | | |
| V214T + L217M + G304Q + E345D + | | | | |
| E346T + D476G + G477S | | | | |
| SEQ ID NO: 2 + | 1.3 | 1.1 | 1.5 | 1.2 |
| R172E + N174D + A204T + M208Y + V213S + | | | | |
| V214T + L217M + G304Q + E345D + | | | | |
| E346T + D476G + G477S | | | | |
| SEQ ID NO: 2 + | 1.3 | 1.2 | 1.2 | 1.0 |
| A51T + R172N + A204V + M208Y + V213T + | | | | |
| V214T + L217M + G304Q + E345D + | | | | |
| E346T + D476G + G477S | | | | |
| SEQ ID NO: 2 + | 1.2 | 1.1 | 1.1 | 0.9 |
| A51T + R172N + A204V + M208Y + V213T + | | | | |
| V214T + L217M + G304Q + E345D + | | | | |
| E346T + E416K + K438N | | | | |
| SEQ ID NO: 2 + | 1.5 | 1.4 | 1.4 | 1.1 |
| A51T + R172N + A204V + M208Y + V213S + | | | | |
| V214T + L217M + G304Q + E345D + | | | | |
| E346T + D476G + G477S | | | | |
| SEQ ID NO: 2 + | 1.5 | 1.0 | 1.3 | 1.1 |
| G50A + R172N + A204T + M208Y + V213T + | | | | |
| V214T + L217M | | | | |
| SEQ ID NO: 2 + | 1.5 | 1.5 | 1.4 | 1.1 |
| G50A + R172N + A204T + M208Y + V213S + | | | | |
| V214T + L217M | | | | |
| SEQ ID NO: 2 + | 1.5 | 1.3 | 1.3 | 1.1 |
| G50A + A51V + W167F + R172E + L173F + | | | | |
| N174T + A204V + M208Y + V213T + | | | | |
| V214T + L217M | | | | |
| SEQ ID NO: 2 + | 1.4 | 0.9 | 1.3 | 1.0 |
| G50A + A51V + W167F + R172E + L173F + | | | | |
| N174T + A204V + M208Y + V213S + | | | | |
| V214T + L217M | | | | |
| SEQ ID NO: 2 + | 1.3 | 1.1 | 1.2 | 1.0 |
| R172K + A204V + M208Y + V213T + V214T + | | | | |
| L217M + S255I + E345D + G477Q | | | | |
| SEQ ID NO: 2 + | 1.4 | 0.8 | 1.3 | 1.0 |
| G50A + A51V + W167F + R172E + L173F + | | | | |
| N174T + A204V + M208Y + V213T + | | | | |
| V214T + L217M + R320A + S323N | | | | |
| SEQ ID NO: 2 + | 1.2 | 0.8 | 1.3 | 1.2 |
| G50A + A51V + R172N + N174T + A204T + | | | | |
| E346T + G477A | | | | |
| SEQ ID NO: 2 + | 1.2 | 0.8 | 1.2 | 1.0 |
| G50A + A51V + R172N + N174T + A204T + | | | | |
| R320V + D476Y | | | | |
| SEQ ID NO: 2 + | 1.2 | 0.9 | 1.2 | 1.0 |
| G50A + A51V + R172N + N174T + A204T + | | | | |
| R320V | | | | |
| SEQ ID NO: 2 + | 1.2 | 1.0 | 1.2 | 1.1 |
| R172N + L173V + E346T + G477A | | | | |
| SEQ ID NO: 2 + | 1.1 | 0.8 | 1.2 | 1.0 |
| G50A + A51T + R172N + L173V + E346T + | | | | |
| G477A | | | | |
| SEQ ID NO: 2 + | 1.3 | 1.0 | 1.3 | 1.1 |
| G50A + A51T + R172N + L173V + R320A + | | | | |
| S323M + D476Y | | | | |
| SEQ ID NO: 2 + | 1.1 | 0.5 | 1.1 | 0.6 |
| G50A + A51T + R172N + L173V + R320V + | | | | |
| D476Y | | | | |
| SEQ ID NO: 2 + | 1.2 | 0.8 | 1.3 | 0.9 |
| G50A + A51T + R172D + R320A + S323N | | | | |
| SEQ ID NO: 2 + | 1.1 | 1.0 | 1.2 | 1.1 |
| G50A + R172Q + L173T + E346T + G477A | | | | |
| SEQ ID NO: 2 + | 1.1 | 0.7 | 1.2 | 0.9 |
| R172Q + L173T + R320V + D476Y | | | | |
| SEQ ID NO: 2 + | 1.2 | 0.8 | 1.0 | 1.0 |
| N174D + A204T + R320V + D476Y | | | | |
| SEQ ID NO: 2 + | 1.3 | 0.9 | 1.3 | 1.1 |
| R172K + A204V + R320A + S323N | | | | |
| SEQ ID NO: 2 + | 1.3 | 1.0 | 1.7 | 1.1 |
| G50A + A51V + W167F + R172E + L173F + | | | | |
| N174T + A204V + E346T + G477A | | | | |
| SEQ ID NO: 2 + | 1.3 | 0.9 | 1.3 | 1.1 |
| G50A + A51V + W167F + R172E + L173F + | | | | |
| N174T + A204V + R320V + D476Y | | | | |
| SEQ ID NO: 2 + | 1.3 | 1.0 | 1.4 | 1.2 |
| R172E + L173F + N174T + A204V + E345D + | | | | |
| E346T + G477A | | | | |
| SEQ ID NO: 2 + | 1.3 | 1.0 | 1.6 | 1.2 |
| G50A + A51V + A113L + R172E + L173F + | | | | |
| N174T + A204V + E346T + G477A | | | | |
| SEQ ID NO: 2 + | 1.3 | 0.8 | 1.2 | 1.0 |
| G50A + A51V + A113L + R172E + L173F + | | | | |
| N174T + A204V + R320V | | | | |
| SEQ ID NO: 2 + | 1.2 | 0.9 | 1.5 | 1.2 |
| T40K + K72R + W167F + E346T + G477A | | | | |
| SEQ ID NO: 2 + | 1.1 | 0.9 | 1.3 | 1.2 |
| T40K + K72R + W167F + R320V + D476Y | | | | |
| SEQ ID NO: 2 + | 1.1 | 0.9 | 1.1 | 1.1 |
| G50A + A51V + R172E + N174S + E346T + | | | | |
| G477A | | | | |
| SEQ ID NO: 2 + | 1.1 | 1.0 | 1.0 | 1.0 |
| G50A + A51V + N174S + R320V + D476Y | | | | |
| SEQ ID NO: 2 + | 1.1 | 0.9 | 1.0 | 1.0 |
| A51V + N174S + R320V + D476Y | | | | |
| SEQ ID NO: 2 + | 1.3 | 0.8 | 1.2 | 1.0 |
| G50A + A51T + R172E + A204T + E346T + | | | | |
| G477A | | | | |
| SEQ ID NO: 2 + | 1.2 | 0.8 | 1.2 | 0.7 |
| G50A + A51T + R172E + A204T + R320V + | | | | |
| D476Y | | | | |
| SEQ ID NO: 2 + | 1.3 | 0.9 | 1.2 | 1.1 |
| G50A + A51T + R172E + A204T + R320V + | | | | |
| N457T + D476Y | | | | |
| SEQ ID NO: 2 + | 1.3 | 0.8 | 1.1 | 0.9 |
| R172D + N174Q + A204V + E346T + G477A | | | | |
| SEQ ID NO: 2 + | 1.5 | 1.1 | 1.5 | 1.2 |
| L173T + A204T + E346T + G477A | | | | |
| SEQ ID NO: 2 + | 1.4 | 1.1 | 1.4 | 1.3 |
| L173T + A204T + R320V + D476Y | | | | |
| SEQ ID NO: 2 + | 1.5 | 1.1 | 1.6 | 1.3 |
| G50A + A51V + R172D + N174E + E346T + | | | | |
| G477A | | | | |
| SEQ ID NO: 2 + | 1.4 | 1.1 | 1.6 | 1.1 |
| G50A + A51V + R172D + N174E + R320V + | | | | |
| D476Y | | | | |
| SEQ ID NO: 2 + | 1.5 | 1.0 | 1.5 | 1.2 |
| G50A + A51V + R172D + N174E + R320S + | | | | |
| S323N + D476Y | | | | |
| SEQ ID NO: 2 + | 1.4 | 1.1 | 1.5 | 1.3 |
| G50A + A51V + W167F + R172E + L173F + | | | | |
| N174T + A204V + G304Q + E345D + | | | | |
| E346T + D476G + G477T | | | | |
| SEQ ID NO: 2 + | 1.3 | 0.8 | 1.3 | 1.0 |
| G50A + R172D + N174Q + R320A + S323N | | | | |
| SEQ ID NO: 2 + | 1.3 | 0.8 | 1.4 | 1.1 |
| G50A + R172D + N174Q + E346T + G477A | | | | |
| SEQ ID NO: 2 + | 1.2 | 0.9 | 1.3 | 1.0 |
| G50A + R172D + N174Q + R320A + S323M + | | | | |
| D476Y | | | | |
| SEQ ID NO: 2 + | 1.4 | 1.0 | 1.3 | 1.1 |
| G50A + R172D + N174Q + R320V + D476Y | | | | |
| SEQ ID NO: 2 + | 1.3 | 1.0 | 1.2 | 1.1 |
| G50A + R172N + A204T + K302Q + E346P + | | | | |
| D476K + G477S | | | | |
| SEQ ID NO: 2 + | 1.4 | 1.2 | 1.4 | 1.1 |
| G7K + T40K + K72R + W167F | | | | |
| SEQ ID NO: 2 + | 1.5 | 0.9 | 1.5 | 1.2 |
| G7Q + T40K + K72R + W167F | | | | |
| SEQ ID NO: 2 + | 1.3 | 0.6 | 1.3 | 1.2 |
| G7E + T40K + K72R + W167F | | | | |
| SEQ ID NO: 2 + | 1.3 | 0.8 | 1.5 | 1.0 |
| W167F + Q169M + R172E + N174S | | | | |
| SEQ ID NO: 2 + | 1.4 | 1.0 | 1.3 | 1.2 |
| T40K + K72R + W167Y | | | | |
| SEQ ID NO: 2 + | 1.3 | 1.0 | 1.4 | 0.9 |
| T40K + K72R + W167H | | | | |
| SEQ ID NO: 2 + | 1.0 | 0.8 | 1.1 | 0.8 |
| W140Y + P146S + N174D + A204T | | | | |
| SEQ ID NO: 2 + | 1.0 | 0.9 | 1.2 | 0.8 |
| W140Y + W167F + R172K + A204V | | | | |
| SEQ ID NO: 2 + | 1.2 | 1.1 | 1.5 | 1.0 |
| W140Y + W167F + A204V | | | | |
| SEQ ID NO: 2 + | 1.1 | 1.0 | 1.3 | 1.0 |
| R172K + M208Y + V213T + V214T + L217M | | | | |
| SEQ ID NO: 2 + | 1.1 | 1.0 | 1.3 | 1.0 |
| R172K + M208Y + V213S + V214T + L217M | | | | |
| SEQ ID NO: 2 + | 1.2 | 0.9 | 1.2 | 0.9 |
| Q98N + W167F + R320A + S323M | | | | |
| SEQ ID NO: 2 + | 1.3 | 0.9 | 1.4 | 0.9 |
| W140Y + W167F + S255I + E345D + G477Q | | | | |
| SEQ ID NO: 2 + | 1.1 | 0.7 | 1.5 | 0.8 |
| G7K + W167F + R320M + H321Q | | | | |
| SEQ ID NO: 2 + | 1.1 | 0.8 | 1.6 | 0.8 |
| G7K + K72R + W167F + R320M + H321Q | | | | |
| SEQ ID NO: 2 + | 1.2 | 1.0 | 1.3 | 1.0 |
| G7K + T40K + W167F + R320M + H321Q | | | | |
| SEQ ID NO: 2 + | 1.0 | 0.8 | 1.4 | 0.9 |
| G7K + T40K + K72R + W167F + R320M + | | | | |
| H321Q | | | | |
| SEQ ID NO: 2 + | 1.1 | 0.8 | 1.1 | 0.7 |
| A51T + W167F + R172N + A204V + G304Q + | | | | |
| E345D + E346T | | | | |
| SEQ ID NO: 2 + | 1.1 | 1.0 | 1.2 | 0.8 |
| A51T + W167F + R172N + A204V + G304Q + | | | | |
| E345D + E346T + D476G + G477S | | | | |
| SEQ ID NO: 2 + | 1.1 | 0.9 | 1.4 | 1.2 |
| A51T + K72R + R172N + A204V + G304Q + | | | | |
| E345D + E346T + D476G + G477S | | | | |
| SEQ ID NO: 2 + | 1.1 | 0.7 | 1.3 | 0.7 |
| T40K + A51T + K72R + W167F + R172N + | | | | |
| A204V + G304Q + E345D + E346T + | | | | |
| D476G + G477S | | | | |
| SEQ ID NO: 2 + | 1.2 | 0.8 | 1.4 | 1.2 |
| T40K + A51T + W167F + R172N + A204V + | | | | |
| G304Q + E345D + E346T + D476G + | | | | |
| G477S | | | | |
| SEQ ID NO: 2 + | 1.2 | 1.0 | 1.2 | 1.3 |
| G50A + A51T + R172N + L173V + G304Q + | | | | |
| E345D + E346T + D476G + G477T | | | | |
| SEQ ID NO: 2 + | 1.1 | 0.9 | 1.2 | 0.6 |
| G50A + A51V + A113L + R172E + L173F + | | | | |
| N174T + A204V + G304Q + E345D + | | | | |
| E346T + D476G + G477T | | | | |
| SEQ ID NO: 2 + | 1.1 | 1.0 | 1.2 | 1.1 |
| G50A + A51V + R172N + N174T + A204T + | | | | |
| G304Q + E345D + E346T + D476G + | | | | |
| G477T | | | | |
| SEQ ID NO: 2 + | 1.0 | 1.0 | 1.4 | 1.5 |
| G50A + R172Q + L173T + G304Q + E345D + | | | | |
| E346T + D476G + G477T | | | | |
| SEQ ID NO: 2 + | 1.1 | 1.0 | 1.4 | 0.9 |
| G50A + A51T + L85F + R172N + L173V + | | | | |
| Q319K + R320A + S323N | | | | |
| SEQ ID NO: 2 + | 1.2 | 0.7 | 1.1 | 0.8 |
| G50A + A51V + A113L + R172E + L173F + | | | | |
| N174T + A204V + R320A + S323N | | | | |
| SEQ ID NO: 2 + SEQ ID NO: 2 + | 1.1 | 0.8 | 1.1 | 1.0 |
| G50A + A51V + R172N + N174T + A204T + | | | | |
| R320A + S323N | | | | |
| SEQ ID NO: 2 + | 0.9 | 0.8 | 1.1 | 1.0 |
| G50A + A51V + N174S + R320A + S323N | | | | |
| SEQ ID NO: 2 + | 1.2 | 0.6 | 1.1 | 0.8 |
| G50A + R172D + N174D + R320V + D476Y | | | | |
| SEQ ID NO: 2 + | 1.1 | 0.7 | 1.2 | 1.1 |
| G50A + R172D + N174D + R320V | | | | |
| SEQ ID NO: 2 + | 1.3 | 1.3 | 1.1 | 1.0 |
| G50A + A51T + R172E + A204T + E346T + | | | | |
| D476K + G477A | | | | |
| SEQ ID NO: 2 + | 1.1 | 1.1 | 0.8 | 0.9 |
| G50A + L173T + A204T + K302Q + E346P + | | | | |
| D476K + G477S | | | | |
| SEQ ID NO: 2 + | 1.3 | 1.1 | 1.2 | 1.0 |
| L173T + A204T + K302Q + E346P + D476K + | | | | |
| G477S | | | | |
| SEQ ID NO: 2 + | 1.5 | 1.3 | 1.5 | 1.2 |
| R172D + N174Q + E346T + D476K + G477A | | | | |
| SEQ ID NO: 2 + | 1.1 | 0.9 | 1.1 | 1.0 |
| L173T + A204T + E346T + D476K + G477A | | | | |
| SEQ ID NO: 2 + | 1.5 | 0.9 | 1.3 | 1.2 |
| G50A + R172D + N174Q + A204V + E345D + | | | | |
| D476K + G477S | | | | |
| SEQ ID NO: 2 + | 1.1 | 1.0 | 1.1 | 1.0 |
| L173T + A204T + E345D + D476K + G477S | | | | |
| SEQ ID NO: 2 + | 1.3 | 0.7 | 1.0 | 1.0 |
| L173T + A204T + S255I + E345D + G477Q | | | | |
| SEQ ID NO: 2 + | 1.2 | 1.1 | 1.2 | 1.1 |
| L173T + A204T + G304S + E345D + D476K + | | | | |
| G477Q | | | | |
| SEQ ID NO: 2 + | 1.1 | 1.1 | 1.1 | 1.2 |
| G50A + R172D + N174Q + A204V + E345D + | | | | |
| E346P + D476G | | | | |
| SEQ ID NO: 2 + | 1.3 | 0.7 | 1.0 | 0.7 |
| G50A + R172D + N174Q + A204V + R320A + | | | | |
| S323N | | | | |
| SEQ ID NO: 2 + | 1.2 | 1.0 | 1.1 | 1.1 |
| G50A + A51T + R172E + A204T + K302Q + | | | | |
| E346P + D476K + G477S | | | | |
| SEQ ID NO: 2 + | 1.2 | 1.0 | 1.2 | 1.0 |
| G50A + A51T + N174S + A204T + S255I + | | | | |
| E345D + G477Q | | | | |
| SEQ ID NO: 2 + | 1.1 | 1.1 | 1.2 | 1.2 |
| G50A + A51T + L85F + R172E + A204T + | | | | |
| E345D + E346P + D476G | | | | |
| SEQ ID NO: 2 + | 1.3 | 1.1 | 1.3 | 1.3 |
| R172D + N174E + K302Q + E346P + D476K + | | | | |
| G477S | | | | |
| SEQ ID NO: 2 + | 1.3 | 1.1 | 1.3 | 1.3 |
| G50A + A51V + R172D + N174E + K302Q + | | | | |
| E346P + D476K + G477S | | | | |
| SEQ ID NO: 2 + | 1.3 | 1.1 | 1.5 | 1.2 |
| G50A + A51V + R172D + N174E + E346T + | | | | |
| D476K + G477A | | | | |
| SEQ ID NO: 2 + | 1.4 | 0.8 | 1.3 | 0.8 |
| G50A + A51V + R172D + N174E + S255I + | | | | |
| E345D + G477Q | | | | |
| SEQ ID NO: 2 + | 1.3 | 1.2 | 1.4 | 1.3 |
| G50A + A51V + R172D + N174E + G304S + | | | | |
| E345D + D476K + G477Q | | | | |
| SEQ ID NO: 2 + | 1.3 | 1.1 | 1.3 | 1.3 |
| G50A + A51V + R172D + N174E + E345D + | | | | |
| E346P + D476G | | | | |
| SEQ ID NO: 2 + | 1.4 | 1.0 | 1.4 | 1.0 |
| G50A + A51V + R172D + N174E + R320A + | | | | |
| S323N | | | | |
| SEQ ID NO: 2 + | 1.4 | 1.0 | 1.3 | 0.9 |
| G50A + A51V + R172D + N174E + G315E + | | | | |
| R320A + S323N | | | | |
| SEQ ID NO: 2 + | 1.1 | 0.8 | 1.3 | 1.3 |
| G50A + A51T + R172E + A204T + E345D + | | | | |
| D476K + G477S | | | | |
| SEQ ID NO: 2 + | 1.2 | 1.0 | 1.5 | 1.0 |
| R172E + A204T + G304S + E345D + D476K + | | | | |
| G477Q | | | | |
| SEQ ID NO: 2 + | 1.4 | 1.2 | 1.6 | 1.4 |
| G50A + R172D + N174D + M208Y + V213S + | | | | |
| V214T + L217M + E346T + D476K + | | | | |
| G477A | | | | |
| SEQ ID NO: 2 + | 1.0 | 1.1 | 1.3 | 1.3 |
| G50A + A51V + N174S + M208Y + V213S + | | | | |
| V214T + L217M | | | | |
| SEQ ID NO: 2 + | 1.3 | 1.0 | 1.4 | 1.4 |
| G50A + A51V + R172E + N174S + M208Y + | | | | |
| V213S + V214T + L217M | | | | |
| SEQ ID NO: 2 + | 1.1 | 0.8 | 1.5 | 1.2 |
| A51V + R172E + N174S + M208Y + V213S + | | | | |
| V214T + L217M | | | | |
| SEQ ID NO: 2 + | 1.4 | 1.1 | 1.7 | 1.5 |
| G50A + R118P + R172D + N174Q + G184S + | | | | |
| E346T + D476K + G477A | | | | |
| SEQ ID NO: 2 + | 1.2 | 0.8 | 1.5 | 0.9 |
| G50A + A51V + R118P + R172E + N174S + | | | | |
| G184S | | | | |
| SEQ ID NO: 2 + | 1.2 | 1.2 | 1.4 | 1.1 |
| G50A + A51V + R118P + N174S + G184S | | | | |
| SEQ ID NO: 2 + | 1.2 | 0.9 | 1.4 | 1.1 |
| G50A + R118P + R172E + G184S + A204T | | | | |
| SEQ ID NO: 2 + | 1.2 | 0.8 | 1.6 | 0.9 |
| G50A + A51T + R118P + R172E + G184S + | | | | |
| A204T | | | | |
| SEQ ID NO: 2 + | 1.3 | 1.0 | 1.7 | 1.0 |
| R118P + L173T + G184S + A204T | | | | |
| SEQ ID NO: 2 + | 1.4 | 1.0 | 1.6 | 1.0 |
| G50A + A51V + R118P + R172D + N174E + | | | | |
| G184S | | | | |
| SEQ ID NO: 2 + | 1.1 | 1.2 | 1.5 | 1.1 |
| T40K + G50A + A51V + W167F + N174S | | | | |
| SEQ ID NO: 2 + | 1.4 | 1.2 | 1.6 | 1.2 |
| T40K + G50A + A51V + K72R + W167F + | | | | |
| R172E + N174S | | | | |
| SEQ ID NO: 2 + | 1.5 | 1.2 | 1.9 | 1.3 |
| T40K + G50A + A51V + K72R + W167F + | | | | |
| R172D + N174E | | | | |
| SEQ ID NO: 2 + | 1.6 | 1.0 | 1.4 | 0.9 |
| T40K + G50A + A51V + W167F + R172D + | | | | |
| N174E | | | | |
| SEQ ID NO: 2 + | 1.4 | 1.0 | 1.3 | 1.1 |
| T40K + W167F + L173T + A204T | | | | |
| SEQ ID NO: 2 + | 1.5 | 1.0 | 1.0 | 1.4 |
| T40K + G50A + K72R + W167F + L173T + | | | | |
| A204V | | | | |
| SEQ ID NO: 2 + | 1.3 | 1.1 | 1.4 | 1.2 |
| G50A + R172D + N174Q + M208Y + V213S + | | | | |
| V214T + L217M + E346T + D476K + | | | | |
| G477A | | | | |
| SEQ ID NO: 2 + | 1.2 | 0.8 | 1.2 | 1.5 |
| G50A + R172S + N174S + E346T + D476K + | | | | |
| G477A | | | | |
| SEQ ID NO: 2 + | 1.7 | 1.1 | 1.2 | 0.7 |
| G50A + A51V + R172D + N174E + M208Y + | | | | |
| V213S + V214T + L217M | | | | |
| SEQ ID NO: 2 + | 1.3 | 1.0 | 1.0 | 0.5 |
| L173T + G184S + A204T | | | | |
| SEQ ID NO: 2 + | 1.6 | 1.0 | 1.3 | 0.8 |
| T40K + G50A + R172D + N174D + E346T + | | | | |
| D476K + G477A | | | | |
| SEQ ID NO: 2 + | 1.5 | 1.0 | 1.5 | 1.0 |
| G50A + R118P + R172D + N174D + G184S + | | | | |
| E346T + D476K + G477A | | | | |
| SEQ ID NO: 2 + | 1.4 | 0.9 | 1.0 | 1.2 |
| G50A + R172T + N174S + E346T | | | | |
| SEQ ID NO: 2 + | 1.4 | 0.8 | 1.2 | 0.7 |
| R172E + L173F + N174D + G304S + E345D + | | | | |
| D476K + G477Q | | | | |
| SEQ ID NO: 2 + | 1.4 | 1.0 | 1.2 | 0.6 |
| R172E + L173F + N174D + E345D + E346P + | | | | |
| D476G | | | | |
| SEQ ID NO: 2 + | 1.5 | 0.9 | 1.2 | 0.1 |
| R172E + L173F + N174D + R320A + S323N | | | | |
| SEQ ID NO: 2 + | 1.3 | 0.9 | 1.1 | 0.3 |
| R172E + L173F + N174D + E346T + A354V + | | | | |
| G477A | | | | |
| SEQ ID NO: 2 + | 1.3 | 0.8 | 1.3 | 1.0 |
| R172E + L173F + N174D + E346T + G477A | | | | |
| SEQ ID NO: 2 + | 1.1 | 0.4 | 1.0 | 0.8 |
| R172E + L173F + N174D + R320V | | | | |
| SEQ ID NO: 2 + | 1.2 | 0.7 | 1.2 | 0.8 |
| R172E + L173F + N174D + R320V + D476Y | | | | |
| SEQ ID NO: 2 + | 1.3 | 1.0 | 1.0 | 0.8 |
| R172N + L173V + K302Q + E346P + D476K + | | | | |
| G477S | | | | |
| SEQ ID NO: 2 + | 1.1 | 1.1 | 1.0 | 0.8 |
| R172N + L173V + E346T + D476K + G477A | | | | |
| SEQ ID NO: 2 + | 1.4 | 0.6 | 1.5 | 1.0 |
| R172N + L173V + E345D + D476K + G477S | | | | |
| SEQ ID NO: 2 + | 1.4 | 0.5 | 1.5 | 1.0 |
| R172N + L173V + S255I + E345D + G477Q | | | | |
| SEQ ID NO: 2 + | 1.2 | 1.2 | 1.2 | 1.0 |
| R172N + L173V + E345D + E346P + D476G | | | | |
| SEQ ID NO: 2 + | 1.3 | 0.4 | 1.2 | 0.8 |
| R172N + L173V + R320A + S323N | | | | |
| SEQ ID NO: 2 + | 1.6 | 1.0 | 1.5 | 1.1 |
| G50A + R118P + R172D + N174Q + E346T + | | | | |
| D476K + G477A | | | | |
| SEQ ID NO: 2 + | 1.2 | 0.8 | 1.0 | 0.9 |
| R172D + N174E + M208Y + V213S + V214T + | | | | |
| L217M | | | | |
| SEQ ID NO: 2 + | 1.4 | 1.3 | 1.7 | 1.2 |
| G50A + R172D + N174D + R320A + S323M + | | | | |
| A442V + D476Y | | | | |
| SEQ ID NO: 2 + | 1.5 | 1.1 | 1.5 | 1.1 |
| G50A + R172D + N174Q + G304S + E345D + | | | | |
| I464T + G465E + D476K + G477Q | | | | |
| SEQ ID NO: 2 + | 1.3 | 1.0 | 1.4 | 1.2 |
| G50A + R172D + N174Q + G304S + E345D + | | | | |
| D476K + G477Q | | | | |
| SEQ ID NO: 2 + | 1.4 | 0.7 | 1.6 | 1.0 |
| G50A + R172D + N174Q + R320A + S323N + | | | | |
| Y482F | | | | |
| SEQ ID NO: 2 + | 1.4 | 1.1 | 1.5 | 1.1 |
| G50A + R172D + N174Q + E346T | | | | |
| SEQ ID NO: 2 + | 1.3 | 0.9 | 1.5 | 0.9 |
| G50A + R172D + N174Q + R320V | | | | |
| SEQ ID NO: 2 + | 1.3 | 1.0 | 1.3 | 1.5 |
| R172E + L173F + N174D + K302Q + E346P + | | | | |
| D476K + G477S | | | | |
| SEQ ID NO: 2 + | 1.3 | 0.9 | 1.4 | 1.1 |
| G57R + R172E + L173F + N174D + K302Q + | | | | |
| E346P | | | | |
| SEQ ID NO: 2 + | 1.3 | 0.9 | 1.3 | 1.5 |
| R172E + L173F + N174D + E346T + D476K + | | | | |
| G477A | | | | |
| SEQ ID NO: 2 + | 1.1 | 0.9 | 1.3 | 1.4 |
| R172E + L173F + N174D + E345D + D476K + | | | | |
| G477S | | | | |
| SEQ ID NO: 2 + | 1.2 | 0.7 | 1.2 | 1.3 |
| R172E + L173F + N174D + S255I + E345D + | | | | |
| G477Q | | | | |
| SEQ ID NO: 2 + | 1.2 | 1.0 | 1.3 | 1.1 |
| R172E + L173F + N174D + T246M + S255I + | | | | |
| E345D + G477Q | | | | |
| SEQ ID NO: 2 + | 1.3 | 1.0 | 1.3 | 1.3 |
| T5K + R172E + L173F + K302Q + E346P + | | | | |
| D476K + G477S | | | | |
| SEQ ID NO: 2 + | 1.3 | 1.0 | 1.3 | 0.9 |
| T5K + R172E + L173F + K302Q + E346P | | | | |
| SEQ ID NO: 2 + | 1.2 | 1.0 | 1.0 | 0.9 |
| T5K + R172E + L173F + E345D + E346P + | | | | |
| D476G | | | | |
| SEQ ID NO: 2 + | 1.2 | 0.8 | 1.2 | 0.7 |
| T5K + R172E + L173F + R320A + S323N | | | | |
| SEQ ID NO: 2 + | 1.2 | 0.8 | 1.2 | 0.7 |
| T5K + R172E + L173F + R320V + D476Y | | | | |
| SEQ ID NO: 2 + | 1.4 | 1.1 | 1.6 | 1.3 |
| G50A + A51T + Q169M + R172E + N174S + | | | | |
| M208Y + V213T + V214T + L217M + | | | | |
| G304Q + E345D + E346T + D476G + | | | | |
| G477S | | | | |
| SEQ ID NO: 2 + | 1.5 | 1.1 | 1.5 | 1.3 |
| G50A + A51T + Q169M + R172E + N174S + | | | | |
| M208Y + V213S + V214T + L217M + | | | | |
| G304Q + E345D + E346T + D476G + | | | | |
| G477S | | | | |
| SEQ ID NO: 2 + | 1.5 | 1.1 | 1.3 | 0.8 |
| R172E + L173F + N174D + M208Y + V213S + | | | | |
| V214T + L217M | | | | |
| SEQ ID NO: 2 + | 1.4 | 0.8 | 1.0 | 0.3 |
| T40K + K72R + R118P + W167F + G184S + | | | | |
| R320A + H321N + S323N | | | | |
| SEQ ID NO: 2 + | 1.6 | 1.1 | 1.5 | 1.0 |
| T40K + K72R + R118P + W167F + G184S + | | | | |
| M208Y + V213S + V214T + L217M | | | | |
| SEQ ID NO: 2 + | 1.4 | 0.8 | 1.2 | 0.5 |
| T40K + K72R + R118P + W167F + G184S + | | | | |
| M208Y + V213T + V214T + L217M + | | | | |
| R320A + H324Q | | | | |
| SEQ ID NO: 2 + | 1.2 | 0.8 | 1.1 | 1.1 |
| G50A + A51T + K72R + W167F + Q169M + | | | | |
| R172E + N174S + G304Q + E345D + | | | | |
| E346T | | | | |
| SEQ ID NO: 2 + | 1.1 | 0.7 | 1.2 | 1.2 |
| R172N + L173F + N174Q + V213S + V214T + | | | | |
| L217M + G304Q + E345D + E346T | | | | |
| SEQ ID NO: 2 + | 1.1 | 0.6 | 1.0 | 1.1 |
| R172E + L173F + N174D + V213T + V214T + | | | | |
| L217M | | | | |
| SEQ ID NO: 2 + | 1.2 | 0.8 | 1.2 | 1.1 |
| G50A + R118P + R172D + N174T + E345D + | | | | |
| E346P + D476G | | | | |
| SEQ ID NO: 2 + | 1.1 | 0.6 | 1.2 | 1.1 |
| R118P + R172E + N174D + G184S + A204T + | | | | |
| G304Q + E345D + E346T + E391L + | | | | |
| D476G + G477S | | | | |
| SEQ ID NO: 2 + | 1.2 | 0.5 | 1.3 | 1.1 |
| G50A + A51T + R118P + R172E + N174S + | | | | |
| G184S + V213T + V214T + L217M + | | | | |
| G304Q + E345D + E346T + D476G + | | | | |
| G477S | | | | |
| SEQ ID NO: 2 + | 1.2 | 0.9 | 1.3 | 1.2 |
| A113T + R172D + N174Q + E346T + D476K | | | | |
| SEQ ID NO: 2 + | 1.3 | 0.9 | 1.5 | 1.2 |
| G50A + R172D + N174D + E346T + D476K | | | | |
| SEQ ID NO: 2 + | 1.2 | 0.9 | 1.1 | 1.1 |
| N174D + E345D + E346P + D476K | | | | |
| SEQ ID NO: 2 + | 1.2 | 0.8 | 1.2 | 1.3 |
| T40K + K72R + W167F + R172N + L173F + | | | | |
| N174Q + G304Q + E345D + E346T + | | | | |
| G465K + D476G | | | | |
| SEQ ID NO: 2 + | 0.9 | 0.9 | 1.1 | 0.9 |
| T40K + K72R + W167F + R172N + L173F + | | | | |
| N174Q + G304Q + E345D + E346T + | | | | |
| D476G | | | | |
| SEQ ID NO: 2 + | 1.0 | 0.9 | 1.1 | 1.1 |
| T40K + K72R + R172N + L173F + N174Q + | | | | |
| G304Q + E345D + E346T + D476G | | | | |
| SEQ ID NO: 2 + | 1.4 | 1.1 | 1.3 | 1.1 |
| G50A + R172N + N174T + E346T + D476K | | | | |
| SEQ ID NO: 2 + | 1.1 | 0.9 | 1.1 | 1.0 |
| K72R + W167F + E346T | | | | |
| SEQ ID NO: 2 + | 1.1 | 0.9 | 1.2 | 1.3 |
| K72R + W167F + E346T + D476K | | | | |
| SEQ ID NO: 2 + R118P + W140Y | 1.3 | 0.6 | 1.3 | 1.1 |
| SEQ ID NO: 2 + | 1.2 | 0.6 | 1.2 | 1.0 |
| G50A + A51V + R118P + R172N + N174T + | | | | |
| G184S + A204T | | | | |
| SEQ ID NO: 2 + | 1.1 | 0.8 | 1.1 | 1.0 |
| R172N + N174T + G184S + A204T | | | | |
| SEQ ID NO: 2 + | 1.0 | 0.9 | 1.1 | 0.9 |
| R118P + E346T + G477A | | | | |
| SEQ ID NO: 2 + | 1.2 | 0.9 | 1.2 | 1.1 |
| G7K + T40K + W167F + R320A + S323N + | | | | |
| P364S | | | | |
| SEQ ID NO: 2 + | 1.3 | 0.7 | 1.2 | 1.0 |
| G7K + T40K + K72R + W167F + R320A + | | | | |
| S323N + P364S | | | | |

1. An alpha-amylase variant comprising
a) a deletion and/or a substitution at two or more positions corresponding to positions R181, G182, H183 and G184 of the mature polypeptide of SEQ ID NO: 1, and
b) a substitution at one or more positions said substitutions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 95% sequence identity but less than 100% sequence identity to the polypeptide having the amino acid sequence of SEQ ID NO: 1 and wherein the variant has alpha-amylase activity.

2. The variant according to claim 1 wherein a) comprises a pairwise deletion of the amino acids corresponding to R181+G182, R181+H183, R181+G184, G182+H183, G182+G184 or H183+G184.

3. The variant according to claim 1 wherein b) comprises two or more of said substitutions.

4. The variant according to claim 1 wherein b) comprises three or more of said substitutions.

5. The variant according to claim 1 wherein b) comprises four or more of said substitutions.

6. The variant according to claim 1 wherein b) comprises five or more of said substitutions.

7. The variant according to claim 1 wherein b) comprises six or more of said substitutions.

8. The variant according to claim 1 wherein b) comprises one or more of said deletions.

9. The variant according to claim 1 wherein b) comprises or consists of the substitutions or deletions selected from the group consisting of: H1*, H1L, H1G, H1W, H1R, H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51 T+N174E, G50A+A51 T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51L+N174S+A447V, G50A+A51T+L173F, G50A+R172Q+L173I, G50A+A51V+L173F+N174D, G50A+R172D+L173T, G50A+A51 T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51T+A87S+R172N+L173T+N174D+G304Q+E345D+E346T+D476G+G477S, G50A+A51T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G 477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51 T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51 T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51 L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174 D+A204T+E346T+D476K+G477A, R172K+A204V+E346T+D476K+G477A, G4S+Y16D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174 D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174 D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174 D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51T+R172E+A204T+E346T+G477A, G50A+A51T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S.

10. The variant according to claim 1 which variant has an improved wash performance in at least one of the conditions of example 1, relative to the polypeptide of SEQ ID NO: 2 or to the polypeptide of SEQ ID NO: 1 having identical alterations of a) as the variant and wherein the improved wash performance is determined according to Example 1.

11. The variant according to claim 1 which variant has an improved wash performance in one or more of the conditions selected from the group consisting of:
a. Model detergent A at 15° C. and 0.2 mg enzyme protein/L wash solution;
b. Model detergent A at 40° C. and 0.05 mg enzyme protein/L wash solution;
c. Model detergent J at 15° C. and 0.2 mg enzyme protein/L wash solution;
d. Model detergent J at 30° C. and 0.05 mg enzyme protein/L wash solution,
wherein the improved wash performance is relative to the polypeptide of SEQ ID NO: 2 or to the polypeptide of SEQ ID NO: 1 having identical alterations of a) as the variant and wherein the improved wash performance is determined according to the experimental conditions listed in example 1.

12. An alpha-amylase variant comprising
a) a pairwise deletion at amino acid positions corresponding to positions R181+G182, R181+G184, G182+H183, or G182+G184 of the mature polypeptide of SEQ ID NO: 1, and
b) a substitution at one or more positions, said positions corresponding to positions 1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173 or 174 of SEQ ID NO: 1, wherein the alpha-amylase variant has at least 70%, such as at least 75%, such as at least 80%, such as at least 85%, such as at least 90%, such as at least 95% sequence identity but less than 100% sequence identity to the polypeptide having the amino acid sequence of SEQ ID NO: 1 and wherein the variant has alpha-amylase activity.

13. A polynucleotide encoding said variant according to claim 1.

14. A nucleic acid construct comprising said polynucleotide according to claim 13.

15. A host cell comprising said polynucleotide according to claim 13.

16. A method of producing an alpha-amylase variant, comprising:
a. cultivating the host cell according to claim 15 under conditions suitable for expression of said variant; and
b. recovering said variant.

17. A method of improving the wash performance of a parent alpha-amylase having the amino acid sequence of SEQ ID NO: 1 or having at least 95% sequence identity thereto, said method comprising the steps of:
a) substituting and/or deleting two or more positions in said parent alpha-amylase said positions corresponding to positions R181, G182, H183 and G184 of the mature polypeptide of SEQ ID NO: 1, and
b) introducing into said parent alpha-amylase a substitution at one or more of the following positions
1, 2, 3, 4, 5, 7, 9, 16, 25, 26, 28, 36, 40, 47, 48, 50, 51, 56, 67, 72, 73, 75, 85, 86, 87, 98, 109, 112, 113, 118, 119, 125, 128, 133, 134, 136, 140, 141, 142, 144, 146, 149, 155, 158, 160, 165, 167, 169, 171, 172, 173, 174, 179, 181, 182, 184, 186, 192, 193, 194, 195, 197, 200, 202, 204, 206, 208, 210, 211, 112, 213, 214, 215, 217, 218, 219, 220, 226, 241, 243, 246, 255, 265, 268, 269, 270, 273, 274, 276, 280, 282, 283, 291, 294, 295, 296, 299, 302, 303, 304, 305, 311, 315, 319, 320, 321, 323, 324, 330, 336, 343, 345, 346, 349, 351, 354, 355, 361, 363, 364, 376, 380, 383, 391, 402, 405, 406, 408, 410, 411, 414, 415, 416, 418, 421, 423, 424, 427, 429, 430, 431, 438, 439, 442, 444, 446, 447, 449, 457, 463, 464, 465, 466, 467, 469, 471, 473, 474, 476, 477, 478, 482, or 483 and/or a deletion at one or more positions corresponding to 1, 2, 3, 4, 5, 172, 173, or 174 of the mature polypeptide of SEQ ID NO: 1, wherein the resulting variant has at least 95%, such as at least 97%, but less than 100% sequence identity with the mature polypeptide of SEQ ID NO: 1, and wherein said resulting variant has alpha-amylase activity and an improved wash performance compared to said parent alpha-amylase.

18. The method according to claim 17 wherein a) comprises or consists of a pairwise deletion of the amino acids corresponding to R181+G182, R181+H183, R181+G184, G182+H183, G182+G184 or H183+G184.

19. The method according to claim 17 wherein a) is a deletion of H183+G184.

20. The method according to claim 17 wherein b) comprises or consists of the substitutions or deletions selected from the group consisting of: H1K, H1S, H1L, H1N, H1V, H2*, H2E, H2V, H2A, H2C, H2Q, N3*, N3C, N3D, N3S, N3C, G4*, G4V, G4S, T5*, T5K, G7S, G7L, G7E, G7Q, G7D, G7N, G7K, G7A, G7P, G7H, M9V, Y16D N25D, R26Q, N28D, N28S, S36D, T40K, T40M, T40I, A47G, A47S, W48L, G50A, G50S, A51T, A51V, A51I, A51L, A51Q, A51P, A51S, A51F, V56A, V56T, G67E, K72S, K72R, K72H, G73V, V75I, V75F, L85F, Q86H, A87S, A87D, Q98V, Q98S, Q98A, Q98N, Q98T, Q98L, Q98K, G109S, D112H, D112N, A113Y, A113H, A113Q, A113S, A113N, A113L, A113T, R118K, R118G, R118P, R118L, R118S, R118D, A119P, N128G, N128V, N128L, N128T, N128H, N128M, N128F, N128Q, G133Q, G133V, E134D, T136N, W140Y, T141S, R142Q, R142K, R142T, R142S, D144Q, D144T, P146S, G149Q, G149R, G149K, G149A, G149L, F155Y, R158G, R158A, Y160H, Y160L, Y160R, Y160Q, Y160G, V165G, V165M, V165A, V165L, V165Q, V165S, W167L, W167F, W167Y, W167H, Q169K, Q169R, Q169G, Q169M, R171E, R171S, R171H, R172*, R172Y, R172L, R172S, R172P, R172T, R172V, R172A, R172M, R172Q, R172K, R172N, R172D, R172E, L173V, L173T, L173F, L173I, N174L, N174Y, N174G, N174Q, N174S, N174E, N174T, N174D, N174H, K179S, K179M, K179P, K179R, K179A, K179T, K179L, R181G, G182N, G182A, G182S, G182N, G182V, G184S, G184N, G184*, A186E, A186V, A186S, A186T, A186M, A186W, A186H, D192E, D192A, D192G, T193V, T193M, T193S, T193G, T193A, T193N, E194S, E194A, N195F, N197A, M200L, M202A, A204T, A204V, A204G, I206Y, M208S, M208G, M208W, M208Y, H210M, H210Y, H210S, H210D, P211E, P211G, P211V, P211D, P211A, P211W, P211T, P211H, P211Q, E212D, V213T, V213S, V214I, V214S, V214T, N215A, N215G, N215D, L217M, L217F, R218K, N219S, N219K, N219E, N219T, N219G, N219R, W220F, N226T, I241F, Y243E, Y243L, Y243I, Y243F, Y243S, Y243G, Y243D, Y243T, Y243P, T246M, S255I, S255N, S255K, A265S, W268F, W268L, K269G, K269S, K269L, K269R, K269N, K269I, K269V, K269M, K269Q, K269H, N270A, N270P, N270G, N270Y, G273V, A274K, A274W, A274T, A274S, A274E, A274C, A274Y, A274L A274M, A274H, A274R, A274V, A274I, E276S, E276N, Q280R, Q280S, Q280K, Q280H, T282G, N283H, N283L, N283D, N283V, N283A, N283R, N283G, V291A, H294G, Y295N, N296Q, N299K, N299G, N299T, K302A, K302R, K302Q, K302N, S303R, S303G, G304R, G304V, G304A, G304S, G304Q, G305S, G305A, G305N, G305R, G305T, N311S, N311L, N311E, N311D, G315E, Q319K, R320K, R320A, R320S, R320M, R320T, R320V, H321V, H321A, H321L, H321Q, H321N, H321W, H321K, S323K, S323E, S323W, S323M, S323N, S323R, S323Q, H324R, H324W, H324L, H324Q, H324A, H324N, D330A, D330S, D330S, D330A, *336aG, *336bH, *336cG, *336dG, E336G, F343W, E345A, E345S, E345D, E346G, E346A, E346P, E346T, E346K, K349M, K349A, L351A, L351C, L351H, L351Q, L351S, L351K, A354V, L355V, Q361S, Q361A, Y363L, P364S, T376K, P380Q, R383K, E391A, E391T, E391M, E391L, E391K, E391H, E391N, H402N, L405V, D406A, P408H, P408Q, V410R, I411M, T414K, R415K, E416K, D418L, D418N, D418E, D418H, K423N, K423W, K423L, K423H, K423P, K423T, S424Q, A427V, L429A, L429F, I430M, I430L, I430E, T431P, K438N, R439G, A442V, N446K, A447V, A447T, E449K, E449G, K463R, N457T, K463R, I464T, I464S, G465L, S466F, D467G, W469L, E471N, E471K, H473S, V474T, V474I, D476G, D476K, D476Q, D476Y, G477A, G477S, G477Q, G477K, G477T, S478A, Y482F, K483R, H1*+H2E, H1*+H2V, H1*+H2A, H1*+N3C, H1L+A113Y+R171E+D192E, H1*+H2*+G7S, H1L+R171E+D192E, H2C, T5*+G4*+N3*+R320A, G4V+E134D+K179L, G7E+R320M+S323M, G7Q+Q98S+R320S+H324Q, G7E+R320A+H324Q, G7L+Q98N+R320A+S323N, G7E+Q98T+R320A, G7L+R320S+S323M, G7D+Q98A+R320M, G7N+Q98L+R320A+S323M, G7E+Q98A+R320M, G7N+R320S, G7K+R320A+S323M+H324L, G7L+P211Q+S303G+R320A, G7N+R320A+S323M, G7Q+R320S+S323M, G7N+R320A, G7E+R320A+S323M, G7A+Q98L+R320M, G7A+R320M, G7P+R320S+S323K, G7Q+R320A+S323N, G7L+Q98A+R320A+S323M, G7Q+R320M+S323N+H324W, G7S+R320A+S323N+H324Q, G7L+R320A, G7K+R320M+H321Q, G7N+R320A+S323N, G7Q+R320S+H321N+S323M, G7Q+R320A+S323M, G7L+R320S+S323M, G7N+Q98S+R320M+S323N, G7Q+R320A+S323R, G7E+Q98A+K302R+R320A+S323N, G7A+R320S+H321W+S323M, G7K+R320A+P408Q, G7K+R320A+H324L, G7L+R320M+S323N, G7E+R320A, G7L+R320A+S323M, G7E+R320A+P320Q+S323M, G7K+R320A+S323N+P364S, G7Q+R320A+H321Q+S323M, G7N+R320M, G7H+Q98S+R320A+S323M+H324L, G7Q+R320S+H321N, G7E+R320M, G7Q+Q98T+R320A+S323M, G7K+R320A, G7K+T246M+R320A+S323M, G7N+M9V+Q98A+R320S+S323M, G7Q+Q98A+R320M+S323M, G7Q+Q86H+R320A+S323M, N28D+G109S+A119P+R172D+L173V+N174S, N28S+R320M+S323N, T40I+A51L+R172D+L173F, T40K+G50A+R172Q+I430E, T40K+E346T, T40K+K72R+W167F, T40K+K72R+W167F+R320A+S323N, T40K+K72R+W140Y+W167F, G50A+R172Q+L173F, G50A+A51T+N174E, G50A+A51T+N174E, G50A+L173F, G50A+A51T+R172Q+N174Q+E471K, G50A+R172Q, G50A+A51V+R172N+N174S, G50A+A51T+R172Q, G50A+L173F+N174Q, G50A+R172Q+N174T, G50A+R172Q+L173T, G50A+R172Q+N174D, G50A+L173F+N174T, G50A+R172N+N174S, G50A+R172N+N174T, G50S+R172K+N174D, G50A+A51T+K72H+R172Q+N174Q+A204V, G50A+L173V+N174S, G50A+R172Q+L173T+N174D, G50A+N174E, G50A+L173T, G50A+A51T+R172Q+L173V, G50A+N174T+A204V, G50A+R172D+N174D, G50A+R172D+N174Q, G50A+L173V+A204T, G50A+A51V+R172N+N174T+A204T, G50A+N174S, G50A+R172Q+L173V+N174S, G50A+R172Q+A204T, G50A+R172Q+N174Q, G50A+A51V+R172D+L173F, G50A+A51I+R172D+N174*, G50A+R172K, G50A+R172Q+N174S, G50A+A51V+R172D+L173F+N174Q+A204T, G50S+R172N+N174Q, G50A+R172Q+L173I+N174T+A204T, G50A+R172E, G50A+R172Q+A204V, G50A+A51V+R172E+L173F+N174T+A204V, G50A+L173V, G50A+R172Q+L444P, G50A+A51T+R172N+L173V, G50A+R172Q+N174S+A204T, G50A+A51V+R172Q, G50A+A51T+R172D, G50A+A51T+R172N+N174D+G182V, G50A+R172E+L173F, G50A+A51T+R172Q+N174E, G50A+R172Q+L173F+N174Q, G50A+A51I+A204V, G50A+L173F+N174S, G50A+R172E+L173V+N174T, G50A+A51L+N174S+A447V, G50A+A51T+L173F, G50A+R172Q+L173I, G50A+A51V+L173F+N174D, G50A+R172D+L173T, G50A+A51 T+R172Q+L173F+N174Q, G50A+R172Q+N174Q+A204T, G50A+R172D+L173T+N174S, G50A+N174D+A204T, G50A+A51T+R172N+L173I+N174Q, G50A+L173T+N174S+A204V, G50A+A51T+R172Q+A204V, G50A+L173F+N174Q+A204V, G50A+A51V+L173F+N174E+E471K, G50A+A51V+R172Q+A204V, G50A+A51Q+R172Q, G50A+A51T+R172E+L173I, G50A+A51V+L173F+N174*, G50A+R172S+N174H, G50A+A51T+A87S+R172N+L173T+N174D, G50A+R172N+L173T+N174D, G50A+R172E+L173F+N174D, G50A+R172D+N174T, G50A+A51T+R172Q+N174T, G50A+R172D, G50A+N174E+A204T, G50A+A51T+L173F+N174S, G50A+A51T+R172E+N174S, G50A+A51T+R172Q+N174Q, G50A+A51T+R172Q+L173F+N174T, G50A+R172Q+N174T+G304Q+E345D+E346T+D476G+G477S, G50A+R172D+N174D+G304Q+E345D+E346T+D476G+G477S, G50A+A51V+R172D+L173F+G304Q+E345D+E346T+D476G+G477S, G50A+A51V+R172D+L173F+N174Q+A204T+G304Q+E345D+E346T+D476G+G477S, G50A+R172Q+L173I+N174T+A204T+G304Q+E345D+E346T+D476G+G477S, G50A+A51Q+R172Q+G304Q+E345D+E346T+D476G+G477S, G50A+A51V+R172Q+A204V+G304Q+E345D+E346T+D476G+G477S, G50A+A51 T+A87S+R172N+L173T+N174D+G304Q+E345D+E346T+D476G+G477S, G50A+A51 T+R172E+N174S+G304Q+E345D+E346T+D476G+G477S, G50A+A51T+R172Q+N174S+A204V, G50A+A51T+L173V+A204V, G50A+R172D+N174S, G50A+R172N, G50A+R172N+L173V, G50A+R172E+L173V+N174S, G50A+A51T+R172E+N174T, G50A+A51T+R172Q+L173I, G50A+R172E+L173T+N174S, G50A+A51T+R172E+A204V, G50A+R172E+N174T, G50A+R172N+A204T, G50A+R172Q+L173V, G50A+A51T+R172E, G50A+A51V+R172D+N174T, G50A+L173I+L173I+N174T+N174T+N219K+W220F, G50A+R172E+L173F+N174Q+A762, G50A+A51V+R172Q+N174Q, G50A+R172N+N174E, G50A+R172Q+L173T+N174Q, G50A+R172E+L173F+N174T, G50A+A51T+R172Q+L173V+A204V, G50A+A51T+R172D+L173F+N174T+K423P, G50A+R172E+L173F+N174Q+A204T, G50A+A113L+R172D+N174Q, G50A+R118D+W167F+R172D+N174Q, G50A+A51S+A113L+R118D+R172D+N174Q, G50A+R118D+R172D+N174Q, G50A+A51V+A113L+W167F+R172N+N174T+A204T, G50A+A51V+R118D+W167F+R172N+N174T+A204T, G50A+A51V+W167F+R172N+N174T+A204T, G50A+A51V+A113L+R118D+R172N+N174T+A204T, G50A+A51V+A113L+R172N+N174T+A204T, G50A+A51V+R118D+R172N+N174T+A204T, G50A+A51V+A113L+R118D+R172D+L173F, G50A+A51V+R118D+R172D+L173F, G50A+A51V+A113L+R118D+W167F+R172D+L173F, G50A+A113L+W167F+R172Q+L173I+N174T+A204T, G50A+W167F+R172Q+L173I+N174T+A204T, G50A+R118D+W167F+R172Q+L173I+N174T+A204T, G50A+A113L+R118D+R172Q+L173I+N174T+A204T, G50A+A113L+R172Q+L173I+N174T+A204T, G50A+R118D+R172Q+L173I+N174T+A204T, G50A+A51V+R118D+W167F+R172E+L173F+N174T+A204V, G50A+A51V+W167F+R172E+L173F+N174T+A204V, G50A+A51V+A113L+R172E+L173F+N174T+A204V, G50A+A51V+A113L+R118D+R172E+L173F+N174T+A204V, G50A+A51V+R118D+R172E+L173F+N174T+A204V, G50A+A51T+A113L+W167F+R172N+L173V, G50A+A51T+W167F+R172N+L173V, G50A+A51T+R118D+W167F+R172N+L173V, G50A+A51T+A113L+R118D+R172N+L173V, G50A+A51T+A113L+R172N+L173V, G50A+A51T+R118D+R172N+L173V, G50A+A113L+W167F+R172Q+A1066, G50A+W167F+R172Q+A1105, G50A+R118D+R172Q, G50A+A113L+R118D+R172Q, G50A+A113L+W167F+172Q, G50A+R172D+L173F+E471K, G50A+A51T+R172D+E345D+E346P+D476G, G50A+R172D+N174Q+E345D+E346P+D476G, G50A+R172D+N174D+E345D+E346P+D476G G50A+A51V+R172N+N174T+A204T+E345D+E346P+D476G, G50A+R172D+N174T+E345D+E346P+D476G, G50A+N174T+E345D+E346P+D476G, G50A+R172N+N174T+E345D+E346P+D476G, G50A+A51V+L173F+N174D+E345D+E346P+D476G, G50A+A51T+R172D+S255I+E345D+G477Q, G50A+A51V+R172E+L173F+N174T+A204V+S255I+E345D+G477Q, G50A+R172D+N174Q+S255I+E345D+G477QG50A+R172D+N174D+S255I+E345D+G477Q, G50A+A51V+R172N+N174T+A204T+S255I+E345D, G50A+R172D+N174T+S255I+E345D+G477Q, G50A+R172N+N174T+S255I+E345D+G477Q, G50A+A51V+L173F+N174D+S255I+E345D+G477Q, G50A+A51V+L173F+N174D+S255I+E345D, G50A+A51T+W140Y+P146S+R172D, G50A+A51T+W140Y+R172D, G50A+A51V+W140Y+R172E+L173F+N174T+A204V, G50A+W140Y+R172D+N174Q, G50A+W140Y+R172D+N174D, G50A+W140Y+P146S+R172D+N174D+E471K, G50A+A51V+W140Y+R172N+N174T+A204T, G50A+A51T+W140Y+R172N+L173V, G50A+W140Y+R172N+N174T, G50A+A51V+W140Y+P146S+L173F+N174D, G50A+A51T+A87S+Q169M+R172N+L173T+N174D+Y206I+G304Q+E345D+E346T+D476G+G477S, G50A+A51T+A87S+Q169M+R172N+L173T+N174D+G304Q+E345D+E346T+A427V+D476G+G477S, G50A+R172D+N174Q+R320A+H321N+S323N, G50A+R172D+N174D+R320A+H321N+S323N, G50A+A51V+R172N+N174T+A204T+R320A+H321N+S323N, G50A+N174T+R320A+H321N+S323N, G50A+R172N+N174T+R320A+H321N+S323N, G50A+A51V+L173F+N174D+R320A+H321N+S323N, G50A+A51T+R172E+N174S+G304Q+R320A+H321N+S323N+E345D+E346T+D476G+G477S, G50A+A51T+R172E+N174S+G304Q+E345D+E346T+E391L+D476G+G477S, G50A+A51T+A113L+R172E+N174S+G304Q+E345D+E346T+V474T, G50A+A51T+A113L+R172E+N174S+G304V+E345D+E346T+D476G+G477S, G50A+A51T+Q169M+R172E+N174S+G304Q+E345D+E346T+D476G+G477S, G50A+A51T+R172E+N174S+M208Y+V213T+V214T+L217M+G304Q+E345D+E346T+D476G+G477S, G50A+A51V+W167F+R172E+L173F+N174T+A204V+R320A+S323N, G50A+A51V+W167F+R172E+L173F+N174T+Y206I+M208Y+V213S+V214T+L217M, G50A+A51V+W167F+R172E+L173F+N174T+M208Y+V213T+V214T+L217M, G50A+A51T+G149A+W167F+R172N+L173V, G50A+A51T+W167F+R172N+L173V+R320A+S323N, G50A+A51T+W167F+R172N+L173V+Y206I+M208Y+V213S+V214T+L217M, G50A+A51T+W167F+R172N+L173V+Y206I+M208Y+V213T+V214T+L217M, A51V+R172D+L173F, A51V+R172Q+A204V, A51I+R172Q+L173F+A204T, A51V+L173I+N174S, A51T+L173T+N174S+A204T, W48L+A51T+R172Q+N174S+A204T, A51I+R172D, A51I+R172Q+N174E, A51I+R172E+N174T, A51V+R172Q+N174S+A204V, A51T+L173F+N174Q+A204V+A521, A51V+N174T+A204V, A51T+R172Q+L173T, A51T+R172N+A204V, A51L+R172N+N174S+A204T, A51T+R172N+L173*, A51T+R172Q, A51I+N174D, A51V+R172Q+L173F+N174S, A51T+R172D+N174Q, A51V+L173V+A204V, A51T+R172N+A204V+G304Q+E345D+E346T+D476G+G477S, A51V+R172E+N174*, A51V+R172D, A51T+R172D+N174Q+G304Q+E345D+E346T+D476G+G477S, A51I+R172E+L173F, A51T+R172D+N174D, A51T+R172E+N174T, A51V+R172Q+L173T, A51V+N174Q+A204V, A51T+N174D+A204V, A51P+R172Q+N174D, A51T+R172N+A204V+G304Q+E345D+E346T+E391L+D476G+G477S, A51T+R172N+A204V+G304Q+R320A+H321N+S323N+E345D+E346T+D476G+G477S, A51T+R172N+Y206I+M208Y+V213S+V214T+L217M+G304Q+E345D+E346T+D476G+G477S, V56T+A113Y+R171E, T40K+V56T+R171E, G67E+D112N+R172D+L173F+N174S+S466F, A47G+G67E+A111T+D112H+R245L+E346P+D476G+G477T, T40K+K72R, K72R+A113L+W167F+R172Q+N215G+Y363L, K72R+A113L+S255I+E345D+G477Q, K72R+A113L+W167F+S255I+E345D+G477Q, G73V+R172Q+N174E, V75I+R320A+S323N, G7Q+Q86H+R320A+S323M, A87D+R320S+S323M, Q98S+R320S, Q98A+R320A+S323N, Q98T+R320A+H324L, Q98A+A265S+R320A, Q98N+R320A+S323N, Q98S+R320A+S323N, Q98L+R320M+S323M, Q98K+K269N+R320A+S323M+H324Q, Q98N+R320S+S323N, Q98S+R320A, Q98A+R320A+S323M, Q98T+R320S+S323M, Q98A+R320S+S323M, Q98N+R320A+S323M+A354V Q98N+R320A+S323M+E391L, Q98N+A113L+R320A+S323M, Q98N+Q169M+R320A+S323M, Q98N+R320A+H321N+S323N, Q98N+Y206I+M208Y+V213S+V214T+L217M+R320A+S323M, Q98N+Y206I+M208Y+V213T+V214T+L217M+R320A+S323M, D112H+G304A+E345D+E346T+D476Q+G477S, D112H+R320A+S323N, K108E+G110D+A111T+D112H+R320S+S323M, D112N+R320M+S323M+E449G, A113Y+R171E, A113Y+R171E+E391L, A113Y+R171E+Y363L+E391L+K423P, A113T+K302Q+E346T+D476G+G477K, A113Y+G149Q+R171E+D192E+Q280S, H1L+A113Y+R171E+D192E, A113T+G304S+E345D+E346P+D476G+G477Q, A113L+R172Q+N174D, A113L+W167F+R172Q+Y363L, A113L+W167F+R172Q+N215G, A113L+S255I+E345D+G477Q, A113L+R118D+W167F+S255I+E345D+G477Q, A113L+W167F+E345D+D476K+G477S, R118S+G182A+T193K, R118P+G182A, R118P+G182S, R118S+E134D, R118S, R118S+E134D+G182A+A727, R118S+G182A, R118S+N128L+G182S, R118D+K179L+G182A, R118D+K179L+G182A, R118P+G182N, R118S+N128Q+K179L, R118D+G182N, R118S+E134D+G182S, R118D+W167F+R172Q+N174D, R118D+R172Q+N174D, R118D+W167F+S255I+E345D+G477Q, N128F+K179L, N128Q+A442V, N128Q+K179L+A186S, E134D+K179L+G182N, G4V+E134D+K179L, E134D+A186E, T136N+R172Q+N174D+A204T, W140Y+R320A, W140Y+R172D+N174T, G149Q+R171E, R158G+N174E, W167F+R172Q+N174D, W167F+R172Q+E346T+Y363L, W167F+R172Q+E346T, W167F+S255I+E345D+G477Q, W167F+E345D+D476K+G477S, R171E+E391L, R171E+Y363L, H1L+R171E+D192E, T40K+R171E, R171E+H324L+Q361A+Y363L, R171E+Q361A+Y363LR171E+D192E+Y363L, R171E+K423P, R171E+D192E+Q280S+Q361A+Y363L+E391L+K423P, R171E+D192E+E345D+E346P+D476G, R171E+D192E+S255I+E345D+G477Q, R172Q+N174Q, R172Q+N174S R172Q+L173F+N174Y, R172Q+N174T+A204T, R172Q+L173I+N174T, R172Q+L173I, R172Q+L173T+N174D, R172K+L173I+N174S, R172Q+L173F+A204T, R172Q+N174T, R172N+L173T+N174Q+S303R, R172K+L173F, R172Q+L173I+N174S, R172D+N174T, R172Q+L173F, R172D+L173F, R172K+L173F+A204T, R172Q+L173T+N174S, R172Q+N174*, R172Q+L173F+N174D, R172N+N174S, R172E+L173F+N174Q, R172Q+N174D, R172E+L173T+N174T, R172N+A204V, R172Q+L173V, R172Q+L173F+N174D+A204T, R172E+L173F+N174D, R172K+A204V, R172E+L173F+A204V, R172E+N174D+A204T, R172N+L173F+N174Q, R172E+L173F, R172Q+L173V+A204T, R172Q+L173V+A204V, R172E+N174S, R172Q+N174D+G304Q+E345D+E346T+D476G+G477S, R172E+N174D+A204T+G304Q+E345D+E346T+D476G+G477S, R172E+N174T, R172E+N174D, R172Q+A204V, R172Q+L173F+N174T, R172N+N174T+A204V, R172K+N174S, L173T+N174S+A204V, R172D+N174S, R172D+L173V+N174D, R172Q+L173T+N174Q, R172Q+N174E, R172D+N174D, L173V+N174D, R172D+L173F+N174D, R172E+L173I+A204T, R172K+L173V+N174S+A204T, R172E+L173T+N174S+A204T, R172D+L173F+A447T+T453N, R172N+N174D, R172N+N174T+H324N, R172K+L173V+N174S+A204T+L429F, R172D+L173V, R172K+L173T, R172E+L173T+N174S, R172D+L173I+N174S, R172Q+L173T, R172K+L173F+N174Q+A204V, R172N+L173I, R172N+L173F+N174Q+G304Q+E345D+E346T+D476G+G477S, R172K+A204V+E345D+E346P+D476G, R172K+A204V+S255I+E345D+G477Q, R172K+A204V+S255I+E345D+K423P+G477Q, R172E+N174D+A204T+G304Q+E345D+E346T+E391L+D476G+G477S R172E+N174D+A204T+G304Q+E345D+E346T+E391L R172E+N174D+A204T+G304Q+R320A+H321N+S323N+E345D+E346T+D476G+G477S, R172N+Y206I+M208Y+V213T+V214T+L217M+G304Q+E345D+E346T+D476G+G477S, R172E+N174S+G304Q+R320A+H321N+S323N+E345D+E346T+D476G+G477S, R172E+N174D+A204T+G304Q+R320A+S323N+E345D+E346T+D476G+G477S, R172N+L173F+N174Q+G304Q+R320A+S323N+E345D+E346T+D476G+G477S, L173T+N174Q+A204V, L173F+N174Q, L173V+N174S, L173I+N174D, L173I+N174S, L173F+A204T, L173V+N174T, L173V+A204V, L173T+N174T+A204T, L173F+N174Q+A204V, L173T+N174S+A204V+T255K, L173F+N174D, L173T+N174T+A204V, N174T+A204T, N174Q+A204V, T40K+K179S, K179L+G182A, K179L+A186S, G182A+V213T+V214S, G182A+H210Y+V214S, G182N+A186E+V214S, G182N+V213S, G182S+E210D+V214I, G182A+V213T+V214I, G182S+A186V, G182A+P432H, D192E+Q280S, N174S+A204T, N174D+A204V, A204T+G304Q+E345D+E346T+D476G+G477S, Y206I+M208Y+V213S+V214T+L217M, H210S+P408Q, H210S+V214T, P211T+V213T, P211H+V214I, V213T+V214T, V213T+V214S, S255I+R320A+H321N+S323N+E345D+G477Q, S255I+E345D+G477Q, K269G+N270G, K269S+N270Y+A274K+Y295N+N299T, K269Q+A274W, K269H+A274E, K269Q+A274C, K269Q+A274Y, 269Q+A274L, K269Q+A274M, K269H+A274S, K269Q+A274S, K269Q+A274C, K269Q+A274Y, K269Q+A274L, K269Q+A274M, K269H+A274S, K269Q+A274SK269L+A274S, K269L+A274L, K269Q+A274G, K269V+A274Y, K269L+A274G, K269L+A274W, K269Q+A274R, K269R+A274G, K269Q+A274H, K269H+A274R, K269Q+A274V, K269H+A274C, K269R+A274W, K269N+A274M, K269N+A274C, N270Y+Y295N, N270Y+Y295N+N299T, G273V+G304S+E346P+D476G+G477T, A274E+G304Q+G305S+E345D+D476K+G477Q, Y295N+N299T, K302Q+E346P+D476K+G477S, K302Q+E345D+D476K+G477K, K302N+E345D+D476K+G477S, K302Q+G304A+E346T+G477K, K302N+E345D+D476Q+G477Q, K302Q+E345D+D476G, K302N+E346T+D476K+G477A, K302N+E345D+E346T+D476Q+G477Q, K302N+E345D+D476G+G477K, K302N+G304Q+E346P+D476K, K302Q+E345D+E346P+D476K+G477Q, K302Q+E345D+E346T+D476K+G477K, K302Q+E346P+D476K+G477Q, K302N+E346P+D476Q+G477K, K302Q+E346P+D476K+G477T, K302Q+G304S+E345D+D476G+G477Q, K302Q+G304A+E345D+E346T+D476Q+G477Q, K302N+D476G, K302Q+D476Q+G477Q, K302N+E346T+D476Q+G477T, K302Q+D476G+G477A, K302Q+D476Q+G477S, K302Q+E345D+D476K+G477Q, K302Q+E346P+D476K+G477A, K302Q+E346P+G477A, K302N+E345D+E346P+D476Q+G477S, K302N+E345D+D476G, K302Q+E346P+D476K, K302Q+E346P+G477Q, K302Q+D476G+G477Q, K302Q+G304Q+D476K+G477K, G304Q+E346P+D476K+G477A, G304S+E345D+E346P+D476K+G477Q, G304Q+E345D+E346P+D476G+G477Q, G304Q+D476G+G477K, G304A+E345D+E346T+D476K+G477S, G304A+E345D+E346P+D476G+G477K, G304Q+E346P+D476G, G304A+E346P+D476K+G477A, G304A+E345D+E346T+D476G+G477K, G304Q+E346P+D476G+G477K, G304A+D476K+G477S, G304A+E345D+E346T+D476G+G477T, G304A+E345D+D476Q+G477A, G304Q+E345D+E346T+D476G+G477S, G304Q+E345D+D476G+G477Q, G304Q+E345D+E346P+D476Q+G477Q, G304Q+E346P+D476K+G477S, G304Q+E346T+D476K+G477Q, G304A+E346K+G477K, G304A+E346P+D476G+G477A, G304A+E345D+E346P+D476K, G304S+E346P+D476K+G477S, G304A+E346T, G304Q+E345D+E346T+D476G+G477T, G304A+E346T+D476G, G304S+E345D+E346T+D476Q+G477A, G304A+E345D+E346T+D476Q+G477Q, G304S+E346P+D476K+G477K, G304A+E345D+E346P+D476K+G477Q, G304A+E345D+D476K+G477S, G304Q+E345D+E346P+D476G, G304Q+E345D+E346T+G477Q, G304A+E345D+E346P+G477T+Y480F, G304A+E345D+D476Q+G477Q, G304Q+E346T, G304Q+E345D+E346P+D476G+G477T, G304Q+E345D+E346T+D476G, G304Q+E345D+E346T+D476Q+G477T, G304A+E346P+D476G+G477S, G304A+E345D, G304A+E346P+D476K+G477Q, G304A+E345D+E346P+D476Q+G477S, G304A+E345D+E346T+D476K+G477A, G304A+E346T+D476K+G477T, G304S+E345D+D476K+G477Q, G304A+E346P+D476Q+G477T, G304S+E346P+D476Q+G477S, G304S+E345D+D476K+G477Q, G304A+E345D+D476Q, G304Q+E345D+E346P+D476K+G477Q, G304S+E345D+E346T+D476K, G304Q+E345D+E346T, A204T+G304Q+E345D+E346T+D476G+G477S, G305S+E346T+D476Q+G477Q, G305S+E346P+D476Q+G477T, G305R+E346P+E449K, G305A+E345D, G305S+E345D+D476K+G477Q, G305S+E345D+E346P+G477Q, G305S+E346P+D476K+G477Q, R320S+H324Q, R320S+S323M, R320A+S323N, R320A+S323M, R320A+H324L, R320S+S323N, R320M+S323N, R320A+H324Q, R320S+S323M+H324L, R320A+H321W+S323M+H324Q, R320S+S323M+H324Q, T5*+G4*+N3*+R320A, R320V+D476Y, R320A+H321N+S323N, R320M+S323M+H324Q, E345D+E346P+D476K+G477S, E345D+D476K+G477S, E345D+E346P+D476G, E345D+E346T+D476G+G477K, E345D+E346P+D476K+G477Q, E345D+E346P+D476K+G477T, E345D+E346P+G477Q, E345D+E346P+D476G+G477T, E345D+D476Q+G477K, E345D+E346T+D476Q+G477Q, E345D+E346T+D476G, E345D+E346T+D476K+G477K, E345D+E346T+D476G+G477A, E345D+E346P+D476Q+G477A, E345D+D476Q+G477S, E345D+E346P+D476Q, E345D+D476Q, E345D+D476Q+G477A, G304Q+E346P+D476G+G477S, E345D+D476G+G477S, E345D+D476K+G477Q, E345D+D476Q+G477Q, E345D+E346P+G477S, E345D+D476G+G477Q, E345D+E346P+D476G+G477S, E345D+E346P, E345D+E346T+D476Q+G477A, G305N+E346P+D476K+G477T, E345D+E346P+D476G+G477A, E345D+E346P+D476Q+G477S, E345D+E346P+G477K, E345D+E346T+D476G+G477S, E346P+G477Q, E346P+G477T, E346P+D476Q+G477T, E346P+D476K+G477S, E346T+D476K+G477A, E346P+D476G+G477K, E346T+D476G+G477T, E346P+D476Q+G477S, E346T+D476K, E346T+D476Q+G477S, E346P+D476Q, E346T+G477K, E346P+G477S, E346T+D476K+G477S, E346P+D476K+G477T, E346T+D476Q+G477A, E346T+D476Q, E346T+D476K+G477Q, E346T+D476Q+G477K, E346T+D476G+G477Q, E346P+D476G+G477Q+Y480F, E346T+G477A, E346P+G477K, E391L+K423P, S466F+D476K, D476G+G477S, D476K+G477Q, G7Q+Q98T+R320A, R118P+E134D, N128Q+E134D+K179L, G50A+L173F+N174D+A204T, G50A+A51V+R172E+N174S, G50A+A51T+R172E+A204T, G50A+N174Q, G50A+R172D+N174Q+A204V, G50A+A51I+L173F, R172K+N174S+A204G, A51I+A204T, G50A+R172D+L173V, G50A+A51V+R172D+N174E, T40K+K72R+W167F+M208Y+V213T+V214T+L217M, T40K+K72R+W167F+M208Y+V213T+V214T+L217M+R320A+H324Q, G50A+R172D+L173F+N174S, R172E+A204V, G50A+A51T+R172E+L173V+N174T, G50A+A51V+N174Q, A51T+L173F, G50A+R172K+L173T+N174T, G50A+L173V+N174H, G50A+L173T+A204T, R172N+L173F+N174Q+Y206I+M208Y+V213S+V214T+L217M+G304Q+E345D+E346T+D476G+G477S, R172N+L173F+N174Q+Y206I+M208Y+V213T+V214T+L217M+G304Q+E345D+E346T+D476G+G477S, T40K+K72R+W167F+R320A+H324Q, T40K+K72R+W167F+R320A+H321N+S321N, T40K+K72R+W167F+Y206I+M208Y+V213S+V214T+L217M, A51V+L173F, G50A+R172 D+N174D+E346T+D476K+G477A, G50A+R172D+N174Q+E346T+D476K+G477A, R172D+N174T+E346T+D476K+G477A, L173F+N174S+A204T+P408Q, G50A+A51I+V117I+R172E, G50A+A51T+L173F+N174D, A51V+L173F+N174Q, A51V+L173V, A41E+A51I+R172D+L173T, G50A+N174Q+A204T, A51 L+R172D, G50A+A111S+L173F+N174T, A51I+L173T+A204T+L297I, L173F, K72R, Y160L, N270G, V165G, N215G, P211T+V213T, P211T, G50A+R172Q+L173V, N174T, G50A+R172N+A204T, R320A, R320S+H321W+S323M, G7A+R320A+S323M, G304A+E346P, G304S+E345D+E346T+D476Q+G477Q, G305S+E346T+D476K+G477K, G7L+A51 S+R320S+S323M, R320A+S323M+H324L, Q98N+R320S, Q98N+R320S+S323M+H324L, Q98N+R320A+S323M, G7N+R320M+S323M, G7L+R320S+S323N+H324W, G50A+R172E+L173F+N174T, R172E+N174Q, R172D+L173I+N174S, R172Q+N174T, R26Q+L173T, R320A+H321N+S323N+E345D+E346P+D476G, Q98N+G304V+R320A+S323M, Q98N+A113L+G304V+R320A+S323M, G50A+A51V+R172E+L173F+N174T+A204V+E345D+E346P+D476G, T40K+K72R+W167F+M208Y+V213T+V214T+L217M+R320A+S323N, A51T+L173T+N174Q+A204T, A51L+N174Q, G50A+A51I+R172D+L173V, G50A+A51L+R172N+L173V+A204T, R172D+L173T+N174D+A204T, A51I+L173F, L173T+A204T, A51I+R172D+N174T, A51T+R172K+L173I+N174S, G50A+R172E+N174S, R172N+L173V, G50A+R172K+N174Q, T5K+R172E+L173F, A47S+G50A+R172K+L173F+N174D, G50A+A51T+R172D+N174S, N25D+R172N+A204T, R172D+L173T+N174D, G50A+A51L+L173T+N174T+A204T, L173F+N174D+A204S, A51I+R172K+L173V+N174H+E449K, G50A+L173T+N174S, G50A+A51T+R172N+A204T, G50A+A51F+R172D, G50A+A51S+R172D+N174Q, G50A+D112N, G50A+A51T+R172N+L173V+E345D+E346P+D476G, G50A+A51T+R172N+L173V+E346T+D476K+G477A, G50A+A51V+A113L+R172E+L173F+N174T+A204V+E345D+E346P+D476G, G50A+A51V+A113L+R172E+L173F+N174T+A204V+E346T+D476K+G477A, N174D+A204T+E345D+E346P+E416K, N174 D+A204T+E346T+D476K+G477A, R172 K+A204V+E346T+D476K+G477A, G4S+Y16 D+T40K+G50A+R172N+N174S+A204T, T40K+G50A+K72R+W167F+R172N+N174S+A204T, G4S+T40K+K72R+Q98N+W167F+R218K+N226T+R320A+S323M, T40K+K72R+Q98N+R320A+S323M, T40K+K72R+A113L+W167F, T40K+K72R+R118D+W167F, T40K+A51T+R172N+A204V+G304Q+E345D+E346T+D476G+G477S, T40K+G50A+W167F+R172N+N174S+A204T, T40K+K72R+E345D+E346P+D476G, K72R+W167F+E346T+D476K+G477A+Y482F, W167F+E346T+D476K+G477A, G50A+A51V+K72R+W167F+R172E+L173F+N174T+A204V, T40K+G50A+A51V+K72R+W167F+R172E+L173F+N174T+A204V, K72R+Q98N+W167F+R320A+S323M, T40K+K72R+Q98N+W167F+R320A+S323M, T40K+G50A+A51V+R172N+N174T+A204T, T40K+G50A+A51V+W167F+R172N+N174T+A204T, T40K+G50A+A51V+K72R+R172N+N174T+A204T, T40K+K72R+R172E+N174D+A204T+G304Q+E345D+E346T+D476G+G477S, T40K+K72R+W167F+R172N+L173F+N174Q+G304Q+E345D+E346T+D476G+G477S, T40K+K72R+R172N+L173F+N174Q+G304Q+E345D+E346T+D476G+G477S, G50A+R172N+N174T+E346T+D476K+G477A, A51V+R172D+N174S+G305N+G465L+D467G, G50A+L173T+N174E+A204T+E345D+S424Q, L173F+N174D+A354V, L173F+N174E+G477S, G50A+A51V+R172D+L173F+A204V+K302Q+D476G+G477S, G50A+A51V+L173Q+R172*+N174T+G305S+E346P+G477T, R172D+L173F+N174D+E345D+E346P+W469L, T40K+N174 D+A204T, T40K+K72R+W167F+N174D+A204T, W140Y+W167F+N174D, G50A+W140Y+W167F+N174D+A204T, T40K+K72R+W167F+R172K+A204V, W140Y+P146S+R172K+A204V, T40K+W167F+R172E+N174D+A204T+G304Q+E345D+E346T+D476G+G477S, T40K+R172E+N174D+A204T+G304Q+E345D+E346T+D476G+G477S, W140Y+R172E+N174 D+A204T+G304Q+E345D+E346T+D476G+G477S, A51T+W140Y+R172N+A204V+G304Q+E345D+E346T+D476G+G477S, T40K+G50A+W167F+R172N+A204T, T40K+G50A+R172N+A204T, G50A+W140Y+R172N+A204T, G50A+W140Y+W167F+R172N+A204T, W167F+R172N+L173F+N174Q+G304Q+E345D+E346T+D476G+G477S, W140Y+R172N+L173F+N174Q+G304Q+E345D+E346T+D476G+G477S, T40K+G50A+W167F+R172D+N174T+E345D+E346P+D476G, T40K+G50A+K72R+W167F+R172D+N174T+E345D+G477Q, G50A+W140Y+R172D+N174T+E345D+E346P+D476G, G50A+W140Y+W167F+R172D+L173F+N174T+G304Q+E345D+E346P+D476G, T40K+K72R+W167F+R172K+A204V+M208Y+V213S+V214T+L217M+S255I+R320A+H324Q+E345D+G477Q, T40K+W167F+R172K+A204V+M208Y+V213S+V214T+L217M+S255I+R320A+H324Q+E345D+G477Q, W140Y+W167F+R172D+N174T+A204V+S255I+E345D+E346P+G477Q, W140Y+W167F+R172D+N174T+A204V+S255N+E345D+G477Q, W167F+R172D+N174T+A204V+S255I+E345D+G477Q, W140Y+W167F+R172D+N174T+A204V+S255I+E345D+E346P+G477Q, T40K+Q98N+W167F+R172K+A204V+M208Y+V213T+V214T+L217M+S255I+R320A+S323M+E345D+T431P, T40K+K72R+Q98N+W167F+A204V+M208Y+V213T+V214T+L217M+S255I+R320A+S323M, Q98N+W140Y+R172K+A204V+M208Y+V213T+V214T+L217M+S255I+R320A+S323M+E345D+V474I, Q98N+W140Y+R172K+A204V+M208Y+V213T+V214T+L217M+S255I+R320A+S323M+E345D+R415K, Q98N+W140Y+W167F+R172K+A204V+M208Y+V213T+V214T+L217M+S255I+R320A+S323M+E345D, T40K+G50A+A51V+K72R+W167F+R172E+L173F+N174T+A204V+R320A+S323N, G50A+A51V+W140Y+P146S+W167F+R172E+L173F+N174T+A204V+R320A+S323N, T40K+G50A+A51V+K72R+W167F+R172E+L173F+N174T+M208Y+V213S+V214T+L217M, G50A+A51V+W140Y+W167F+R172E+L173F+N174T+M208Y+V213S+V214T+L217M, G50A+A51T+R172D+K302Q+E346P+D476K+G477S, G50A+A51T+R172D+E345D+D476K+G477S, G50A+A51T+R172N+L173V+E345D+D476K+G477S, G50A+A51V+A113L+R172E+L173F+N174T+A204V+K302Q+E346P+D476K+G477S, G50A+A51V+A113L+R172E+L173F+N174T+A204V+E345D+L405V+T414K, G50A+A51V+R172N+N174T+A204T+K302Q+E346P+D476K+G477S, G50A+A51V+R172N+N174T+A204T+E345D+D476K+G477S, N174D+A204T+K302Q+E346P+D476K+G477S, G50A+R172D+N174Q+K302Q+E346P+D476K+G477S, G50A+R172D+N174Q+E345D+D476K+G477S, R172D+N174T+K302Q+E346P+D476K+G477S, G50S+R172N+N174Q+E345D+E346P+D476G, R172N+N174Q+E345D+D406A+I411M+N446K, G50S+R172N+N174Q+E345D, G50A+N174D+A204T+E345D+D476K+G477S, G50A+N174D+A204T+S255I+E345D+G477Q, G50A+N174 D+A204T+G304S+E345D+D476K+G477Q, N174D+A204T+G304S+E345D+D476K+G477Q, R172K+A204V+K302Q+E346P+D476K+G477S, R172K+A204V+E345D+D476K, R172K+A204V+E345D+D476K+G477S, R172K+A204V+G304S+E345D+D476K+G477Q, G50A+R172N+A204T+E346T+D476K+G477A, G50A+R172N+A204T+E346T+D432A+D476K+G477A, G50A+R172N+A204T+E345D+D476K+G477S, G50A+R172N+A204T+G304S+E345D+D476K+G477Q, G50A+R172N+A204T+E345D+E346P+D476G, G50A+A51V+W167F+R172E+L173F+N174T+A204V+S255I+E345D+G477Q, T40K+K72R+W167F+S255I+E345D+G477Q, T40K+K72R+W167F+G304S+E345D+D476K+G477Q, T40K+K72R+W167F+K302Q+E346P+D476K, G50A+A51V+W167F+R172E+L173F+N174T+M208Y+V213S+V214T+L217M+S255I+E345D+G477Q, G50A+A51V+W167F+R172E+L173F+N174T+M208Y+V213S+V214T+L217M+E345D+D476K+G477S, G50A+A51V+W167F+R172E+L173F+N174T+M208Y+V213S+V214T+L217M+E345D+E346P+D476G, G50A+A51V+W167F+R172E+L173F+N174T+M208Y+V213S+V214T+L217M+E346T+D476K+G477A, G50A+A51V+R172N+N174T+A204T+M208Y+V213T+V214T+L217M, G50A+A51V+R172N+N174T+A204T+M208Y+V213S+V214T+L217M, N174D+A204V+M208Y+V213T+V214T+L217M, R172E+N174D+A204T+M208Y+V213T+V214T+L217M+G304Q+E345D+E346T+D476G+G477S, R172E+N174D+A204T+M208Y+V213S+V214T+L217M+G304Q+E345D+E346T+D476G+G47 7S, A51T+R172N+A204V+M208Y+V213T+V214T+L217M+G304Q+E345D+E346T+D476G+G477S, A51T+R172N+A204V+M208Y+V213T+V214T+L217M+G304Q+E345D+E346T+E416K+K438N A51T+R172N+A204V+M208Y+V213S+V214T+L217M+G304Q+E345D+E346T+D476G+G477S, G50A+R172N+A204T+M208Y+V213T+V214T+L217M, G50A+R172N+A204T+M208Y+V213S+V214T+L217M, G50A+A51V+W167F+R172E+L173F+N174T+A204V+M208Y+V213T+V214T+L217M, G50A+A51V+W167F+R172E+L173F+N174T+A204V+M208Y+V213S+V214T+L217M, R172K+A204V+M208Y+V213T+V214T+L217M+S255I+E345D+G477Q, G50A+A51V+W167F+R172E+L173F+N174T+A204V+M208Y+V213T+V214T+L217M+R320A+S323N, G50A+A51V+R172N+N174T+A204T+E346T+G477A, G50A+A51V+R172N+N174T+A204T+R320V+D476Y, G50A+A51V+R172N+N174T+A204T+R320V, R172N+L173V+E346T+G477A, G50A+A51T+R172N+L173V+E346T+G477A, G50A+A51T+R172N+L173V+R320A+S323M+D476Y, G50A+A51T+R172N+L173V+R320V+D476Y, G50A+A51T+R172D+R320A+S323N, G50A+R172Q+L173T+E346T+G477A, R172Q+L173T+R320V+D476Y, N174D+A204T+R320V+D476Y, R172K+A204V+R320A+S323N, G50A+A51V+W167F+R172E+L173F+N174T+A204V+E346T+G477A, G50A+A51V+W167F+R172E+L173F+N174T+A204V+R320V+D476Y, R172E+L173F+N174T+A204V+E345D+E346T+G477A, G50A+A51V+A113L+R172E+L173F+N174T+A204V+E346T+G477A, G50A+A51V+A113L+R172E+L173F+N174T+A204V+R320V, T40K+K72R+W167F+E346T+G477A, T40K+K72R+W167F+R320V+D476Y, G50A+A51V+R172E+N174S+E346T+G477A, G50A+A51V+N174S+R320V+D476Y, A51V+N174S+R320V+D476Y, G50A+A51T+R172E+A204T+E346T+G477A, G50A+A51 T+R172E+A204T+R320V+D476Y, G50A+A51T+R172E+A204T+R320V+N457T+D476Y, R172D+N174Q+A204V+E346T+G477A, L173T+A204T+E346T+G477A, L173T+A204T+R320V+D476Y, G50A+A51V+R172D+N174E+E346T+G477A, G50A+A51V+R172D+N174E+R320V+D476Y, G50A+A51V+R172D+N174E+R320S+S323N+D476Y, G50A+A51V+W167F+R172E+L173F+N174T+A204V+G304Q+E345D+E346T+D476G+G477T, G50A+R172D+N174Q+R320A+S323N, G50A+R172D+N174Q+E346T+G477A, G50A+R172D+N174Q+R320A+S323M+D476Y, G50A+R172D+N174Q+R320V+D476Y, G50A+R172N+A204T+K302Q+E346P+D476K+G477S, G7K+T40K+K72R+W167F, G7Q+T40K+K72R+W167F, G7E+T40K+K72R+W167F, W167F+Q169M+R172E+N174S, T40K+K72R+W167Y, T40K+K72R+W167H, W140Y+P146S+N174D+A204T, W140Y+W167F+R172K+A204V, W140Y+W167F+A204V, R172K+M208Y+V213T+V214T+L217M, R172K+M208Y+V213S+V214T+L217M, Q98N+W167F+R320A+S323M, W140Y+W167F+S255I+E345D+G477Q, G7K+W167F+R320M+H321Q, G7K+K72R+W167F+R320M+H321Q, G7K+T40K+W167F+R320M+H321Q, G7K+T40K+K72R+W167F+R320M+H321Q, A51T+W167F+R172N+A204V+G304Q+E345D+E346T, A51T+W167F+R172N+A204V+G304Q+E345D+E346T+D476G+G477S, A51T+K72R+R172N+A204V+G304Q+E345D+E346T+D476G+G477S, T40K+A51T+K72R+W167F+R172N+A204V+G304Q+E345D+E346T+D476G+G477S, T40K+A51T+W167F+R172N+A204V+G304Q+E345D+E346T+D476G+G477S, G50A+A51T+R172N+L173V+G304Q+E345D+E346T+D476G+G477T, G50A+A51V+A113L+R172E+L173F+N174T+A204V+G304Q+E345D+E346T+D476G+G477T, G50A+A51V+R172N+N174T+A204T+G304Q+E345D+E346T+D476G+G477T, G50A+R172Q+L173T+G304Q+E345D+E346T+D476G+G477T, G50A+A51T+L85F+R172N+L173V+Q319K+R320A+S323N, G50A+A51V+A113L+R172E+L173F+N174T+A204V+R320A+S323N, G50A+A51V+R172N+N174T+A204T+R320A+S323N, G50A+A51V+N174S+R320A+S323N, G50A+R172D+N174D+R320V+D476Y, G50A+R172D+N174D+R320V, G50A+A51T+R172E+A204T+E346T+D476K+G477A, G50A+L173T+A204T+K302Q+E346P+D476K+G477S, L173T+A204T+K302Q+E346P+D476K+G477S, R172D+N174Q+E346T+D476K+G477A, L173T+A204T+E346T+D476K+G477A, G50A+R172D+N174Q+A204V+E345D+D476K+G477S, L173T+A204T+E345D+D476K+G477S, L173T+A204T+S255I+E345D+G477Q, L173T+A204T+G304S+E345D+D476K+G477Q, G50A+R172D+N174Q+A204V+E345D+E346P+D476G, G50A+R172D+N174Q+A204V+R320A+S323N, G50A+A51T+R172E+A204T+K302Q+E346P+D476K+G477S, G50A+A51T+N174S+A204T+S255I+E345D+G477Q, G50A+A51T+L85F+R172E+A204T+E345D+E346P+D476G, R172D+N174E+K302Q+E346P+D476K+G477S, G50A+A51V+R172D+N174E+K302Q+E346P+D476K+G477S, G50A+A51V+R172D+N174E+E346T+D476K+G477A, G50A+A51V+R172D+N174E+S255I+E345D+G477Q, G50A+A51V+R172D+N174E+G304S+E345D+D476K+G477Q, G50A+A51V+R172D+N174E+E345D+E346P+D476G, G50A+A51V+R172D+N174E+R320A+S323N, G50A+A51V+R172D+N174E+G315E+R320A+S323N, G50A+A51T+R172E+A204T+E345D+D476K+G477S, R172E+A204T+G304S+E345D+D476K+G477Q, G50A+R172D+N174D+M208Y+V213S+V214T+L217M+E346T+D476K+G477A, G50A+A51V+N174S+M208Y+V213S+V214T+L217M, G50A+A51V+R172E+N174S+M208Y+V213S+V214T+L217M, A51V+R172E+N174S+M208Y+V213S+V214T+L217M, G50A+R118P+R172D+N174Q+G184S+E346T+D476K+G477A, G50A+A51V+R118P+R172E+N174S+G184S, G50A+A51V+R118P+N174S+G184S, G50A+R118P+R172E+G184S+A204T, G50A+A51T+R118P+R172E+G184S+A204T, R118P+L173T+G184S+A204T, G50A+A51V+R118P+R172D+N174E+G184S, T40K+G50A+A51V+W167F+N174S, T40K+G50A+A51V+K72R+W167F+R172E+N174S, T40K+G50A+A51V+K72R+W167F+R172D+N174E, T40K+G50A+A51V+W167F+R172D+N174E, T40K+W167F+L173T+A204T, T40K+G50A+K72R+W167F+L173T+A204V, G50A+R172D+N174Q+M208Y+V213S+V214T+L217M+E346T+D476K+G477A, G50A+R172S+N174S+E346T+D476K+G477A, G50A+A51V+R172D+N174E+M208Y+V213S+V214T+L217M, L173T+G184S+A204T, T40K+G50A+R172D+N174D+E346T+D476K+G477A, G50A+R118P+R172D+N174D+G184S+E346T+D476K+G477A, G50A+R172T+N174S+E346T, R172E+L173F+N174D+G304S+E345D+D476K+G477Q, R172E+L173F+N174D+E345D+E346P+D476G, R172E+L173F+N174D+R320A+S323N, R172E+L173F+N174D+E346T+A354V+G477A, R172E+L173F+N174D+E346T+G477A, R172E+L173F+N174D+R320V, R172E+L173F+N174D+R320V+D476Y, R172N+L173V+K302Q+E346P+D476K+G477S, R172N+L173V+E346T+D476K+G477A, R172N+L173V+E345D+D476K+G477S, R172N+L173V+S255I+E345D+G477Q, R172N+L173V+E345D+E346P+D476G, R172N+L173V+R320A+S323N, G50A+R118P+R172D+N174Q+E346T+D476K+G477A, R172D+N174E+M208Y+V213S+V214T+L217M, G50A+R172D+N174D+R320A+S323M+A442V+D476Y, G50A+R172D+N174Q+G304S+E345D+I464T+G465E+D476K+G477Q, G50A+R172D+N174Q+G304S+E345D+D476K+G477Q, G50A+R172D+N174Q+R320A+S323N+Y482F, G50A+R172D+N174Q+E346T, G50A+R172D+N174Q+R320V, R172E+L173F+N174D+K302Q+E346P+D476K+G477S, G57R+R172E+L173F+N174D+K302Q+E346P, R172E+L173F+N174D+E346T+D476K+G477A, R172E+L173F+N174D+E345D+D476K+G477S, R172E+L173F+N174D+S255I+E345D+G477Q, R172E+L173F+N174D+T246M+S255I+E345D+G477Q, T5K+R172E+L173F+K302Q+E346P+D476K+G477S, T5K+R172E+L173F+K302Q+E346P, T5K+R172E+L173F+E345D+E346P+D476G, T5K+R172E+L173F+R320A+S323N, T5K+R172E+L173F+R320V+D476Y, G50A+A51T+Q169M+R172E+N174S+M208Y+V213T+V214T+L217M+G304Q+E345D+E346T+D476G+G477S, G50A+A51T+Q169M+R172E+N174S+M208Y+V213S+V214T+L217M+G304Q+E345D+E346T+D476G+G477S, R172E+L173F+N174D+M208Y+V213S+V214T+L217M, T40K+K72R+R118P+W167F+G184S+R320A+H321N+S323N, T40K+K72R+R118P+W167F+G184S+M208Y+V213S+V214T+L217M, T40K+K72R+R118P+W167F+G184S+M208Y+V213T+V214T+L217M+R320A+H324Q, G50A+A51 T+K72R+W167F+Q169M+R172E+N174S+G304Q+E345D+E346T, R172N+L173F+N174Q+V213S+V214T+L217M+G304Q+E345D+E346T, R172E+L173F+N174D+V213T+V214T+L217M, G50A+R118P+R172D+N174T+E345D+E346P+D476G, R118P+R172E+N174D+G184S+A204T+G304Q+E345D+E346T+E391L+D476G+G477S, G50A+A51T+R118P+R172E+N174S+G184S+V213T+V214T+L217M+G304Q+E345D+E346T+D476G+G477S, A113T+R172D+N174Q+E346T+D476K, G50A+R172D+N174D+E346T+D476K, N174D+E345D+E346P+D476K, T40K+K72R+W167F+R172N+L173F+N174Q+G304Q+E345D+E346T+G465K+D476G, T40K+K72R+W167F+R172N+L173F+N174Q+G304Q+E345D+E346T+D476G, T40K+K72R+R172N+L173F+N174Q+G304Q+E345D+E346T+D476G, G50A+R172N+N174T+E346T+D476K, K72R+W167F+E346T, K72R+W167F+E346T+D476K, R118P+W140Y, G50A+A51V+R118P+R172N+N174T+G184S+A204T, R172N+N174T+G184S+A204T, R118P+E346T+G477A, G7K+T40K+W167F+R320A+S323N+P364S, or G7K+T40K+K72R+W167F+R320A+S323N+P364S.

21. A composition comprising a variant according to claim 1.

22. The composition according to claim 21, which is a detergent composition, such as a liquid or powder detergent composition.

23. The composition according to claim 21 which is a liquid laundry or liquid dishwash composition, such as an automatic dishwash liquid detergent composition.

24. The composition according to claim 21 further comprising a surfactant.

25. (canceled)
